(19) United States
(12) Patent Application Publication
(10) Pub. No.: US 2026/0159486 A1
(43) Pub. Date: Jun. 11, 2026

(54) BENZO[H]QUINAZOLINE-4-AMINE DERIVATIVES FOR THE TREATMENT OF CANCER

(71) Applicant: Redona Therapeutics, Inc., Watertown, MA (US)

(72) Inventors: Sven Weiler, Allschwil (CH); Bérangère Gaucher, Allschwil (CH); Florian Richalet, Allschwil (CH); Thomas Radimerski, Allschwil (CH); Andrea Aloia, Allschwil (CH)

(21) Appl. No.: 18/704,023

(22) PCT Filed: Oct. 25, 2022
(86) PCT No.: PCT/EP2022/079738

§ 371 Date: Apr. 23, 2024

(30) Foreign Application Priority Data
Oct. 25, 2021 (EP) 21204520.7
Apr. 7, 2022 (EP) 22167146.4
Jul. 21, 2022 (EP) 22186200.6

(51) Int. Cl. C07D 239/94; A61K 31/517; A61K 31/5377; A61K 31/5383; A61P 35/00; C07D 401/04; C07D 403/04; C07D 405/12; C07D 413/12; C07D 498/04

(52) U.S. Cl. CPC C07D 239/94; A61K 31/517; A61K 31/5377; A61K 31/5383; A61P 35/00; C07D 401/04; C07D 403/04; C07D 405/12; C07D 413/12; C07D 498/04

(57) ABSTRACT

The invention provides compounds of formula (I) (I) and pharmaceutically acceptable salts thereof; wherein Ra and Rb are both —CH3 or Ra and Rb together form a —CH2-CH2-CH2- or a-CH2-CH2-CH2-CH2-bridging moiety; A is-CH2- or —C(═O)—; T is —N(R11)-, —N(R11)—C(═O)—, —N(R11)—S(O2)-, —O—, —C(R12)(R13)-, —C═C(R12)2, —S—, —S(O)—, —S(O2)-, —S(O2)—N(R14)- or —C(═O)—N(R14)-; and wherein R1, R2, R11, R12, R13 and R14 are as defined in the claims, as well as methods of using the compounds for the treatment of neoplastic diseases such as cancer.

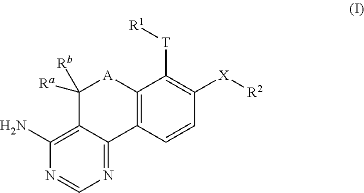

The present invention relates to compounds targeting the family of CDC2-like kinases (CLKs) and their use in the treatment of neoplastic diseases such as cancer.

Recurrent mutations in components of the splicing machinery have been reported in diseases like cancer. CLKs belong to the CMGC group of kinases and include four family members: CLK1, -2, -3 and -4. CLKs are known to play a key role in regulating alternative splicing (AS) through phosphorylation of splicing factors of the serine-arginine-rich (SR) family. Alternative splicing of transcripts is an important regulatory mechanism in eukaryotes that allows a single gene to generate multiple protein isoforms with distinct functions. CLK-mediated phosphorylation of SR proteins is implicated in their redistribution from speckles to a diffuse nucleoplasmic distribution pattern (Colwill et al., EMBO J. 1996, 15(2):265-75). SR protein phosphorylation has to be tightly regulated for nuclear import, spliceosome assembly, and ultimately for correct splicing, in particular to promote exon inclusion. Small molecule inhibitors of CLKs have been described to be efficacious in preclinical animal models of cancer (Yoshida T et al., Cancer Res. 2015, 75(7):1516-26; Iwai K et al., EMBO Mol Med. 2018 10(6):e8289; Zhu D et al., Mol Cancer Ther. 2018, 17(8):1727-1738). Therefore, targeting of CLKs offers potential for anti-cancer therapy, for instance in splicing factor mutant cancers, where there is a compelling rationale to exploit aberrant splicing as a vulnerability and therapeutic opportunity.

In a first aspect the present invention provides compounds of formula (I)

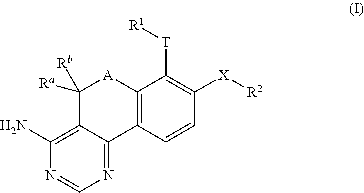

and pharmaceutically acceptable salts thereof; wherein
Ra and Rb are both —CH₃ or Ra and Rb together form a —CH₂—CH₂—CH₂— or a —CH₂—CH₂—CH₂—CH₂-bridging moiety;
A is —CH₂— or —C(═O)—;
T is —N(R11)-, —N(R11)-C(═O)—, —N(R11)-S(O₂)—, —O—, —C(R12)(R13)-, —C═C(R12)₂, —S—, —S(O)—, —S(O₂)—, —S(O₂)—N(R14)- or —C(═O)—N(R14)-;
R11, when not forming a ring with R1 or R2, is hydrogen, C1-C4alkylene-R11a, C1-C4haloalkylene-R11a or C3-C6cycloalkyl, wherein the cycloalkyl is optionally substituted by one or two R11b; each R12, when not forming a ring with R2, is independently hydrogen, C1-C4alkylene-R12a, C1-C4haloalkylene-R12a or C3-C6cycloalkyl, wherein the cycloalkyl is optionally substituted by one or two R12b;
R14, when not forming a ring with R1 or R2, is hydrogen, C1-C4alkylene-R14a, C1-C4haloalkylene-R14a or C3-C6cycloalkyl, wherein the cycloalkyl is optionally substituted by one or two R14b;
each R11a, R12a and R14a are independently hydrogen, halogen, —OH, —CN, —NH₂, —O—C1-C2alkyl, —O—C1-C2haloalkyl, —NH(C1-C2alkyl) or —N(C1-C2alkyl)₂;
each R11b, R12b and R14b are independently halogen, —OH, —CN, —NH₂, —O—C1-C2alkyl, —O—C1-C2haloalkyl, —NH(C1-C2alkyl) or —N(C1-C2alkyl)₂;
R13 is hydrogen, C1-C4alkyl or C1-C4haloalkyl;
R2 and R11, R2 and R12 or R2 and R14 may together form a partially unsaturated 6- to 7-membered heterocyclic ring containing no additional heteroatoms as ring members, wherein the heterocyclic ring is optionally substituted by one or two R2a;
each R2a is independently halogen, —OH, —CN, —NH₂, —O—C1-C2alkyl, —O—C1-C2haloalkyl, —NH(C1-C2alkyl) or —N(C1-C2alkyl)₂;
R1 and R11 or R1 and R14 may together form a saturated or partially unsaturated 4- to 7-membered heterocyclic ring optionally containing one or two additional heteroatoms as ring members selected from N, O and S, wherein the heterocyclic ring is optionally substituted by one to three R1a, or R1 and R11 or R1 and R14 may together form a 5- to 6-membered heteroaryl ring optionally containing one to three additional heteroatoms as ring members selected from N and O, wherein the heteroaryl ring is optionally substituted by one to three R1b;
each R1a is independently halogen, —OH, —CN, —NH₂, C1-C4alkylene-R1b, C1-C4haloalkylene-R1b, —O—C1-C4alkyl, —O—C1-C4haloalkyl, —NH(C1-C4alkyl), —N(C1-C4alkyl)₂, —C(═O)NH₂, —C(═O)NH(C1-C4alkyl), —C(═O)N(C1-C4alkyl)₂ or oxo;
each R1b is independently halogen, —OH, —CN, —NH₂, C1-C4alkylene-R1c, C1-C4haloalkylene-R1c, —O—C1-C4alkyl, —O—C1-C4haloalkyl, —NH(C1-C4alkyl) or —N(C1-C4alkyl)₂, —C(═O)NH₂, —C(═O)NH(C1-C4alkyl) or —C(═O)N(C1-C4alkyl)₂;
each R1c is independently hydrogen, halogen, —OH, —CN, —NH₂, —O—C1-C2alkyl, —O—C1-C2haloalkyl, —NH(C1-C2alkyl) or —N(C1-C2alkyl)₂;
R1, when not forming a ring with R11 or R14, is hydrogen or —Y—R3;
Y is a bond or C1-C6alkylene wherein one non-terminal —CH₂— moiety may be replaced by —O— and wherein the alkylene moiety may be substituted by one or two moieties independently selected from —OH, —O—C1-C4alkyl and —O—C1-C4haloalkyl and wherein the alkylene moiety may include a 3- to 5-membered saturated or partially unsaturated carbocyclic ring as part of the moiety;
R3 is hydrogen, —CN, —OH, C1-C4haloalkyl, C2-C4alkenylene-R4, C2-C4alkynylene-R4, —O—C1-C4alkylene-R4, —O—C1-C4haloalkyl, —NH₂, —NH(C1-C4alkylene-R4), —N(C1-C4alkylene-R4)₂, —C(═O)—OH, —C(═O)—O—C1-C4alkylene-R4, —C(═O)—NH₂, —C(═O)—NH(C1-C4alkylene-R4), —C(═O)—N(C1-C4alkylene-R4)₂, —NH—C(═O)—C1-C4alkylene-R4, —N(C1-C4alkyl)-C(═O)—C1-C4alkylene-R4, Cycle P, Cycle Q, —O—Cycle P, —O—Cycle Q, —NH—S(O₂)—C1-C4alkylene-R4, —N(C1-C4alkyl)-S(O₂)—C1-C4alkylene-R4, —S—C1-C4alkylene-R4 or —S(O₂)—C1-C4alkyl-R4, providing that the atom in R3 connected to Y is not a heteroatom when (i) Y is a bond and (ii) T is not —N(R11)-, —C(R12)(R13)- or —C═C(R12)₂;
R4 is hydrogen, halogen, —NH₂, —NH(C1-C2alkyl) or —N(C1-C2alkyl)₂;
Cycle P is a 3- to 6-membered saturated or partially unsaturated carbocyclic ring optionally substituted by one to three R5 or is a 3- to 6-membered saturated or partially unsaturated heterocyclic ring containing one or two heteroatoms selected from N and O optionally substituted by one to three R5;
each R5 is independently halogen, —NH₂, —OH, —CN, C1-C4alkylene-R5a, —O—C1-C4alkylene-R5a or oxo;
each R5a is independently hydrogen, halogen, —NH₂, —OH, —CN or —O—C1-C2alkyl;
Cycle Q is phenyl optionally substituted by one to three R6, or is a 5- to 6-membered heteroaryl ring containing one or two heteroatoms selected from N, S and O optionally substituted by one to three R6;
each R6 is independently halogen, —NH₂, —OH, —CN, C1-C4alkylene-R6a or —O—C1-C4alkylene-R6a;
each R6a is independently hydrogen, halogen, —NH₂, —OH, —CN or —O—C1-C2alkyl; X is —O—, —C(═O)—, —C(═CH₂)—, —N(R10a)- or —CH(R10b)- and wherein X is a bond when R2 is halogen or —CN and wherein when X is —C(═O)— then R2 is not hydrogen, or wherein X—R2 is hydrogen;
R10a, when not forming a ring with R2, is hydrogen or C1-C4alkyl;
R10b is hydrogen or C1-C4alkyl;
R2 and R10a may together form a 4- to 7-membered saturated or partially unsaturated heterocyclic ring optionally containing an —N(R9)- moiety as ring member in addition to the nitrogen atom of X and otherwise containing only carbon atoms as ring members, optionally substituted by one or two R8;
R2 and R10b may together form a 4- to 7-membered saturated or partially unsaturated carbocyclic ring or a 4- to 7-membered saturated or partially unsaturated heterocyclic ring containing an —N(R9)- moiety as ring member and otherwise containing only carbon atoms as ring members optionally substituted by one or two R8;
R2, when not forming a ring with R10a, R10b, R11, R12 or R14, is hydrogen, halogen, —CN, C1-C4alkyl optionally substituted by one or two R7, or is a 4- to 7-membered saturated or partially unsaturated carbocyclic ring optionally substituted by one or two R8, or is a 4- to 7-membered saturated or partially unsaturated heterocyclic ring containing one —N(R9)- moiety as ring member and otherwise containing only carbon atoms as ring members;
each R7 is independently —CN, —OH, halogen, —N(R7a)R7b or —NH(—C(═O)—C1-C4alkyl);
each R7a and R7b, when not together forming a ring, is independently hydrogen, C1-C4alkylene-R7c or C1-C4haloalkylene-R7c;
each R7c is independently hydrogen, halogen, —O—C1-C2alkyl or —O—C1-C2haloalkyl;
R7a and R7b may together form a 4- to 7-membered saturated or partially unsaturated heterocyclic ring optionally containing one or two additional heteroatoms as ring members selected from N, O and S, and wherein the heterocyclic ring is optionally substituted by one or two R7d;
each R7d is independently halogen, —OH, —CN, —NH₂, C1-C2alkyl, C1-C2haloalkyl, —O—C1-C2alkyl, —O—C1-C2haloalkyl, —NH(C1-C2alkyl) or —N(C1-C2alkyl)₂;
each R8 is independently —CN, —OH, halogen, —N(R8a)R8b, —NH(—C(═O)—C1-C4alkyl), C1-C4alkyl or C1-C4haloalkyl;
each R8a and R8b, when not together forming a ring, is independently hydrogen, C1-C4alkylene-R8c or C1-C4haloalkylene-R8c;
R8c is hydrogen, halogen, —O—C1-C2alkyl or —O—C1-C2haloalkyl;
R8a and R8b may together form a 4- to 7-membered saturated or partially unsaturated heterocyclic ring optionally containing one or two additional heteroatoms as ring members selected from N, O and S, and wherein the heterocyclic ring is optionally substituted by one or two R8d;
each R8d is independently halogen, —OH, —CN, —NH₂, C1-C2alkyl, C1-C2haloalkyl, —O—C1-C2alkyl, —O—C1-C2haloalkyl, —NH(C1-C2alkyl) or —N(C1-C2alkyl)₂;
R9 is hydrogen, C1-C4alkylene-R9a, C1-C4haloalkylene-R9a or —C(═O)—C1-C4alkyl; and
R9a is hydrogen, halogen, —O—C1-C2alkyl or —O—C1-C2haloalkyl.

In a further aspect, the invention provides compounds of formula (I) and pharmaceutically acceptable salts thereof for use in the treatment of neoplastic diseases in a subject selected from a mammal, in particular a human.

In a further aspect, the invention provides use of compounds of formula (I) and pharmaceutically acceptable salts thereof in the manufacture of a medicament for the treatment of neoplastic diseases in a subject selected from a mammal, in particular a human.

In a further aspect, the invention provides methods of treating neoplastic diseases in a subject selected from a mammal, in particular a human, comprising administering a compound of formula (I) or pharmaceutically acceptable salt thereof, e.g. in a therapeutically effective amount, to said subject. In a further aspect, the invention provides pharmaceutical compositions comprising a compound of formula (I) or pharmaceutically acceptable salt thereof and optionally one or more pharmaceutically acceptable excipients.

Each alkyl moiety either alone or as part of a larger group such as alkoxy is a straight or branched chain. Examples include methyl, ethyl, n-propyl, prop-2-yl, n-butyl, but-2-yl, 2-methyl-prop-1-yl or 2-methyl-prop-2-yl.

Each alkylene moiety either alone or part of a larger group is a straight or branched chain and is, for example, —CH₂—, —CH₂—CH₂—, —CH(CH₃)—, —CH₂—CH₂—CH₂—, —CH(CH₃)—CH₂—, —CH(CH₂CH₃)— or —CH₂CH(CH₃)CH₂—. Each alkenyl moiety either alone or as part of a larger group such as alkenyloxy is a straight or branched chain. Each moiety can be of either the (E)- or (Z)-configuration. Examples include vinyl and allyl.

Each alkenylene moiety either alone or as part of a larger group such as alkenyloxy is a straight or branched chain and is, for example, —CH₂—CH═CH—. Each moiety can be of either the (E)- or (Z)-configuration.

Each alkynyl moiety either alone or as part of a larger group such as alkynyloxy is a straight or branched chain and is for example ethynyl or propargyl.

Each alkynylene moiety either alone or as part of a larger group is a straight or branched chain and is, for example, —CH₂—C═C—.

Each haloalkyl moiety either alone or as part of a larger group such as haloalkoxy is an alkyl group substituted by one or more of the same or different halogen atoms. Examples include difluoromethyl, trifluoromethyl, chlorodifluoromethyl and 2,2,2-trifluoro-ethyl. Haloalkyl moieties include for example 1 to 5 halo substituents, or 1 to 3 halo substituents.

Each cycloalkyl moiety (also referred to as carbocyclic ring moieties) may be saturated or partially unsaturated (unless otherwise stated) and can be in mono- or bi-cyclic form, preferably in monocyclic form. Examples of monocyclic cycloalkyl groups include cyclobutyl, cyclopentyl and cyclohexyl. An example of a bicyclic cycloalkyl group is bicyclo[2.2.1]heptan-2-yl.

Halogen is fluorine, chlorine, bromine or iodine.

The term “heteroaryl ring” refers to an aromatic ring system containing the stated number of heteroatoms selected from nitrogen, oxygen and sulfur as ring members. Heteroaryl rings do not contain adjacent oxygen atoms, adjacent sulfur atoms, or adjacent oxygen and sulfur atoms within the ring or together with the atom outside the ring connecting the ring to the rest of the molecule.

The term “heterocyclic ring” refers to a saturated or partially unsaturated carbocyclic ring (unless otherwise stated) additionally containing the stated number of heteroatoms selected from nitrogen, oxygen and sulfur as ring members. Nitrogen as a ring member is present as an —NH— moiety unless otherwise substituted. Such rings do not contain adjacent oxygen atoms, adjacent sulfur atoms, or adjacent oxygen and sulfur atoms within the ring or together with the atom outside the ring connecting the ring to the rest of the molecule.

Oxo is an ═O group. Where a moiety is said to be “substituted by oxo” it is counted as substitution by one substituent, e.g. when Cycle P may be substituted by one to three R5 and R5 may be oxo, then Cycle P may be substituted by oxo and two further R5 groups. Usually a ring will not be substituted by more than one oxo group.

Where a group is said to be optionally substituted, it may be unsubstituted or substituted with the specified number of substituents.

Where ring nomenclature is provided for a specific moiety it is applied assuming no (other) substituents on the ring. For example piperidin-1-yl places the nitrogen atom at the point of attachment irrespective of the identity of any substituents which might be present on the piperidine moiety. Cyclohex-4-ylamine places the amino substituent at the 4 position on the cyclohexyl relative to the point of attachment, irrespective of the identity of any additional substituent which might be present on the cyclohexyl moiety.

Where (E) and (Z) isomers are possible, e.g. when R3 is alkenylene, the compounds of the invention include all (E) and (Z) isomers as well as mixtures thereof in any ratio. Likewise, where cis and trans isomers are possible, e.g. when R2 is cyclohex-4-ylamine, both cis and trans isomers are included in the scope of formula (I). Whenever compounds of formula (I) contain one or two or more centers of chirality, e.g. when Y is branched alkylene, such compounds may be provided as pure enantiomers or pure diastereoisomers as well as mixtures thereof in any ratio and all such isomers are included within the scope of the compounds of formula (I). The compounds of the invention also include all tautomeric forms of the compounds of formula (I) where such possibilities exist, e.g. when a saturated ring is substituted by oxo. Isotopically labeled compounds including deuterium substitutions as well as carbon-13 and/or carbon-14 labels are also included within the scope of compounds of formula (I).

The compounds of formula (I) may also be solvated, especially hydrated, which are also included in the scope of compounds of formula (I). Solvation and hydration may take place during the preparation process. Reference to compounds of the invention includes pharmaceutically acceptable salts of said compounds. Such salts may also exist as hydrates and solvates. Examples of pharmacologically acceptable salts of the compounds of formula (I) are salts of physiologically acceptable mineral acids, such as hydrochloric acid, sulfuric acid and phosphoric acid, or salts of organic acids, such as methanesulfonic acid, p-toluenesulfonic acid, lactic acid, formic acid, acetic acid, trifluoroacetic acid, citric acid, succinic acid, fumaric acid, maleic acid and salicylic acid. Further examples of pharmacologically acceptable salts of the compounds of formula (I) are alkali metal and alkaline earth metal salts such as, for example, sodium, potassium, lithium, calcium or magnesium salts, ammonium salts or salts of organic bases such as, for example, methylamine, dimethylamine, triethylamine, piperidine, ethylenediamine, lysine, choline hydroxide, meglumine, morpholine or arginine salts. The following examples of substituent definitions and embodiments may be combined in any combination where possible.

Ra and Rb are both —CH₃ or Ra and Rb together form a —CH₂—CH₂—CH₂— or a —CH₂—CH₂—CH₂—CH₂-bridging moiety.

Preferably Ra and Rb are both —CH₃ or together form a —CH₂—CH₂—CH₂—CH₂— bridging moiety, e.g. as depicted in Example 37.

A is —CH₂— or —C(═O)—. Preferably A is —CH₂—.

T is —N(R11)-, —N(R11)-C(═O)—, —N(R11)-S(O₂)—, —O—, —C(R12)(R13)-, —C═C(R12)₂, —S—, —S(O)—, —S(O₂)—, —S(O₂)—N(R14)- or —C(═O)—N(R14)-.

Preferably T is —N(R11)-, —N(R11)-S(O₂)—, —O—, —C(R12)(R13)-, —C═C(R12)₂, —S—, —S(O₂)—, —S(O₂)—N(R14)- or —C(═O)—N(R14)-.

More preferably T is —N(R11)-, —N(R11)-S(O₂)—, —O—, —C(R12)(R13)-, —C═C(R12)₂, —S— or —C(═O)—N(R14)-.

Even more preferably T is —N(R11)- or —N(R11)-S(O₂)—.

In the moiety —C═C(R12)₂ R1 is connected to the first carbon atom as shown here:

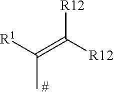

Specific examples of T (when R1 does not form a ring with R11 or R14, and R2 does not form a ring with R11, R12 or R14) include —CH₂—, —CH(CH₃)—, —C(CH₃)=CH—, —N(CH₃)—C(═O)—, —N(CH₃)—S(O₂)—, —N-(cyclopropyl)-, —N(CH₂CH₂CN)—, —N(CH₂CH₂CH₃)—, —N(CH₂CH₂OH)—, —N(CH₂CH₃)—, —N(CH₃)—, —NH—, —NH—C(═O)—, —NH—S(O₂)—, —O—, —S—, —S(O₂)—N(CH₃)—, —S(O₂)—NH—, —S(O₂)— and —C(═O)—NH—

R11 is hydrogen, C1-C4alkylene-R11a, C1-C4haloalkylene-R11a or C3-C6cycloalkyl, wherein the cycloalkyl is optionally substituted by one or two R11b (when R11 does not form a ring with R1 or R2).

Preferably R11 (when R11 does not form a ring with R1 or R2) is hydrogen, C1-C4alkylene-R11a, C1-C4haloalkylene-R11a or C3-C6cycloalkyl.

More preferably R11 (when R11 does not form a ring with R1 or R2) is hydrogen, C1-C4alkylene-R11a, C1-C4haloalkylene-R11a or C3-C4cycloalkyl.

Specific examples of R1 (when R11 does not form a ring with R1 or R2) include —CH₃, cyclopropyl, —CH₂CH₂CH₃, —CH₂CH₂CN, —CH₂CH₂OH, —CH₂CH₃ and hydrogen.

R11a is hydrogen, halogen, —OH, —CN, —NH₂, —O—C1-C2alkyl, —O—C1-C2haloalkyl, —NH(C1-C2alkyl) or —N(C1-C2alkyl)₂.

Preferably R11a is hydrogen, halogen, —CN or —OH.

Specific examples of R1a include hydrogen, —CN and —OH.

Each R11b is independently halogen, —OH, —CN, —NH₂, —O—C1-C2alkyl, —O—C1-C2haloalkyl, —NH(C1-C2alkyl) or —N(C1-C2alkyl)₂.

Each R12 is independently hydrogen, C1-C4alkylene-R12a, C1-C4haloalkylene-R12a or C3-C6cycloalkyl, wherein the cycloalkyl is optionally substituted by one or two R12b (when R12 does not form a ring with R2).

Preferably each R12 (when R12 does not form a ring with R2) is independently hydrogen or C1-C4alkylene-R12a,

Specific examples of R12 include hydrogen, —CH₃ and —CH₂OH.

Each R12a is independently hydrogen, halogen, —OH, —CN, —NH₂, —O—C1-C2alkyl, —O—C1-C2haloalkyl, —NH(C1-C2alkyl) or —N(C1-C2alkyl)₂.

Preferably each R12a is independently hydrogen, halogen, —CN or —OH.

Specific examples of R12a include hydrogen and —OH.

Each R12b is independently halogen, —OH, —CN, —NH₂, —O—C1-C2alkyl, —O—C1-C2haloalkyl, —NH(C1-C2alkyl) or —N(C1-C2alkyl)₂.

R13 is hydrogen, C1-C4alkyl or C1-C4haloalkyl.

Specific examples of R13 include hydrogen.

R14 is hydrogen, C1-C4alkylene-R14a, C1-C4haloalkylene-R14a or C3-C6cycloalkyl, wherein the cycloalkyl is optionally substituted by one or two R14b (when R14 does not form a ring with R1 or R2).

Preferably R14 (when R14 does not form a ring with R1 or R2) is hydrogen or C1-C4alkyl. Specific examples of R14 include hydrogen and —CH₃.

R14a is hydrogen, halogen, —OH, —CN, —NH₂, —O—C1-C2alkyl, —O—C1-C2haloalkyl, —NH(C1-C2alkyl) or —N(C1-C2alkyl)₂.

Each R14b is independently halogen, —OH, —CN, —NH₂, —O—C1-C2alkyl, —O—C1-C2haloalkyl, —NH(C1-C2alkyl) or —N(C1-C2alkyl)₂.

R2 and R11 may together form a partially unsaturated 6- to 7-membered heterocyclic ring containing no additional heteroatoms as ring members, wherein the heterocyclic ring is optionally substituted by one or two R2a.

Preferably R2 and R11 may together form a partially unsaturated 6-membered heterocyclic ring containing no additional heteroatoms when T is —N(R11)- and X is —O— and otherwise do not form a heterocyclic ring.

Specific examples of a bridging moiety formed by R2 and R11 include —CH₂CH₂— when T is —N(R11)- and X is —O—, namely —N(R1)CH₂CH₂O—, e.g. —N(H)CH₂CH₂O—, —N(CH₂CH₂CN)CH₂CH₂O— and —N(CH₃)CH₂CH₂O— including the T and X substituents (T on the left hand side, X on the right side of the bridging moiety as depicted).

R2 and R12 may together form a partially unsaturated 6- to 7-membered heterocyclic ring containing no additional heteroatoms as ring members, wherein the heterocyclic ring is optionally substituted by one or two R2a.

R2 and R14 may together form a partially unsaturated 6- to 7-membered heterocyclic ring containing no additional heteroatoms as ring members, wherein the heterocyclic ring is optionally substituted by one or two R2a.

Each R2a is independently halogen, —OH, —CN, —NH₂, —O—C1-C2alkyl, —O—C1-C2haloalkyl, —NH(C1-C2alkyl) or —N(C1-C2alkyl)₂.

R1 and R11 may together form a saturated or partially unsaturated 4- to 7-membered heterocyclic ring optionally containing one or two additional heteroatoms as ring members selected from N, O and S, wherein the heterocyclic ring is optionally substituted by one to three R1a, or R1 and R11 may together form a 5- to 6-membered heteroaryl ring optionally containing one to three additional heteroatoms as ring members selected from N and O, wherein the heteroaryl ring is optionally substituted by one to three R1b.

Preferably R1 and R11 may together form a 4- to 6-membered saturated or partially unsaturated heterocyclic ring optionally containing one additional heteroatom as a ring member selected from N and O, e.g. a ring selected from piperidinyl, morpholinyl, pyrrolidinyl, azetidinyl, 2,5-dihydropyrrolyl and 1,1-dioxide-1,2-thiazolidin-2-yl, wherein the heterocyclic ring is optionally substituted by one or two R1a, or R1 and R11 may together form a 5-membered heteroaryl ring optionally containing one additional heteroatom as a ring member selected from N, e.g. selected from pyrrolyl, and wherein the heteroaryl ring is optionally substituted by one or two R1b.

More preferably R1 and R11 may together form a 5- to 6-membered saturated heterocyclic ring containing no additional heteroatoms as ring members, e.g. a ring selected from piperidinyl, pyrrolidinyl and 1,1-dioxide-1,2-thiazolidin-2-yl, wherein the heterocyclic ring is optionally substituted by one R1a, or R1 and R11 may together form a 5-membered heteroaryl ring containing no additional heteroatoms as ring members, e.g. selected from pyrrolyl, and wherein the heteroaryl ring is optionally substituted by one R1b.

Specific examples of heterocyclic rings formed by R1 and R11 when T is —N(R11)-include piperidinyl (including e.g. 4-hydroxy-piperidinyl), morpholinyl, pyrrolidinyl (including e.g. 3-hydroxy-pyrrolidinyl, 3-methoxy-pyrrolidinyl, 3-cyano-pyrrolidinyl, 3-carboxamide-pyrolindinyl), azetidinyl (including e.g. 3-hydroxy-azetidinyl, 3-hydroxymethyl-azetidinyl), 2,5-dihydropyrrolyl (including e.g. 2H-pyrrolyl-5-one) and 1,1-dioxide-1,2-thiazolidinyl.

Specific examples of a heteroaryl ring formed by R1 and R11 when T is —N(R11)-include pyrrolyl (including e.g. 3,4-dimethoxypyrrolyl and 3-methoxypyrrolyl).

R1 and R14 may together form a saturated or partially unsaturated 4- to 7-membered heterocyclic ring optionally containing one or two additional heteroatoms as ring members selected from N, O and S, wherein the heterocyclic ring is optionally substituted by one to three R1a, or R1 and R14 may together form a 5- to 6-membered heteroaryl ring optionally containing one to three additional heteroatoms as ring members selected from N and O, wherein the heteroaryl ring is optionally substituted by one to three R1b.

Each R1a is independently halogen, —OH, —CN, —NH₂, C1-C4alkylene-R1c, C1-C4haloalkylene-R1c, —O—C1-C4alkyl, —O—C1-C4haloalkyl, —NH(C1-C4alkyl), —N(C1-C4alkyl)₂, —C(═O)NH₂, —C(═O)NH(C1-C4alkyl), —C(═O)N(C1-C4alkyl)₂ or oxo.

Preferably each R1a is independently halogen, —OH, —CN, C1-C4alkylene-R1c, —O—C1-C4haloalkyl, —C(═O)NH₂, or oxo.

More preferably each R1a is independently halogen, —OH, —CN or —C(═O)NH₂. Specific examples of R1a include —OH, —CN, —OCH₃, —CH₂OH, —C(═O)NH₂ and oxo.

Each R1b is independently halogen, —OH, —CN, —NH₂, C1-C4alkylene-Ric, C1-C4haloalkylene-Ric, —O—C1-C4alkyl, —O—C1-C4haloalkyl, —NH(C1-C4alkyl) or —N(C1-C4alkyl)₂, —C(═O)NH₂, —C(═O)NH(C1-C4alkyl) or —C(═O)N(C1-C4alkyl)₂.

Preferably each R1b is independently halogen, —OH, —CN, C1-C4alkylene-R1c or —O—C1-C4haloalkyl.

More preferably each R1b is halogen, —OH or —CN.

Specific examples of R1b include —OH, —CN, —OCH₃ and —CH₂OH.

Each R1c is independently hydrogen, halogen, —OH, —CN, —NH₂, —O—C1-C2alkyl, —O—C1-C2haloalkyl, —NH(C1-C2alkyl) or —N(C1-C2alkyl)₂.

Preferably each R1c is independently hydrogen or —OH.

Specific examples of R1c include hydrogen and —OH.

R1 is hydrogen or —Y—R3 (when R1 does not form a ring with R11 or R14).

Preferably R1 (when R1 does not form a ring with R11 or R14) is hydrogen, C1-C6alkylene-R3, wherein the alkylene moiety may be substituted by one —OH, wherein the alkylene moiety may include a 3-membered saturated carbocyclic ring as part of the moiety, and wherein there are no more than three carbon atom spacers between the connection to T and the connection to R3, or wherein R1 is Cycle P or Cycle Q.

More preferably R1 (when R1 does not form a ring with R11 or R14) is hydrogen, C1-C3alkylene-R3, Cycle P or Cycle Q.

Specific examples of R1 when not forming a ring with R11 or R14, include hydrogen, —CH₂-tetrahydropyran-4-yl, —CH₂C(—CH₂CH₂—)—CN, —CH₂C(CH₃)₂—CN, —CH₂C(CH₃)₂—OH, —CH₂—C(═O)NHCH₃, —CH₂CH(OH)CH₂—OH, —CH₂CH₂—CF₃, —CH₂CH₂CH₂—CN, —CH₂CH₂—CN, —CH₂CH₂—NHC(═O)CH₃, —CH₂CH₂—NHS(O₂)CH₃, —CH₂CH₂—OCF₃, —CH₂CH₂—OCH₃, —CH₂CH₂—OH, —CH₂CH₂-oxazolidin-5-yl-2-one, —CH₂CH₂—S(O₂)CH₃, —CH₂CH₂—SCH₃, —CH₂CH₃, —CH₂—CN, —CH₂—OH, —CH₂-oxazolidin-5-yl-2-one, —CH₃, —CH₂CH₂—OCH₂CH₃, —CH₂CH₂CH₃, 4-fluorophenyl, —CH(CH₃)₂, —CH₂-2-(1-fluoroeth-1yl)-1,3,4-oxadiazol-4-yl, —CH₂—C(═O)OCH₂CH₃, —CH₂C(═O)OH, —CH₂CH(CH₃)₂, —CH₂CH₂C(CH₃)₂OH, —CH₂CH₂C(═O)OH, —CH₂CH₂CH(CH₃)₂, —CH₂CH₂CH₂CH₃, —CH₂CH₂NH₂, —CH₂-imidazolyl (e.g. —CH₂-imidazol-4-yl), —CH₂-oxazolyl (e.g. —CH₂-oxazol-4-yl), —CH₂-pyridin-3-yl (e.g. —CH₂-pyridin-3-yl and —CH₂-pyridin-4-yl), cyclopropyl, —CH₂CH₂C(═O)NH₂, —CH₂C(CH₃)₂C(═O)NH₂ and —CH₂C(CH₃)₂CH₂OH.

Y is a bond or C1-C6alkylene wherein one non-terminal —CH₂— moiety may be replaced by —O— and wherein the alkylene moiety may be substituted by one or two moieties independently selected from —OH, —O—C1-C4alkyl and —O—C1-C4haloalkyl and wherein the alkylene moiety may include a 3- to 5-membered saturated or partially unsaturated carbocyclic ring as part of the moiety. Reference to a “non-terminal —CH₂— moiety” refers a —CH₂— moiety which is not at either end of the alkylene moiety (i.e. not the terminal —CH₂— moiety which is proximal relative to the connection to T and not the terminal —CH₂— moiety which is distal relative to the connection to T).

Preferably Y is a bond or C1-C6alkylene wherein the alkylene moiety may be substituted by one —OH, wherein the alkylene moiety may include a 3-membered saturated carbocyclic ring as part of the moiety and wherein there are no more than three carbon atom spacers between the connection to T and the connection to R3.

More preferably Y is C1-C6alkylene wherein there are no more than three carbon atom spacers between the connection to T and the connection to R3 and wherein one CH₂ moiety is replaced by C(—CH₂CH₂—).

In some embodiments Y is C1-C3alkylene.

Specific examples of Y include a bond, —CH₂—, —CH₂C(—CH₂CH₂—)-, —CH₂C(CH₃)₂—, —CH₂CH(OH)CH₂—, —CH₂CH₂—, —CH₂CH₂CH₂—, —C(CH₃)₂—, —CH₂CH₂C(CH₃)₂—, —CH₂CH₂CH₂CH₂—, —CH₂C(CH₃)₂— and —CH₂C(CH₃)₂CH₂—.

The unit —C(—CH₂CH₂—)— corresponds to the moiety:

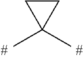

R3 is hydrogen, —CN, —OH, C1-C4haloalkyl, C2-C4alkenylene-R4, C2-C4alkynylene-R4, —O—C1-C4alkylene-R4, —O—C1-C4haloalkyl, —NH₂, —NH(C1-C4alkylene-R4), —N(C1-C4alkylene-R4)₂, —C(═O)—OH, —C(═O)—O—C1-C4alkylene-R4, —C(═O)—NH₂, —C(═O)—NH(C1-C4alkylene-R4), —C(═O)—N(C1-C4alkylene-R4)₂, —NH—C(═O)—C1-C4alkylene-R4, —N(C1-C4alkyl)-C(═O)—C1-C4alkylene-R4, Cycle P, Cycle Q, —O—Cycle P, —O—Cycle Q, —NH—S(O₂)—C1-C4alkylene-R4, —N(C1-C4alkyl)-S(O₂)—C1-C4alkylene-R4, —S—C1-C4alkylene-R4 or —S(O₂)—C1-C4alkyl-R4, providing that the atom in R3 connected to Y is not a heteroatom when (i) Y is a bond and (ii) T is not —N(R11)-, —C(R12)(R13)- or —C═C(R12)₂.

Preferably R3 is hydrogen, —CN, —OH, C1-C4haloalkyl, —O—C1-C4alkylene-R4, —O—C1-C4haloalkyl, —NH₂, —NH(C1-C4alkylene-R4), —N(C1-C4alkylene-R4)₂, —C(═O)—OH, —C(═O)—O—C1-C4alkylene-R4, —C(═O)—NH₂, —C(═O)—NH(C1-C4alkylene-R4), —C(═O)—N(C1-C4alkylene-R4)₂, —NH—C(═O)—C1-C4alkylene-R4, —N(C1-C4alkyl)-C(═O)—C1-C4alkylene-R4, Cycle P, Cycle Q, —NH—S(O₂)—C1-C4alkylene-R4, —N(C1-C4alkyl)-S(O₂)—C1-C4alkylene-R4, —S—C1-C4alkylene-R4 or —S(O₂)—C1-C4alkyl-R4.

More preferably R3 is hydrogen, —CN, —OH, C1-C4haloalkyl, —O—C1-C4alkylene-R4, —O—C1-C4haloalkyl, —NH₂, —NH(C1-C4alkylene-R4), —N(C1-C4alkylene-R4)₂, —C(═O)—OH, —C(═O)—NH₂, Cycle P or Cycle Q. Specific examples of R3 include hydrogen, tetrahydropyran-4-yl, —CN, —OH, —C(═O)NHCH₃, —CF₃, —NHC(═O)CH₃, —NHS(O₂)CH₃, —OCF₃, —OCH₃, -oxazolidin-5-yl-2-one, —S(O₂)CH₃, —SCH₃, —OCH₂CH₃, 4-fluorophenyl, 1,3,4-oxadiazol-4-yl (e.g. 2-(1-fluoroeth-1yl)-1,3,4-oxadiazol-4-yl), —C(═O)OCH₂CH₃, —C(═O)OH, —NH₂, imidazolyl (e.g. imidazol-4-yl), oxazolyl (e.g. oxazol-4-yl), pyridinyl (e.g. pyridin-3-yl and pyridin-4-yl), cyclopropyl and —C(═O)NH₂.

R4 is hydrogen, halogen, —NH₂, —NH(C1-C2alkyl) or —N(C1-C2alkyl)₂.

Preferably R4 is hydrogen.

Specific examples of R4 include hydrogen.

Cycle P is a 3- to 6-membered (e.g. 5- to 6-membered) saturated or partially unsaturated carbocyclic ring optionally substituted by one to three R5 or is a 3- to 6-membered (e.g. 5- to 6-membered) saturated or partially unsaturated heterocyclic ring containing one or two heteroatoms selected from N and O optionally substituted by one to three R5.

Preferably Cycle P is a 3- to 6-membered (e.g. 6-membered) saturated carbocyclic ring optionally substituted by one to two R5 or is a 5- or 6-membered saturated heterocyclic ring containing one heteroatom selected from N and O optionally substituted by one to two R5.

More preferably Cycle P is oxazolidinyl optionally substituted by one or two R5, in particular oxazolidinyl-2-one optionally substituted by one R5 wherein R5 is not oxo.

Specific examples of Cycle P include cyclopropyl, tetrahydropyranyl (e.g. tetrahydropyran-4-yl) and oxazolidinyl (e.g. oxazolidin-5-yl-2-one).

Each R5 is independently halogen, —NH₂, —OH, —CN, C1-C4alkylene-R5a, —O—C1-C4alkylene-R5a or oxo.

Preferably each R5 is independently —NH₂, C1-C2alkyl, —C1-C2alkyl-R5a or oxo.

More preferably each R5 is independently C1-C2alkyl, —C1-C2alkyl-R5a or oxo. Preferably when a moiety is substituted by R5 no more than one R5 is oxo. In particular, preferably an oxazolidinyl-2-one moiety is not further substituted by oxo.

Each R5a is independently hydrogen, halogen, —NH₂, —OH, —CN or —O—C1-C2alkyl.

Cycle Q is phenyl optionally substituted by one to three R6, or is a 5- to 6-membered heteroaryl ring containing one or two heteroatoms selected from N, S and O optionally substituted by one to three R6.

Preferably Cycle Q is phenyl optionally substituted by one or two R6, or is a 5- to 6-membered heteroaryl ring containing one to two heteroatoms selected from N, S and O optionally substituted by one or two R6.

More preferably Cycle Q is phenyl optionally substituted by one R6, or is a 5- to 6-membered heteroaryl ring containing one to two heteroatoms selected from N and O optionally substituted by one R6. Specific examples of Cycle Q include phenyl (e.g. 4-fluorophenyl), 1,3,4-oxadiazol (e.g. -1-fluoroeth-1-yl)-(1,3,4-oxadiazol-4-yl), imidazolyl (e.g. imidazole-4-yl), oxazolyl (e.g. oxazol-4-yl) and pyridinyl (e.g. pyridin-3-yl and pyridine-4-yl).

Each R6 is independently halogen, —NH₂, —OH, —CN, C1-C4alkylene-R6a or —O—C1-C4alkylene-R6a. Preferably each R6 is independently —NH₂, —OH, —CN, C1-C2alkylene-R6a (e.g. —CH₃) or —O—C1-C2alkyl (e.g. —O—CH₃).

Specific examples of R6 include fluoro and 1-fluoro-eth-1-yl.

Each R6a is independently hydrogen, halogen, —NH₂, —OH, —CN or —O—C1-C2alkyl. Preferably each R6a is independently hydrogen, halogen or —CN.

Specific examples of R6a include hydrogen and fluoro.

X is —O—, —C(═O)—, —C(═CH₂)—, —N(R10a)- or —CH(R10b)- and wherein X is a bond when R2 is halogen or —CN and wherein when X is —C(═O)— then R2 is not hydrogen, or wherein X—R2 is hydrogen.

Preferably X is —O— or —N(R10a)- or X—R2 is halogen or hydrogen.

More preferably X is —O— or —N(R10a)- or X—R2 is halogen.

Specific examples of X include —O— and —N(R10a)-, wherein R10a and R2 may together form a piperazine ring, e.g. 4-methylpiperazinyl, as well as bromo and hydrogen as X—R2. R10a is hydrogen or C1-C4alkyl or R2 and R10a may together form a 4- to 7-membered saturated or partially unsaturated heterocyclic ring optionally containing an —N(R9)- moiety as ring member in addition to the nitrogen atom of X and otherwise containing only carbon atoms as ring members optionally substituted by one or two R8.

Preferably R10a is hydrogen or —CH₃, or R2 and R10a may together form a 4- to 7-membered saturated heterocyclic ring optionally containing an —N(R9)- moiety as ring member in addition to the nitrogen atom of X and otherwise containing only carbon atoms as ring members and wherein when not containing an —N(R9)- moiety the heterocyclic ring is optionally substituted by one R8. More preferably R10a is hydrogen or —CH₃, or R2 and R10a may together form a 6- to 7-membered saturated heterocyclic ring containing one —N(R9)- moiety as a ring member at the 3 position with respect to the nitrogen atom of X (the position of the nitrogen atom of X is position 0) and otherwise containing only carbon atoms as ring members and wherein when not containing an —N(R9)- moiety the heterocyclic ring is substituted by one R8 at the 3 position with respect to the nitrogen atom of X.

In some embodiments R10a is hydrogen or —CH₃, or R2 and R10a may together form a 6-membered saturated heterocyclic ring containing one —N(R9)- moiety as a ring member at the 3 (“para”) position with respect to the nitrogen atom of X or substituted by one R8 at the 3 (“para”) position with respect to the nitrogen atom of X. Specific examples of ring formed from R2 and R10a include piperazinyl, e.g. 1-methylpiperazin-4-yl.

R10b is hydrogen or C1-C4alkyl or R2 and R10b may together form a 4- to 7-membered saturated or partially unsaturated carbocyclic ring or a 4- to 7-membered saturated or partially unsaturated heterocyclic ring containing an —N(R9)- moiety as ring member and otherwise containing only carbon atoms as ring members optionally substituted by one or two R8.

Preferably R10b is hydrogen.

R2 is hydrogen, halogen, —CN, C1-C4alkyl optionally substituted by one or two R7, or is a 4- to 7-membered saturated or partially unsaturated carbocyclic ring optionally substituted by one or two R8, or is a 4- to 7-membered saturated or partially unsaturated heterocyclic ring containing one —N(R9)- moiety as ring a member and otherwise containing only carbon atoms as ring members (when R2 does not form a ring with R10a, R10b, R11, R12 or R14).

Preferably R2 (when R2 does not form a ring with R10a, R10b, R11, R12 or R14) is C1-C4alkyl optionally substituted by R7 or is a 4- to 7-membered saturated carbocyclic ring (e.g. a 6- to 7-membered saturated carbocyclic ring) optionally substituted by one R8, or is a 4- to 7-membered saturated heterocyclic ring (e.g. a 6- to 7-membered saturated heterocyclic ring) containing one —N(R9)- moiety as a ring member and otherwise containing only carbon atoms as ring members, or wherein X—R2 is halogen or hydrogen.

More preferably R2 (when R2 does not form a ring with R10a, R10b, R11, R12 or R14) is C1-C4alkyl optionally substituted by R7 or is a 6- to 7-membered saturated carbocyclic ring substituted by one R8 at the 3 position with respect to X (the atom of the carbocyclic ring connected to X is position 0) or R2 is a 6- to 7-membered saturated heterocyclic ring containing one —N(R9)- moiety as a ring member at the 3 position with respect to X and otherwise containing only carbon atoms as ring members, or wherein X—R2 is halogen or hydrogen;

In some embodiments R2 (when R2 does not form a ring with R10a, R10b, R11, R12 or R14) is C1-C4alkyl optionally substituted by R7 or is a 6-membered saturated carbocyclic ring substituted by one R8 at the 3 (“para”) position with respect to X or R2 is a 6-membered saturated heterocyclic ring containing one —N(R9)- moiety as a ring member at the 3 (“para”) position with respect to X and otherwise containing only carbon atoms as ring members, or wherein X—R2 is bromine. Specific examples of R2, when not forming a ring with R10a, B10b, R11, R12 or R14, include azepan-4-yl, —CH₃, —CH₂CH₂N(CH₃)₂, 4-amino-cyclohexyl (e.g. cis-4-amino-cyclohexyl and trans-4-amino-cyclohexyl), 4-morpholin-4-yl-cyclohexyl (e.g. trans-4-morpholin-4-yl-cyclohexyl), 4—NH(CH₂CH₂OCH₃)-cyclohexyl (e.g. trans-4-N(CH₂CH₂OCH₃)-cyclohexyl), 4-N(CH₃)₂-cyclohexyl (e.g. trans-4-N(CH₃)₂-cyclohexyl), 4-NH(CH₃)-cyclohexyl (e.g. trans-4-NH(CH₃)-cyclohexyl) and piperidin-4-yl.

Specific examples of —X—R2 (when R2 is not forming a ring with R10a, R10b, R11, R12 or R14) include hydrogen, bromo, —O—CH₃, —O-azepan-4-yl, —O—CH₃, —O-(4-amino-cyclohexyl) (e.g. —O-(cis-4-amino-cyclohexyl), —O-(trans-4-amino-cyclohexyl)), —O—CH₂CH₂N(CH₃)₂, —O-(4-(morpholin-4-yl)-cyclohexyl) (e.g. —O-(trans-4-morpholin-4-yl-cyclohexyl)), —O-(4-NH(CH₂CH₂OCH₃)-cyclohexyl) (e-g. —O-(trans-4-NH(CH₂CH₂OCH₃)-cyclohexyl)), —O-(4-N(CH₃)₂-cyclohexyl) (e.g. —O-(trans-4-N(CH₃)₂-cyclohexyl)), —O-(4-NH(CH₃)-cyclohexyl) (e.g. —O-(trans-4-NH(CH₃)-cyclohexyl)) and —O-piperidin-4-yl.

Each R7 is independently —CN, —OH, halogen, —N(R7a)R7b or —NH(—C(═O)—C1-C4alkyl).

In some embodiments R7 is —N(R7a)R7b.

Specific examples of R7 include —N(CH₃)₂. Each R7a is independently hydrogen, C1-C4alkylene-R7c or C1-C4haloalkylene-R7c (when R7a does not form a ring with R7b).

In some embodiments each R7a, when not forming a ring with R7b, is independently hydrogen or C1-C4alkyl-R7c.

Specific examples of R7a include —CH₃.

Each R7b is independently hydrogen, C1-C4alkylene-R7c or C1-C4haloalkylene-R7c (when R7b does not form a ring with R7a).

In some embodiments each R7b, when not forming a ring with R7a, is independently hydrogen or C1-C4alkyl. Specific examples of R7b include —CH₃.

Each R7c is independently hydrogen, halogen, —O—C1-C2alkyl or —O—C1-C2haloalkyl.

R7a and R7b may together form a 4- to 7-membered saturated or partially unsaturated heterocyclic ring optionally containing one or two additional heteroatoms as ring members selected from N, O and S, and wherein the heterocyclic ring is optionally substituted by one or two R7d.

Each R7d is independently halogen, —OH, —CN, —NH₂, C1-C2alkyl, C1-C2haloalkyl, —O—C1-C2alkyl, —O—C1-C2haloalkyl, —NH(C1-C2alkyl) or —N(C1-C2alkyl)₂.

Each R8 is independently —CN, —OH, halogen, —N(R8a)R8b, —NH(—C(═O)—C1-C4alkyl), C1-C4alkyl or C1-C4haloalkyl.

Preferably each R8 is —N(R8a)R8b.

Specific examples of R8 include —NH₂, —NH(CH₃), —N(CH₃)₂, NH(CH₂CH₂OCH₃) and morpholin-4-yl.

Each R8a is independently hydrogen, C1-C4alkylene-R8c or C1-C4haloalkylene-R8c (when R8a does not form a ring with R8b).

Preferably each R8a, when not forming a ring with R8b, is independently hydrogen or C1-C4alkyl-R8c.

Specific examples of R8a include hydrogen, —CH₃ and —CH₂CH₂OCH₃.

Each R8b is independently hydrogen, C1-C4alkylene-R8c or C1-C4haloalkylene-R8c (when R8b does not form a ring with R8a).

Preferably R8b, when not forming a ring with R8a, is independently hydrogen or C1-C4alkyl.

Specific examples of R8b include hydrogen and —CH₃.

Each R8c is independently hydrogen, halogen, —O—C1-C2alkyl or —O—C1-C2haloalkyl.

Specific examples of R8c include hydrogen.

R8a and R8b may together form a 4- to 7-membered saturated or partially unsaturated heterocyclic ring optionally containing one or two additional heteroatoms as ring members selected from N, O and S, and wherein the heterocyclic ring is optionally substituted by one or two R8d.

Preferably R8a and R8b may together form a 6-membered saturated heterocyclic ring optionally containing one or two additional heteroatoms as ring members selected from N and O.

Specific examples of a ring formed by R8a and R8b include morpholin-4-yl.

Each R8d is independently halogen, —OH, —CN, —NH₂, C1-C2alkyl, C1-C2haloalkyl, —O—C1-C2alkyl, —O—C1-C2haloalkyl, —NH(C1-C2alkyl) or —N(C1-C2alkyl)₂.

R9 is hydrogen, C1-C4alkylene-R9a, C1-C4haloalkylene-R9a or —C(═O)—C1-C4alkyl.

Preferably R9 is hydrogen or C1-C4alkyl.

Specific examples of R9 include —CH₃.

R9a is hydrogen, halogen, —O—C1-C2alkyl or —O—C1-C2haloalkyl.

In some embodiments Ra and Rb are both —CH₃.

In some embodiments Ra and Rb together form a —CH₂—CH₂—CH₂—CH₂— bridging moiety.

In some embodiments A is —CH₂—.

In some embodiments A is —C(═O)—.

In some embodiments T is —N(R11)-.

In some embodiments T is —N(R11)-C(═O)—.

In some embodiments T is —N(R11)-S(O₂)—.

In some embodiments T is —O—.

In some embodiments T is —C(R12)(R13)-.

In some embodiments T is —C═C(R12)₂.

In some embodiments T is —C(═O)—N(R14)-.

In some embodiments T is —S—.

In some embodiments T is —S(O₂)—.

In some embodiments T is —S(O₂)—N(R14)-.

In some embodiments X is —O—.

In some embodiments X is —N(R10a)-.

In some embodiments X—R2 is hydrogen.

In some embodiments X—R2 is halogen.

In some embodiments T is —N(R11)- and X is —O—.

In some embodiments T is —N(R11)-C(═O)— and X is —O—.

In some embodiments T is —N(R11)-S(O₂)— and X is —O—.

In some embodiments T is —N(R11)- and X is —NH(R10a)-.

In some embodiments T is —N(R11)- and X—R2 is hydrogen.

In some embodiments T is —N(R11)- and X—R2 is halogen.

In some embodiments T is —O— and X is —O—.

In some embodiments T is —C(R12)(R13)- and X is —O—.

In some embodiments T is —C═C(R12)₂ and X is —O—.

In some embodiments T is —C(═O)—N(R14)- and X is —O—.

In some embodiments T is —O— and X is —O—.

In some embodiments T is —S— and X is —O—.

In some embodiments T is —S(O₂)— and X is —O—.

In some embodiments T is —S(O₂)—N(R14)- and X is —O—.

In some embodiments T is —N(R11)- and X is —N(R10a)-.

In some embodiments A is —CH₂— and T is —N(R11)-.

In some embodiments A is —CH₂— and T is —N(R11)-C(═O)—.

In some embodiments A is —CH₂— and T is —N(R11)-S(O₂)—.

In some embodiments A is —CH₂— and T is —O—.

In some embodiments A is —CH₂— and T is —C(R12)(R13)-.

In some embodiments A is —CH₂— and T is —C═C(R12)₂.

In some embodiments A is —CH₂— and T is —C(═O)—N(R14)-.

In some embodiments A is —CH₂— and T is —S—.

In some embodiments A is —CH₂— and T is —S(O₂)—.

In some embodiments A is —CH₂— and T is —S(O₂)—N(R14)-.

In some embodiments A is —C(═O)— and T is —N(R11)-.

In some embodiments A is —CH₂— and X is —O—.

In some embodiments A is —CH₂— and X is —N(R10a)-.

In some embodiments A is —CH₂— and X—R2 is hydrogen.

In some embodiments A is —CH₂— and X—R2 is halogen.

In some embodiments A is —C(═O)— and X is —O—.

In some embodiments A is —CH₂—, T is —N(R11)- and X is —O—.

In some embodiments A is —CH₂—, T is —N(R11)-C(═O)— and X is —O—.

In some embodiments A is —CH₂—, T is —N(R11)-S(O₂)— and X is —O—.

In some embodiments A is —CH₂—, T is —O— and X is —O—.

In some embodiments A is —CH₂—, T is —C(R12)(R13)- and X is —O—.

In some embodiments A is —CH₂—, T is —N(R11)- and —N(R10a)-.

In some embodiments A is —CH₂—, T is —C═C(R12)₂ and X is —O—, In some embodiments A is —CH₂—, T is —C(═O)—N(R14)- and X is —O—.

In some embodiments A is —CH₂—, T is —O— and X is —O—.

In some embodiments A is —CH₂—, T is —S— and X is —O—.

In some embodiments A is —CH₂—, T is —S(O₂)— and X is —O—.

In some embodiments A is —CH₂—, T is —S(O₂)—N(R14)- and X is —O—.

In some embodiments A is —C(═O)—, T is —N(R11)- and X is-NHR10a)-.

In some embodiments Ra and Rb are both —CH₃ and A is —CH₂—.

In some embodiments Ra and Rb together form a —CH₂—CH₂—CH₂—CH₂— bridging moiety and A is —CH₂—.

In some embodiments Ra and Rb are both —CH₃ and A is —C(═O)—.

In some embodiments Ra and Rb are both —CH₃ and T is —N(R11)-.

In some embodiments Ra and Rb are both —CH₃ and T is —N(R11)-C(═O)—.

In some embodiments Ra and Rb are both —CH₃ and T is —N(R11)-S(O₂)—.

In some embodiments Ra and Rb are both —CH₃ and T is —O—.

In some embodiments Ra and Rb are both —CH₃ and T is —C(R12)(R13)-.

In some embodiments Ra and Rb are both —CH₃ and T is —C═C(R12)₂.

In some embodiments Ra and Rb are both —CH₃ and T is —C(═O)—N(R14)-.

In some embodiments Ra and Rb are both —CH₃ and T is —S—.

In some embodiments Ra and Rb are both —CH₃ and T is —S(O₂)—.

In some embodiments Ra and Rb are both —CH₃ and T is —S(O₂)—N(R14)-.

In some embodiments Ra and Rb together form a —CH₂—CH₂—CH₂—CH₂— bridging moiety and T is —N(R11)—.

In some embodiments Ra and Rb are both —CH₃, A is —CH₂— and T is —N(R11)-.

In some embodiments Ra and Rb are both —CH₃, A is —CH₂— and T is —N(R11)-C(═O)—.

In some embodiments Ra and Rb are both —CH₃, A is —CH₂— and T is —N(R11)-S(O₂)—.

In some embodiments Ra and Rb are both —CH₃, A is —CH₂— and T is —O—.

In some embodiments Ra and Rb are both —CH₃, A is —CH₂— and T is —C(R12)(R13)-.

In some embodiments Ra and Rb are both —CH₃, A is —CH₂— and T is —C═C(R12)₂.

In some embodiments Ra and Rb are both —CH₃, A is —CH₂— and T is —C(═O)—N(R14)-.

In some embodiments Ra and Rb are both —CH₃, A is —CH₂— and T is —S—.

In some embodiments Ra and Rb are both —CH₃, A is —CH₂— and T is —S(O₂)—.

In some embodiments Ra and Rb are both —CH₃, A is —CH₂— and T is —S(O₂)—N(R14)-.

In some embodiments Ra and Rb together form a —CH₂—CH₂—CH₂—CH₂— bridging moiety, A is —CH₂— and T is —N(R11)-.

In some embodiments Ra and Rb are both —CH₃, A is —C(═O)— and T is —N(R11)-.

In some embodiments, the substituent R1 is not hydrogen when T is —N(R11), —O—, —C(R12)(R13)-, —S—, —S(O)— or —S(O₂)— (i.e. for the avoidance of doubt this means that neither R1 nor the moiety Y—R3 can be hydrogen in this case).

In some embodiments the substituent R1 is not hydrogen.

In some embodiments the substituent R1 contains at least 1 atom other than hydrogen.

In some embodiments the substituent R1 contains at least 2 atoms other than hydrogen.

In some embodiments the substituent R1 contains at least 3 atoms other than hydrogen.

In some embodiments the substituent R1 contains at least 4 atoms other than hydrogen.

In some embodiments the substituent R1 contains at least 5 atoms other than hydrogen.

In some embodiments R1, when not forming a ring with R11 or R14, is —Y—R3, providing that Y is not a bond when and R3 is hydrogen.

In some embodiments R1, when not forming a ring with R11 or R14, is —Y—R3;
Y is C1-C6alkylene wherein one non-terminal —CH₂— moiety may be replaced by —O— and wherein the alkylene moiety may be substituted by one or two moieties independently selected from —OH, —O—C1-C4alkyl and —O—C1-C4haloalkyl and wherein the alkylene moiety may include a 3-to 5-membered saturated or partially unsaturated carbocyclic ring as part of the moiety, or wherein R1 is Cycle P or Cycle Q.

In some embodiments R1, when not forming a ring with R11 or R14, is C1-C6alkylene-R3, wherein the alkylene moiety may be substituted by one —OH, wherein the alkylene moiety may include a 3-membered saturated carbocyclic ring as part of the moiety, and wherein there are no more than three carbon atom spacers between the connection to T and the connection to R3, or wherein R1 is Cycle P or Cycle Q.

In some embodiments R1, when not forming a ring with R11 or R14, is C1-C3alkylene-R3, Cycle P or Cycle Q.

In some embodiments R1 is C1-C3alkylene-R3;
R3 is hydrogen, —CN, —OH, C1-C4haloalkyl, —O—C1-C4alkylene-R4, —O—C1-C4haloalkyl, —NH₂, —NH(C1-C4alkylene-R4) or —N(C1-C4alkylene-R4)₂, —C(═O)—OH, —C(═O)—NH₂, Cycle P or Cycle Q; R11 is hydrogen, C1-C4alkylene-R11a, C1-C4haloalkylene-R11a or C3-C4cycloalkyl;
Cycle P is oxazolidinyl optionally substituted by one or two R5;
each R5 is independently C1-C2alkyl,-C1-C2alkyl-R5a or oxo, wherein Cycle P is not substituted by two oxo;
each R5a is independently hydrogen, halogen, —NH₂, —OH, —CN or —O—C1-C2alkyl.

In some embodiments X is —O—, —C(═O)—, —N(R10a)- or —CH(R10b)- and wherein X is a bond when R2 is halogen or —CN and wherein when X is —C(═O)— then R2 is not hydrogen, or wherein X—R2 is hydrogen.

In some embodiments R2 and R10a together form a 4- to 7-membered saturated or partially unsaturated heterocyclic ring optionally containing an —N(R9)- moiety as ring member in addition to the nitrogen atom of X and otherwise containing only carbon atoms as ring members optionally substituted by one or two R8.

In some embodiments R2 and R10a do not together form a heterocyclic ring.

In some embodiments R2 and R10b may together form a 4- to 7-membered saturated or partially unsaturated carbocyclic ring or a 4- to 7-membered saturated or partially unsaturated heterocyclic ring containing an —N(R9)- moiety as ring member and otherwise containing only carbon atoms as ring members optionally substituted by one or two R8.

In some embodiments R2 and R10b do not together form a heterocyclic ring.

In some embodiments R2 and R11 together form a partially unsaturated 6- to 7-membered heterocyclic ring containing no additional heteroatoms as ring members, wherein the heterocyclic ring is optionally substituted by one or two R2a.

In some embodiments R2 and R11 do not together form a heterocyclic ring.

In some embodiments R2 and R12 together form a partially unsaturated 6- to 7-membered heterocyclic ring containing no additional heteroatoms as ring members, wherein the heterocyclic ring is optionally substituted by one or two R2a.

In some embodiments R2 and R12 do not together form a heterocyclic ring.

In some embodiments R2 and R14 together form a partially unsaturated 6- to 7-membered heterocyclic ring containing no additional heteroatoms as ring members, wherein the heterocyclic ring is optionally substituted by one or two R2a.

In some embodiments R2 and R14 do not together form a heterocyclic ring.

In some embodiments R2 and R11, R2 and R12 and R2 and R14 do not do not together form a heterocyclic ring.

In some embodiments R1 and R11 together form a saturated or partially unsaturated 4- to 7-membered heterocyclic ring optionally containing one or two additional heteroatoms as ring members selected from N, O and S, wherein the heterocyclic ring is optionally substituted by one to three R1a, or R1 and R11 together form a 5- to 6-membered heteroaryl ring optionally containing one to three additional heteroatoms as ring members selected from N and O, wherein the heteroaryl ring is optionally substituted by one to three R1b.

In some embodiments R1 and R11 do not together form a heterocyclic ring or heteroaryl ring.

In some embodiments R1 and R14 together form a saturated or partially unsaturated 4- to 7-membered heterocyclic ring optionally containing one or two additional heteroatoms as ring members selected from N, O and S, wherein the heterocyclic ring is optionally substituted by one to three R1a, or R1 and R14 together form a 5- to 6-membered heteroaryl ring optionally containing one to three additional heteroatoms as ring members selected from N and O, wherein the heteroaryl ring is optionally substituted by one to three R1b.

In some embodiments R1 and R14 do not together form a heterocyclic ring or heteroaryl ring.

In some embodiments R2 and R10a, R2 and R10b, R2 and R11, R2 and R12, R2 and R14, R1 and R11, and R1 and R14 do not together form a heterocyclic ring or heteroaryl ring.

In some embodiments R1 and R11 may together form a heterocyclic ring or heteroaryl ring as defined herein (in particular when T is —N(R11)-), but R2 and R10a, R2 and R10b, R2 and R11, R2 and R12, R2 and R14, and R1 and R14 do not together form a heterocyclic ring.

In some embodiments R2 is C1-C4alkyl or is a 6-membered saturated carbocyclic ring substituted by one R8 at the 3 (“para”) position with respect to X and R8 is —N(R8a)R8b.

In some embodiments
X is —O— or —NH(R10a)-, or X—R2 is halogen or hydrogen;
R10a is hydrogen or —CH₃;
R2 is C1-C4alkyl or is a 6-membered saturated carbocyclic ring substituted by one R8 at the 3 (“para”) position with respect to X;
R8 is —N(R8a)R8b;
R8a, when not forming a ring with R8b, is hydrogen or C1-C4alkyl-R8c;
R8b, when not forming a ring with R8a, is hydrogen or C1-C4alkyl;
R8a and R8b may together form a 6-membered saturated heterocyclic ring optionally containing one or two additional heteroatoms as ring members selected from N and O; and
R8c is hydrogen, halogen, —O—C1-C2alkyl or —O—C1-C2haloalkyl.

In some embodiments:
T is —N(R11)-, —N(R11)-C(═O)— or —N(R11)-S(O₂)—;
R1, when not forming a ring with R11, is —Y—R3;
or wherein R1 and R11 may together form a saturated or partially unsaturated 4- to 7-membered heterocyclic ring optionally containing one or two additional heteroatoms as ring members selected from N, O and S, wherein the heterocyclic ring is optionally substituted by one to three R1a, or R1 and R11 may together form a 5- to 6-membered heteroaryl ring optionally containing one to three additional heteroatoms as ring members selected from N and O, wherein the heteroaryl ring is optionally substituted by one to three R1b; and
R11 is C1-C4alkylene-R11a, C1-C4haloalkylene-R11a or C3-C6cycloalkyl, wherein the cycloalkyl is optionally substituted by one or two R11b.

In some embodiments:
T is —N(R11)-, —N(R11)-C(═O)— or —N(R11)-S(O₂)—;
R1, when not forming a ring with R11, is —Y—R3;
Y is C1-C6alkylene wherein one non-terminal —CH₂— moiety may be replaced by —O— and wherein the alkylene moiety may be substituted by one or two moieties independently selected from —OH, —O—C1-C4alkyl and —O—C1-C4haloalkyl and wherein the alkylene moiety may include a 3- to 5-membered saturated or partially unsaturated carbocyclic ring as part of the moiety;
or wherein R1 and R11 may together form a saturated or partially unsaturated 4- to 7-membered heterocyclic ring optionally containing one or two additional heteroatoms as ring members selected from N, O and S, wherein the heterocyclic ring is optionally substituted by one to three R1a, or R1 and R11 or may together form a 5- to 6-membered heteroaryl ring optionally containing one to three additional heteroatoms as ring members selected from N and O, wherein the heteroaryl ring is optionally substituted by one to three R1b; and
R11 is C1-C4alkylene-R11a, C1-C4haloalkylene-R11a or C3-C6cycloalkyl, wherein the cycloalkyl is optionally substituted by one or two R11b.

In some embodiments:
T is —N(R11)- or —N(R11)-S(O₂)—;
R11, when not forming a ring with R1, is hydrogen, C1-C4alkylene-R11a, C1-C4haloalkylene-R11a or C3-C6cycloalkyl;
R11a is hydrogen, halogen, —CN or —OH;
R1 and R11 may together form a 4- to 6-membered saturated or partially unsaturated heterocyclic ring optionally containing one additional heteroatom as a ring member selected from N and O, wherein the heterocyclic ring is optionally substituted by one or two R1a, or R1 and R11 may together form a 5-membered heteroaryl ring optionally containing one additional heteroatom as a ring member selected from N, and wherein the heteroaryl ring is optionally substituted by one or two R1b;
each R1a is independently halogen, —OH, —CN, C1-C4alkylene-Ric, —O—C1-C4haloalkyl, —C(═O)NH₂ or oxo;
each R1b is independently halogen, —OH, —CN, C1-C4alkylene-R1c or —O—C1-C4haloalkyl;
each R1c is independently hydrogen or —OH;
R1, when not forming a ring with R11, is —Y—R3;
Y is C1-C6alkylene wherein the alkylene moiety may be substituted by one —OH, wherein the alkylene moiety may include a 3-membered saturated carbocyclic ring as part of the moiety and wherein there are no more than three carbon atom spacers between the connection to T and the connection to R3;
R3 is hydrogen, —CN, —OH, C1-C4haloalkyl, —O—C1-C4alkylene-R4, —O—C1-C4haloalkyl, —NH₂, —NH(C1-C4alkylene-R4), —N(C1-C4alkylene-R4)₂, —C(═O)—NH₂, —C(═O)—NH(C1-C4alkylene-R4), —C(═O)—OH, —C(═O)—O—C1-C4alkylene-R4, —C(═O)—N(C1-C4alkylene-R4)₂, —NH—C(═O)—C1-C4alkylene-R4, —N(C1-C4alkyl)-C(═O)—C1-C4alkylene-R4, Cycle P, Cycle Q, —NH—S(O₂)—C1-C4alkylene-R4, —N(C1-C4alkyl)-S(O₂)—C1-C4alkylene-R4, —S—C1-C4alkylene-R4 or —S(O₂)—C1-C4alkyl-R4;
R4 is hydrogen;
Cycle P is a 3- to 6-membered saturated carbocyclic ring optionally substituted by one to two R5 or is a 5- or 6-membered saturated heterocyclic ring containing one heteroatom selected from N and O optionally substituted by one to two R5;
each R5 is independently —NH₂, C1-C2alkyl,-C1-C2alkyl-R5a or oxo;
each R5a is independently hydrogen, halogen, —NH₂, —OH, —CN or —O—C1-C2alkyl;
Cycle Q is phenyl optionally substituted by one or two R6, or is a 5- to 6-membered heteroaryl ring containing one to two heteroatoms selected from N, S and O optionally substituted by one or two R6;
each R6 is independently —NH₂, —OH, —CN, C1-C2alkylene-R6a or —O—C1-C2alkyl; and
each R6a is independently hydrogen, halogen or —CN.

In some embodiments:
T is —N(R11)- or —N(R11)-S(O₂)—;
R11, when not forming a ring with R1, is C1-C4alkylene-R11a, C1-C4haloalkylene-R11a or C3-C4cycloalkyl;
R11a is hydrogen, halogen, —CN or —OH;
R1 and R11 may together form a 5- to 6-membered saturated heterocyclic ring containing no additional heteroatoms as ring members, wherein the heterocyclic ring is optionally substituted by one R1a, or R1 and R11 may together form a 5-membered heteroaryl ring containing no additional heteroatoms as ring members, and wherein the heteroaryl ring is optionally substituted by one R1b;
R1a is halogen, —OH or —CN;
R1b is halogen, —OH or —CN;
R1, when not forming a ring with R11, is C1-C3alkylene-R3, Cycle P or Cycle Q;
R3 is hydrogen, —CN, —OH, C1-C4haloalkyl, —O—C1-C4alkylene-R4, —O—C1-C4haloalkyl, —NH₂, —NH(C1-C4alkylene-R4) or —N(C1-C4alkylene-R4)₂, —C(═O)—OH, —C(═O)—NH₂, Cycle P or Cycle Q;
Cycle P is oxazolidinyl optionally substituted by one or two R5;
each R5 is independently C1-C2alkyl,-C1-C2alkyl-R5a or oxo, wherein Cycle P is not substituted by two oxo (e.g. Cycle P is optionally substituted only by one R5 when R5 is oxo);
Cycle Q is phenyl optionally substituted by one R6, or is a 5- to 6-membered heteroaryl ring containing one to two heteroatoms selected from N and O optionally substituted by one R6;
R6 is independently —NH₂, —OH, —CN, C1-C2alkylene-R6a or —O—C1-C2alkyl; and
R6a is independently hydrogen, halogen or —CN.

In some embodiments T is —N(R11)-, and R11, when not forming a ring with R1, is C1-C4alkyl, e.g. methyl or ethyl.

In an embodiment (Embodiment A1) the compound is a compound of formula (I), wherein
Ra and Rb are both —CH₃ or together form a —CH₂—CH₂—CH₂—CH₂— bridging moiety;
A is —CH₂— or —C(═O)—;
T is —N(R11)-, —N(R11)-S(O₂)—, —O—, —C(R12)(R13)-, —C═C(R12)₂, —S—, —S(O₂)—, —S(O₂)—N(R14)- or —C(═O)—N(R14)-;
R11, when not forming a ring with R1 or R2, is hydrogen, C1-C4alkylene-R11a, C1-C4haloalkylene-R11a or C3-C6cycloalkyl;
R11a is hydrogen, halogen, —CN or —OH;
each R12 is independently hydrogen or C1-C4alkylene-R12a;
each R12a is independently hydrogen, halogen, —CN or —OH;
R13 is hydrogen;
R14 is hydrogen or C1-C4alkyl;
R2 and R11 may together form a partially unsaturated 6-membered heterocyclic ring containing no additional heteroatoms when T is —N(R11)- and X is —O—;
R1 and R11 may together form a 4- to 6-membered saturated or partially unsaturated heterocyclic ring optionally containing one additional heteroatom as a ring member selected from N and O, e.g. a ring selected from piperidinyl, morpholinyl, pyrrolidinyl, azetidinyl, 2,5-dihydropyrrolyl and 1,1-dioxide-1,2-thiazolidin-2-yl, wherein the heterocyclic ring is optionally substituted by one or two R1a, or R1 and R11 may together form a 5-membered heteroaryl ring optionally containing one additional heteroatom as a ring member selected from N, e.g. selected from pyrrolyl, and wherein the heteroaryl ring is optionally substituted by one or two R1b;
each R1a is independently halogen, —OH, —CN, C1-C4alkylene-Ric, —O—C1-C4haloalkyl, —C(═O)NH₂ or oxo;
each R1b is independently halogen, —OH, —CN, C1-C4alkylene-R1c or —O—C1-C4haloalkyl;
each R1c is independently hydrogen or —OH;
R1, when not forming a ring with R11, is —Y—R3;
Y is a bond or C1-C6alkylene wherein the alkylene moiety may be substituted by one —OH, wherein the alkylene moiety may include a 3-membered saturated carbocyclic ring as part of the moiety and wherein there are no more than three carbon atom spacers between the connection to T and the connection to R3;
R3 is hydrogen, —CN, —OH, C1-C4haloalkyl, —O—C1-C4alkylene-R4, —O—C1-C4haloalkyl, —NH₂, —NH(C1-C4alkylene-R4), —N(C1-C4alkylene-R4)₂, —C(═O)—NH₂, —C(═O)—NH(C1-C4alkylene-R4), —C(═O)—OH, —C(═O)—O—C1-C4alkylene-R4, —C(═O)—N(C1-C4alkylene-R4)₂, —NH—C(═O)—C1-C4alkylene-R4, —N(C1-C4alkyl)-C(═O)—C1-C4alkylene-R4, Cycle P, Cycle Q, —NH—S(O₂)—C1-C4alkylene-R4, —N(C1-C4alkyl)-S(O₂)—C1-C4alkylene-R4, —S—C1-C4alkylene-R4 or —S(O₂)—C1-C4alkyl-R4;
R4 is hydrogen;
Cycle P is a 3- to 6-membered saturated carbocyclic ring optionally substituted by one to two R5 or is a 5- or 6-membered saturated heterocyclic ring containing one heteroatom selected from N and O optionally substituted by one to two R5;
each R5 is independently —NH₂, C1-C2alkyl,-C1-C2alkyl-R5a or oxo;
each R5a is independently hydrogen, halogen, —NH₂, —OH, —CN or —O—C1-C2alkyl;
Cycle Q is phenyl optionally substituted by one or two R6, or is a 5- to 6-membered heteroaryl ring containing one to two heteroatoms selected from N, S and O optionally substituted by one or two R6;
each R6 is independently —NH₂, —OH, —CN, C1-C2alkylene-R6a or —O—C1-C2alkyl;
each R6a is independently hydrogen, halogen or —CN;
X is —O— or —N(R10a)- or X—R2 is halogen or hydrogen;
R10a is hydrogen or —CH₃ or R2 and R10a may together form a 4- to 7-membered saturated heterocyclic ring optionally containing an —N(R9)- moiety as ring member in addition to the nitrogen atom of X and otherwise containing only carbon atoms as ring members and wherein when not containing an —N(R9)-moiety the heterocyclic ring is optionally substituted by one R8;
R2, when not forming a ring with R10a or R11, is C1-C4alkyl optionally substituted by R7 or is a 4- or 7-membered saturated carbocyclic ring optionally substituted by one R8, or is a 4- or 7-membered saturated heterocyclic ring containing one —N(R9)- moiety as a ring member and otherwise containing only carbon atoms as ring members, or X—R2 is halogen or hydrogen;
R7 is —N(R7a)R7b;
R7a is hydrogen or C1-C4alkyl-R7c;
R7b is hydrogen or C1-C4alkyl;
R7c is hydrogen, halogen, —O—C1-C2alkyl or —O—C1-C2haloalkyl;
R8 is —N(R8a)R8b;
R8a, when not forming a ring with R8b, is hydrogen or C1-C4alkyl-R8c;
R8b when not forming a ring with R8a, is hydrogen or C1-C4alkyl;
R8a and R8b may together form a 6-membered saturated heterocyclic ring optionally containing one or two additional heteroatoms as ring members selected from N and O;
R8c is hydrogen, halogen, —O—C1-C2alkyl or —O—C1-C2haloalkyl; and
R9 is hydrogen or C1-C4alkyl.

In an embodiment (Embodiment A2) the compound is a compound of formula (I), wherein Ra and Rb are both —CH₃ or together form a —CH₂—CH₂—CH₂—CH₂— bridging moiety;
A is —CH₂— or —C(═O)—;
T is —N(R11)-, —N(R11)-S(O₂)—, —O—, —C(R12)(R13)-, —C═C(R12)₂, —S—, —S(O₂)—, —S(O₂)—N(R14)- or —C(═O)—N(R14)-;
R11, when not forming a ring with R1 or R2, is hydrogen, C1-C4alkylene-R11a, C1-C4haloalkylene-R11a or C3-C6cycloalkyl;
R11a is hydrogen, halogen, —CN or —OH;
each R12 is independently hydrogen or C1-C4alkylene-R12a;
each R12a is independently hydrogen, halogen, —CN or —OH;
R13 is hydrogen;
R14 is hydrogen or C1-C4alkyl;
R2 and R11 may together form a partially unsaturated 6-membered heterocyclic ring containing no additional heteroatoms when T is —N(R11)- and X is —O—;
R1 and R11 may together form a 4- to 6-membered saturated or partially unsaturated heterocyclic ring optionally containing one additional heteroatom as a ring member selected from N and O, e.g. a ring selected from piperidinyl, morpholinyl, pyrrolidinyl, azetidinyl, 2,5-dihydropyrrolyl and 1,1-dioxide-1,2-thiazolidin-2-yl, wherein the heterocyclic ring is optionally substituted by one or two R1a, or a 5-membered heteroaryl ring optionally containing one additional heteroatom as a ring member selected from N, e.g. selected from pyrrolyl, and wherein the heteroaryl ring is optionally substituted by one or two R1b;
each R1a is independently halogen, —OH, —CN, C1-C4alkylene-R1c, —O—C1-C4haloalkyl, —C(═O)NH₂ or oxo;
each R1b is independently halogen, —OH, —CN, C1-C4alkylene-R1c or —O—C1-C4haloalkyl;
each R1c is independently hydrogen or —OH;
R1, when not forming a ring with R11, is hydrogen or C1-C6alkylene-R3, wherein the alkylene moiety may be substituted by one —OH, wherein the alkylene moiety may include a 3-membered saturated carbocyclic ring as part of the moiety and wherein there are no more than three carbon atom spacers between the connection to T and the connection to R3 or wherein R1 is Cycle P or Cycle Q;
R3 is hydrogen, —CN, —OH, C1-C4haloalkyl, —O—C1-C4alkylene-R4, —O—C1-C4haloalkyl, —NH₂, —NH(C1-C4alkylene-R4), —N(C1-C4alkylene-R4)₂, —C(═O)—NH₂, —C(═O)—NH(C1-C4alkylene-R4), —C(═O)—OH, —C(═O)—O—C1-C4alkylene-R4, —C(═O)—N(C1-C4alkylene-R4)₂, —NH—C(═O)—C1-C4alkylene-R4, —N(C1-C4alkyl)-C(═O)—C1-C4alkylene-R4, Cycle P, Cycle Q, —NH—S(O₂)—C1-C4alkylene-R4, —N(C1-C4alkyl)-S(O₂)—C1-C4alkylene-R4, —S—C1-C4alkylene-R4 or —S(O₂)—C1-C4alkyl-R4;
R4 is hydrogen;
Cycle P is a 3- to 6-membered saturated carbocyclic ring optionally substituted by one to two R5 or is a 5- or 6-membered saturated heterocyclic ring containing one heteroatom selected from N and O optionally substituted by one to two R5;
each R5 is independently —NH₂, C1-C2alkyl,-C1-C2alkyl-R5a or oxo;
each R5a is independently hydrogen, halogen, —NH₂, —OH, —CN or —O—C1-C2alkyl;
Cycle Q is phenyl optionally substituted by one or two R6, or is a 5- to 6-membered heteroaryl ring containing one to two heteroatoms selected from N, S and O optionally substituted by one or two R6;
each R6 is independently —NH₂, —OH, —CN, C1-C2alkylene-R6a or —O—C1-C2alkyl;
each R6a is independently hydrogen, halogen or —CN;
X is —O— or —NH(R10a)- or X—R2 is halogen or hydrogen;
R10a is hydrogen or —CH₃ or R2 and R10a may together form a 4- to 7-membered saturated heterocyclic ring optionally containing an —N(R9)- moiety as ring member in addition to the nitrogen atom of X and otherwise containing only carbon atoms as ring members and wherein when not containing an —N(R9)-moiety the heterocyclic ring is optionally substituted by one R8;
R2, when not forming a ring with R10a or R11, is C1-C4alkyl optionally substituted by R7 or is a 4- to 7-membered saturated carbocyclic ring optionally substituted by one R8, or is a 4- to 7-membered saturated heterocyclic ring containing one —N(R9)- moiety as a ring member and otherwise containing only carbon atoms as ring members or X—R2 is halogen or hydrogen;
R7 is —N(R7a)R7b;
R7a is hydrogen or C1-C4alkyl-R7c;
R7b is hydrogen or C1-C4alkyl;
R7c is hydrogen, halogen, —O—C1-C2alkyl or —O—C1-C2haloalkyl; R8 is —N(R8a)R8b;
R8a, when not forming a ring with R8b, is hydrogen or C1-C4alkyl-R8c;
R8b, when not forming a ring with R8a, is hydrogen or C1-C4alkyl;
R8a and R8b may together form a 6-membered saturated heterocyclic ring optionally containing one or two additional heteroatoms as ring members selected from N and O;
R8c is hydrogen, halogen, —O—C1-C2alkyl or —O—C1-C2haloalkyl; and
R9 is hydrogen or C1-C4alkyl.

In an embodiment (Embodiment B) the compound is a compound of formula (I), wherein
Ra and Rb are both —CH₃;
A is —CH₂—;
T is —N(R11)-, —N(R11)-S(O₂)—, —C═C(R12)₂, —S— or —C(═O)—N(R14)-;
R11, when not forming a ring with R1, is hydrogen, C1-C4alkylene-R11a, C1-C4haloalkylene-R11a or C3-C4cycloalkyl;
R11a is hydrogen, halogen, —CN or —OH;
each R12 is independently hydrogen or C1-C4alkylene-R12a;
each R12a is independently hydrogen, halogen, —CN or —OH;
R14 is hydrogen or C1-C4alkyl;
R1 and R11 may together form a 5- to 6-membered saturated heterocyclic ring containing no additional heteroatoms as ring members, e.g. a ring selected from piperidinyl, pyrrolidinyl and 1,1-dioxide-1,2-thiazolidin-2-yl, wherein the heterocyclic ring is optionally substituted by one R1a, or R1 and R11 may together form a 5-membered heteroaryl ring containing no additional heteroatoms as ring members, e.g. selected from pyrrolyl, and wherein the heteroaryl ring is optionally substituted by one R1b;
R1a is halogen, —OH or —CN;
R1b is halogen, —OH or —CN;
R1, when not forming a ring with R11, is hydrogen, C1-C3alkylene-R3, Cycle P or Cycle Q;
R3 is hydrogen, —CN, —OH, C1-C4haloalkyl, —O—C1-C4alkylene-R4, —O—C1-C4haloalkyl, —NH₂, —NH(C1-C4alkylene-R4) or —N(C1-C4alkylene-R4)₂, —C(═O)—OH, —C(═O)—NH₂, Cycle P or Cycle Q;
R4 is hydrogen;
Cycle P is oxazolidinyl optionally substituted by one or two R5;
each R5 is independently C1-C2alkyl,-C1-C2alkyl-R5a or oxo, wherein Cycle P is not substituted by two oxo (e.g. Cycle P is optionally substituted only by one R5 when R5 is oxo);
each R5a is independently hydrogen, halogen, —NH₂, —OH, —CN or —O—C1-C2alkyl;
Cycle Q is phenyl optionally substituted by one R6, or is a 5- to 6-membered heteroaryl ring containing one to two heteroatoms selected from N and O optionally substituted by one R6;
R6 is independently —NH₂, —OH, —CN, C1-C2alkylene-R6a or —O—C1-C2alkyl;
R6a is independently hydrogen, halogen or —CN;
X is —O— or —N(R10a)- or X—R2 is halogen;
R10a is hydrogen or —CH₃ or R2 and R10a may together form a 6- to 7-membered saturated heterocyclic ring containing one —N(R9)- moiety as a ring member at the 3 position with respect to the nitrogen atom of X and otherwise containing only carbon atoms as ring members and wherein when not containing an —N(R9)— moiety the heterocyclic ring is substituted by one R8 at the 3 position with respect to the nitrogen atom of X;
R2, when not forming a ring with R10a, is C1-C4alkyl optionally substituted by R7, or is a 6-to 7-membered saturated carbocyclic ring substituted by one R8 at the 3 position with respect to X or R2 is a 6- to 7-membered saturated heterocyclic ring containing one —N(R9)- moiety as a ring member at the 3 position with respect to X and otherwise containing only carbon atoms as ring members, or wherein X—R2 is halogen or hydrogen;
R7 is —N(R7a)R7b;
R7a is hydrogen or C1-C4alkyl-R7c;
R7b is hydrogen or C1-C4alkyl;
R7c is hydrogen, halogen, —O—C1-C2alkyl or —O—C1-C2haloalkyl;
R8 is —N(R8a)R8b;
R8a, when not forming a ring with R8b, is hydrogen or C1-C4alkyl-R8c;
R8a, when not forming a ring with R8a, is hydrogen or C1-C4alkyl;
R8a and R8b may together form a 6-membered saturated heterocyclic ring optionally containing one or two additional heteroatoms as ring members selected from N and O;
R8c is hydrogen, halogen, —O—C1-C2alkyl or —O—C1-C2haloalkyl; and
R9 is hydrogen or C1-C4alkyl.

In an embodiment (Embodiment C) the compound is a compound of formula (I), wherein
Ra and Rb are both —CH₃ or Ra and Rb together form a —CH₂—CH₂—CH₂—CH₂— bridging moiety;
A is —CH₂— or —C(═O)—;
T is —N(R11)-, —N(R11)-C(═O)—, —N(R11)-S(O₂)—, —O— or —C(R12)(R13)-, —C═C(R12)₂, —S—, —S(O₂)—, —S(O₂)—N(R14)- or —C(═O)—N(R14)-;R11, when not forming a ring with R1 or R2, is —CH₃, cyclopropyl, —CH₂CH₂CH₃, —CH₂CH₂CN, —CH₂CH₂OH, —CH₂CH₃ or hydrogen;
each R12 is independently hydrogen, —CH₃ or —CH₂OH;
R13 is hydrogen;
R14 is hydrogen or —CH₃;
R2 and R11 may together form a —CH₂CH₂— bridging moiety when T is —N(R11)- and X is —O—;
R1 and R11 may together form a heterocyclic ring when T is —N(R11)-selected from piperidinyl, morpholinyl, pyrrolidinyl azetidinyl, 2,5-dihydropyrrolyl and 1,1-dioxide-1,2-thiazolidin-2-yl, each optionally substituted by one to two R1a;
each R1a is independently —OH, —CN, —OCH₃, —CH₂OH, —C(═O)NH₂, or oxo;
R1 and R11 may together form a heteroaryl ring when T is —N(R11)-selected from pyrrolyl optionally substituted by one or two R1b;
each R1b is independently selected from —OH, —CN, —OCH₃ and —CH₂OH;
R1, when not forming a ring with R11, is hydrogen or —Y—R3;
Y is bond, —CH₂—, —CH₂C(—CH₂CH₂—)-, —CH₂C(CH₃)₂—, —CH₂CH(OH)CH₂—, —CH₂CH₂— or —CH₂CH₂CH₂—, —C(CH₃)₂—, —CH₂CH₂C(CH₃)₂—, —CH₂CH₂CH₂CH₂—, —CH₂C(CH₃)₂— or —CH₂C(CH₃)₂CH₂—;
R3 is hydrogen, 1-tetrahydropyran-4-yl, —CN, —OH, —C(═O)NHCH₃, —CF₃, —NHC(═O)CH₃, —NHS(O₂)CH₃, —OCF₃, —OCH₃, -oxazolidin-5-yl-2-one, —S(O₂)CH₃, —SCH₃, —OCH₂CH₃, 4-fluorophenyl, 1,3,4-oxadiazol-4-yl, —C(═O)OCH₂CH₃, —C(═O)OH, —NH₂, imidazolyl, oxazolyl, pyridinyl, cyclopropyl or —C(═O)NH₂;
X is —O—, or —N(R10a)-, or X—R2 is bromo or hydrogen;
R2 and R10a may together form a piperazinyl ring; and
R2, when not forming a ring with R10a or R11, is azepan-4-yl, —CH₃, —CH₂CH₂N(CH₃)₂, 4-amino-cyclohexyl (e.g. cis-4-amino-cyclohexyl and trans-4-amino-cyclohexyl), 4-morpholin-4-yl-cyclohexyl (e.g. trans-4-morpholin-4-yl-cyclohexyl), 4-NH(CH₂CH₂OCH₃)-cyclohexyl (e.g., trans-4-N(CH₂CH₂OCH₃)-cyclohexyl), 4-N(CH₃)₂-cyclohexyl (e.g. trans-4-N(CH₃)₂-cyclohexyl), 4-NH(CH₃)-cyclohexyl (e.g. trans-4-NH(CH₃)-cyclohexyl) or piperidin-4-yl, or wherein X—R2 is bromo or hydrogen.

In further embodiments the invention provides the following compounds and pharmaceutically acceptable salts thereof:
8-methoxy-5,5,7-trimethyl-6H-benzo[h]quinazolin-4-amine;
8-methoxy-5,5-dimethyl-6H-benzo[h]quinazoline-4,7-diamine;
6,6-dimethyl-2,3,4,5-tetrahydroquinazolino[7,8-f][1,4]benzoxazin-7-amine;
4,6,6-trimethyl-3,5-dihydro-2H-quinazolino[7,8-f][1,4]benzoxazin-7-amine;
8-(trans-4-aminocyclohexoxy)-5,5-dimethyl-6H-benzo[h]quinazoline-4,7-diamine;
N-[4-amino-8-(trans-4-aminocyclohexoxy)-5,5-dimethyl-6H-benzo[h]quinazolin-7-yl]acetamide;
8-(trans-4-aminocyclohexoxy)-N7,N7-diethyl-5,5-dimethyl-6H-benzo[h]quinazoline-4,7-diamine;
8-(trans-4-aminocyclohexoxy)-N7-ethyl-5,5-dimethyl-6H-benzo[h]quinazoline-4,7-diamine;
8-(trans-4-aminocyclohexoxy)-5,5-dimethyl-7-(1-piperidyl)-6H-benzo[h]quinazolin-4-amine;
8-(trans-4-aminocyclohexoxy)-5,5-dimethyl-7-morpholino-6H-benzo[h]quinazolin-4-amine;
2-[[4-amino-8-(trans-4-aminocyclohexoxy)-5,5-dimethyl-6H-benzo[h]quinazolin-7-yl]-(2-hydroxyethyl)amino]ethanol;
2-[[4-amino-8-(trans-4-aminocyclohexoxy)-5,5-dimethyl-6H-benzo[h]quinazolin-7-yl]-methyl-amino]ethanol;
8-(trans-4-aminocyclohexoxy)-N7-(2-methoxyethyl)-N7,5,5-trimethyl-6H-benzo[h]quinazoline-4,7-diamine;
2-[[4-amino-8-(trans-4-aminocyclohexoxy)-5,5-dimethyl-6H-benzo[h]quinazolin-7-yl]amino]ethanol;
1-[4-amino-8-(trans-4-aminocyclohexoxy)-5,5-dimethyl-6H-benzo[h]quinazolin-7-yl]pyrrolidin-3-ol—isomer 1;
1-[4-amino-8-(trans-4-aminocyclohexoxy)-5,5-dimethyl-6H-benzo[h]quinazolin-7-yl]pyrrolidin-3-ol—isomer 2;
8-(trans-4-aminocyclohexoxy)-5,5-dimethyl-7-pyrrol-1-yl-6H-benzo[h]quinazolin-4-amine;
8-(trans-4-aminocyclohexoxy)-N7-(2-methoxyethyl)-5,5-dimethyl-6H-benzo[h]quinazoline-4,7-diamine;
8-(trans-4-aminocyclohexoxy)-5,5-dimethyl-7-propoxy-6H-benzo[h]quinazolin-4-amine;
8-(trans-4-aminocyclohexoxy)-N7,N7,5,5-tetramethyl-6H-benzo[h]quinazoline-4,7-diamine;
1-(4-amino-8-methoxy-5,5-dimethyl-6H-benzo[h]quinazolin-9-yl)piperidin-4-ol;
2-[[4-amino-8-(cis-4-aminocyclohexoxy)-5,5-dimethyl-6H-benzo[h]quinazolin-7-yl]-ethyl-amino]ethanol;
2-[[4-amino-8-(cis-4-aminocyclohexoxy)-5,5-dimethyl-6H-benzo[h]quinazolin-7-yl]-propyl-amino]ethanol;
2-[[4-amino-8-(cis-4-aminocyclohexoxy)-5,5-dimethyl-6H-benzo[h]quinazolin-7-yl]-methyl-amino]ethanol;
1-(4-amino-8-methoxy-5,5-dimethyl-6H-benzo[h]quinazolin-7-yl)azetidin-3-ol;
2-[[4-amino-8-(cis-4-aminocyclohexoxy)-5,5-dimethyl-6H-benzo[h]quinazolin-7-yl]-methyl-amino]-N-methyl-acetamide;
3-[(4-amino-8-methoxy-5,5-dimethyl-6H-benzo[h]quinazolin-7-yl)amino]propane-1,2-diol;
[1-(4-amino-8-methoxy-5,5-dimethyl-6H-benzo[h]quinazolin-7-yl)azetidin-3-yl]methanol;
8-(cis-4-aminocyclohexoxy)-N7,5,5-trimethyl-N7-(tetrahydropyran-4-ylmethyl)-6H-benzo[h]quinazoline-4,7-diamine;
2-[[4-amino-8-(cis-4-aminocyclohexoxy)-5,5-dimethyl-6H-benzo[h]quinazolin-7-yl]-methyl-amino]acetonitrile;
8-(cis-4-aminocyclohexoxy)-N7,5,5-trimethyl-N7-(2-methylsulfanylethyl)-6H-benzo[h]quinazoline-4,7-diamine;
8-(cis-4-aminocyclohexoxy)-N7,5,5-trimethyl-N7-(2-methylsulfonylethyl)-6H-benzo[h]quinazoline-4,7-diamine;
2-[[4-amino-8-(trans-4-aminocyclohexoxy)-5,5-dimethyl-6H-benzo[h]quinazolin-7-yl]-methyl-amino]acetonitrile;
4,7-diamino-8-(trans-4-aminocyclohexoxy)-5,5-dimethyl-benzo[h]quinazolin-6-one;
2-[[4-amino-8-(trans-4-aminocyclohexoxy)-5,5-dimethyl-6H-benzo[h]quinazolin-7-yl]amino]acetonitrile;
8-(trans-4-aminocyclohexoxy)-N7-(2-ethoxyethyl)-N7,5,5-trimethyl-6H-benzo[h]quinazoline-4,7-diamine;
8-(trans-4-aminocyclohexoxy)spiro[6H-benzo[h]quinazoline-5,1′-cyclopentane]-4,7-diamine;
N-[2-[[4-amino-8-(trans-4-aminocyclohexoxy)-5,5-dimethyl-6H-benzo[h]quinazolin-7-yl]-methyl-amino]ethyl]acetamide;
N-[2-[[4-amino-8-(trans-4-aminocyclohexoxy)-5,5-dimethyl-6H-benzo[h]quinazolin-7-yl]-methyl-amino]ethyl]methanesulfonamide;
1-[[4-amino-8-(trans-4-aminocyclohexoxy)-5,5-dimethyl-6H-benzo[h]quinazolin-7-yl]-methyl-amino]-2-methyl-propan-2-ol;
N-[4-amino-8-(trans-4-aminocyclohexoxy)-5,5-dimethyl-6H-benzo[h]quinazolin-7-yl]-2-hydroxy-N-methyl-acetamide;
N-[4-amino-8-(trans-4-aminocyclohexoxy)-5,5-dimethyl-6H-benzo[h]quinazolin-7-yl]-2-hydroxy-ethanesulfonamide;
8-(trans-4-aminocyclohexoxy)-N7,5,5-trimethyl-N7-(3,3,3-trifluoropropyl)-6H-benzo[h]quinazoline-4,7-diamine;
N-[4-amino-8-(4-trans-aminocyclohexoxy)-5,5-dimethyl-6H-benzo[h]quinazolin-7-yl]-2-hydroxy-N-methyl-ethanesulfonamide;
N-[4-amino-8-(trans-4-aminocyclohexoxy)-5,5-dimethyl-6H-benzo[h]quinazolin-7-yl]-2-hydroxy-acetamide;
3-[[4-amino-8-(trans-4-aminocyclohexoxy)-5,5-dimethyl-6H-benzo[h]quinazolin-7-yl]-methyl-amino]propanenitrile;
8-(trans-4-aminocyclohexoxy)-N7,5,5-trimethyl-N7-[2-(trifluoromethoxy)ethyl]-6H-benzo[h]quinazoline-4,7-diamine. 1-[[[4-amino-8-(trans-4-aminocyclohexoxy)-5,5-dimethyl-6H-benzo[h]quinazolin-7-yl]-methyl-amino]methyl]cyclopropanecarbonitrile;
1-[4-amino-8-(trans-4-aminocyclohexoxy)-5,5-dimethyl-6H-benzo[h]quinazolin-7-yl]pyrrolidine-3-carbonitrile—isomer 1;
1-[4-amino-8-(trans-4-aminocyclohexoxy)-5,5-dimethyl-6H-benzo[h]quinazolin-7-yl]pyrrolidine-3-carbonitrile—isomer 2;
3-[[4-amino-8-(trans-4-aminocyclohexoxy)-5,5-dimethyl-6H-benzo[h]quinazolin-7-yl]-cyclopropyl-amino]propanenitrile;
3-[[4-amino-5,5-dimethyl-8-(trans-4-morpholinocyclohexoxy)-6H-benzo[h]quinazolin-7-yl]-methyl-amino]propanenitrile;
3-[[4-amino-8-[trans-4-(2-methoxyethylamino)cyclohexoxy]-5,5-dimethyl-6H-benzo[h]quinazolin-7-yl]-methyl-amino]propanenitrile;
8-(trans-4-aminocyclohexoxy)-N7-but-3-ynyl-N7,5,5-trimethyl-6H-benzo[h]quinazoline-4,7-diamine;
1-(4-amino-8-methoxy-5,5-dimethyl-6H-benzo[h]quinazolin-7-yl)pyrrolidine-3-carbonitrile.
1-[4-amino-8-(trans-4-aminocyclohexoxy)-5,5-dimethyl-6H-benzo[h]quinazolin-7-yl]-2H-pyrrol-5-one;
8-(trans-4-aminocyclohexoxy)-7-(3-methoxypyrrol-1-yl)-5,5-dimethyl-6H-benzo[h]quinazolin-4-amine;
4-[[4-amino-8-(4-trans-aminocyclohexoxy)-5,5-dimethyl-6H-benzo[h]quinazolin-7-yl]-methyl-amino]butanenitrile;
2-[[4-amino-8-(4-trans-aminocyclohexoxy)spiro[6H-benzo[h]quinazoline-5,1′-cyclopentane]-7-yl]-methyl-amino]ethanol;
2-[[4-amino-8-(4-trans-aminocyclohexoxy)spiro[6H-benzo[h]quinazoline-5,1′-cyclopentane]-7-yl]amino]ethanol;
8-methoxy-7-(3-methoxypropyl)-5,5-dimethyl-6H-benzo[h]quinazolin-4-amine;
8-(trans-4-aminocyclohexoxy)-N7-(2-methoxyethyl)spiro[6H-benzo[h]quinazoline-5,1′-cyclopentane]-4,7-diamine;
8-(trans-4-aminocyclohexoxy)-7-(3-methoxypyrrolidin-1-yl)-5,5-dimethyl-6H-benzo[h]quinazolin-4-amine—isomer 1;
N-[4-amino-8-(trans-4-aminocyclohexoxy)-5,5-dimethyl-6H-benzo[h]quinazolin-7-yl]-2-methoxy-N-methyl-ethanesulfonamide;
ethyl 2-[[4-amino-8-(cis-4-aminocyclohexoxy)spiro[6H-benzo[h]quinazoline-5,1′-cyclopentane]-7-yl]amino]acetate;
1-[4-amino-8-(trans-4-aminocyclohexoxy)-5,5-dimethyl-6H-benzo[h]quinazolin-7-yl]pyrrolidine-3-carbonitrile;
8-(trans-4-aminocyclohexoxy)-N7-ethyl-N7,5,5-trimethyl-6H-benzo[h]quinazoline-4,7-diamine;
8-(trans-4-aminocyclohexoxy)-N7,5,5-trimethyl-6H-benzo[h]quinazoline-4,7-diamine;
3-[[4-amino-8-(trans-4-aminocyclohexoxy)-5,5-dimethyl-6H-benzo[h]quinazolin-7-yl]-methyl-sulfamoyl]propanoic acid;
2-[[4-amino-8-(cis-4-aminocyclohexoxy)spiro[6H-benzo[h]quinazoline-5,1′-cyclopentane]-7-yl]amino]ethanol;
3-[[4-amino-8-(trans-4-aminocyclohexoxy)-5,5-dimethyl-6H-benzo[h]quinazolin-7-yl]oxy]propanenitrile;
8-(trans-4-aminocyclohexoxy)-N7-(2-methoxyethyl)-N7-methyl-spiro[6H-benzo[h]quinazoline-5,1′-cyclopentane]-4,7-diamine;
8-(trans-4-aminocyclohexoxy)-7-(3-methoxypyrrolidin-1-yl)-5,5-dimethyl-6H-benzo[h]quinazolin-4-amine—isomer 2;
N-[4-amino-8-(trans-4-aminocyclohexoxy)-5,5-dimethyl-6H-benzo[h]quinazolin-7-yl]-2-cyano-N-methyl-ethanesulfonamide;
8-(cis-4-aminocyclohexoxy)-N7-(2-methoxyethyl)-N7-methyl-spiro[6H-benzo[h]quinazoline-5,1′-cyclopentane]-4,7-diamine;
1-[[[4-amino-8-(trans-4-aminocyclohexoxy)-5,5-dimethyl-6H-benzo[h]quinazolin-7-yl]amino]methyl]cyclopropanecarbonitrile;
1-[4-amino-8-(trans-4-aminocyclohexoxy)-5,5-dimethyl-6H-benzo[h]quinazolin-7-yl]pyrrolidine-3-carboxamide;
3-[[4-amino-8-(azepan-4-yloxy)-5,5-dimethyl-6H-benzo[h]quinazolin-7-yl]-methyl-amino]propanenitrile;
2-[[4-amino-8-(cis-4-aminocyclohexoxy)spiro[6H-benzo[h]quinazoline-5,1′-cyclopentane]-7-yl]amino]acetic acid;
8-(trans-4-aminocyclohexoxy)-N7-(2-aminoethyl)-N7,5,5-trimethyl-6H-benzo[h]quinazoline-4,7-diamine;
4-[4-amino-8-(trans-4-aminocyclohexoxy)-5,5-dimethyl-6H-benzo[h]quinazolin-7-yl]butanenitrile;
(5R)-5-[[(4-amino-8-methoxy-5,5-dimethyl-6H-benzo[h]quinazolin-7-yl)amino]methyl]oxazolidin-2-one;
2-[[4-amino-8-(cis-4-aminocyclohexoxy)spiro[6H-benzo[h]quinazoline-5,1′-cyclopentane]-7-yl]-methyl-amino]ethanol;
8-(trans-4-aminocyclohexoxy)-N7-cyclopropyl-5,5-dimethyl-6H-benzo[h]quinazoline-4,7-diamine;
3-[[4-amino-8-(trans-4-aminocyclohexoxy)-5,5-dimethyl-6H-benzo[h]quinazolin-7-yl]-methyl-amino]propanoic acid;
3-[[4-amino-8-(trans-4-aminocyclohexoxy)-5,5-dimethyl-6H-benzo[h]quinazolin-7-yl]-methyl-sulfamoyl]propanamide;
8-(cis-4-aminocyclohexoxy)-N7-(2-methoxyethyl)spiro[6H-benzo[h]quinazoline-5,1′-cyclopentane]-4,7-diamine;
3-[[4-amino-8-(trans-4-aminocyclohexoxy)-5,5-dimethyl-6H-benzo[h]quinazolin-7-yl]-methyl-amino]-2,2-dimethyl-propanamide;
(5R)-5-[2-[(4-amino-8-methoxy-5,5-dimethyl-6H-benzo[h]quinazolin-7-yl)amino]ethyl]oxazolidin-2-one;
4-amino-8-(trans-4-aminocyclohexoxy)-N-(cyanomethyl)-5,5-dimethyl-6H-benzo[h]quinazoline-7-carboxamide;
3-[[4-amino-8-(trans-4-aminocyclohexoxy)-5,5-dimethyl-6H-benzo[h]quinazolin-7-yl]-methyl-amino]-2,2-dimethyl-propanenitrile;
3-[4-amino-8-(trans-4-aminocyclohexoxy)-5,5-dimethyl-6H-benzo[h]quinazolin-7-yl]butan-1-ol;
3-[[4-amino-5,5-dimethyl-8-(4-piperidyloxy)-6H-benzo[h]quinazolin-7-yl]-methyl-amino]propanenitrile;
4-amino-8-(trans-4-aminocyclohexoxy)-N-(cyanomethyl)-N,5,5-trimethyl-6H-benzo[h]quinazoline-7-carboxamide;
3-[(4-amino-8-methoxy-5,5-dimethyl-6H-benzo[h]quinazolin-7-yl)-methyl-amino]propanenitrile;
3-[[4-amino-8-trans-4-(dimethylamino)cyclohexoxy]-5,5-dimethyl-6H-benzo[h]quinazolin-7-yl]-methyl-amino]propanenitrile;
4-[4-amino-8-(trans-4-aminocyclohexoxy)-5,5-dimethyl-6H-benzo[h]quinazolin-7-yl]pentanenitrile;
(E)-3-[4-amino-8-(trans-4-aminocyclohexoxy)-5,5-dimethyl-6H-benzo[h]quinazolin-7-yl]but-2-en-1-ol;
3-[[4-amino-5,5-dimethyl-8-[trans-4-(methylamino)cyclohexoxy]-6H-benzo[h]quinazolin-7-yl]-methyl-amino]propanenitrile;
8-(trans-4-aminocyclohexoxy)-N7-butyl-N7,5,5-trimethyl-6H-benzo[h]quinazoline-4,7-diamine;
8-(trans-4-aminocyclohexoxy)-N7,5,5-trimethyl-N7-(4-pyridylmethyl)-6H-benzo[h]quinazoline-4,7-diamine;
8-(trans-4-aminocyclohexoxy)-N7-isopropyl-N7,5,5-trimethyl-6H-benzo[h]quinazoline-4,7-diamine;
8-(trans-4-aminocyclohexoxy)-N7-(1H-imidazol-4-ylmethyl)-N7,5,5-trimethyl-6H-benzo[h]quinazoline-4,7-diamine;
8-(trans-4-aminocyclohexoxy)-N7-isopentyl-N7,5,5-trimethyl-6H-benzo[h]quinazoline-4,7-diamine;
8-(trans-4-aminocyclohexoxy)-N7,5,5-trimethyl-N7-(oxazol-4-ylmethyl)-6H-benzo[h]quinazoline-4,7-diamine;
8-(trans-4-aminocyclohexoxy)-N7-isobutyl-N7,5,5-trimethyl-6H-benzo[h]quinazoline-4,7-diamine;
8-(trans-4-aminocyclohexoxy)-N7,5,5-trimethyl-N7-(3-pyridylmethyl)-6H-benzo[h]quinazoline-4,7-diamine;
3-[[4-amino-8-(trans-4-aminocyclohexoxy)-5,5-dimethyl-6H-benzo[h]quinazolin-7-yl]sulfanyl]propanenitrile;
3-[(4-amino-5,5-dimethyl-6H-benzo[h]quinazolin-7-yl)-methyl-amino]propanenitrile;
3-[[4-amino-8-(trans-4-aminocyclohexoxy)-5,5-dimethyl-6H-benzo[h]quinazolin-7-yl]-methyl-amino]-2,2-dimethyl-propan-1-ol;
4-[[4-amino-8-(trans-4-aminocyclohexoxy)-5,5-dimethyl-6H-benzo[h]quinazolin-7-yl]-methyl-amino]-2-methyl-butan-2-ol;
3-[[4-amino-8-(trans-4-aminocyclohexoxy)-5,5-dimethyl-6H-benzo[h]quinazolin-7-yl]sulfonyl]propanenitrile;
4-amino-8-(trans-4-aminocyclohexoxy)-N-(cyanomethyl)-N,5,5-trimethyl-6H-benzo[h]quinazoline-7-sulfonamide;
N-[4-amino-8-(trans-4-aminocyclohexoxy)-5,5-dimethyl-6H-benzo[h]quinazolin-7-yl]-4-fluoro-N-methyl-benzenesulfonamide;
3-[(4-amino-8-bromo-5,5-dimethyl-6H-benzo[h]quinazolin-7-yl)-methyl-amino]propanenitrile;
4-amino-8-(trans-4-aminocyclohexoxy)-N-(cyanomethyl)-5,5-dimethyl-6H-benzo[h]quinazoline-7-sulfonamide;
3-(7-amino-6,6-dimethyl-3,5-dihydro-2H-quinazolino[7,8-f][1,4]benzoxazin-4-yl)propanenitrile;
3-[[4-amino-5,5-dimethyl-8-(4-methylpiperazin-1-yl)-6H-benzo[h]quinazolin-7-yl]-methyl-amino]propanenitrile;
8-(trans-4-aminocyclohexoxy)-7-(1,1-dioxo-1,2-thiazolidin-2-yl)-5,5-dimethyl-6H-benzo[h]quinazolin-4-amine;
1-(4-amino-8-bromo-5,5-dimethyl-6H-benzo[h]quinazolin-7-yl)pyrrolidine-3-carbonitrile;
(5R)-5-[[(4-amino-8-bromo-5,5-dimethyl-6H-benzo[h]quinazolin-7-yl)amino]methyl]oxazolidin-2-one;
N7-[[5-(1-fluoroethyl)-1,3,4-oxadiazol-2-yl]methyl]-8-methoxy-5,5-dimethyl-6H-benzo[h]quinazoline-4,7-diamine;
3-[[4-amino-8-[2-(dimethylamino)ethoxy]-5,5-dimethyl-6H-benzo[h]quinazolin-7-yl]-methyl-amino]propanenitrile;
3-[(4-amino-8-methoxy-5,5-dimethyl-6H-benzo[h]quinazolin-7-yl)-[[5-(1-fluoroethyl)-1,3,4-oxadiazol-2-yl]methyl]amino]propanenitrile;
3-[(4-amino-5,5-dimethyl-8-piperazin-1-yl-6H-benzo[h]quinazolin-7-yl)-methyl-amino]propanenitrile;
(5S)-5-[[(4-amino-8-bromo-5,5-dimethyl-6H-benzo[h]quinazolin-7-yl)-methyl-amino]methyl]oxazolidin-2-one;
(5R)-5-[[(4-amino-8-bromo-5,5-dimethyl-6H-benzo[h]quinazolin-7-yl)amino]methyl]-3-(2-hydroxyethyl)oxazolidin-2-one;
(5R)-5-[[[4-amino-8-(cis-4-aminocyclohexoxy)-5,5-dimethyl-6H-benzo[h]quinazolin-7-yl]amino]methyl]oxazolidin-2-one;
(5R)-5-[[[4-amino-8-(trans-4-aminocyclohexoxy)-5,5-dimethyl-6H-benzo[h]quinazolin-7-yl]amino]methyl]oxazolidin-2-one;
(5R)-5-[[[4-amino-5,5-dimethyl-8-(cis-4-morpholinocyclohexoxy)-6H-benzo[h]quinazolin-7-yl]amino]methyl]oxazolidin-2-one;
(5R)-5-[[[4-amino-5,5-dimethyl-8-(trans-4-morpholinocyclohexoxy)-6H-benzo[h]quinazolin-7-yl]amino]methyl]oxazolidin-2-one; and
3-[(4-amino-8-bromo-5,5-dimethyl-6H-benzo[h]quinazolin-7-yl)-[[(5S)-2-oxooxazolidin-5-yl]methyl]amino]propanenitrile.

Some intermediates useful for the preparation of compounds of formula (I) are new and form further aspects of the invention. Accordingly, in a further aspect the invention provides compounds of formula (B)

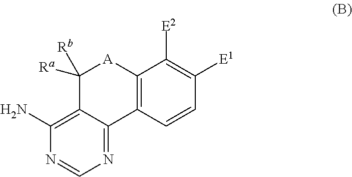

and salts thereof, wherein
A, Ra and Rb are as defined for compounds of formula (I), including preferred definitions thereof (including as defined in Embodiment A1, Embodiment A2, Embodiment B or Embodiment C);
E1 is —OH, -halogen, —O—C1-C4alkyl, boronic acid, a boronic ester (e.g. the dimethyl or pinacol boronic ester) or O-L1;
L1 is methanesulfonyl, p-toluenesulfonyl or trifluoromethanesulfonyl; and
E2 is —OH, halogen, —NO₂ or —SO₂C1.

In a further aspect the invention provides compounds of formula (C)

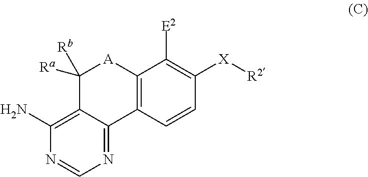

and salts thereof, wherein
A, X, Ra and Rb are as defined for compounds of formula (I), including preferred definitions thereof (including as defined in Embodiment A1, Embodiment A2, Embodiment B or Embodiment C);
E2 is —OH, halogen, —NO₂ or —SO₂C1; and
R2′ is as defined for R2 for compounds of formula (I), including preferred definitions thereof (including as defined in Embodiment A1, Embodiment A2, Embodiment B or Embodiment C), wherein any functional group within R2 is optionally protected with a suitable protecting group.

Preferably in compounds of formula (C), within the R2′ substituent, an amino group may optionally be protected with a suitable amino protecting group, such as allyloxycarbonyl, benzyloxycarbonyl, 9-fluorenylmethylcarbonyl, tert-butoxycarbonyl or benzyl and/or a hydroxyl group may optionally be protected with a suitable hydroxyl protecting group, such as methyl, benzyl or para-methoxybenzyl.

In a further aspect the invention provides compounds of formula (D)

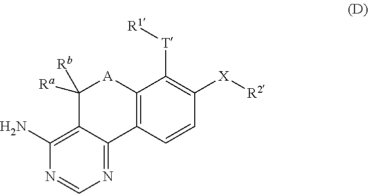

and salts thereof, wherein
A, X, Ra and Rb are as defined for compounds of formula (I), including preferred definitions thereof (including as defined in Embodiment A1, Embodiment A2, Embodiment B or Embodiment C);
T′ is —N(R11′)—, —N(R11′)—C(═O)—, —N(R11′)—S(O₂)—, —O—, —C(R12′)(R13)-, —C═C(R12′)₂, —S—, —S(O)—, —S(O₂)—, —S(O₂)—N(R14′)— or —C(═O)—N(R14′)—;
R11′ is as defined for R11 in compounds of formula (I), including preferred definitions thereof (including as defined in Embodiment A1, Embodiment A2, Embodiment B or Embodiment C), wherein any functional group within R11 is optionally protected with a suitable protecting group;
R12′ is as defined for R12 in compounds of formula (I), including preferred definitions thereof (including as defined in Embodiment A1, Embodiment A2, Embodiment B or Embodiment C), wherein any functional group within R12 is optionally protected with a suitable protecting group;
R13 is as defined for compounds of formula (I), including preferred definitions thereof (including as defined in Embodiment A1, Embodiment A2, Embodiment B or Embodiment C);
R14′ is as defined for R14 in compounds of formula (I), including preferred definitions thereof (including as defined in Embodiment A1, Embodiment A2, Embodiment B or Embodiment C), wherein any functional group within R14 is optionally protected with a suitable protecting group;
R1′ is as defined for R1 in compounds of formula (I), including preferred definitions thereof (including as defined in Embodiment A1, Embodiment A2, Embodiment B or Embodiment C), wherein any functional group within R1 is optionally protected with a suitable protecting group; and
R2′ is as defined for R2 in compounds of formula (I), including preferred definitions thereof (including as defined in Embodiment A1, Embodiment A2, Embodiment B or Embodiment C), wherein any functional group within R2 is optionally protected with a suitable protecting group. Preferably compounds of formula (D) include at least one protecting group.

Preferably in compounds of formula (D), within one or more of the substituents R1′, R2′, R1′, R12′ and R14′, an amino group may optionally be protected with a suitable amino protecting group, such as allyloxycarbonyl, benzyloxycarbonyl, 9-fluorenylmethylcarbonyl, tert-butoxycarbonyl or benzyl and/or a hydroxyl group may optionally be protected with a suitable hydroxyl protecting group, such as methyl, benzyl or para-methoxybenzyl.

In a further aspect the invention provides compounds of formula (E)

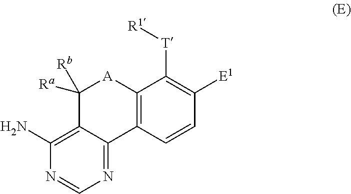

and salts thereof, wherein
A, Ra and Rb are as defined for compounds of formula (I), including preferred definitions thereof (including as defined in Embodiment A1, Embodiment A2, Embodiment B or Embodiment C);
T′ is —N(R11′)—, —N(R11′)—C(═O)—, —N(R11′)—S(O₂)—, —O—, —C(R12′)(R13)-, —C═C(R12′)₂, —S—, —S(O)—, —S(O₂)—, —S(O₂)—N(R14′)— or —C(═O)—N(R14′)—;
R11′ is as defined for R11 in compounds of formula (I), including preferred definitions thereof (including as defined in Embodiment A1, Embodiment A2, Embodiment B or Embodiment C), wherein any functional group within R11 is optionally protected with a suitable protecting group;
R12′ is as defined for R12 in compounds of formula (I), including preferred definitions thereof (including as defined in Embodiment A1, Embodiment A2, Embodiment B or Embodiment C), wherein any functional group within R12 is optionally protected with a suitable protecting group;
R13 is as defined for compounds of formula (I), including preferred definitions thereof (including as defined in Embodiment A1, Embodiment A2, Embodiment B or Embodiment C);
R14′ is as defined for R14 in compounds of formula (I), including preferred definitions thereof (including as defined in Embodiment A1, Embodiment A2, Embodiment B or Embodiment C), wherein any functional group within R14 is optionally protected with a suitable protecting group;
R1′ is as defined for R1 in compounds of formula (I), including preferred definitions thereof (including as defined in Embodiment A1, Embodiment A2, Embodiment B or Embodiment C), wherein any functional group within R1 is optionally protected with a suitable protecting group;
E1 is —OH, -halogen, —O—C1-C4alkyl, boronic acid, a boronic ester (e.g. the dimethyl or pinacol boronic ester) or O-L1; and
L1 is methanesulfonyl, p-toluenesulfonyl or trifluoromethanesulfonyl.

Preferably in compounds of formula (E), within one or more of the substituents R1′, R11′, R12′ and R14′, an amino group may optionally be protected with a suitable amino protecting group, such as allyloxycarbonyl, benzyloxycarbonyl, 9-fluorenylmethylcarbonyl, tert-butoxycarbonyl or benzyl and/or a hydroxyl group may optionally be protected with a suitable hydroxyl protecting group, such as methyl, benzyl or para-methoxybenzyl.

The present invention relates also to pharmaceutical compositions that comprise a compound of formula (I) as active ingredient or a pharmaceutically acceptable salt thereof, which can be used especially in the treatment of neoplastic diseases, in particular cancer, as described herein. Compositions may be formulated for non-parenteral administration, such as nasal, buccal, rectal, pulmonary, vaginal, sublingual, topical, transdermal, ophthalmic, or, especially, for oral administration, e.g. in the form of oral solid dosage forms, e.g. granules, pellets, powders, tablets, film or sugar-coated tablets, effervescent tablets, hard and soft gelatin or hydroxypropylmethylcellulose (HPMC) capsules, coated as applicable, orally disintegrating tablets, oral solutions, lipid emulsions or suspensions, or for parenteral administration, such as intravenous, intramuscular, or subcutaneous, intrathecal, intradermal or epidural administration, to mammals, especially humans, e.g. in the form of solutions, lipid emulsions or suspensions containing microparticles or nanoparticles. The compositions may comprise the active ingredient alone or, preferably, together with a pharmaceutically acceptable carrier.

The compounds of formula (I) or pharmaceutically acceptable salts thereof can be processed with pharmaceutically inert, inorganic or organic excipients for the production of oral solid dosage forms, e.g. granules, pellets, powders, tablets, film or sugar coated tablets, effervescent tablets, hard gelatin or HPMC capsules or orally disintegrating tablets. Fillers e.g. lactose, cellulose, mannitol, sorbitol, calcium phosphate, starch or derivatives thereof, binders e.g. cellulose, starch, polyvinylpyrrolidone, or derivatives thereof, glidants e.g. talcum, stearic acid or its salts, flowing agents e.g. fumed silica, can be used as such excipients for formulating and manufacturing of oral solid dosage forms, such as granules, pellets, powders, tablets, film or sugar-coated tablets, effervescent tablets, hard gelatin or HPMC capsules, or orally disintegrating tablets. Suitable excipients for soft gelatin capsules are e.g. vegetable oils, waxes, fats, semisolid and liquid polyols etc.

Suitable excipients for the manufacture of oral solutions, lipid emulsions or suspensions are e.g. water, alcohols, polyols, saccharose, invert sugar, glucose etc. Suitable excipients for parenteral formulations are e.g. water, alcohols, polyols, glycerol, vegetable oils, lecithin, surfactants etc. Moreover, the pharmaceutical preparations can contain preservatives, solubilizers (e.g. cyclodextrin), stabilizers, wetting agents, emulsifiers, sweeteners, colorants, flavorants, salts for varying the osmotic pressure, buffers, masking agents or antioxidants. They can also contain other therapeutically valuable substances. Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor® EL or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. For intravenous injection of strongly lipophilic molecules it can be advantageous to include solubilizers in the formulation, for example surfactants, polymeric surfactants, polymers, complexing agents and/or co-solvents, which may significantly increase the solubility of the compounds in water. Examples of solubilizers include polyethylene glycol, propylene glycol, ethanol, glycerol and cyclodextrins (e.g. sulfobutyl ether-p-cyclodextrins).

The dosage can vary within wide limits and will, of course, be fitted to the individual requirements in each particular case. In general, in the case of oral administration a daily dosage of about 1 to 1000 mg per person of a compound of general formula (I) should be appropriate, although the above lower or upper limit can also be exceeded when necessary.

The compounds of formula (I) can also be used in combination with one or more other pharmaceutically active compounds, which are either effective against the same disease, preferably using a different mode of action, or which reduce or prevent possible undesired side effects of the compounds of formula (I). The combination partners can be administered in such a treatment either simultaneously, e.g. by incorporating them into a single pharmaceutical formulation, or consecutively by administration of two or more different dosage forms, each containing one or more than one of the combination partners.

Compounds of formula (I) according to the invention as described above or pharmaceutically acceptable salts thereof are particularly useful for the treatment of neoplastic diseases such as cancer, in particular when administered in therapeutically effective amounts, e.g. to a subject in need thereof. In some embodiments, the cancer to be treated by the compounds of the present invention may be treatable by inhibiting one or more CLK enzymes. In some embodiments, the cancer to be treated by the compounds of the present invention may be treatable by inhibiting aberrant splicing. The cancer may be driven by aberrant splicing, e.g. the cancer may be a splicing factor mutant cancer. The cancer may be dependent on an oncogenic splice variant.

In some embodiments the compounds of the invention are potent CLK kinase inhibitors and show high kinase selectivity, e.g. as shown by low selectivity score. In some embodiments the compounds of the invention exhibit potent cellular activity, e.g. as shown by anti-proliferative activity and/or cell death. In some embodiments the compounds of the invention potently supress SR protein phosphorylation. In some embodiments the compounds of the invention exhibit long CLK enzyme residence time as shown by target engagement assay, e.g. resulting in potent anti-proliferative activity against cancer cells even after short term exposure. In some embodiments the compounds of the invention show good metabolic stability. In some embodiments the compounds of the invention show good permeability, e.g. as shown by Caco-2 permeability assay. In some embodiments the compounds of the invention show good avoidance of efflux. In some embodiments the compounds of the invention show high oral bioavailability.

In some embodiments the compound of the invention is one which inhibits CLK3 in an in vitro assay with an IC50 of at most 100 nM. In some embodiments the compound of the invention is one which inhibits CLK3 in an in vitro assay with an IC50 of at most 30 nM. The in vitro assay to determine inhibition of CLK3 is described in the Examples below (CLK3 kinase assay).

Examples of neoplastic diseases for treatment by compounds of the invention include, but are not limited to, epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumours, naevi and melanomas, soft tissue tumours and sarcomas, fibromatous neoplasms, myxomatous neoplasms, lipomatous neoplasms, myomatous neoplasms, complex mixed and stromal neoplasms, fibroepithelial neoplasms, synovial-like neoplasms, mesothelial neoplasms, germ cell neoplasms, trophoblastic neoplasms, mesonephromas, blood vessel tumours, lymphatic vessel tumours, osseous and chondromatous neoplasms, giant cell tumours, miscellaneous bone tumours, odontogenic tumours, gliomas, neuroepitheliomatous and neuroendocrine neoplasms, meningiomas, nerve sheath tumours, granular cell tumours and alveolar soft part sarcomas, Hodgkin's and non-Hodgkin's lymphomas, B-cell lymphoma, T-cell lymphoma, hairy-cell lymphoma, Burkitts lymphoma and other lymphoreticular neoplasms, plasma cell tumours, mast cell tumours, immunoproliferative diseases, leukemias, miscellaneous myeloproliferative disorders, lymphoproliferative disorders and myelodysplastic syndromes.

Examples of cancers in terms of the organs and parts of the body affected include, but are not limited to, the breast, cervix, ovaries, colon, rectum (including colon and rectum i.e. colorectal cancer), lung (including small cell lung cancer, non-small cell lung cancer, large cell lung cancer and mesothelioma), endocrine system, bone, adrenal gland, thymus, liver, stomach (gastric cancer), intestine, pancreas, bone marrow, hematological malignancies (such as lymphoma, leukemia, myeloma or lymphoid malignancies), bladder, urinary tract, kidneys, skin, thyroid, brain, head, neck, prostate and testis. Preferably the cancer is selected from the group consisting of breast cancer, prostate cancer, cervical cancer, ovarian cancer, gastric cancer, colorectal cancer, pancreatic cancer, liver cancer, brain cancer, neuroendocrine cancer, lung cancer, kidney cancer, bladder cancer, mesothelioma, hematological malignancies, melanomas and sarcomas.

Further examples of neoplastic diseases for treatment by compounds of the invention are hematological malignancies, e.g. acute myeloid leukemia, myelodysplastic syndromes, chronic myelomonocytic leukemia, chronic lymphocytic leukemia, non-Hodgkin's lymphoma; solid tumors, e.g. mucosal melanoma, uveal melanoma, medulloblastoma, hepatocellular carcinoma, endometrial carcinoma, bladder cancer, cutaneous melanoma, lung adenocarcinoma, pancreatic cancer, breast cancer; cancers with splicing gene mutations, e.g. cancers with splicing factor amplification or fusions, cancers driven by oncogenic transcription factor signaling, cancers driven by oncogenic splice variants, cancers with partial or complete deletion of splicing factors and/or genes implicated in splicing regulation.

The term “treatment” or “treating” as used herein in the context of treating a disease or disorder, pertains generally to treatment and therapy, whether of a human or an animal (e.g. in veterinary applications), in which some desired therapeutic effect is achieved, for example, the inhibition of the progress of the disease or disorder, and includes a reduction in the rate of progress, a halt in the rate of progress, alleviation of symptoms of the disease or disorder, amelioration of the disease or disorder, and cure of the disease or disorder. Treatment as a prophylactic measure (i.e. prophylaxis) is also included. For example, use with patients who have not yet developed the disease or disorder, but who are at risk of developing the disease or disorder, is encompassed by the term “treatment”. For example, treatment includes the prophylaxis of cancer, reducing the incidence of cancer, alleviating the symptoms of cancer, etc.

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

The term “pharmaceutical composition” is defined herein to refer to a solid or liquid formulation containing at least one therapeutic agent to be administered to a subject, e.g. a mammal or human, optionally with one or more pharmaceutically acceptable excipients, in order to prevent or treat a particular disease or condition affecting the mammal.

The term “pharmaceutically acceptable” as used herein refers to items such as compounds and salts thereof, materials, compositions and/or dosage forms, which are, within the scope of sound medical judgment, suitable for contact with the tissues of a warm-blooded animal, e.g., a mammal or human, without excessive toxicity or other complications commensurate with a reasonable benefit/risk ratio.

The compounds of formula (I) can be synthesized by methods given below or by analogous methods, e.g. as shown in Scheme I, and also by using synthetic routes via intermediate (E) as described in the claims as will be clear to the person skilled in the art. The scheme described herein is not intended to present an exhaustive list of methods for preparing the compounds of formula (I); rather, additional techniques of which the skilled chemist is aware may be also used for the compound synthesis.

It is understood by one skilled in the art of organic synthesis that optimum reaction conditions may vary with the particular reactants or solvents used, but such conditions can be determined by routine optimization procedures. In some cases, the order of performing the following reaction schemes, and/or reaction steps, may be varied to facilitate the reaction or to avoid the formation of unwanted side products. In addition, the functionality present at various positions of the molecule must be compatible with the reagents and reactions proposed. Such restrictions to the substituents, which are compatible with the reaction conditions, will be readily apparent to one skilled in the art and alternate methods must then be used. Furthermore in some of the reactions mentioned herein it may be necessary or desirable to protect any sensitive groups in compounds and it will be assumed that such protecting groups (PG) as necessary are in place. Conventional protecting groups may be used in accordance with standard practice, well known in the art (for illustration see Wuts P. G. M, Greene's Protective Groups in Organic Synthesis, 5th Edition, Publisher: John Wiley & Sons, 2014). The protecting groups may be removed at any convenient stage in the synthesis using conventional techniques well known in the art, or they may be removed during a later reaction step or work-up.

In the general sequence of reactions outlined below in Scheme 1, the abbreviations A, T and X and the generic groups, Ra, Rb, R1 and R2 are as defined for formula (I), unless otherwise specified. The generic group E1 is as defined in the claims and is generally a —OH or a methoxy group or a halogen atom, a boronic acid, a boronic ester or —O-L1 and —O-L1 is a leaving group in which L1 is selected from a perfluoroalkylsulfonyl such as triflyl (trifluoromethanesulfonyl) and a sulfonyl such as tosyl (p-toluenesulfonyl) or mesyl (methanesulfonyl). The generic group E2 is a halogen atom, —OH, —NO₂ or —SO₂C1. The generic groups R1′, R2′, R11′, R12′, and R14′ are as defined by the claims for R1, R2, R11, R12 and R14 respectively, with any convenient PG or functional groups, enabling the formation of R1, R2, R11, R12 and R14 respectively and in steps 4 of the synthetic Scheme 1. Other abbreviations used herein are explicitly defined, or are as defined in the experimental section.

The compounds according to the present invention, pharmaceutically acceptable salts, solvates, and hydrates thereof can be prepared according to the general sequence of reactions outlined below in Scheme 1, followed, if necessary, by:
manipulation of substituents to give a new final product. These manipulations may include, but are not limited to, reduction, oxidation, alkylation, acylation, substitution, coupling including transition-metal catalyst coupling and hydrolysis reactions which are commonly known by those skilled in the art;
removing any protecting groups;
forming a pharmaceutically acceptable salt; or
forming a pharmaceutically acceptable solvate or hydrate.

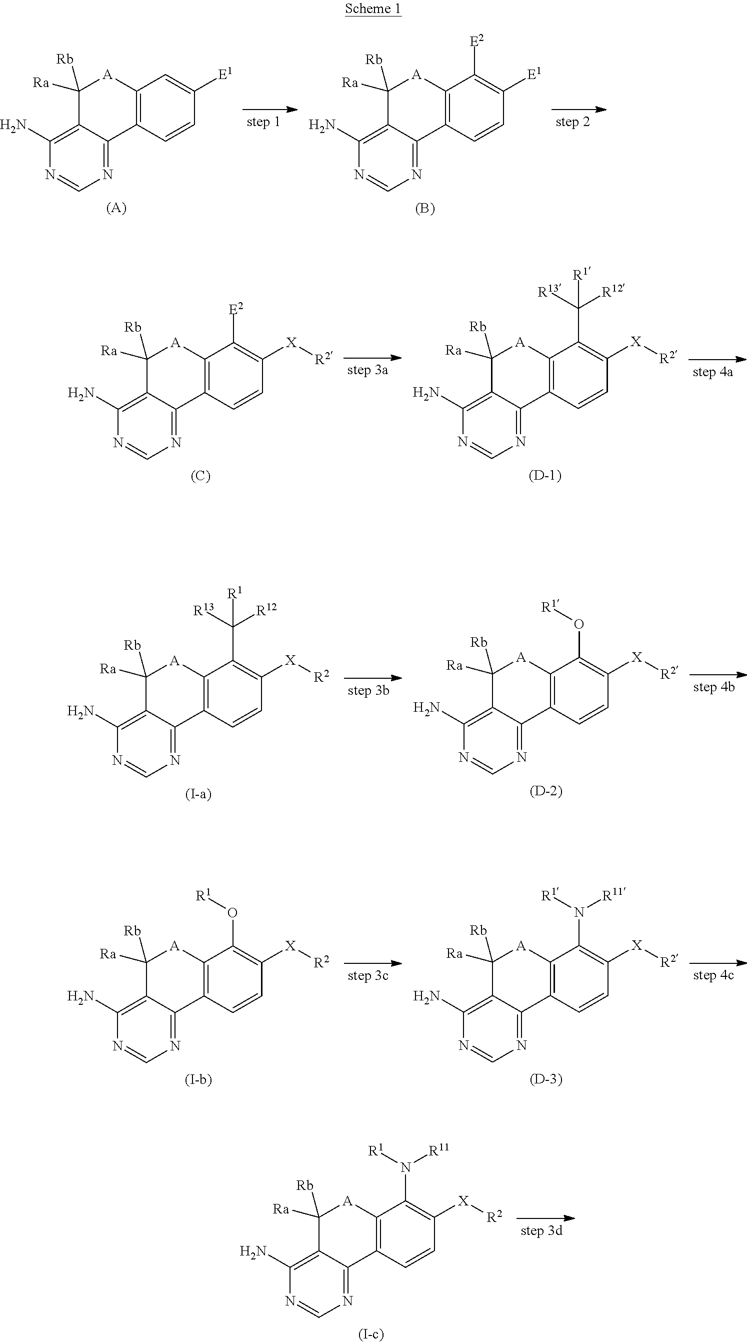

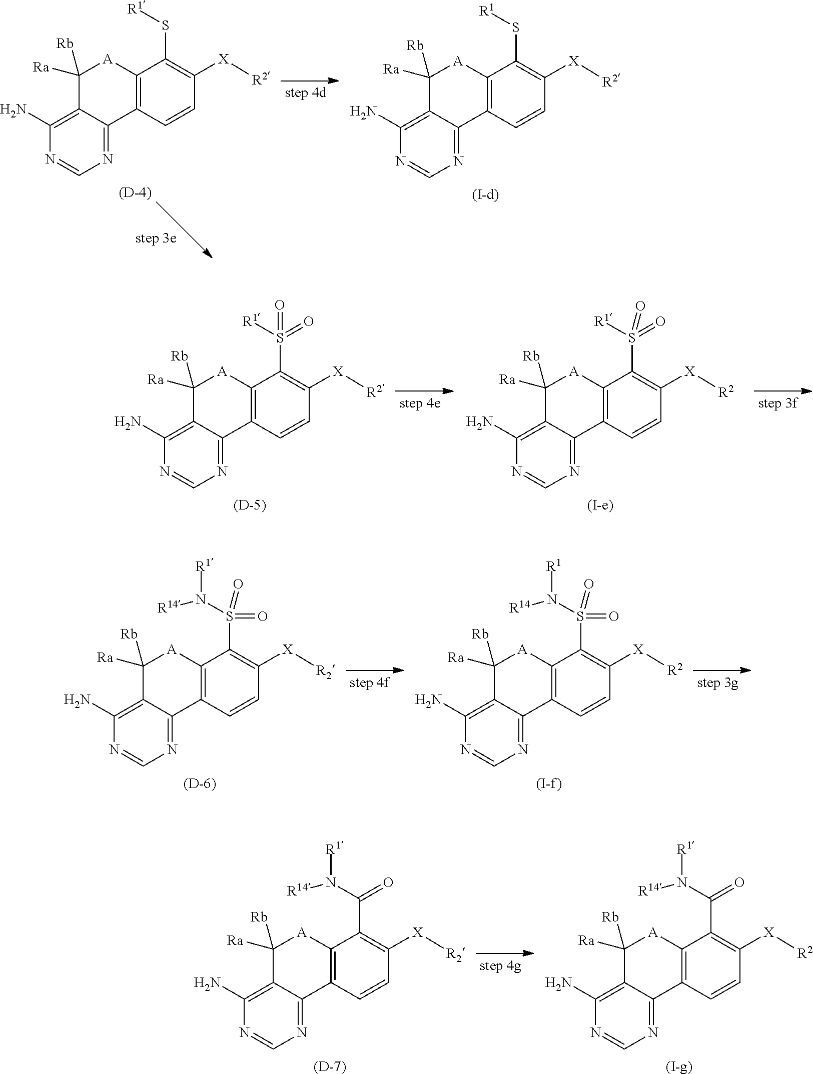

The necessary starting materials for the synthetic methods as described herein, if not commercially available, may be made by procedures which are described in the scientific literature or may be made from commercially available compounds using adaptations of processes reported in the scientific literature. The reader is further referred to e.g. March J., Smith M., Advanced Organic Chemistry, 7th Edition, Publisher: John Wiley & Sons, 2013 for general guidance on reaction conditions and reagents.

Compounds of formula (A) wherein A is —CH₂— are prepared following procedures described in literature (see e.g. Fukuda et al., Bioorg. Med. Chem. Lett. 2018, 28(20), 3333-3337) or by procedures known by a person skilled in the art.

Compounds of formula (A) wherein A is —C(═O)—, can be prepared from compounds of formula (A) wherein A is —CH₂— via oxidation performed at a temperature between 0° C. and 100° C. using an oxidative agent such as potassium permanganate or tert-butyl hydroperoxide in a polar solvent like water, acetone or acetonitrile or a mixture of solvents thereof.

Compounds of formula (A) wherein E1 is —OH, can be prepared from compounds of formula (A) wherein E1 is a methoxy group via demethylation performed at a temperature between 0° C. and 100° C. in presence of a Lewis acid such as aluminum chloride, boron tribromide or boron trifluoride in a solvent like dichloromethane.

Compounds of formula (A) wherein E1 is a halogen atom, can be prepared from compounds of formula (A) wherein E1 is an —O-L1 group via a transition-metal catalyst coupling reaction. Typical catalysts include palladium(II) acetate, tris(dibenzylideneacetone)dipalladium(0) or the like. The reaction is typically run at a temperature from 0° C. to 150° C., more frequently from 80° C. to 120° C. Usually the reaction is performed in the presence of a ligand such as di-tert-butyl-[3,6-dimethoxy-2-(2,4,6-triisopropylphenyl)phenyl]phosphane, di-tert-butyl-[2,3,4,5-tetramethyl-6-(2,4,6-triisopropylphenyl)phenyl]phosphane, 2-(dicyclohexylphosphino)biphenyl, 4,5-bis(diphenylphospheno)-9,9-dimethylxanthene or the like and a base such as sodium tert-butylate, cesium carbonate, potassium carbonate, more frequently cesium carbonate in a large variety of inert solvents such as toluene, tetrahydrofuran, dioxane, 1,2-dichloroethane, N,N-dimethylformamide, dimethylsulfoxide, water and acetonitrile, or a mixture of solvents, more frequently in dioxane. As there are numerous components in transition-metal catalyst coupling reactions such as the particular palladium catalyst, the ligand, additives, solvent, temperature, numerous protocols have been identified. One skilled in the art will be able to identify a satisfactory protocol without undue experimentation.

Alternatively, compounds of formula (A) wherein E1 is a halogen atom can be prepared from compounds of formula (A) wherein E1 is a boronic acid or a boronic ester via a halogenodeboronation reaction. The reaction is typically performed in presence of copper(II) halides in methanol at a temperature ranging from 0° C. to 100° C., more frequently at 90° C.

### Step 1

Compounds of formula (B) wherein E2 is a halogen atom and E1 is —OH or a methoxy group, can be prepared from compounds of formula (A) wherein E1 is —OH or a methoxy group, via an electrophilic aromatic halogenation reaction. The reaction is typically conducted in a mixture of acetic acid and trifluoroacetic acid using NCS for chlorination, NBS for bromination and NIS for iodination.

Compounds of formula (B) wherein E2 is —OH, can be prepared from compounds of formula (B) wherein E2 is a halogen atom, via a transition-metal catalyst coupling reaction, using conditions previously described.

Compounds of formula (B) wherein E2 is —NO₂, can be prepared from compounds of formula (A), via an aromatic nitration reaction. Usually, the nitration reaction is performed using fuming nitric acid in an acidic solvent such as acetic acid or sulfuric acid.

Compounds of formula (B) wherein E2 is —SO₂C1, can be prepared from compounds of formula (A) wherein and E1 is —OH or a methoxy group, via aromatic sulfonylation reaction, usually carried out between −10° C. and 110° C. using chlorosulfonic acid.

### Step 2

Compounds of formula (C) wherein X is —O— can be prepared from compounds of formula (B) wherein E1 is —OH via a substitution reaction with compounds of formula R2′-E3, wherein E3 is a halogen atom or a leaving group —O-L1, wherein L1 is a methanesulfonyl, p-toluenesulfonyl or trifluoromethanesulfonyl group. The reaction is generally performed at a temperature between −20° C. and 100° C. in a dry aprotic solvent like dichloromethane, acetonitrile, N,N-dimethylformamide, dimethyl sulfoxide or tetrahydrofuran without or with an inorganic base such as potassium carbonate or cesium carbonate, or an organic base such as triethylamine or NN-diisopropylethylamine. Formation of the mesylate, tosylate or triflate compound can be achieved by reacting the corresponding alcohol with methanesulfonyl chloride or methanesulfonic anhydride, p-toluenesulfonyl chloride, trifluoromethanesulfonyl chloride or trifluoromethanesulfonic anhydride, respectively, in presence of a base such as triethylamine or the like in a dry aprotic solvent such as pyridine, acetonitrile, tetrahydrofuran or dichloromethane between −30° C. and 80° C.

Alternatively, compounds of formula (C) wherein X is —O— can be prepared from compounds of formula (B) wherein E1 is —OH and an alcohol via Mitsunobu coupling (as reviewed in O. Mitsunobu, Synthesis, Vol. 1, pages 1-28, 1981). The reaction is performed in the presence of diethyl or diisopropyl azodicarboxylate and triphenylphosphine, in a wide range of solvents such as N,N-dimethylformamide, tetrahydrofuran, 1,2-dimethoxyethane or dichloromethane and within a wide range of temperatures, e.g. between −20° C. and 60° C. The reaction might also be performed using polymer-supported triphenylphosphine.

Compounds of formula (C) wherein X is —NH— or —N(C1-C4alkyl)- can be prepared from compounds of formula (B) wherein E1 is a halogen atom or —O-L1 and an amine via a substitution reaction, using conditions previously described.

Alternatively, compounds of formula (C) wherein X is —NH— or —N(C1-C4alkyl)- can be prepared from compounds of formula (B) wherein E1 is a halogen atom or —O-L1 and an amine via a transition-metal catalyst coupling reaction, using conditions previously described.

Alternatively, compounds of formula (C) wherein X is —N(C1-C4alkyl)- can be prepared from compounds of formula (C) wherein X is —NH— and an alkyl halide via a substitution reaction, using conditions previously described.

Alternatively, compounds of formula (C) wherein X is —N(C1-C4alkyl)- can be prepared from compounds of formula (C) wherein X is —NH— and an aldehyde via a reductive amination. The reaction is conducted in a solvent system allowing the removal of the formed water through physical or chemical means (e.g. distillation of the solvent-water azeotrope or presence of drying agents such as molecular sieves, magnesium sulfate or sodium sulfate). Such solvent is typically toluene, n-hexane, tetrahydrofuran, dichloromethane N,N-dimethylformamide, N,N-dimethylacetamide, acetonitrile, 1,2-dichloroethane or mixture of solvents such as methanol-1,2-dichloroethane. The reaction can be catalyzed by traces of acid (usually acetic acid). The intermediate imine is reduced subsequently or simultaneously with a suitable reducing agent (e.g. sodium borohydride, sodium cyanoborohydride, sodiumtriacetoxyborohydride; R. O. and M. K. Hutchins, Comprehensive Organic Synthesis, B. M. Trost, I. Fleming, Eds; Pergamon Press. New York (1991), vol. 8, p. 25-78) or through hydrogenation over a noble metal catalyst such as palladium on activated carbon. The reaction is usually carried out between −10° C. and 110° C., preferably between 0° C. and 60° C. The reaction can also be carried out in one pot. It can also be performed in protic solvents such as methanol or water in presence of a picoline-borane complex (Tetrahedron, 2004, 60, 7899).

Compounds of formula (C) wherein X is —CH₂— or —C(═CH₂)— can be prepared from compounds of formula (B) wherein E1 is a halogen atom or —O-L1 and a boronic acid via Suzuki reaction. The Suzuki reaction is a palladium-catalyzed cross coupling between organoboronic acids and aryl or vinyl halides or triflates. Typical catalysts include palladium(II) acetate, tetrakis(triphenylphosphine)palladium(0), bis(triphenylphosphine)palladium(II) dichloride and [1,1′bis(diphenylphosphino)ferrocene]dichloropalladium(II). The reaction can be carried out in a variety of organic solvents including toluene, tetrahydrofuran, dioxane, 1,2-dichloroethane, N,N-dimethylformamide, dimethylsulfoxide and acetonitrile, aqueous solvents and under biphasic conditions. Reactions are typically run from room temperature to 150° C. Additives such as cesium fluoride, potassium fluoride, potassium hydroxide or sodium ethylate frequently accelerate the coupling. Potassium trifluoroborates and organoboranes or boronate esters may be used in place of boronic acids. Although there are numerous components in the Suzuki reaction such as the particular palladium catalyst, the ligand, additives, solvent, temperature, numerous protocols have been identified. One skilled in the art will be able to identify a satisfactory protocol without undue experimentation.

Alternatively, compounds of formula (C) wherein X is —CH₂— or —C(═CH₂)— can be prepared from compounds of formula (B) wherein E1 is a boronic acid and compounds of formula R2′-E3 wherein E3 is a halogen or a triflate via a Suzuki reaction, using conditions previously described.

Compounds of formula (C) wherein X is —C(═O)— or —CN can be prepared from compounds of formula (B) wherein E1 is a halogen atom via a transition-metal catalyst coupling reaction, using conditions previously described.

### Step 3a

Compounds of formula (D-1) wherein R12′ and R13 are as defined by the claims for R12 and R13 with any convenient PG or functional groups, enabling the formation of R12 later in the synthesis, can be prepared from compounds of formula (C) wherein E2 is a halogen atom via Suzuki reaction, using conditions previously described.

### Step 3b

Compounds of formula (D-2) wherein R1′ is not —H can be prepared from compounds of formula (C) wherein E2 is —OH via substitution reaction, using conditions previously described.

### Step 3c

Compounds of formula (D-3) wherein R1′ and R11′ are —H can be prepared from compounds of formula (C) wherein E2 is —NO₂ via reduction of the nitro group. Typical reducing agents which can be used for such reaction are an alkali metal hydride such as lithium aluminum hydride or sodium borohydride in presence of cobalt(II) chloride or nickel(II) chloride, or a metal such as iron or zinc in acidic medium such as hydrochloric acid or acetic acid. Alternatively, the nitro group can be reduced to the amine by hydrogenation over a noble metal catalyst such as palladium on activated carbon, Raney nickel or platinum oxide. The catalytic hydrogenation reaction can be carried out in a solvent such as ethanol, methanol or ethyl acetate at ambient temperature. In addition further reagents such as aluminum amalgam or ferrous sulphate may also be used for the nitro group reduction.

Compounds of formula (D-3) wherein R1′ is as defined by the claims for R1 with any convenient PG or functional groups, enabling the formation of R11ater in the synthesis, and R11′ is —H can be prepared from compounds of formula (D-3) wherein R1′ and R11′ are —H via reductive amination, substitution or coupling reactions.

In case of amide coupling reactions, the reaction occurs with a carboxylic acid derivative in the presence of an activating agent such as N,N′-dicyclohexylcarbodiimide or N-(3-dimethylaminopropyl)-N′-ethylcarbodiimide hydrochloride, with the optional addition of 1-hydroxybenzotriazole. Other suitable coupling agents may be used such as O-(7-azabenzotriazol-1-yl)-N,N,N′,N′-tetramethyluronium hexafluorophosphate, 2-ethoxy-1-ethoxycarbonyl-1,2-dihydroquinoline, carbonyldiimidazole or diethylphosphorylcyanide. Optionally, a base like triethylamine, N,N-diisopropylethylamine or pyridine can be added to perform the coupling. The amide coupling is conducted at a temperature between −20° C. and 100° C., in an inert solvent, preferably a dry aprotic solvent like dichloromethane, acetonitrile, N,N-dimethylformamide and chloroform. Alternatively, the carboxylic acid can be activated by conversion into its corresponding acid chloride (by reaction with oxalyl chloride or thionyl chloride) or its corresponding activated ester, such as the N-hydroxysuccinimidyl ester (Org. Process Res. & Dev., 2002, 863) or the benzothiazolyl thioester (J. Antibiotics, 2000, 1071). The generated activated entity can react at a temperature between −20° C. and 100° C. with a compound of formula (D-3) wherein R1′ and R11′ are —H, in an aprotic solvent like dichloromethane, chloroform, acetonitrile, N,N-dimethylformamide and tetrahydrofuran. Optionally, a base like triethylamine, N,N-diisopropylethylamine, pyridine, sodium hydroxide, sodium carbonate or potassium carbonate can be added to perform the coupling.

Compounds of formula (D-3) wherein R1′ and R11′ are as defined by the claims for R1 and R11 with any convenient PG or functional groups, enabling the formation of R1 and R11 later in the synthesis, can be prepared from compounds of formula (D-3) wherein R1′ and R11′ are —H via reductive amination, substitution or coupling reactions.

Additionally, compounds of formula (D-3) wherein R1′ is —H and R11′ is as defined by the claims for R11 with any convenient PG or functional groups, enabling the formation of R11 later in the synthesis, can react with sulfonyl chlorides. Further removal of any PG or modification of functional groups can lead to the preparation of compounds of formula (I) wherein T is —N(R11)-S(O)₂—.

### Step 3d

Compounds of formula (D-4) can be prepared from compounds of formula (C) wherein E2 is a halogen atom via substitution or transition-metal catalyst coupling reaction, using conditions previously described. Compound of formula (D-4) wherein R1′ is not —H can be prepared from compounds of formula (D-4) wherein R1′ is —H, via a substitution reaction.

### Step 3e

In compounds of formula (D-5), R1′ is not —H. Compounds of formula (D-5) can be prepared from compounds of formula (D-4) wherein R1′ is not —H, via oxidation reaction performed at a temperature between 0° C. and 100° C. using oxidative agents such as hydrogen peroxide, potassium permanganate, sodium perborate, potassium hydrogen persulfate in polar solvents such as water, acetone, acetonitrile, dichloromethane or ethanol. Depending on the oxidation conditions also the corresponding sulfoxide analogues of (D-5) and (I-e) can be prepared.

### Step 3f

Compounds of formula (D-6) wherein R1′ and R14′ are as defined by the claims for R1 and R14 with any convenient PG or functional groups enabling the formation of R1 and R14 can be prepared from compounds of formula (C) wherein E2 is —SO₂C1 and an amine via coupling reaction.

### Step 3g

Compounds of formula (D-7) wherein R1′ and R14′ are as defined by the claims for R1 and R14 with any convenient PG or functional groups enabling the formation of R1 and R14 can be prepared from compounds of formula (C) wherein E2 is a halogen atom via aryl carbonylation reaction and followed by a coupling reaction with an amine, using conditions previously described.

### Step 4a-g

Compounds of formula (I-a-g) can be prepared from compounds of formula (D-1-7) respectively after removal of any protecting groups or further modification of the substituents R1′, R2′, R11′, R12′ and R14′.

All aspects and embodiments of the invention described herein may be combined in any combination where possible.

A number of publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Particular embodiments of the invention are described in the following Examples, which serve to illustrate the invention in more detail and should not be construed as limiting the invention in any way.

### BRIEF DESCRIPTION OF THE FIGURES

FIG. 1: FIG. 1 provides a representative Western blot showing reduction of phosphorylated-SRSF6 (P—SRSF6) in MDA-MB 468 cells by compounds of the invention. GAPDH was probed for by Western blotting to control for equal protein loading and for normalization of samples. The numbers below the blots show percent inhibition of the P—SRSF6 signal at 100 nM (A) or 10 nM (B) compared to DMSO control.

FIG. 2: FIG. 2 shows tumor volume (TVol) and body weight (BW) changes of female NOD/SCID mice treated with vehicle or 45 mg/kg of Example 13 BID*BIW on days 1-10 and 50 mg/kg of Example 13 BID*BIW on days 11-22. NOD/SCID mice of circa 23 g of weight were inoculated with 1×10⁷ SW480 human colorectal cancer cells in 50% Matrigel™ subcutaneously in a flank. A pair match was performed when tumors reached an average volume of 100-150 mm³ to distribute animals into treatment group and vehicle control (n=8/group). Tumor volumes (expressed in mm³) were measured twice weekly using a caliper on days 1, 4, 8, 11, 15, 18, 22 and 23 of the treatment period. (A) The average TVol (±standard error of mean (SEM)) of each treatment group are shown. Volumes of the tumors treated with 45 mg/kg of Example 13 BID*BIW are consistently smaller during the treatment period compared with the tumor volumes of the vehicle-treated group. (B) Changes in BW in percentage (expressed in g, ±SEM) on days 1, 4, 8, 11, 15, 18, 22 and 23 of the treatment period. (C) Changes in vehicle- and Example 13-treated TVol expressed in mm³. TVol ratio of Example 13-treated vs vehicle control (T/C) is indicated.

### PREPARATION OF EXAMPLES

All reagents and solvents are generally used as received from the commercial supplier; reactions are routinely performed with anhydrous solvents in well-dried glassware under an argon or nitrogen atmosphere, unless otherwise specified;
evaporations are carried out by rotary evaporation under reduced pressure and work-up procedures are carried out after removal of residual solids by filtration;
all temperatures are given in degree Celsius (° C.) and are approximate temperatures; unless otherwise noted, operations are carried out at room temperature (rt), that is typically in the range 18° C.-25° C.; column chromatography (by the flash procedure) is used to purify compounds;
classical flash chromatography is often replaced by automated systems. This does not change the separation process per se. A person skilled in the art will be able to replace a classical flash chromatography process by an automated one, and vice versa. Typical automated systems can be used, as they are provided by Buchi or Isco (combiflash) for instance;
reaction mixture can often be separated by preparative HPLC using e.g. water and acetonitrile as system of eluents, unless otherwise stated. A person skilled in the art will find suitable conditions for each separation; the compounds are isolated after purification as a parent compound or in a form of the corresponding trifluoroacetic acid (TFA) salt or the respective formic acid salt;
reactions, which required higher temperature, are usually performed using classical heating instruments; but can also be performed using microwave apparatus (CEM Explorer) at a power of 250 W, unless otherwise noted;
hydrogenation or hydrogenolysis reactions can be performed using hydrogen gas in balloon or using Parr-apparatus system or other suitable hydrogenation equipment;
concentration of solutions and drying of solids are performed under reduced pressure unless otherwise stated;
in general, the course of reactions is followed by TLC, HPLC, or LC/MS and reaction times are given for illustration only; yields are given for illustration only and are not necessarily the maximum attainable; the structure and purity of the final products of the invention are generally confirmed by NMR spectroscopy, HPLC and mass spectral techniques.

Proton NMR spectra are recorded on a Brucker 400 MHz spectrometer. Chemical shifts (6) are reported in ppm relative to Me₄Si or the solvent peak as internal standard, and NMR coupling constants (J values) are in Hertz (Hz). Each peak is denoted as a broad singlet (br), singlet (s), doublet (d), triplet (t), quadruplet (q), doublet of doublets (dd), triplet of doublets (td) or multiplet (m).

HPLC of the final products are generated using a Dionex Ultimate 3000 instrument coupled with Dionex MSQ ESI mode and the following conditions:

| Mobile Phase A: | Water with 0.1% Formic acid | |
|---|---|---|
| Mobile Phase B: | Acetonitrile with 0.1% Formic acid | |
| Column: | YMC triart C18 5 μm 100 mm × 4.6 mm | |
| Column Temperature: | 25° C. | |
| Detection: | UV 250 nm | |
| Injection: | 2 μL of 10 mM sample DMSO solution | |
| Flow: | 1.6 mL/min | |
| | Time (min) | % Mobile Phase B |
| Gradient | 0 | 5 |
| | 8 | 95 |
| | 10 | 95 |
| | 10.1 | 5 equilibration |
| | 13 | 5 equilibration |

Mass spectra are generated using a q-Tof Ultima (Waters AG or Thermo Scientific MSQ Plus) mass spectrometer in the positive or negative ESI mode. The system is equipped with the standard Lockspray interface;
each intermediate is purified to the standard required for the subsequent stage and is characterized in sufficient detail to confirm that the assigned structure is correct;
analytical and preparative HPLC on non-chiral phases are performed using RP-C18 based columns; the following abbreviations may be used (reference can also be made to The Journal of Organic Chemistry Guidelines for Authors, for a comprehensive list of standard abbreviations and acronyms):
Ac₂O Acetic anhydride
AcOH Acetic acid
ACN Acetonitrile
Boc tert-Butoxy carbonyl group
t-BuBrettPhos Di-tert-butyl(2′,4′,6′-triisopropyl-3,6-dimethoxy-[1,1′-biphenyl]-2-yl)phosphine
t-BuOH tert-Butanol
t-BuOK Potassium tert-butoxide
CAN Diammonium cerium (IV) nitrate
CAS compound having Chemical Abstracts Services registry number
CDCl₃ Deuterated chloroform
Cu(OAc)₂ Cupric acetate
DABCO 1,4-Diazabicyclo(2,2,2)octane
DCE 1,2-Dichloroethane
DCM Dichloromethane
Diox 1,4-Dioxane
DIPEA N,N-Diisopropylethylamine
DMAP 4-Dimethylaminopyridine
DMF N,N-Dimethylformamide
DMSO Dimethyl sulfoxide
DMSO-d₆ Deuterated dimethyl sulfoxide
EA Ethyl acetate
EDCI 1-(3-Dimethylaminopropyl)-3-ethylcarbodiimide hydrochloride
ELSD Evaporative light scattering detection
EtOH Ethanol
Ex. Example
HATU 1-[Bis(dimethylamino)methylene]-1H-1,2,3-triazolo[4,5-b]pyridinium 3-oxide hexafluorophosphate
HPLC High performance liquid chromatography
LC/MS Liquid chromatography coupled to mass spectroscopy
Mel Methyl iodide
MeOH Methanol
Me₄Si Tetramethylsilane
MMNO 4-Methylmorpholine N-oxide
MS Mass spectrometry
Ms Methanesulfonyl
MsCl Methanesulfonyl chloride
MW Microwave
NaBH₃CN Sodium cyanoborohydride
NaBH(OAc)₃ Sodium triacetoxyborohydride
NaOAc Sodium acetate
NBS N-Bromosuccinimide
NCS N-Chlorosuccinimide
NIS N-Iodosuccinimide
NMM 4-Methylmorpholine
NMR Nuclear magnetic resonance
Ns Nosyl (4-nitrobenzenesulfonyl)
NsCl 4-Nitrobenzenesulfonyl chloride
Pd/C Palladium on activated carbon
Pd₂(dba)₃ Tris(dibenzylideneacetone)dipalladium(0)
Pd(dppf)Cl₂ [1,1′-Bis(diphenylphosphino)ferrocene]palladium(II) dichloride
PE Petroleum Ether
PMB para-Methoxybenzyl
PMB-Cl para-Methoxybenzyl chloride
Py Pyridine
rt Room temperature
SiHEt₃ Triethylsilane
TBAF Tetrabutylammonium fluoride
TBAI Tetrabutylammonium iodide
TBDPS tert-Butyldiphenylsilyl
TBDPSCl tert-Butyldiphenylsilyl chloride
TBHP tert-Butyl hydroperoxide
TBS tert-Butyldimethylsilyl
TBSCl tert-Butyldimethylsilyl chloride
TEA Triethylamine
TFA Trifluoroacetic acid
Tf Triflate
TfOH Trifluoromethanesulfonic acid
THF Tetrahydrofuran
TMS Trimethylsilyl
Ts Tosyl (4-toluenesulfonyl)
TsCE 4-Toluenesulfonyl chloride
v/v volume ratio
Xant-Phos 4,5-Bis(diphenylphosphino)-9,9-dimethylxanthene

The following Examples refer to the compounds of formula (I) as indicated in Table 1.

The Examples listed in the following table can be prepared using procedures described above, and detailed synthesis methodology is described in detail below. The Example numbers used in the leftmost column are used in the application text for identifying the respective compounds.

TABLE 1

| | | **Reference | | HPLC | |
|---|---|---|---|---|---|
| | | for | ¹H-NMR | Retention | MS m/z |
| Ex. | Formula | Preparation | (400 MHz) δ ppm | time (min) | (+ESI) |
| 1 | 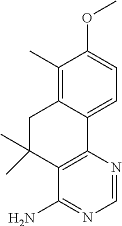 | — | DMSO-d₆ + D₂O: 8.28 (s, 1H), 7.89 (d, J = 8.8 Hz, 1H), 6.94 (d, J = 8.8 Hz, 1H), 3.82 (s, 3H), 2.75 (s, 2H), 2.13 (s, 3H), 1.27 (s, 6H) | 3.99 | 270.1 [M + H]⁺ |
| 2 | 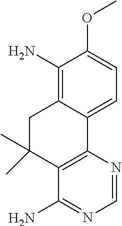 | — | DMSO-d₆ + D₂O: 8.53 (s, 1H), 7.19 (d, J = 8.4 Hz, 1H), 6.96 (d, J = 8.4 Hz, 1H), 3.87 (s, 3H), 2.72 (s, 2H), 1.29 (s, 6H) | 2.83 | 271.1 [M + H]⁺ |
| 3 | 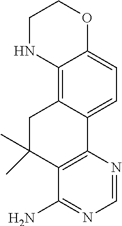 [SMILES: CC1(C)Cc2c(ccc3c2NCCO3)-c2ncnc(N)c21] | — | DMSO-d₆ + D₂O: 8.52 (s, 1H), 7.10 (d, J = 8.4 Hz, 1H), 6.79 (d, J = 8.4 Hz, 1H), 4.18 (t, J = 4.4 Hz, 2H), 3.33 (t, J = 4.4 Hz, 2H), 2.66 (s, 2H), 1.29 (s, 6H) | 3.08 | 283.2 [M + H]⁺ |
| 4 | 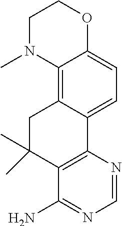 [SMILES: CN1CCOc2ccc3c(c21)CC(C)(C)c1c(N)ncnc1-3] | — | DMSO-d₆ + D₂O: 8.54 (s, 1H), 7.42 (d, J = 8.4 Hz, 1H), 6.92 (d, J = 8.4 Hz, 1H), 4.20 (t, J = 4.4 Hz, 2H), 3.09 (t, J = 4.4 Hz, 2H), 2.84 (s, 2H), 2.63 (s, 3H), 1.28 (s, 6H) | 3.45 | 297.2 [M + H]⁺ |
| 5 | 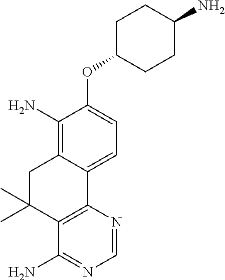 [SMILES: CC1(C)Cc2c(ccc(O[C@H]3CC[C@H](N)CC3)c2N)-c2ncnc(N)c21] | — | DMSO-d₆ + D₂O: 8.54 (s, 1H), 7.18 (d, J = 8.8 Hz, 1H), 7.04 (d, J = 8.8 Hz, 1H), 4.39 (m, 1H), 3.09 (m, 1H), 2.72 (s, 2H), 2.12 (m, 2H), 2.00 (m, 2H), 1.50 (m, 4H), 1.29 (s, 6H) | 2.13 | 354.3 [M + H]⁺ |
| 6 | 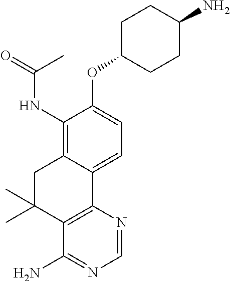 [SMILES: CC(=O)Nc1c(O[C@H]2CC[C@H](N)CC2)ccc2c1CC(C)(C)c1c(N)ncnc1-2] | — | DMSO-d₆ + D₂O: 8.52 (s, 1H), 7.77 (d, J = 8.8 Hz, 1H), 7.21 (d, J = 8.8 Hz, 1H), 4.39 (m, 1H), 3.09 (m, 1H), 2.62 (s, 2H), 2.07 (m, 2H), 2.04 (s, 3H), 1.97 (m, 2H), 1.48 (m, 4H), 1.26 (s, 6H) | 1.89 | 396.2 [M + H]⁺ |
| 7 | 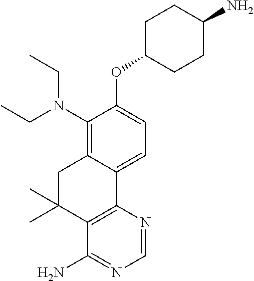 [SMILES: CCN(CC)c1c(O[C@H]2CC[C@H](N)CC2)ccc2c1CC(C)(C)c1c(N)ncnc1-2] | — | DMSO-d₆ + D₂O: 8.54 (s, 1H), 7.71 (d, J = 8.8 Hz, 1H), 7.20 (d, J = 8.8 Hz, 1H), 4.49 (m, 1H), 3.20 (m, 2H), 3.11 (m, 1H), 2.98 (m, 4H), 2.14 (m, 2H), 1.98 (m, 2H), 1.50 (m, 4H), 1.26 (s, 6H), 0.84 (t, J = 7.2 Hz, 6H) | 2.17 | 410.3 [M + H]⁺ |
| 8 | 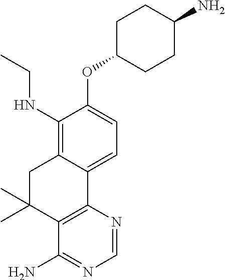 [SMILES: CCNc1c(O[C@H]2CC[C@H](N)CC2)ccc2c1CC(C)(C)c1c(N)ncnc1-2] | — | DMSO-d₆ + D₂O: 8.50 (s, 1H), 7.68 (d, J = 8.8 Hz, 1H), 7.25 (d, J = 8.8 Hz, 1H), 4.49 (m, 1H), 3.12 (m, 3H), 2.86 (s, 2H), 2.13 (m, 2H), 2.00 (m, 2H), 1.52 (m, 4H), 1.28 (s, 6H), 1.10 (t, J = 7.2 Hz, 3H) | 1.91 | 382.2 [M + H]⁺ |
| 9 | 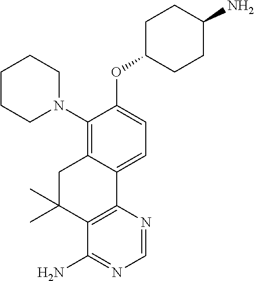 [SMILES: CC1(C)Cc2c(ccc(O[C@H]3CC[C@H](N)CC3)c2N2CCCCC2)-c2ncnc(N)c21] | — | DMSO-d₆ + D₂O: 8.20 (s, 1H), 7.79 (d, J = 8.8 Hz, 1H), 6.96 (d, J = 8.8 Hz, 1H), 4.34 (m, 1H), 3.20 (m, 2H), 3.10 (m, 1H), 2.87 (s, 2H), 2.67 (m, 2H), 2.13 (m, 2H), 1.99 (m, 2H), 1.75 (m, 1H), 1.52 (m, 9H), 1.24 (s, 6H) | 3.52 | 422.4 [M + H]⁺ |
| 10 | 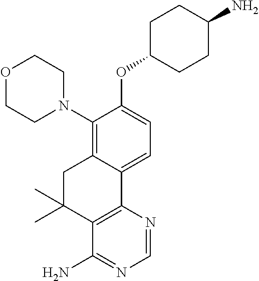 [SMILES: CC1(C)Cc2c(ccc(O[C@H]3CC[C@H](N)CC3)c2N2CCOCC2)-c2ncnc(N)c21] | Example 9 using CAS 7456-83-9 | DMSO-d₆ + D₂O: 8.20 (s, 1H), 7.83 (d, J = 8.8 Hz, 1H), 6.99 (d, J = 8.8 Hz, 1H), 4.37 (m, 1H), 3.73 (m, 3H), 3.61 (m, 3H), 3.44 (m, 2H), 3.09 (m, 1H), 2.91 (s, 2H), 2.14 (m, 2H), 1.99 (m, 2H), 1.50 (m, 4H), 1.24 (s, 6H) | 2.85 | 424.4 [M + H]⁺ |
| 11 | 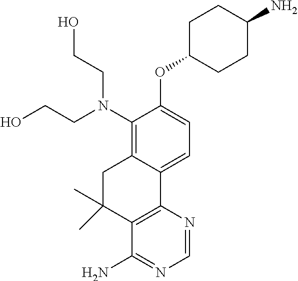 [SMILES: CC1(C)Cc2c(ccc(O[C@H]3CC[C@H](N)CC3)c2N(CCO)CCO)-c2ncnc(N)c21] | — | DMSO-d₆ + D₂O: 8.55 (s, 1H), 7.73 (d, J = 8.8 Hz, 1H), 7.20 (d, J = 8.8 Hz, 1H), 4.49 ( m, 1H), 3.32 (m, 4H), 3.09 (m, 5H), 3.02 (s, 2H), 2.16 (m, 2H), 2.00 (m, 2H), 1.53 (m, 4H), 1.27 (s, 6H) | 2.06 | 442.4 [M + H]⁺ |
| 12 | 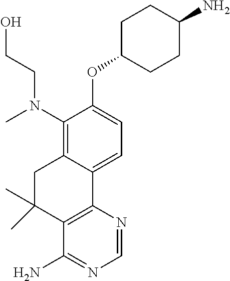 [SMILES: CN(CCO)c1c(O[C@H]2CC[C@H](N)CC2)ccc2c1CC(C)(C)c1c(N)ncnc1-2] | Examples 8 and 11 using CAS 50-00-0 | DMSO-d₆ + D₂O: 8.55 (s, 1H), 7.78 (d, J = 8.8 Hz, 1H), 7.24 (d, J = 8.8 Hz, 1H), 4.51 (m, 1H), 3.36 (m, 2H), 3.11 (m, 4H), 2.94 (m, 1H), 2.74 (s, 3H), 2.16 (m, 2H), 2.00 (m, 2H), 1.54 (m, 4H), 1.27 (s, 6H) | 2.24 | 412.4 [M + H]⁺ |
| 13 | 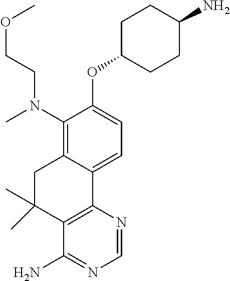 [SMILES: COCCN(C)c1c(O[C@H]2CC[C@H](N)CC2)ccc2c1CC(C)(C)c1c(N)ncnc1-2] | Example 8 using CAS 10312-83-1 and 50-00-0 | DMSO-d₆ + D₂O: 8.43 (s, 1H), 7.74 (d, J = 8.8 Hz, 1H), 7.12 (d, J = 8.8 Hz, 1H), 4.44 (m, 1H), 3.28 (t, J = 5.6 Hz, 2H), 3.17 (s, 3H), 3.10 (m, 4H), 2.84 (m, 1H), 2.65 (s, 3H), 2.15 (m, 2H), 1.99 (m, 2H), 1.51 (m, 4H), 1.29 (s, 3H), 1.22 (s, 3H) | 2.72 | 426.3 [M + H]⁺ |
| 14 | 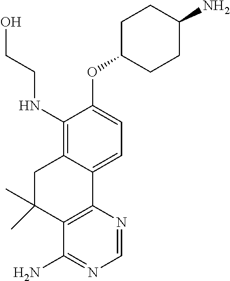 [SMILES: CC1(C)Cc2c(ccc(O[C@H]3CC[C@H](N)CC3)c2NCCO)-c2ncnc(N)c21] | Examples 7 and 11 using CAS 102191-92-4 | DMSO-d₆ + D₂O: 8.39 (s, 1H), 7.61 (d, J = 8.8 Hz, 1H), 6.94 (d, J = 8.8 Hz, 1H), 4.30 (m, 1H), 3.45 (m, 2H), 3.02 (m, 1H), 2.94 (m, 2H), 2.71 (s, 2H), 2.10 (m, 2H), 1.96 (m, 2H), 1.47 (m, 4H), 1.25 (s, 6H) | 1.88 | 398.3 [M + H]⁺ |
| 15 | 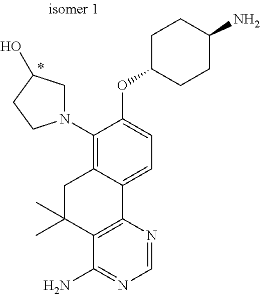 [SMILES: CC1(C)Cc2c(ccc(O[C@H]3CC[C@H](N)CC3)c2N2CCC(O)C2)-c2ncnc(N)c21] | | DMSO-d₆ + D₂O: 8.55 (s, 1H), 7.72 (d, J = 8.8 Hz, 1H), 7.22 (d, J = 8.8 Hz, 1H), 4.48 (m, 1H), 4.40 (m, 1H), 3.26 (m, 2H), 3.09 (m, 2H), 2.96 (m, 3H), 2.12 (m, 3H), 1.99 (m, 2H), 1.82 (m, 1H), 1.52 (m, 4H), 1.26 (s, 6H) | 2.39 | 424.4 [M + H]⁺ |
| 16 | 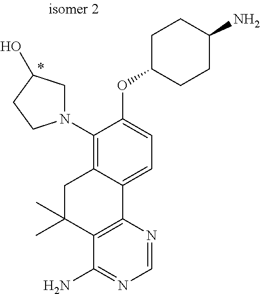 [SMILES: CC1(C)Cc2c(ccc(O[C@H]3CC[C@H](N)CC3)c2N2CCC(O)C2)-c2ncnc(N)c21] | — | DMSO-d₆ + D₂O: 8.56 (s, 1H), 7.74 (d, J = 8.8 Hz, 1H), 7.23 (d, J = 8.8 Hz, 1H), 4.48 (m, 1H), 4.39 (m, 1H), 3.26 (m, 2H), 3.08 (m, 2H), 2.96 (m, 3H), 2.12 (m, 3H), 1.99 (m, 2H), 1.82 (m, 1H), 1.53 (m, 4H), 1.26 (s, 6H) | 2.38 | 424.4 [M + H]⁺ |
| 17 | 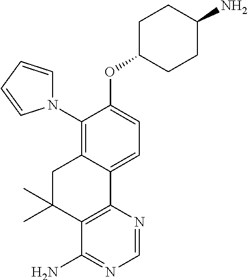 [SMILES: CC1(C)Cc2c(ccc(O[C@H]3CC[C@H](N)CC3)c2-n2cccc2)-c2ncnc(N)c21] | — | DMSO-d₆ + D₂O: 8.26 (s, 1H), 8.07 (d, J = 8.8 Hz, 1H), 7.15 (d, J = 8.8 Hz, 1H), 6.68 (t, J = 2.0 Hz, 2H), 6.19 (t, J = 2.0 Hz, 2H), 4.22 (m, 1H), 2.55 (m, 1H), 2.34 (s, 2H), 1.90 (m, 2H), 1.70 (m, 2H), 1.16 (m, 10H) | 3.11 | 404.3 [M + H]⁺ |
| 18 | 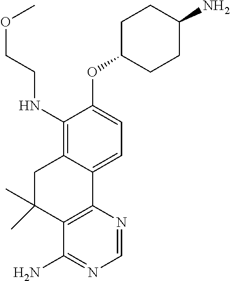 [SMILES: COCCNc1c(O[C@H]2CC[C@H](N)CC2)ccc2c1CC(C)(C)c1c(N)ncnc1-2] | Example 8 using CAS 10312-83-1 | DMSO-d₆ + D₂O: 8.56 (s, 1H), 7.59 (d, J = 8.8 Hz, 1H), 7.21 (d, J = 8.8 Hz, 1H), 4.48 (m, 1H), 3.42 (t, J = 5.2 Hz, 2H), 3.25 (s, 3H), 3.16 (t, J = 5.2 Hz, 2H), 3.09 (m, 1H), 2.86 (s, 2H), 2.14 (m, 2H), 2.01 (m, 2H), 1.53 (m, 4H), 1.28 (s, 6H) | 2.30 | 412.3 [M + H]⁺ |
| 19 | 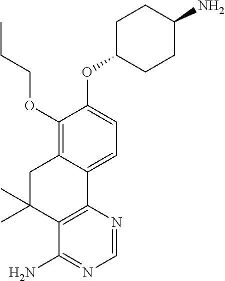 [SMILES: CCCOc1c(O[C@H]2CC[C@H](N)CC2)ccc2c1CC(C)(C)c1c(N)ncnc1-2] | — | DMSO-d₆ + D₂O: 8.18 (s, 1H), 7.15 (d, J = 8.8 Hz, 1H), 7.00 (d, J = 8.8 Hz, 1H), 4.32 (m, 1H), 3.82 (t, J = 6.8 Hz, 2H), 3.06 (m, 1H), 2.73 (s, 2H), 2.11 (m, 2H), 1.97 (m, 2H), 1.66 (qt, J1 = 7.2 Hz, J2 = 6.8 Hz, 2H), 1.46 (m, 4H), 1.24 (s, 6H), 0.96 (t, J = 7.2 Hz 3H) | 3.05 | 397.3 [M + H]⁺ |
| 20 | 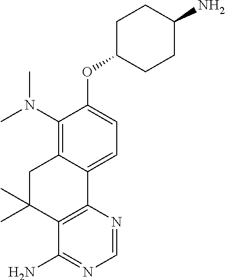 [SMILES: CN(C)c1c(O[C@H]2CC[C@H](N)CC2)ccc2c1CC(C)(C)c1c(N)ncnc1-2] | Example 7 using CAS 50-00-0 | DMSO-d₆ + D₂O: 8.40 (s, 1H), 7.83 (d, J = 8.8 Hz, 1H), 6.98 (d, J = 8.8 Hz, 1H), 4.35 (m, 1H), 3.03 (m, 1H), 2.85 (s, 2H), 2.69 (s, 6H), 2.15 (m, 2H), 1.96 (m, 2H), 1.48 (m, 4H), 1.25 (s, 6H) | 2.30 | 382.3 [M + H]⁺ |
| 21 | 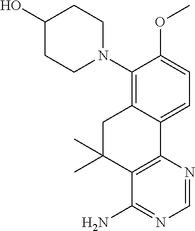 [SMILES: COc1ccc2c(c1N1CCC(O)CC1)CC(C)(C)c1c(N)ncnc1-2] | — | DMSO-d₆ + D₂O: 8.22 (s, 1H), 7.84 (d, J = 8.4 Hz, 1H), 6.90 (d, J = 8.8 Hz, 1H), 3.79 (s, 3H), 3.46 (m, 1H), 3.23 (m, 2H), 2.88 (s, 2H), 2.68 (m, 2H), 1.8 (m, 2H), 1.49 (m, 2H), 1.25 (s, 6H) | 3.29 | 355.3 [M + H] ⁺ |
| 22 | 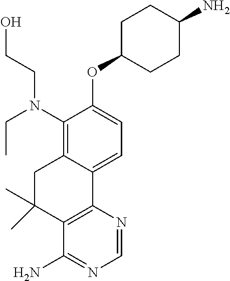 [SMILES: CCN(CCO)c1c(O[C@H]2CC[C@@H](N)CC2)ccc2c1CC(C)(C)c1c(N)ncnc1-2] | — | DMSO-d₆ + D₂O: 8.19 (s, 1H), 7.84 (d, J = 8.8 Hz, 1H), 6.89 (d, J = 8.8 Hz, 1H), 4.62 (m, 1H), 3.35 (m, 2H), 3.04 (m, 7H), 1.98 (m, 2H), 1.70 (m, 6H), 1.24 (s, 6H), 0.84 (t, J = 7.2 Hz, 3H) | 2.34 | 426.5 [M + H]⁺ |
| 23 | 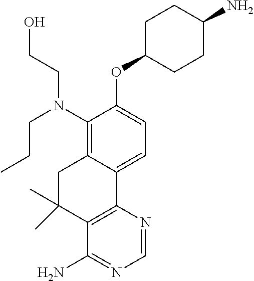 [SMILES: CCCN(CCO)c1c(O[C@H]2CC[C@@H](N)CC2)ccc2c1CC(C)(C)c1c(N)ncnc1-2] | Example 22 using CAS 123-38-6 | DMSO-d₆ + D₂O: 8.21 (s, 1H), 7.85 (d, J = 8.8 Hz, 1H), 6.89 (d, J = 8.8 Hz, 1H), 4.62 (m, 1H), 3.35 (m, 2H), 3.02 (m, 7H), 1.99 (m, 2H), 1.71 (m, 6H), 1.25 (m, 2H), 1.24 (s, 6H), 0.83 (t, J = 7.2 Hz, 3H) | 2.71 | 440.4 [M + H]⁺ |
| 24 | 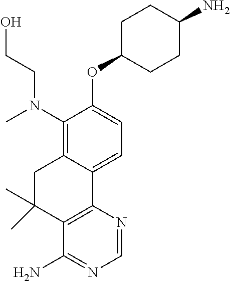 [SMILES: CN(CCO)c1c(O[C@H]2CC[C@@H](N)CC2)ccc2c1CC(C)(C)c1c(N)ncnc1-2] | Example 22 using CAS 50-00-0 | DMSO-d₆ + D₂O: 8.20 (s, 1H), 7.84 (d, J = 8.8 Hz, 1H), 6.89 (d, J = 8.8 Hz, 1H), 4.63 (m, 1H), 3.38 (m, 2H), 3.11 (m, 4H), 2.81 (m, 1H), 2.72 (s, 3H), 2.01 (m, 2H), 1.70 (m, 6H), 1.25 (m, 6H) | 2.34 | 412.3 [M + H]⁺ |
| 25 | 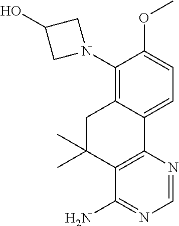 [SMILES: COc1ccc2c(c1N1CC(O)C1)CC(C)(C)c1c(N)ncnc1-2] | — | DMSO-d₆ + D₂O: 8.21 (s, 1H), 7.55 (d, J = 8.4 Hz, 1H), 6.85 (d, J = 8.8 Hz, 1H), 4.32 (m, 1H), 4.26 (m, 2H), 3.77 (m, 2H), 3.74 (s, 3H), 2.66 (s, 2H), 1.22 (s, 6H) | 2.62 | 327.3 [M + H]⁺ |
| 26 | 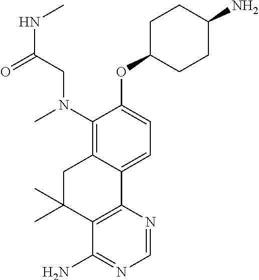 [SMILES: CNC(=O)CN(C)c1c(O[C@H]2CC[C@@H](N)CC2)ccc2c1CC(C)(C)c1c(N)ncnc1-2] | — | DMSO-d₆ + D₂O: 8.21 (s, 1H), 7.86 (d, J = 8.8 Hz, 1H), 6.93 (d, J = 8.8 Hz, 1H), 4.66 (m, 1H), 3.64 (m, 1H), 3.45 (m, 1H), 3.09 (m, 1H), 2.94 (s, 2H), 2.73 (s, 3H), 2.68 (s, 3H), 2.03 (m, 2H), 1.72 (m, 6H), 1.29 (s, 3H), 1.27 (s, 3H) | 2.52 | 439.4 [M + H]⁺ |
| 27 | 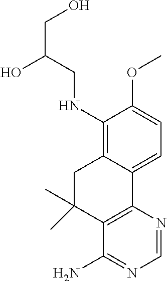 [SMILES: COc1ccc2c(c1NCC(O)CO)CC(C)(C)c1c(N)ncnc1-2] | Example 25 using CAS 556-52-5 | DMSO-d₆ +D₂O: 8.56 (s, 1H), 7.61 (d, J = 8.4 Hz, 1H), 7.15 (d, J = 8.8 Hz, 1H), 3.92 (s, 3H), 3.62 (m, 1H), 3.39 (m, 1H), 3.31 (m, 1H), 3.20 (m, 1H), 2.90 (m, 1H), 2.87 (s, 2H), 1.28 (d, J = 3.2 Hz, 6H) | 1.98 | 345.2 [M + H]⁺ |
| 28 | 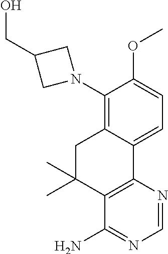 [SMILES: COc1ccc2c(c1N1CC(CO)C1)CC(C)(C)c1c(N)ncnc1-2] | — | DMSO-d₆ + D₂O: 8.20 (s, 1H), 7.53 (d, J = 8.8 Hz, 1H), 6.84 (d, J = 8.4 Hz, 1H), 4.08 (m, 2H), 3.78 (m, 2H), 3.73 (s, 3H), 3.55 (m, 2H), 2.69 (s, 2H), 2.57 (m, 1H), 1.21 (s, 6H) | 2.25 | 341.2 [M + H]⁺ |
| 29 | 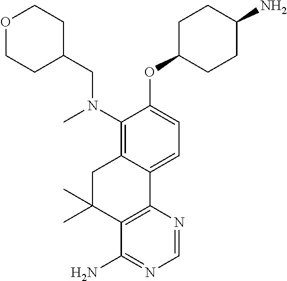 [SMILES: CN(CC1CCOCC1)c1c(O[C@H]2CC[C@@H](N)CC2)ccc2c1CC(C)(C)c1c(N)ncnc1-2] | Examples 5 and 8 using CAS 111300-06-2, 50674-18-8 and 50-00-0 | DMSO-d₆ + D₂O: 8.21 (s, 1H), 7.84 (d, J = 8.8 Hz, 1H), 6.90 (d, J = 8.8 Hz, 1H), 4.63 (m, 1H), 3.80 (m, 2H), 3.20 (m, 2H), 3.10 (m, 1H), 2.80 (m, 4H), 2.68 (s, 3H), 2.00 (m, 2H), 1.70 (m, 9H), 1.29 (s, 3H), 1.20 (s, 3H), 1.14 (m, 2H) | 2.87 | 466.4 [M + H]⁺ |
| 30 | 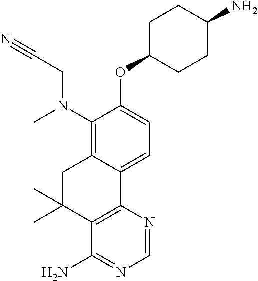 [SMILES: CN(CC#N)c1c(O[C@H]2CC[C@@H](N)CC2)ccc2c1CC(C)(C)c1c(N)ncnc1-2] | Examples 5 and 22 using CAS 111300-06-2, 590-17-0 and 50-00-0 | DMSO-d₆ + D₂O: 8.22 (s, 1H), 7.91 (d, J = 8.8 Hz, 1H), 6.97 (d, J = 8.8 Hz, 1H), 4.67 (m, 1H), 4.15 (m, 2H), 3.08 (m, 1H), 2.87 (s, 2H), 2.80 (s, 3H), 2.04 (m, 2H), 1.71 (m, 6H), 1.27 (s, 6H) | 2.64 | 407.3 [M + H]⁺ |
| 31 | 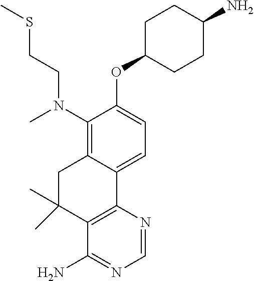 [SMILES: CSCCN(C)c1c(O[C@H]2CC[C@@H](N)CC2)ccc2c1CC(C)(C)c1c(N)ncnc1-2] | Examples 5, 8 and 9 using CAS 111300-06-2, 23328-62-3 and 50-00-0 | DMSO-d₆ + D₂O: 8.21 (s, 1H), 7.85 (d, J = 8.8 Hz, 1H), 6.91 (d, J = 8.8 Hz, 1H), 4.63 (m, 1H), 3.15 (m, 5H), 2.74 (m, 2H), 2.71 (s, 3H), 2.01 (s, 3H), 2.00 (m, 2H), 1.70 (m, 6H), 1.30 (s, 3H), 1.21 (s, 3H) | 3.07 | 442.5 [M + H]⁺ |
| 32 | 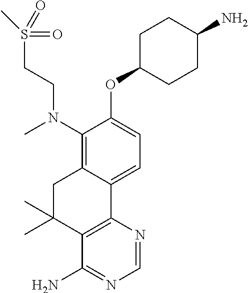 [SMILES: CN(CCS(C)(=O)=O)c1c(O[C@H]2CC[C@@H](N)CC2)ccc2c1CC(C)(C)c1c(N)ncnc1-2] | — | DMSO-d₆ + D₂O: 8.22 (s, 1H), 7.88 (d, J = 8.8 Hz, 1H), 6.94 (d, J = 8.8 Hz, 1H), 4.65 (m, 1H), 3.53 (m, 2H), 3.15 (m, 4H), 2.98 (s, 3H), 2.78 (s, 1H), 2.75 (s, 3H), 2.01 (m, 2H), 1.72 (m, 6H), 1.28 (s, 3H), 1.25 (s, 3H) | 2.60 | 474.4 [M + H]⁺ |
| 33 | 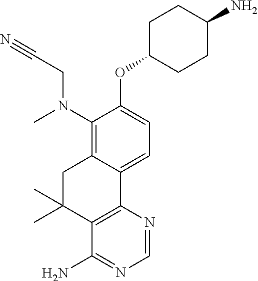 [SMILES: CN(CC#N)c1c(O[C@H]2CC[C@H](N)CC2)ccc2c1CC(C)(C)c1c(N)ncnc1-2] | Examples 5, 8 and 22 using CAS 167081-25-6, 590-17-0 and 50-00-0 | DMSO-d₆ + D₂O: 8.22 (s, 1H), 7.91 (d, J = 8.8 Hz, 1H), 7.05 (d, J = 8.8 Hz, 1H), 4.41 (m, 1H), 4.08 (m, 2H), 3.03 (m, 1H), 2.84 (s, 2H), 2.74 (s, 3H), 2.14 (m, 2H), 1.97 (m, 2H), 1.51 (m, 4H), 1.26 (s, 6H | 2.89 | 407.2 [M + H]⁺ |
| 34 | 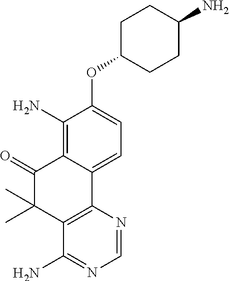 [SMILES: CC1(C)C(=O)c2c(ccc(O[C@H]3CC[C@H](N)CC3)c2N)-c2ncnc(N)c21] | — | DMSO-d₆ + D₂O: 8.30 (s, 1H), 7.82 (d, J = 8.4 Hz, 1H), 7.26 (d, J = 8.4 Hz, 1H), 4.37 (m, 1H), 3.01 (m, 1H), 2.11 (m, 2H), 1.97 (m, 2H), 1.48 (m, 10H) | 2.55 | 368.3 [M + H]⁺ |
| 35 | 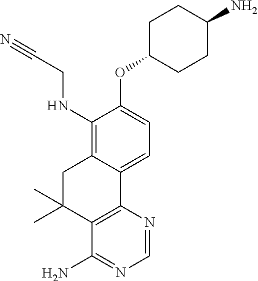 [SMILES: CC1(C)Cc2c(ccc(O[C@H]3CC[C@H](N)CC3)c2NCC#N)-c2ncnc(N)c21] | Examples 5 and 22 using CAS 167081-25-6 and 590-17-0 | DMSO-d₆ + D₂O: 8.23 (s, 1H), 7.72 (d, J = 8.8 Hz, 1H), 6.97 (d, J = 8.8 Hz, 1H), 4.33 (m, 1H), 4.09 (s, 2H), 2.71 (s, 2H), 2.62 (m, 1H), 2.06 (m, 2H), 1.81 (m, 2H), 1.47 (m, 2H), 1.27 (s, 6H), 1.21 (m, 2H) | 2.28 | 393.3 [M + H]⁺ |
| 36 | 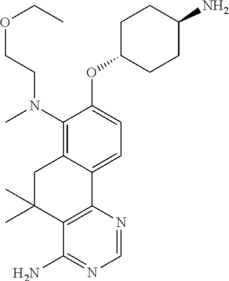 [SMILES: CCOCCN(C)c1c(O[C@H]2CC[C@H](N)CC2)ccc2c1CC(C)(C)c1c(N)ncnc1-2] | Examples 5 and 8 using CAS 167081-25-6, 22056-82-2 and 50-00-0 | DMSO-d₆ + D₂O: 8.21 (s, 1H), 7.85 (d, J = 8.8 Hz, 1H), 6.98 (d, J = 8.8 Hz, 1H), 4.37 (m, 1H), 3.32 (m, 4H), 3.16 (m, 1H), 3.02 (m, 3H), 2.78 (m, 1H), 2.65 (s, 3H), 2.14 (m, 2H), 1.97 (m, 2H), 1.48 (m, 4H), 1.27 (s, 3H), 1.22 (s, 3H), 1.06 (t, J= 7.2 Hz, 3H) | 2.99 | 440.5 [M + H]⁺ |
| 37 | 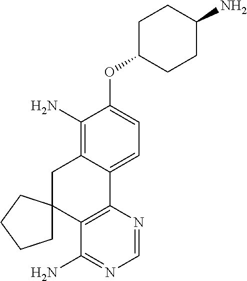 [SMILES: Nc1ncnc2c1C1(CCCC1)Cc1c-2ccc(O[C@H]2CC[C@H](N)CC2)c1N] | Examples 1, 2 and 5 using CAS 824-98- 6, 5660-83-3 and 167081- 25-6 | DMSO-d₆ + D₂O: 8.22 (s, 1H), 7.38 (d, J = 8.4 Hz, 1H), 6.84 (d, J = 8.4 Hz, 1H), 4.24 (m, 1H), 3.03 (m, 1H), 2.64 (s, 2H), 2.09 (m, 2H), 1.98 (m, 4H), 1.80 (m, 4H), 1.47 (m, 6H) | 2.49 | 380.3 [M + H]⁺ |
| 38 | 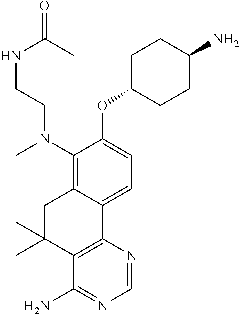 [SMILES: CC(=O)NCCN(C)c1c(O[C@H]2CC[C@H](N)CC2)ccc2c1CC(C)(C)c1c(N)ncnc1-2] | — | DMSO-d₆ + D₂O: 8.20 (s, 1H), 7.84 (d, J = 8.8 Hz, 1H), 6.99 (d, J = 8.8 Hz, 1H), 4.36 (m, 1H), 3.02 (m, 5H), 2.86 (m, 2H), 2.63 (s, 3H), 2.14 (m, 2H), 1.97 (m, 2H), 1.77 (s, 3H), 1.48 (m, 4H), 1.25 (s, 3H), 1.23 (s, 3H) | 2.53 | 453.5 [M + H]⁺ |
| 39 | 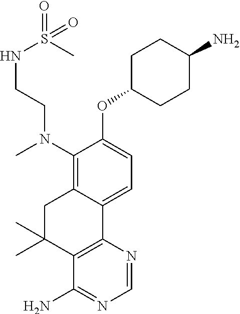 [SMILES: CN(CCNS(C)(=O)=O)c1c(O[C@H]2CC[C@H](N)CC2)ccc2c1CC(C)(C)c1c(N)ncnc1-2] | — | DMSO-d₆ + D₂O: 8.22 (s, 1H), 7.86 (d, J = 8.8 Hz, 1H), 7.00 (d, J = 8.8 Hz, 1H), 4.38 (m, 1H), 2.99 (m, 5H), 2.79 (m, 2H), 2.85 (s, 3H), 2.65 (s, 3H), 2.15 (m, 2H), 1.97 (m, 2H), 1.49 (m, 4H), 1.28 (s, 3H), 1.24 (s, 3H) | 2.57 | 489.3 [M + H]⁺ |
| 40 | 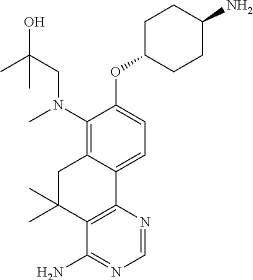 [SMILES: CN(CC(C)(C)O)c1c(O[C@H]2CC[C@H](N)CC2)ccc2c1CC(C)(C)c1c(N)ncnc1-2] | Examples 5 and 8 using CAS 167081-25-6, 20818-81-9 and 50-00-0 | DMSO-d₆ + D₂O: 8.21 (s, 1H), 7.83 (d, J = 8.8 Hz, 1H), 6.99 (d, J = 8.8 Hz, 1H), 4.36 (m, 1H), 3.02 (m, 2H), 2.85 (m, 3H), 2.68 (s, 3H), 2.15 (m, 2H), 1.98 (m, 2H), 1.50 (m, 4H), 1.26 (s, 6H), 1.14 (s, 3H), 1.01 (s, 3H) | 2.67 | 440.4 [M + H]⁺ |
| 41 | 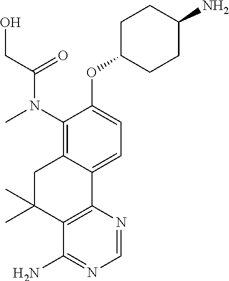 [SMILES: CN(C(=O)CO)c1c(O[C@H]2CC[C@H](N)CC2)ccc2c1CC(C)(C)c1c(N)ncnc1-2] | Examples 5, 6, 19 and 22 using CAS 167081-25-6, 88920-24-5 and 74-88-4 | DMSO-d₆ + D₂O: 8.26 (s, 1H), 8.07 (d, J = 8.8 Hz, 1H), 7.18 (d, J = 8.8 Hz, 1H), 4.40 (m, 1H), 3.54 (d, J = 4.0 Hz, 2H), 2.98 (m, 4H), 2.62 (s, 2H), 2.06 (m, 2H), 1.92 (m, 2H), 1.42 (m, 4H), 1.27 (d, J = 2.4 Hz, 6H) | 2.08 | 426.3 [M + H]⁺ |
| 42 | 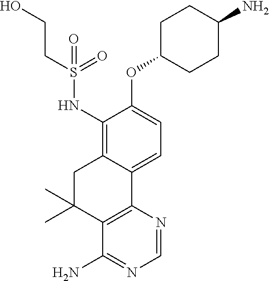 [SMILES: CC1(C)Cc2c(ccc(O[C@H]3CC[C@H](N)CC3)c2NS(=O)(=O)CCO)-c2ncnc(N)c21] | — | DMSO-d₆ + D₂O: 8.24 (s, 1H), 8.00 (d, J = 8.8 Hz, 1H), 7.11 (d, J = 8.8 Hz, 1H), 4.42 (m, 1H), 3.82 (t, J = 7.2 Hz, 2H), 3.26 (t, J = 7.2 Hz, 2H), 3.02 (m, 1H), 2.85 (s, 2H), 2.14 (m, 2H), 2.00 (m, 2H), 1.51 (m, 4H), 1.25 (s, 6H) | 1.94 | 462.3 [M + H]⁺ |
| 43 | 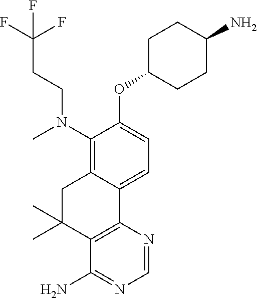 [SMILES: CN(CCC(F)(F)F)c1c(O[C@H]2CC[C@H](N)CC2)ccc2c1CC(C)(C)c1c(N)ncnc1-2] | Examples 5 and 8 using CAS 167081-25-6, 460-40-2 and 50-00-0 | DMSO-d₆ + D₂O: 8.22 (s, 1H), 7.88 (d, J = 8.8 Hz, 1H), 7.02 (d, J = 8.8 Hz, 1H), 4.39 (m, 1H), 3.28 (m, 1H), 3.19 (m, 1H), 3.04 (m, 1H), 2.93 (m, 1H), 2.74 (m, 1H), 2.64 (s, 3H), 2.30 (m, 2H), 2.15 (m, 2H), 1.97 (m, 2H), 1.48 (m, 4H), 1.28 (s, 3H), 1.20 (s, 3H) | 3.35 | 464.4 [M + H]⁺ |
| 44 | 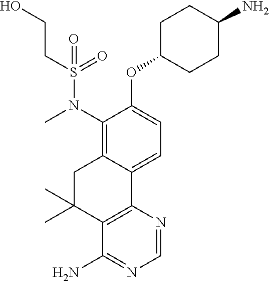 [SMILES: CN(c1c(O[C@H]2CC[C@H](N)CC2)ccc2c1CC(C)(C)c1c(N)ncnc1-2)S(=O)(=O)CCO] | Examples 4, 5, 22 and 42 using CAS 167081-25-6, 51517-01-2 and 74-88-4 | DMSO-d₆ + D₂O: 8.24 (s, 1H), 8.03 (d, J = 8.8 Hz, 1H), 7.16 (d, J = 8.8 Hz, 1H), 4.48 (m, 1H), 3.80 (m, 2H), 3.28 (m, 2H), 3.07 (s, 3H), 3.05 (m, 1H), 2.77 (s, 2H), 2.20 (m, 2H), 1.98 (m, 2H), 1.54 (m, 4H), 1.33 (s, 3H), 1.18 (s, 3H) | 2.21 | 476.3 [M + H]⁺ |
| 45 | 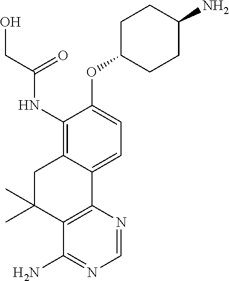 [SMILES: CC1(C)Cc2c(ccc(O[C@H]3CC[C@H](N)CC3)c2NC(=O)CO)-c2ncnc(N)c21] | Examples 5, 6 and 19 using CAS 167081-25-6 and 88920- 24-5 | DMSO-d₆ + D₂O: 8.24 (s, 1H), 7.96 (d, J = 8.8 Hz, 1H), 7.07 (d, J = 8.8 Hz, 1H), 4.28 (m, 1H), 4.01 (s, 2H), 3.03 (m, 1H), 2.56 (s, 2H), 2.06 (m, 2H), 1.94 (m, 2H), 1.44 (m, 4H), 1.22 (s, 6H) | 1.79 | 412.3 [M + H]⁺ |
| 46 | 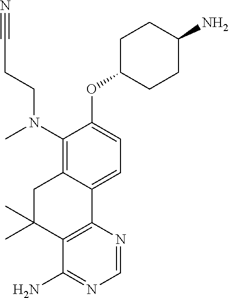 [SMILES: CN(CCC#N)c1c(O[C@H]2CC[C@H](N)CC2)ccc2c1CC(C)(C)c1c(N)ncnc1-2] | Examples 5 and 8 using CAS 167081-25-6, 6162-76-1 and 50-00-0 | DMSO-d₆ + D₂O: 8.21 (s, 1H), 7.87 (d, J = 8.8 Hz, 1H), 7.02 (d, J = 8.8 Hz, 1H), 4.38 (m, 1H), 3.21 (m, 2H), 3.09 (m, 3H), 2.75 (m, 1H), 2.66 (m, 1H), 2.64 (s, 3H), 2.15 (m, 2H), 1.97 (m, 2H), 1.49 (m, 4H), 1.30 (s, 3H), 1.21 (s, 3H) | 2.77 | 421.3 [M + H]+ |
| 47 | 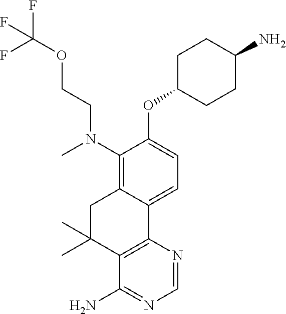 [SMILES: CN(CCOC(F)(F)F)c1c(O[C@H]2CC[C@H](N)CC2)ccc2c1CC(C)(C)c1c(N)ncnc1-2] | Examples 5, 8 and 22 using CAS 167081-25-6, 1645-93-8 and 50-00-0 | DMSO-d₆ + D₂O: 8.22 (s, 1H), 7.87 (d, J = 8.8 Hz, 1H), 7.01 (d, J = 8.8 Hz, 1H), 4.38 (m, 1H), 4.12 (m, 1H), 3.96 (m, 1H), 3.26 (t, J= 5.2 Hz, 2H), 3.01 (m, 2H), 2.75 (m, 1H), 2.65 (s, 3H), 2.14 (m, 2H), 1.97 (m, 2H), 1.47 (m, 4H), 1.30 (s, 3H), 1.16 (s, 3H) | 3.78 | 480.3 [M + H]₊ |
| 48 | 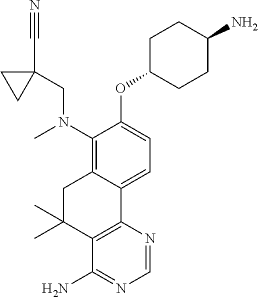 [SMILES: CN(CC1(C#N)CC1)c1c(O[C@H]2CC[C@H](N)CC2)ccc2c1CC(C)(C)c1c(N)ncnc1-2] | — | DMSO-d₆ + D₂O: 8.22 (s, 1H), 7.87 (d, J = 8.8 Hz, 1H), 7.00 (d, J = 8.8 Hz, 1H), 4.35 (m, 1H), 3.24 (m, 2H), 3.06 (m, 1H), 2.86 (m, 2H), 2.69 (s, 3H), 2.12 (m, 2H), 1.96 (m, 2H), 1.48 (m, 4H), 1.31 (s, 3H), 1.25 (m, 1H), 1.23 (s, 3H), 1.03 (m, 1H), 0.94 (m, 1H), 0.66 (m, 1H) | 3.25 | 447.2 [M + H]⁺ |
| 49 | 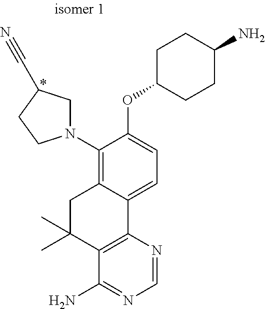 [SMILES: CC1(C)Cc2c(ccc(O[C@H]3CC[C@H](N)CC3)c2N2CCC(C#N)C2)-c2ncnc(N)c21] | — | DMSO-d₆ + D₂O: 8.27 (s, 1H), 7.86 (d, J = 8.8 Hz, 1H), 7.07 (d, J = 8.8 Hz, 1H), 4.41 (m, 1H), 3.41 (overlapped with water peak, 2H), 3.21 (m, 2H), 3.11 (m, 2H), 2.86 (m, 2H), 2.32 (m, 1H), 2.15 (m, 3H), 1.98 (m, 2H), 1.51 (m, 4H), 1.26 (s, 3H), 1.23 (s, 3H) | 3.09 | 433.2 [M + H]⁺ |
| 50 | 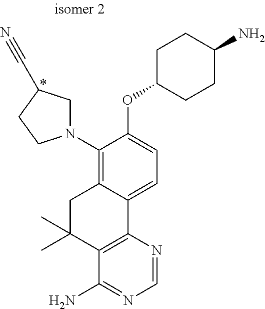 [SMILES: CC1(C)Cc2c(ccc(O[C@H]3CC[C@H](N)CC3)c2N2CCC(C#N)C2)-c2ncnc(N)c21] | — | DMSO-d₆ + D₂O: 8.53 (s, 1H), 7.77 (d, J = 8.8 Hz, 1H), 7.23 (d, J = 8.8 Hz, 1H), 4.48 (m, 1H), 3.41 (overlapped with water peak, 2H), 3.20 (m, 2H), 3.11 (m, 2H), 2.94 (m, 2H), 2.33 (m, 1H), 2.16 (m, 3H), 1.99 (m, 2H), 1.53 (m, 4H), 1.28 (s, 3H), 1.25 (s, 3H) | 3.13 | 433.3 [M + H]⁺ |
| 51 | 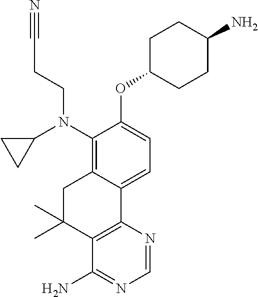 [SMILES: CC1(C)Cc2c(ccc(O[C@H]3CC[C@H](N)CC3)c2N(CCC#N)C2CC2)-c2ncnc(N)c21] | — | DMSO-d₆ + D₂O: 8.22 (s, 1H), 7.91 (d, J = 8.8 Hz, 1H), 7.04 (d, J = 8.8 Hz, 1H), 4.42 (m, 1H), 3.29 (t, J = 6.0 Hz, 2H), 3.01 (m, 1H), 2.88 (m, 2H), 2.66 (m, 2H), 2.40 (m, 1H), 2.16 (m, 2H), 1.95 (m, 2H), 1.48 (m, 4H), 1.23 (s, 3H), 1.21 (s, 3H), 0.56 (m, 2H), 0.26 (m, 1H), 0.14 (m, 1H) | 3.24 | 447.3 [M + H]⁺ |
| 52 | 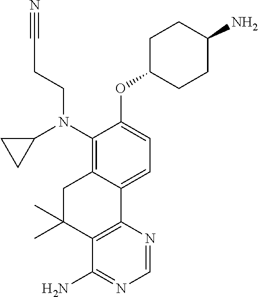 | — | DMSO-d₆ + D₂O: 8.22 (s, 1H), 7.88 (d, J = 8.8 Hz, 1H), 6.98 (d, J = 8.8 Hz, 1H), 4.37 (m, 1H), 3.56 (m, 4H), 3.23 (t, J = 6.4 Hz, 2H), 2.97 (m, 2H), 2.65 (s, 3H), 2.46 (m, 6H), 2.26 (m, 1H), 2.14 (m, 2H), 1.85 (m, 2H), 1.42 (m, 4H), 1.31 (s, 3H), 1.23 (s, 3H) | 3.11 | 491.3 [M + H]⁺ |
| 53 | 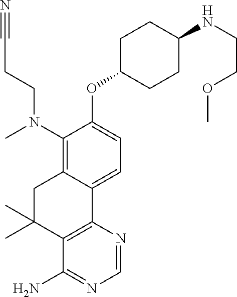 [SMILES: COCCN[C@H]1CC[C@H](Oc2ccc3c(c2N(C)CCC#N)CC(C)(C)c2c(N)ncnc2-3)CC1] | Examples 8, 9, 22, 48, 51 using CAS 107-20-0, 50-00-0, 98-74-8 and 6482-24-2 | DMSO-d₆ + D₂O: 8.51 (s, 1H), 7.80 (d, J = 8.8 Hz, 1H), 7.21 (d, J = 8.8 Hz, 1H), 4.46 (m, 1H), 3.61 (t, J = 5.2 Hz, 2H), 3.32 (s, 3H), 3.16 (m, 7H), 2.84 (m, 1H), 2.68 (m, 1H), 2.64 (s, 3H), 2.20 (m, 2H), 2.12 (m, 2H), 1.54 (m, 4H), 1.32 (s, 3H), 1.24 (s, 3H) | 3.22 | 479.3 [M + H]⁺ |
| 54 | 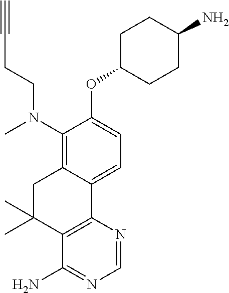 [SMILES: C#CCCN(C)c1c(O[C@H]2CC[C@H](N)CC2)ccc2c1CC(C)(C)c1c(N)ncnc1-2] | — | DMSO-d₆ + D₂O: 8.21 (s, 1H), 7.85 (d, J = 8.8 Hz, 1H), 6.99 (d, J = 8.8 Hz, 1H), 4.37 (m, 1H), 3.15 (m, 1H), 3.06 (m, 3H), 2.78 (m, 1H), 2.71 (t, J = 2.4 Hz, 1H), 2.64 (s, 3H), 2.17 (m, 4H), 1.97 (m, 2H), 1.48 (m, 4H), 1.27 (s, 3H), 1.21 (s, 3H) | 3.42 | 420.2 [M + H]⁺ |
| 55 | 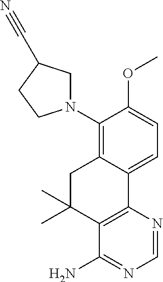 [SMILES: COc1ccc2c(c1N1CCC(C#N)C1)CC(C)(C)c1c(N)ncnc1-2] | Examples 2, 9 and 49 using CAS 50634-05-4 | DMSO-d₆ + D₂O: 8.27 (s, 1H), 7.87 (d, J = 8.8 Hz, 1H), 6.99 (d, J = 8.8 Hz, 1H), 3.84 (s, 3H), 3.41 (m, 2H), 3.20 (m, 2H), 3.12 (m, 1H), 2.88 (m, 2H), 2.32 (m, 1H), 2.16 (m, 1H), 1.27 (s, 3H), 1.23 (s, 3H) | 4.26 | 350.5 [M + H]⁺ |
| 56 | 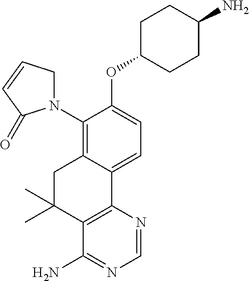 [SMILES: CC1(C)Cc2c(ccc(O[C@H]3CC[C@H](N)CC3)c2N2CC=CC2=O)-c2ncnc(N)c21] | — | DMSO-d₆ + D₂O: 8.50 (s, 1H), 7.92 (d, J = 8.8 Hz, 1H), 7.52 (d, J = 6.0 Hz, 1H), 7.29 (d, J = 8.8 Hz, 1H), 6.27 (dt, J = 6.0 Hz, 1.6 Hz, 1H), 4.39 (m, 2H), 4.13 (d, J = 16.0 Hz, 1H), 3.07 (m, 1H), 2.57 (d, J = 7.2 Hz, 2H), 2.02 (m, 4H), 1.44 (m, 4H), 1.31 (s, 3H), 1.18 (s, 3H) | 2.31 | 420.4 [M + H]⁺ |
| 57 | 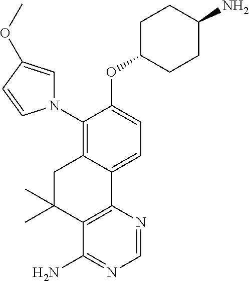 [SMILES: COc1ccn(-c2c(O[C@H]3CC[C@H](N)CC3)ccc3c2CC(C)(C)c2c(N)ncnc2-3)c1] | — | DMSO-d₆ + D₂O: 8.26 (s, 1H), 8.06 (d, J = 8.8 Hz, 1H), 7.18 (d, J = 8.8 Hz, 1H), 6.48 (t, J = 2.4 Hz, 1H), 6.31 (t, J = 2.0 Hz, 1H), 5.90 (t, J = 2.0 Hz, 1H), 4.23 (m, 1H), 3.62 (s, 3H), 2.96 (m, 1H), 2.41 (s, 2H), 1.99 (m, 2H), 1.87 (m, 2H), 1.38 (m, 4H), 1.18 (s, 6H) | 3.06 | 434.3 [M + H]⁺ |
| 58 | 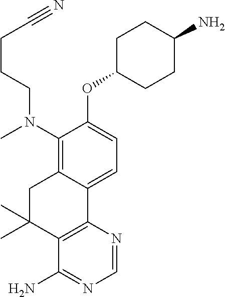 [SMILES: CN(CCCC#N)c1c(O[C@H]2CC[C@H](N)CC2)ccc2c1CC(C)(C)c1c(N)ncnc1-2] | Examples 9 and 22 using CAS 5332- 06-9 and 50-00-0 | DMSO-d₆ + D₂O: 8.22 (s, 1H), 7.87 (d, J = 8.8 Hz, 1H), 7.00 (d, J = 8.8 Hz, 1H), 4.37 (m, 1H), 3.08 (m, 2H), 2.93 (m, 2H), 2.82 (m, 1H), 2.61 (s, 3H), 2.52 (overlapped with DMSO peak, 2H), 2.14 (m, 2H), 1.94 (m, 2H), 1.61 (m, 2H), 1.47 (m, 4H), 1.28 (s, 3H), 1.25 (s, 3H) | 3.11 | 435.5 [M + H]⁺ |
| 59 | 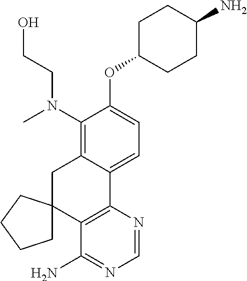 [SMILES: CN(CCO)c1c(O[C@H]2CC[C@H](N)CC2)ccc2c1CC1(CCCC1)c1c(N)ncnc1-2] | Examples 1- 5, 9 and 22 using CAS 5660-83-3, 105-36-2 and 50-00-0 | DMSO-d₆ + D₂O: 8.24 (s, 1H), 7.83 (d, J = 8.8 Hz, 1H), 6.98 (d, J = 8.8 Hz, 1H), 4.37 (m, 1H), 3.35 (m, 2H), 3.05 (m, 4H), 2.79 (m, 1H), 2.64 (s, 3H), 2.14 (m, 3H), 1.97 (m, 2H), 1.78 (m, 5H), 1.48 (m, 6H) | 2.59 | 438.5 [M + H]⁺ |
| 60 | 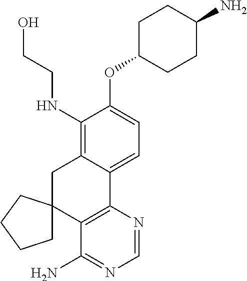 [SMILES: Nc1ncnc2c1C1(CCCC1)Cc1c-2ccc(O[C@H]2CC[C@H](N)CC2)c1NCCO] | Examples 1- 5, 9 and 22 using CAS 5660-83-3 and 105-36-2 | DMSO-d₆ + D₂O: 8.24 (s, 1H), 7.61 (d, J = 8.8 Hz, 1H), 6.94 (d, J = 8.8 Hz, 1H), 4.30 (m, 1H), 3.48 (t, J = 5.6 Hz, 2H), 3.04 (m, 1H), 2.96 (t, J = 5.6 Hz, 2H), 2.76 (s, 2H), 2.09 (m, 2H), 1.99 (m, 4H), 1.77 (m, 4H), 1.47 (m, 6H) | 2.33 | 424.3 [M + H]⁺ |
| 61 | 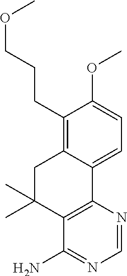 [SMILES: COCCCc1c(OC)ccc2c1CC(C)(C)c1c(N)ncnc1-2] | — | DMSO-d₆ + D₂O: 8.23 (s, 1H), 7.95 (d, J = 8.8 Hz, 1H), 6.92 (d, J = 8.8 Hz, 1H), 3.81 (s, 3H), 3.35 (t, J = 6.4 Hz, 2H), 3.24 (s, 3H), 2.74 (s, 2H), 2.67 (m, 2H), 1.63 (m, 2H), 1.27 (s, 6H) | 4.16 | 328.4 [M + H]⁺ |
| 62 | 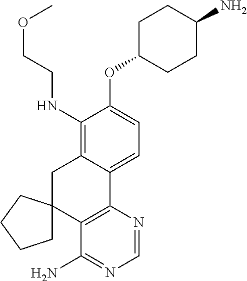 [SMILES: COCCNc1c(O[C@H]2CC[C@H](N)CC2)ccc2c1CC1(CCCC1)c1c(N)ncnc1-2] | Examples 1- 5, 9 and 22 using CAS 5660-83-3 and 10312- 83-1 | DMSO-d₆ + D₂O: 8.25 (s, 1H), 7.63 (d, J = 8.8 Hz, 1H), 6.94 (d, J = 8.8 Hz, 1H), 4.31 (m, 1H), 3.36 (overlapped with water peak, 2H), 3.26 (s, 3H), 3.06 (t, J = 5.6 Hz, 2H), 2.97 (m, 1H), 2.76 (s, 2H), 2.04 (m, 6H), 1.78 (m, 4H), 1.45 (m, 6H) | 2.72 | 438.4 [M + H]⁺ |
| 63 | 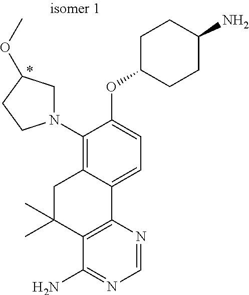 [SMILES: COC1CCN(c2c(O[C@H]3CC[C@H](N)CC3)ccc3c2CC(C)(C)c2c(N)ncnc2-3)C1] | Examples 9 and 15 using CAS 5281- 75-4 -[α]_{D}²⁶ = −38° (c = 0.1, MeOH). | DMSO-d₆ + D₂O: 8.21 (s, 1H), 7.84 (d, J = 8.8 Hz, 1H), 6.99 (d, J = 8.8 Hz, 1H), 4.36 (m, 1H), 4.05 (m, 1H), 3.26 (m, 4H), 3.19 (m, 1H), 3.05 (m, 3H), 2.82 (s, 2H), 2.13 (m, 3H), 1.96 (m, 2H), 1.86 (m, 1H), 1.46 (m, 4H), 1.24 (s, 3H), 1.23 (s, 3H) | 3.01 | 438.3 [M + H]⁺ |
| 64 | 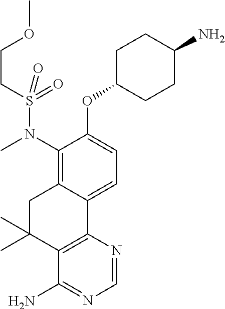 [SMILES: COCCS(=O)(=O)N(C)c1c(O[C@H]2CC[C@H](N)CC2)ccc2c1CC(C)(C)c1c(N)ncnc1-2] | Examples 4, 9 and 42 using CAS 51517-01-2 and 74-88-4 | DMSO-d₆ + D₂O 8.24 (s, 1H), 8.03 (d, J = 8.8 Hz, 1H), 7.15 (d, J = 8.8 Hz, 1H), 4.48 (m, 1H), 3.72 (m, 2H), 3.48 (m, 2H), 3.26 (s, 3H), 3.07 (s, 3H), 3.03 (m, 1H), 2.77 (d, J = 3.2 Hz, 2H), 2.16 (m, 2H), 1.98 (m, 2H), 1.49 (m, 4H), 1.32 (s, 3H), 1.19 (s, 3H) | 2.64 | 490.4 [M + H]⁺ |
| 65 | 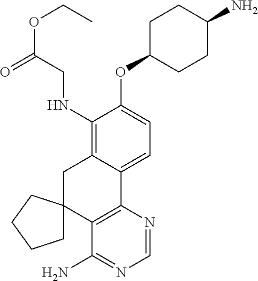 [SMILES: CCOC(=O)CNc1c(O[C@H]2CC[C@@H](N)CC2)ccc2c1CC1(CCCC1)c1c(N)ncnc1-2] | Examples 1, 2, 5, 9, 22, 34 and 61 using CAS 5660- 83-3, 111300-06-2 and 105-36-2 | DMSO-d₆ + D₂O: 8.23 (s, 1H), 7.60 (d, J = 8.8 Hz, 1H), 6.87 (d, J = 8.8 Hz, 1H), 4.59 (m, 1H), 4.06 (q, J = 7.2 Hz, 2H), 3.93 (s, 2H), 3.07 (m, 1H), 2.78 (s, 2H), 2.00 (m, 4H), 1.73 (m, 10H), 1.50 (m, 2H), 1.14 (t, J = 7.2 Hz, 3H) | 3.01 | 466.3 [M + H]⁺ |
| 66 | 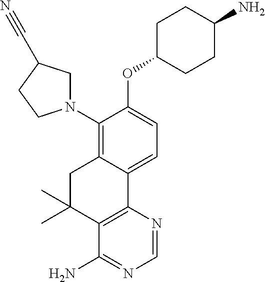 [SMILES: CC1(C)Cc2c(ccc(O[C@H]3CC[C@H](N)CC3)c2N2CCC(C#N)C2)-c2ncnc(N)c21] | Example 9 using CAS 1823050-70-9 | DMSO-d₆ + D₂O: 8.22 (s, 1H), 7.88 (d, J = 8.8 Hz, 1H), 7.03 (d, J = 8.8 Hz, 1H), 4.39 (m, 1H), 3.40 (overlapped with water peak, 2H), 3.22 (m, 2H), 3.13 (m, 1H), 3.03 (m, 1H), 2.85 (m, 2H), 2.32 (m, 1H), 2.15 (m, 3H), 1.95 (m, 2H), 1.49 (m, 4H), 1.26 (s, 3H), 1.23 (s, 3H) | 3.09 | 433.2 [M + H]⁺ |
| 67 | 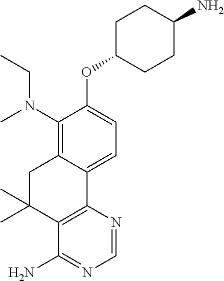 [SMILES: CCN(C)c1c(O[C@H]2CC[C@H](N)CC2)ccc2c1CC(C)(C)c1c(N)ncnc1-2] | Example 8 using CAS 75-07-0 | DMSO-d₆ + D₂O: 8.22 (s, 1H), 7.85 (d, J = 8.8 Hz, 1H), 6.97 (d, J = 8.8 Hz, 1H), 4.36 (m, 1H), 3.15 (m, 1H), 2.90 (m, 4H), 2.64 (s, 3H), 2.13 (m, 2H), 1.96 (m, 2H), 1.45 (m, 4H), 1.24 (s, 6H), 0.86 (t, J = 7.2 Hz, 3H) | 2.30 | 396.3 [M + H]⁺ |
| 68 | 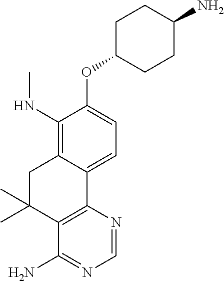 [SMILES: CNc1c(O[C@H]2CC[C@H](N)CC2)ccc2c1CC(C)(C)c1c(N)ncnc1-2] | Example 8 using CAS 50-00-0 | DMSO-d₆ + D₂O: 8.22 (s, 1H), 7.61 (d, J = 8.8 Hz, 1H), 6.95 (d, J = 8.8 Hz, 1H), 4.28 (m, 1H), 3.02 (m, 1H), 2.72 (s, 2H), 2.64 (s, 3H), 2.10 (m, 2H), 1.96 (m, 2H), 1.48 (m, 4H), 1.26 (s, 6H) | 1.77 | 368.2 [M + H]⁺ |
| 69 | 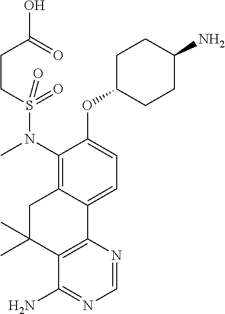 [SMILES: CN(c1c(O[C@H]2CC[C@H](N)CC2)ccc2c1CC(C)(C)c1c(N)ncnc1-2)S(=O)(=O)CCC(=O)O] | Examples 9, 22, 26 and 42 using CAS 15441-07-3 and 74-88-4 | DMSO-d₆ + D₂O: 8.24 (s, 1H), 8.03 (d, J = 8.8 Hz, 1H), 7.16 (d, J = 8.8 Hz, 1H), 4.48 (m, 1H), 3.31 (m, 2H), 3.10 (s, 3H), 3.03 (m, 1H), 2.78 (m, 2H), 2.43 (m, 2H), 2.24 (m, 1H), 2.12 (m, 1H), 1.99 (m, 2H), 1.55 (m, 4H), 1.34 (s, 3H), 1.18 (s, 3H) | 2.59 | 504.3 [M + H]+ |
| 70 | 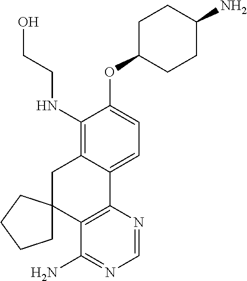 [SMILES: Nc1ncnc2c1C1(CCCC1)Cc1c-2ccc(O[C@H]2CC[C@@H](N)CC2)c1NCCO] | Examples 1, 2, 5, 9 and 22 using CAS 5660-83-3 and 105-36-2 | DMSO-d₆ + D₂O: 8.24 (s, 1H), 7.62 (d, J = 8.8 Hz, 1H), 6.88 (d, J = 8.8 Hz, 1H), 4.62 (m, 1H), 3.53 (overlapped with water peak, 2H), 3.08 (m, 1H), 2.98 (t, J = 5.6 Hz, 2H), 2.78 (s, 2H), 2.01 (m, 4H), 1.72 (m, 10H), 1.47 (m, 2H) | 2.37 | 424.3 [M + H]⁺ |
| 71 | 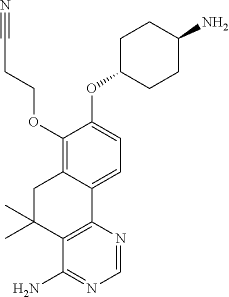 [SMILES: CC1(C)Cc2c(ccc(O[C@H]3CC[C@H](N)CC3)c2OCCC#N)-c2ncnc(N)c21] | Examples 1, 3, 5, 9, 19 and 61 using CAS 111300-06-2 and 2417-90-5 | DMSO-d₆ + D₂O: 8.23 (s, 1H), 7.81 (d, J = 8.8 Hz, 1H), 7.07 (d, J = 8.8 Hz, 1H), 4.35 (m, 1H), 4.06 (t, J = 6.0 Hz, 2H), 3.02 (m, 1H), 2.92 (t, J = 6.0 Hz, 2H), 2.82 (s, 2H), 2.13 (m, 2H), 1.97 (m, 2H), 1.49 (m, 4H), 1.28 (s, 6H) | 2.61 | 408.2 [M + H]⁺ |
| 72 | 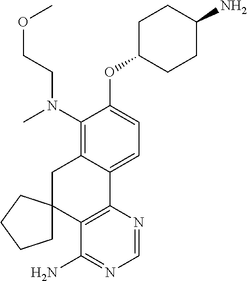 [SMILES: COCCN(C)c1c(O[C@H]2CC[C@H](N)CC2)ccc2c1CC1(CCCC1)c1c(N)ncnc1-2] | Examples 1, 2, 5, 8 and 9 using CAS 5660-83-3 and 10312-83-1 | DMSO-d₆ + D₂O: 8.24 (s, 1H), 7.84 (d, J = 8.8 Hz, 1H), 6.99 (d, J = 8.8 Hz, 1H), 4.37 (m, 1H), 3.29 (m, 2H), 3.17 (s, 3H), 3.06 (m, 4H), 2.78 (m, 1H), 2.64 (s, 3H), 2.14 (m, 3H), 1.96 (m, 2H), 1.79 (m, 5H), 1.46 (m, 6H) | 3.23 | 452.3 [M + H]⁺ |
| 73 | 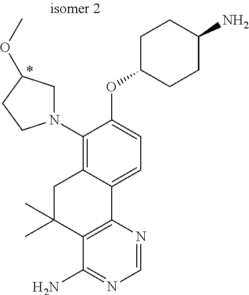 [SMILES: COC1CCN(c2c(O[C@H]3CC[C@H](N)CC3)ccc3c2CC(C)(C)c2c(N)ncnc2-3)C1] | Examples 9 and 15 using CAS 5281- 75-4 -[α]_{D}²⁶ = +22° (c = 0.1, MeOH). | DMSO-d₆ + D₂O: 8.21 (s, 1H), 7.83 (d, J = 8.8 Hz, 1H), 6.99 (d, J = 8.8 Hz, 1H), 4.36 (m, 1H), 4.05 (m, 1H), 3.26 (m, 4H), 3.19 (m, 1H), 3.05 (m, 3H), 2.82 (s, 2H), 2.13 (m, 3H), 1.96 (m, 2H), 1.86 (m, 1H), 1.47 (m, 4H), 1.24 (s, 3H), 1.23 (s, 3H) | 3.00 | 438.3 [M + H]⁺ |
| 74 | 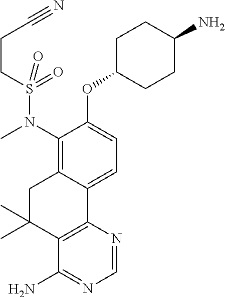 [SMILES: CN(c1c(O[C@H]2CC[C@H](N)CC2)ccc2c1CC(C)(C)c1c(N)ncnc1-2)S(=O)(=O)CCC#N] | Examples 22, 42 and 49 using CAS 27869-05-2 and 74-88-4 | DMSO-d₆ + D₂O: 8.24 (s, 1H), 8.05 (d, J = 8.8 Hz, 1H), 7.18 (d, J = 8.8 Hz, 1H), 4.49 (m, 1H), 3.64 (m, 1H), 3.47 (m, 1H), 3.10 (s, 3H), 2.99 (m, 3H), 2.77 (m, 2H), 2.19 (m, 2H), 1.99 (m, 2H), 1.50 (m, 4H), 1.33 (s, 3H), 1.19 (s, 3H) | 2.64 | 485.3 [M + H]⁺ |
| 75 | 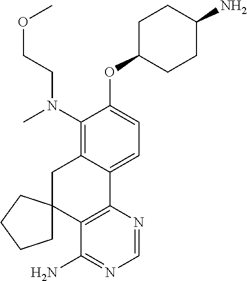 [SMILES: COCCN(C)c1c(O[C@H]2CC[C@@H](N)CC2)ccc2c1CC1(CCCC1)c1c(N)ncnc1-2] | Examples 1, 2, 5, 8 and 9 using CAS 5660-83-3, 111300-06-2, 10312-83-1 and 50-00-0 | DMSO-d₆ + D₂O: 8.24 (s, 1H), 7.84 (d, J = 8.8 Hz, 1H), 6.90 (d, J = 8.8 Hz, 1H), 4.62 (m, 1H), 3.32 (m, 2H), 3.18 (m, 6H), 3.03 (m, 1H), 2.79 (m, 1H), 2.71 (s, 3H), 2.18 (m, 1H), 1.99 (m, 2H), 1.80 (m, 7H), 1.68 (m, 4H), 1.46 (m, 2H) | 3.09 | 452.4 [M + H]⁺ |
| 76 | 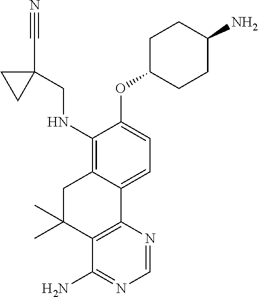 [SMILES: CC1(C)Cc2c(ccc(O[C@H]3CC[C@H](N)CC3)c2NCC2(C#N)CC2)-c2ncnc(N)c21] | Examples 4, 9 and 48 using CAS 98730-78-0 | DMSO-d₆ + D₂O: 8.23 (s, 1H), 7.69 (d, J = 8.8 Hz, 1H), 6.96 (d, J = 8.8 Hz, 1H), 4.34 (m, 1H), 3.04 (s, 2H), 2.98 (m, 1H), 2.78 (s, 2H), 2.13 (m, 2H), 1.96 (m, 2H), 1.48 (m, 4H), 1.27 (s, 6H), 1.11 (m, 2H), 0.92 (m, 2H) | 2.63 | 433.3 [M + H]⁺ |
| 77 | 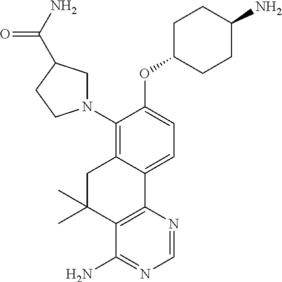 [SMILES: CC1(C)Cc2c(ccc(O[C@H]3CC[C@H](N)CC3)c2N2CCC(C(N)=O)C2)-c2ncnc(N)c21] | Examples 9 and 49 using CAS 1823050-70-9 | DMSO-d₆ + D₂O: 8.21 (s, 1H), 7.84 (d, J = 8.8 Hz, 1H), 7.00 (d, J = 8.8 Hz, 1H), 4.37 (m, 1H), 3.20 (m, 2H), 3.13 (m, 2H), 3.02 (m, 2H), 2.83 (m, 2H), 2.13 (m, 3H), 1.98 (m, 3H), 1.48 (m, 4H), 1.26 (s, 3H), 1.23 (s, 3H) | 2.42 | 451.3 [M + H]⁺ |
| 78 | 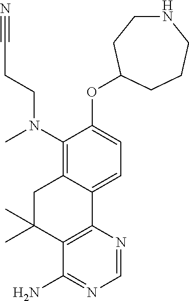 [SMILES: CN(CCC#N)c1c(OC2CCCNCC2)ccc2c1CC(C)(C)c1c(N)ncnc1-2] | Examples 2, 5, 8, 9, 22 and 34 using CAS 478832-21-1, 6162-76-1 and 50-00-0 | DMSO-d₆ + D₂O: 8.22 (s, 1H), 7.88 (d, J = 8.8 Hz, 1H), 6.95 (d, J = 8.8 Hz, 1H), 4.75 (m, 1H), 3.18 (m, 4H), 3.06 (m, 3H), 2.73 (m, 2H), 2.66 (s, 3H), 2.45 (m, 1H), 2.20 (m, 1H), 2.04 (m, 2H), 1.87 (m, 2H), 1.72 (m, 1H), 1.30 (s, 3H), 1.22 (s, 3H) | 2.86 | 421.2 [M + H]⁺ |
| 79 | 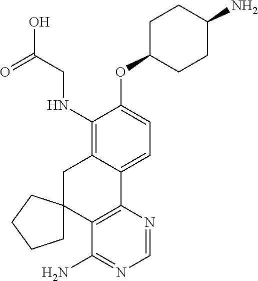 [SMILES: Nc1ncnc2c1C1(CCCC1)Cc1c-2ccc(O[C@H]2CC[C@@H](N)CC2)c1NCC(=O)O] | Examples 1, 2, 5, 9, 22 and 26 using CAS 5660- 83-3, 111300-06-2 and 105-36-2 | DMSO-d₆ + D₂O: 8.19 (s, 1H), 7.52 (d, J = 8.8 Hz, 1H), 6.89 (d, J = 8.8 Hz, 1H), 4.81 (m, 1H), 3.31 (s, 2H), 3.09 (m, 1H), 2.78 (s, 2H), 1.97 (m, 6H), 1.78 (m, 4H), 1.63 (m, 2H), 1.51 (m, 4H) | 2.57 | 438.3 [M + H]⁺ |
| 80 | 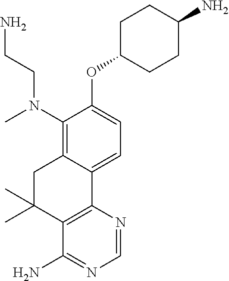 [SMILES: CN(CCN)c1c(O[C@H]2CC[C@H](N)CC2)ccc2c1CC(C)(C)c1c(N)ncnc1-2] | Examples 8, 9 and 22 using CAS 50-00-0 and 89711-08-0 | DMSO-d₆ + D₂O: 8.21 (s, 1H), 7.87 (d, J = 8.8 Hz, 1H), 7.02 (d, J = 8.8 Hz, 1H), 4.38 (m, 1H), 3.15 (m, 2H), 3.03 (m, 1H), 2.88 (m, 2H), 2.77 (m, 2H), 2.63 (s, 3H), 2.15 (m, 2H), 1.98 (m, 2H), 1.49 (m, 4H), 1.30 (s, 3H), 1.22 (s, 3H) | 1.98 | 411.3 [M + H]⁺ |
| 81 | 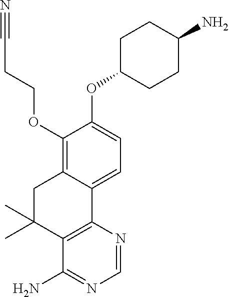 [SMILES: CC1(C)Cc2c(ccc(O[C@H]3CC[C@H](N)CC3)c2OCCC#N)-c2ncnc(N)c21] | Examples 9 and 61 using CAS 238088-16-9 | DMSO-d₆ + D₂O: 8.22 (s, 1H), 7.93 (d, J = 8.8 Hz, 1H), 6.99 (d, J = 8.8 Hz, 1H), 4.36 (m, 1H), 3.06 (m, 1H), 2.74 (m, 4H), 2.52 (m, 2H), 2.12 (m, 2H), 1.98 (m, 2H), 1.67 (m, 2H), 1.49 (m, 4H), 1.27 (s, 6H) | 2.90 | 406.2 [M + H]⁺ |
| 82 | 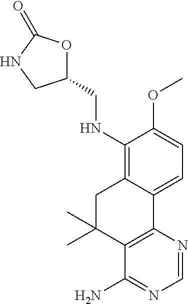 [SMILES: COc1ccc2c(c1NC[C@@H]1CNC(=O)O1)CC(C)(C)c1c(N)ncnc1-2] | Examples 2 and 28 using CAS 169048-83-3 | DMSO-d₆ + D₂O: 8.22 (s, 1H), 7.69 (d, J = 8.8 Hz, 1H), 6.91 (d, J = 8.8 Hz, 1H), 4.59 (m, 1H), 3.83 (s, 3H), 3.48 (t, J = 8.8 Hz, 1H), 3.23 (m, 1H), 3.13 (m, 2H), 2.74 (s, 2H), 1.27 (s, 3H), 1.25 (s, 3H) | 2.75 | 370.3 [M + H]⁺ |
| 83 | 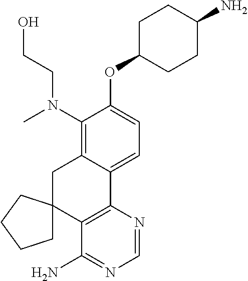 [SMILES: CN(CCO)c1c(O[C@H]2CC[C@@H](N)CC2)ccc2c1CC1(CCCC1)c1c(N)ncnc1-2] | Examples 1, 2, 5, 9 and 22 using CAS 5660-83-3, 111300-06-2 and 105-36-2 | DMSO-d₆ + D₂O: 8.23 (s, 1H), 7.82 (d, J = 8.8 Hz, 1H), 6.90 (d, J = 8.8 Hz, 1H), 4.63 (m, 1H), 3.40 (m, 2H), 3.11 (m, 4H), 2.80 (m, 1H), 2.71 (s, 3H), 2.12 (m, 1H), 2.01 (m, 2H), 1.77 (m, 11H), 1.46 (m, 2H) | 2.69 | 438.2 [M + H]⁺ |
| 84 | 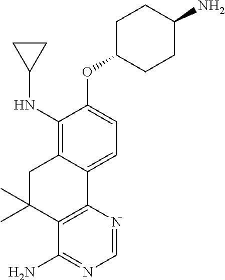 [SMILES: CC1(C)Cc2c(ccc(O[C@H]3CC[C@H](N)CC3)c2NC2CC2)-c2ncnc(N)c21] | Examples 9 and 51 using CAS 411235-57-9 | DMSO-d₆ + D₂O: 8.22 (s, 1H), 7.63 (d, J = 8.8 Hz, 1H), 6.93 (d, J = 8.8 Hz, 1H), 4.29 (m, 1H), 3.01 (m, 1H), 2.84 (s, 2H), 2.59 (m, 1H), 2.07 (m, 2H), 1.96 (m, 2H), 1.45 (m, 4H), 1.27 (s, 6H), 0.53 (m, 2H), 0.39 (m, 2H) | 2.78 | 394.1 [M + H]⁺ |
| 85 | 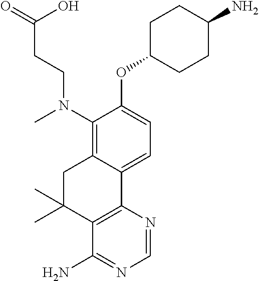 [SMILES: CN(CCC(=O)O)c1c(O[C@H]2CC[C@H](N)CC2)ccc2c1CC(C)(C)c1c(N)ncnc1-2] | Examples 8, 9, 22 and 26 using CAS 50-00-0 and 34780-29-5 | DMSO-d₆ + D₂O: 8.22 (s, 1H), 7.86 (d, J = 8.8 Hz, 1H), 7.00 (d, J = 8.8 Hz, 1H), 4.38 (m, 1H), 3.27 (m, 1H), 3.13 (m, 2H), 2.90 (m, 1H), 2.70 (m, 1H), 2.63 (s, 3H), 2.25 (m, 2H), 2.16 (m, 2H), 1.98 (m, 2H), 1.50 (m, 4H), 1.26 (s, 3H), 1.22 (s, 3H) | 2.65 | 440.2 [M + H]⁺ |
| 86 | 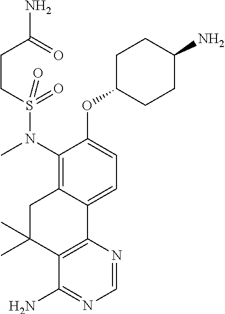 [SMILES: CN(c1c(O[C@H]2CC[C@H](N)CC2)ccc2c1CC(C)(C)c1c(N)ncnc1-2)S(=O)(=O)CCC(N)=O] | Examples 4, 9, 42 and 49 using CAS 27869-05-2 and 74-88-4 | DMSO-d₆ + D₂O: 8.25 (s, 1H), 8.04 (d, J = 8.8 Hz, 1H), 7.16 (d, J = 8.8 Hz, 1H), 4.49 (m, 1H), 3.40 (overlapped with water peak, 2H), 3.10 (s, 3H), 2.98 (m, 1H), 2.78 (m, 2H), 2.56 (m, 2H), 2.22 (m, 1H), 2.13 (m, 1H), 1.97 (m, 2H), 1.51 (m, 4H), 1.34 (s, 3H), 1.19 (s, 3H) | 2.36 | 503.3 [M + H]⁺ |
| 87 | 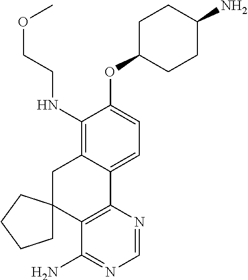 [SMILES: COCCNc1c(O[C@H]2CC[C@@H](N)CC2)ccc2c1CC1(CCCC1)c1c(N)ncnc1-2] | Examples 1, 2, 5, 8 and 9 using CAS 5660-83-3, 111300-06-2 and 10312- 83-1 | DMSO-d₆ + D₂O: 8.24 (s, 1H), 7.63 (d, J = 8.8 Hz, 1H), 6.88 (d, J = 8.8 Hz, 1H), 4.60 (m, 1H), 3.42 (t, J = 5.2 Hz, 2H), 3.26 (s, 3H), 3.09 (m, 3H), 2.78 (s, 2H), 2.00 (m, 4H), 1.71 (m, 10H), 1.47 (m, 2H) | 2.74 | 438.2 [M + H]⁺ |
| 88 | 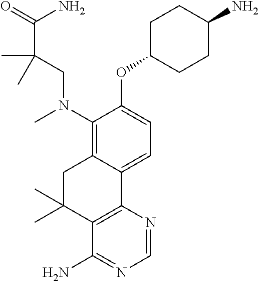 [SMILES: CN(CC(C)(C)C(N)=O)c1c(O[C@H]2CC[C@H](N)CC2)ccc2c1CC(C)(C)c1c(N)ncnc1-2] | Examples 8, 9, 22 and 49 using CAS 37647-55-5 and 50-00-0 | DMSO-d₆ + D₂O: 8.21 (s, 1H), 7.83 (d, J = 8.8 Hz, 1H), 6.99 (d, J = 8.8 Hz, 1H), 4.36 (m, 1H), 3.16 (d, J = 16.0 Hz, 1H), 3.08 (m, 1H), 3.00 (m, 2H), 2.77 (d, J = 16.0 Hz, 1H), 2.58 (s, 3H), 2.16 (m, 2H), 1.98 (m, 2H), 1.51 (m, 4H), 1.30 (s, 3H), 1.24 (s, 3H), 1.02 (d, J = 9.2 Hz, 6H) | 2.76 | 467.3 [M + H]⁺ |
| 89 | 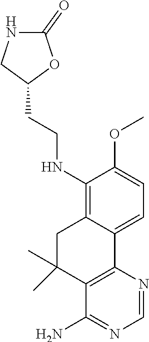 [SMILES: COc1ccc2c(c1NCC[C@@H]1CNC(=O)O1)CC(C)(C)c1c(N)ncnc1-2] | — | DMSO-d₆ + D₂O: 8.22 (s, 1H), 7.67 (d, J = 8.8 Hz, 1H), 6.90 (d, J = 8.8 Hz, 1H), 4.67 (m, 1H), 3.82 (s, 3H), 3.54 (t, J = 8.4 Hz, 1H), 3.10 (t, J = 8.0 Hz, 1H), 3.00 (m, 2H), 2.72 (s, 2H), 1.78 (m, 2H), 1.26 (s, 3H), 1.25 (s, 3H) | 2.55 | 384.1 [M + H]⁺ |
| 90 | 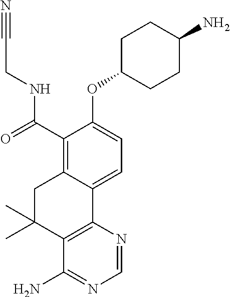 [SMILES: CC1(C)Cc2c(ccc(O[C@H]3CC[C@H](N)CC3)c2C(=O)NCC#N)-c2ncnc(N)c21] | — | DMSO-d₆ + D₂O: 9.03 (t, J = 5.6 Hz, 1H), 8.55 (s, 1H), 7.98 (d, J = 8.4 Hz, 1H), 7.30 (d, J = 8.4 Hz, 1H), 4.46 (m, 1H), 4.31 (d, J = 5.6 Hz, 2H), 3.08 (m, 1H), 2.66 (s, 2H), 2.10 (m, 2H), 1.98 (m, 2H), 1.49 (m, 4H), 1.26 (s, 6H) | 2.02 | 421.1 [M + H]⁺ |
| 91 | 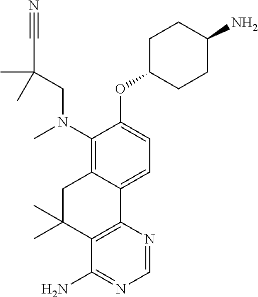 [SMILES: CN(CC(C)(C)C#N)c1c(O[C@H]2CC[C@H](N)CC2)ccc2c1CC(C)(C)c1c(N)ncnc1-2] | Examples 8, 9 and 22 using CAS 37647-55-5 and 50-00-0 | DMSO-d₆ + D₂O: 8.22 (s, 1H), 7.87 (d, J = 8.8 Hz, 1H), 7.03 (d, J = 8.8 Hz, 1H), 4.39 (m, 1H), 3.10 (m, 4H), 2.85 (d, J = 16.0 Hz, 1H), 2.73 (s, 3H), 2.17 (m, 2H), 1.99 (m, 2H), 1.51 (m, 4H), 1.30 (d, J= 10.8 Hz, 6H), 1.23 (d, J = 11.6 Hz, 6H) | 3.33 | 449.3 [M + H]⁺ |
| 92 | 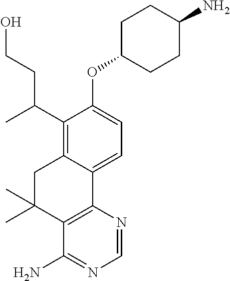 [SMILES: CC(CCO)c1c(O[C@H]2CC[C@H](N)CC2)ccc2c1CC(C)(C)c1c(N)ncnc1-2] | — | DMSO-d₆ + D₂O: 8.22 (s, 1H), 7.90 (d, J = 8.8 Hz, 1H), 6.95 (d, J = 8.8 Hz, 1H), 4.37 (m, 1H), 3.24 (m, 3H), 3.02 (m, 1H), 2.83 (m, 1H), 2.71 (m, 1H), 2.13 (m, 3H), 1.95 (m, 2H), 1.76 (m, 1H), 1.45 (m, 4H), 1.25 (m, 9H) | 2.72 | 411.2 [M + H]⁺ |
| 93 | 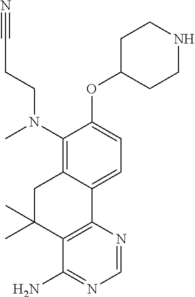 [SMILES: CN(CCC#N)c1c(OC2CCNCC2)ccc2c1CC(C)(C)c1c(N)ncnc1-2] | Examples 5, 8, 9 and 22 using CAS 118811-07-7, 2417-90-5 and 50-00-0 | DMSO-d₆ + D₂O: 8.22 (s, 1H), 7.89 (d, J = 8.8 Hz, 1H), 7.00 (d, J = 8.8 Hz, 1H), 4.65 (m, 1H), 3.25 (t, J = 6.0 Hz, 2H), 3.12 (m, 3H), 2.90 (m, 2H), 2.74 (m, 2H), 2.68 (s, 3H), 2.46 (m, 1H), 2.05 (m, 2H), 1.74 (m, 2H), 1.31 (s, 3H), 1.23 (s, 3H) | 2.70 | 407.2 [M + H]⁺ |
| 94 | 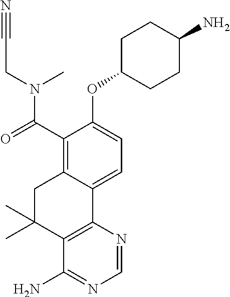 [SMILES: CN(CC#N)C(=O)c1c(O[C@H]2CC[C@H](N)CC2)ccc2c1CC(C)(C)c1c(N)ncnc1-2] | Example 90 using CAS 5616-32-0 | DMSO-d₆ + D₂O: 8.52 (s, 1H), 7.97 (d, J = 8.8 Hz, 1H), 7.30 (d, J = 8.8 Hz, 1H), 4.81 (d, J = 17.2 Hz, 1H), 4.49 (m, 1H), 4.39 (d, J= 17.2 Hz, 1H), 3.08 (m, 1H), 2.83 (s, 3H), 2.62 (m, 2H), 2.12 (m, 2H), 1.95 (m, 2H), 1.47 (m, 4H), 1.31 (s, 3H), 1.20 (s, 3H) | 2.29 | 435.2 [M + H]⁺ |
| 95 | 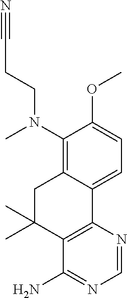 [SMILES: COc1ccc2c(c1N(C)CCC#N)CC(C)(C)c1c(N)ncnc1-2] | Examples 2, 8 and 22 using CAS 2417-90-5 and 50-00-0 | DMSO-d₆ + D₂O: 8.23 (s, 1H), 7.92 (d, J = 8.8 Hz, 1H), 6.96 (d, J = 8.8 Hz, 1H), 3.83 (s, 3H), 3.22 (t, J = 8.8 Hz, 2H), 2.55 (m, 2H), 2.65 (s, 3H), 2.67 (m, 1H), 2.46 (m, 1H), 1.31 (s, 3H), 1.22 (s, 3H) | 4.19 | 338.2 [M + H]⁺ |
| 96 | 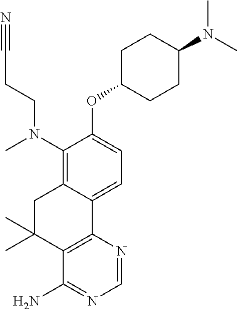 | Examples 8, 9, 22 and 52 using CAS 2417-90-5 and 50-00-0 | DMSO-d₆ + D₂O: 8.22 (s, 1H), 7.88 (d, J = 8.8 Hz, 1H), 6.99 (d, J = 8.8 Hz, 1H), 4.38 (m, 1H), 3.23 (t, J = 6.4 Hz, 2H), 2.96 (m, 2H), 2.68 (m, 1H), 2.64 (s, 3H), 2.57 (m, 1H), 2.43 (m, 1H), 2.36 (s, 6H), 2.16 (m, 2H), 1.89 (m, 2H), 1.46 (m, 4H), 1.30 (s, 3H), 1.22 (s, 3H) | 3.09 | 449.2 [M + H]⁺ |
| 97 | 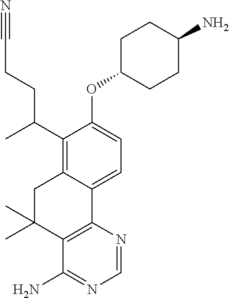 [SMILES: CC(CCC#N)c1c(O[C@H]2CC[C@H](N)CC2)ccc2c1CC(C)(C)c1c(N)ncnc1-2] | — | DMSO-d₆ + D₂O: 8.22 (s, 1H), 7.94 (d, J = 8.8 Hz, 1H), 6.99 (d, J = 8.8 Hz, 1H), 4.39 (m, 1H), 3.30 (m, 1H), 3.04 (m, 1H), 2.79 (m, 2H), 2.28 (m, 3H), 2.15 (m, 2H), 1.96 (m, 3H), 1.48 (m, 4H), 1.26 (m, 9H) | 3.06 | 420.2 [M + H]⁺ |
| 98 | 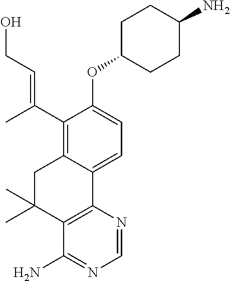 [SMILES: C/C(=C\CO)c1c(O[C@H]2CC[C@H](N)CC2)ccc2c1CC(C)(C)c1c(N)ncnc1-2] | Examples 9 and 92 | DMSO-d₆ + D₂O: 8.23 (s, 1H), 7.94 (d, J = 8.8 Hz, 1H), 6.99 (d, J = 8.8 Hz, 1H), 5.20 (td, J = 6.4 Hz, 1.2 Hz, 1H), 4.30 (m, 1H), 4.19 (m, 1H), 4.06 (m, 1H), 2.98 (m, 1H), 2.67 (m, 2H), 2.06 (m, 2H), 1.92 (m, 2H), 1.80 (s, 3H), 1.42 (m, 4H), 1.29 (s, 3H), 1.17 (s, 3H) | 2.56 | 409.1 [M + H]⁺ |
| 99 | 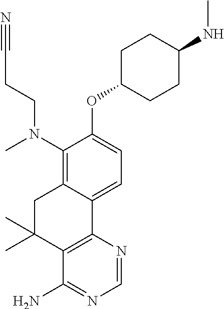 | Examples 8, 9, 22 and 52 using CAS 2417-90-5 and 50-00-0 | DMSO-d₆ + D₂O: 8.52 (s, 1H), 7.78 (d, J = 8.8 Hz, 1H), 7.21 (d, J = 8.8 Hz, 1H), 4.49 (m, 1H), 3.21 (m, 3H), 3.03 (m, 1H), 2.85 (m, 1H), 2.68 (m, 1H), 2.63 (s, 3H), 2.54 (s, 3H), 2.46 (m, 1H), 2.20 (m, 2H), 2.09 (m, 2H), 1.51 (m, 4H), 1.32 (s, 3H), 1.24 (s, 3H) | 3.06 | 435.3 [M + H]⁺ |
| 100 | 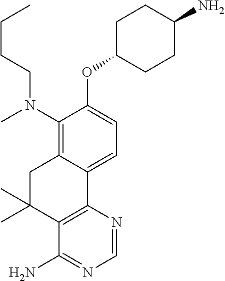 [SMILES: CCCCN(C)c1c(O[C@H]2CC[C@H](N)CC2)ccc2c1CC(C)(C)c1c(N)ncnc1-2] | Examples 8, 9 and 22 using CAS 109-65-9 and 50-00-0 | DMSO-d₆ + D₂O: 8.21 (s, 1H), 7.84 (d, J = 8.8 Hz, 1H), 6.97 (d, J = 8.8 Hz, 1H), 4.36 (m, 1H), 3.06 (m, 2H), 2.90 (m, 2H), 2.78 (m, 1H), 2.61 (s, 3H), 2.14 (m, 2H), 1.97 (m, 2H), 1.47 (m, 4H), 1.25 (m, 10H), 0.81 (t, J = 6.4 Hz, 3H) | 3.04 | 424.2 [M + H]⁺ |
| 101 | 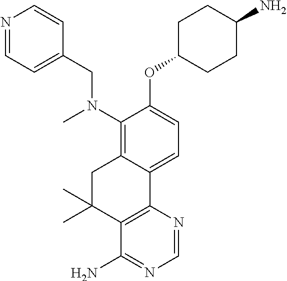 [SMILES: CN(Cc1ccncc1)c1c(O[C@H]2CC[C@H](N)CC2)ccc2c1CC(C)(C)c1c(N)ncnc1-2] | Examples 8, 9 and 22 using CAS 54751-01-8 and 50-00-0 | DMSO-d₆ + D₂O: 8.45 (d, J = 6.0 Hz, 2H), 8.20 (s, 1H), 7.84 (d, J = 8.8 Hz, 1H), 7.32 (d, J = 6.0 Hz, 2H), 7.00 (d, J = 8.8 Hz, 1H), 4.40 (m, 1H), 4.18 (d, J = 6.0 Hz, 2H), 3.09 (m, 1H), 2.88 (d, J = 8.8 Hz, 2H), 2.63 (s, 3H), 2.19 (m, 2H), 2.00 (m, 2H), 1.54 (m, 4H), 1.27 (s, 3H), 1.15 (s, 3H) | 2.30 | 459.3 [M + H]⁺ |
| 102 | 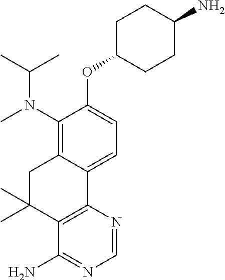 [SMILES: CC(C)N(C)c1c(O[C@H]2CC[C@H](N)CC2)ccc2c1CC(C)(C)c1c(N)ncnc1-2] | Examples 8, 9 and 22 using CAS 75-26-3 and 50-00-0 | DMSO-d₆ + D₂O: 8.21 (s, 1H), 7.83 (d, J = 8.8 Hz, 1H), 6.95 (d, J = 8.8 Hz, 1H), 4.36 (m, 1H), 3.36 (m, 1H), 3.05 (m, 1H), 2.86 (m, 2H), 2.60 (s, 3H), 2.14 (m, 2H), 1.96 (m, 2H), 1.48 (m, 4H), 1.25 (s, 3H), 1.23 (s, 3H). 1.12 (d, J = 6.0 Hz, 3H). 0.76 (d, J = 6.0 Hz, 3H) | 2.86 | 410.2 [M + H]⁺ |
| 103 | 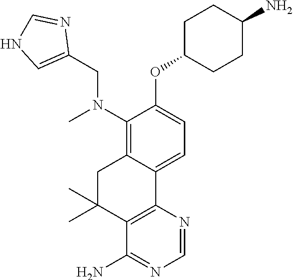 [SMILES: CN(Cc1c[nH]cn1)c1c(O[C@H]2CC[C@H](N)CC2)ccc2c1CC(C)(C)c1c(N)ncnc1-2] | Examples 8, 9 and 22 using CAS 80733-10-4 and 50-00-0 | DMSO-d₆ + D₂O: 9.02 (s, 1H), 8.50 (s, 1H), 7.77 (d, J = 8.8 Hz, 1H), 7.40 (s, 1H), 7.20 (d, J = 8.8 Hz, 1H), 4.51 (m, 1H), 4.24 (m, 2H), 3.12 (m, 1H), 2.88 (s, 2H), 2.69 (s, 3H), 2.21 (m, 2H), 2.03 (m, 2H), 1.58 (m, 4H), 1.27 (s, 3H), 1.1 (s, 3H) | 2.11 | 448.3 [M + H]⁺ |
| 104 | 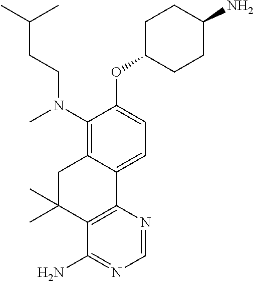 [SMILES: CC(C)CCN(C)c1c(O[C@H]2CC[C@H](N)CC2)ccc2c1CC(C)(C)c1c(N)ncnc1-2] | Examples 8, 9 and 22 using CAS 107-82-4 and 50-00-0 | DMSO-d₆ + D₂O: 8.21 (s, 1H), 7.84 (d, J = 8.8 Hz, 1H), 6.97 (d, J = 8.8 Hz, 1H), 4.36 (m, 1H), 3.09 (m, 2H), 2.91 (m, 2H), 2.78 (m, 1H), 2.61 (s, 3H), 2.14 (m, 2H), 1.96 (m, 2H), 1.58 (m, 1H), 1.47 (m, 4H), 1.28 (s, 3H), 1.20 (s, 3H), 1.14 (m, 2H), 0.82 (d, J = 6.4 Hz, 3H), 0.79 (d, J = 6.4 Hz, 3H) | 3.30 | 438.3 [M + H]⁺ |
| 105 | 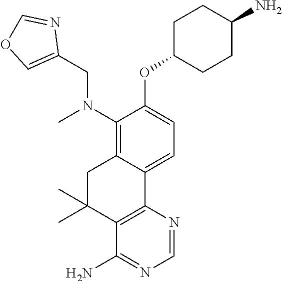 [SMILES: CN(Cc1cocn1)c1c(O[C@H]2CC[C@H](N)CC2)ccc2c1CC(C)(C)c1c(N)ncnc1-2] | Examples 8, 9 and 22 using CAS 1065073-37- 1 and 50-00-0 | DMSO-d₆ + D₂O: 8.50 (s, 1H), 8.20 (s, 1H), 7.85 (s, 1H), 7.67 (d, J = 8.8 Hz, 1H), 7.16 (d, J = 8.8 Hz, 1H), 4.48 (m, 1H), 4.03 (d, J = 8.4 Hz, 2H), 3.13 (m, 1H), 3.02 (m, 1H), 2.85 (m, 1H), 2.66 (s, 3H), 2.17 (m, 2H), 2.01 (m, 2H), 1.55 (m, 4H), 1.29 (s, 3H), 1.11 (s, 3H) | 2.93 | 449.2 [M + H]⁺ |
| 106 | 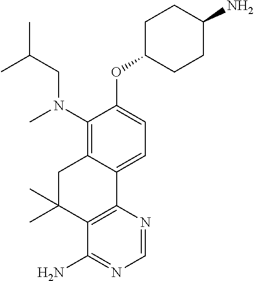 [SMILES: CC(C)CN(C)c1c(O[C@H]2CC[C@H](N)CC2)ccc2c1CC(C)(C)c1c(N)ncnc1-2] | Examples 8, 9 and 22 using CAS 78-77-3 and 50-00-0 | DMSO-d₆ + D₂O: 8.21 (s, 1H), 7.83 (d, J = 8.8 Hz, 1H), 6.97 (d, J = 8.8 Hz, 1H), 4.35 (m, 1H), 3.09 (m, 1H), 2.98 (m, 1H), 2.77 (m, 3H), 2.58 (s, 3H), 2.14 (m, 2H), 1.97 (m, 2H), 1.58 (m, 1H), 1.48 (m, 4H), 1.28 (s, 3H), 1.21 (s, 3H), 0.87 (d, J = 6.4 Hz, 3H), 0.83 (d, J = 6.4 Hz, 3H) | 3.49 | 424.2 [M + H]⁺ |
| 107 | 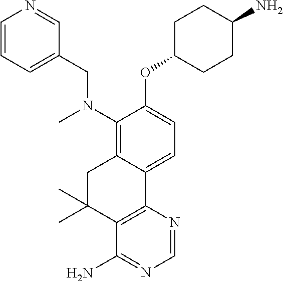 [SMILES: CN(Cc1cccnc1)c1c(O[C@H]2CC[C@H](N)CC2)ccc2c1CC(C)(C)c1c(N)ncnc1-2] | Examples 8, 9 and 22 using CAS 69966-55-8 and 50-00-0 | DMSO-d₆ + D₂O: 8.72 (s, 1H), 8.67 (d, J = 4.8 Hz, 1H), 8.53 (s, 1H), 8.27 (d, J = 8.0 Hz, 1H), 7.82 (dd, J = 4.8, 8.0 Hz, 1H), 7.69 (d, J = 8.8 Hz, 1H), 7.21 (d, J = 8.8 Hz, 1H), 4.51 (m, 1H), 4.31 (s, 2H), 3.13 (m, 1H), 2.92 (s, 2H), 2.65 (s, 3H), 2.20 (m, 2H), 2.02 (m, 2H), 1.58 (m, 4H), 1.27 (s, 3H), 1.16 (s, 3H) | 2.41 | 459.3 [M + H]⁺ |
| 108 | 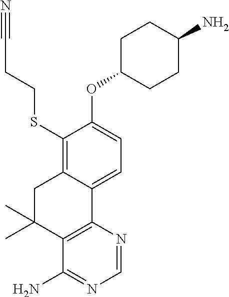 [SMILES: CC1(C)Cc2c(ccc(O[C@H]3CC[C@H](N)CC3)c2SCCC#N)-c2ncnc(N)c21] | — | DMSO-d₆ + D₂O: 8.24 (s, 1H), 8.08 (d, J = 8.8 Hz, 1H), 7.12 (d, J = 8.8 Hz, 1H), 4.46 (m, 1H), 3.09 (s, 2H), 3.06 (m, 1H), 3.02 (t, J = 6.4 Hz, 2H), 2.55 (t, J = 6.4 Hz, 2H), 2.15 (m, 2H), 1.97 (m, 2H), 1.49 (m, 4H), 1.28 (s, 6H) | 2.92 | 424.2 [M + H]⁺ |
| 109 | 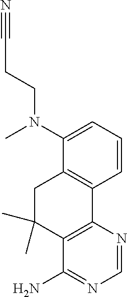 [SMILES: CN(CCC#N)c1cccc2c1CC(C)(C)c1c(N)ncnc1-2] | — | DMSO-d₆ + D₂O: 8.27 (s, 1H), 7.81 (d, J = 6.8 Hz, 1H), 7.26 (m, 2H), 3.12 (t, J = 6.4 Hz, 2H), 2.88 (s, 2H), 2.66 (t, J = 6.4 Hz, 2H), 2.63 (s, 3H), 1.26 (s, 6H) | 3.67 | 308.2 [M + H]⁺ |
| 110 | 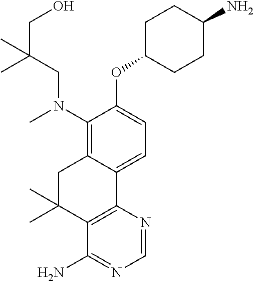 [SMILES: CN(CC(C)(C)CO)c1c(O[C@H]2CC[C@H](N)CC2)ccc2c1CC(C)(C)c1c(N)ncnc1-2] | Examples 8, 9 and 22 using CAS 597-31-9 and 50-00-0 | DMSO-d₆ + D₂O: 8.21 (s, 1H), 7.82 (d, J = 8.8 Hz, 1H), 6.98 (d, J = 8.8 Hz, 1H), 4.36 (m, 1H), 3.22 (d, J = 10.0 Hz, 1H), 3.09 (m, 2H), 3.00 (d, J = 15.8 Hz, 1H), 2.91 (d, J = 14.4 Hz, 1H), 2.79 (m, 2H), 2.61 (s, 3H), 2.15 (m, 2H), 1.98 (m, 2H), 1.50 (m, 4H), 1.28 (s, 3H), 1.24 (s, 3H), 0.76 (s, 6H) | 3.03 | 454.3 [M + H]⁺ |
| 111 | 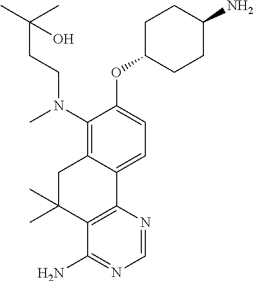 [SMILES: CN(CCC(C)(C)O)c1c(O[C@H]2CC[C@H](N)CC2)ccc2c1CC(C)(C)c1c(N)ncnc1-2] | Examples 8, 9 and 22 using CAS 35979-69-2 and 50-00-0 | DMSO-d₆ + D₂O: 8.21 (s, 1H), 7.84 (d, J = 8.8 Hz, 1H), 6.97 (d, J = 8.8 Hz, 1H), 4.37 (m, 1H), 3.18 (m, 1H), 3.06 (m, 1H), 2.91 (m, 2H), 2.78 (m, 1H), 2.63 (s, 3H), 2.15 (m, 2H), 1.98 (m, 2H), 1.49 (m, 5H), 1.24 (m, 7H), 1.02 (s, 6H) | 2.47 | 454.3 [M + H]⁺ |
| 112 | 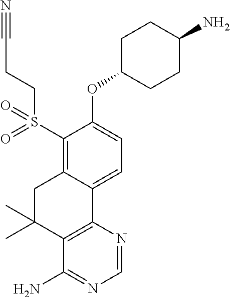 [SMILES: CC1(C)Cc2c(ccc(O[C@H]3CC[C@H](N)CC3)c2S(=O)(=O)CCC#N)-c2ncnc(N)c21] | Examples 9, 32 and 108 | DMSO-d₆ + D₂O: 8.35 (d, J = 8.8 Hz, 1H), 8.27 (s, 1H), 7.36 (d, J = 8.8 Hz, 1H), 4.62 (m, 1H), 3.75 (t, J = 6.4 Hz, 2H), 3.40 (s, 2H), 3.03 (m, 1H), 2.91 (t, J = 6.4 Hz, 2H), 2.15 (m, 2H), 1.97 (m, 2H), 1.50 (m, 4H), 1.27 (s, 6H) | 2.49 | 456.2 [M + H]⁺ |
| 113 | 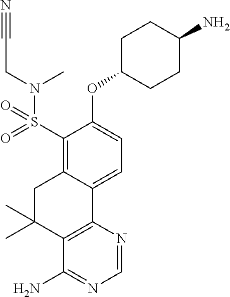 [SMILES: CN(CC#N)S(=O)(=O)c1c(O[C@H]2CC[C@H](N)CC2)ccc2c1CC(C)(C)c1c(N)ncnc1-2] | — | DMSO-d₆ + D₂O: 8.31 (d, J = 8.8 Hz, 1H), 8.27 (s, 1H), 7.35 (d, J = 8.8 Hz, 1H), 4.56 (m, 1H), 4.43 (s, 2H), 3.30 (s, 2H), 3.00 (m, 1H), 2.87 (s, 3H), 2.12 (m, 2H), 1.97 (m, 2H), 1.60 (m, 2H), 1.47 (m, 2H), 1.26 (s, 6H) | 2.84 | 471.1 [M + H]⁺ |
| 114 | 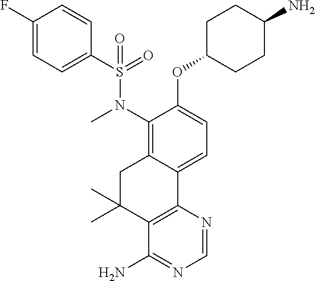 [SMILES: CN(c1c(O[C@H]2CC[C@H](N)CC2)ccc2c1CC(C)(C)c1c(N)ncnc1-2)S(=O)(=O)c1ccc(F)cc1] | Examples 5, 9, 22, 42 and 48 using CAS 98-74- 8, 74-88-4 and 50-00-0 | DMSO-d₆ + D₂O: 8.51 (s, 1H), 7.89 (d, J = 8.8 Hz, 1H), 7.83 (dd, J = 8.8 Hz, 5.2 Hz, 2H), 7.47 (t, J = 8.8 Hz, 2H), 7.20 (d, J = 8.8 Hz, 1H), 4.26 (m, 1H), 3.08 (s, 3H), 2.87 (m, 3H), 1.83 (m, 4H), 1.40 (m, 5H), 1.16 (s, 3H), 0.91 (m, 2H) | 3.39 | 526.2 [M + H]⁺ |
| 115 | 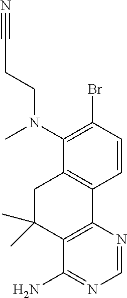 [SMILES: CN(CCC#N)c1c(Br)ccc2c1CC(C)(C)c1c(N)ncnc1-2] | — | DMSO-d₆ + D₂O: 8.27 (s, 1H), 7.82 (d, J = 8.4 Hz, 1H), 7.55 (d, J = 8.4 Hz, 1H), 3.41 (m, 1H), 3.34 (m, 1H), 2.96 (m, 2H), 2.78 (s, 3H), 2.70 (m, 1H), 2.59 (m, 1H), 1.30 (s, 3H), 1.28 (s, 3H) | 4.6 | 386.0, 388.1 [M + H]^{⁺} |
| 116 | 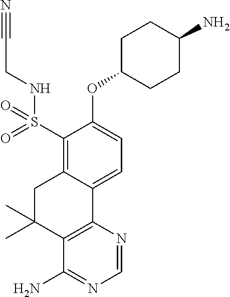 [SMILES: CC1(C)Cc2c(ccc(O[C@H]3CC[C@H](N)CC3)c2S(=O)(=O)NCC#N)-c2ncnc(N)c21] | Examples 9 and 113 using CAS 6011-14-9 | DMSO-d₆ + D₂O: 8.28 (d, J = 8.8 Hz, 1H), 8.26 (s, 1H), 7.31 (d, J = 8.8 Hz, 1H), 4.56 (m, 1H), 4.08 (s, 2H), 3.30 (s, 2H), 3.01 (m, 1H), 2.12 (m, 2H), 1.98 (m, 2H), 1.68 (m, 2H), 1.46 (m, 2H), 1.26 (s, 6H) | 2.57 | 457.1 [M + H]⁺ |
| 117 | 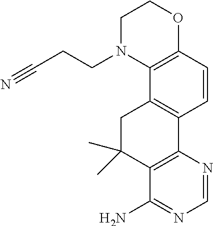 [SMILES: CC1(C)Cc2c(ccc3c2N(CCC#N)CCO3)-c2ncnc(N)c21] | Examples 3 and 22 using CAS 2417- 90-5 | DMSO-d₆ + D₂O: 8.26 (s, 1H), 7.67 (d, J = 8.8 Hz, 1H), 6.77 (d, J = 8.4 Hz, 1H), 4.14 (t, J = 4.0 Hz, 2H), 3.13 (t, J = 4.0 Hz, 2H), 2.97 (m, 2H), 2.90 (m, 4H), 1.28 (s, 6H) | 3.56 | 336.3 [M + H]⁺ |
| 118 | 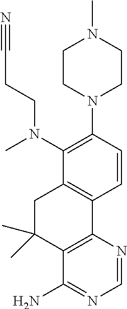 [SMILES: CN1CCN(c2ccc3c(c2N(C)CCC#N)CC(C)(C)c2c(N)ncnc2-3)CC1] | — | DMSO-d₆ + D₂O: 8.24 (s, 1H), 7.83 (d, J = 8.4 Hz, 1H), 7.06 (d, J = 8.4 Hz, 1H), 3.61 (m, 1H), 3.31 (m, 1H), 2.97 (m, 4H), 2.80 (s, 3H), 2.66 (m, 8H), 2.25 (s, 3H), 1.38 (s, 3H), 1.17 (s, 3H) | 2.49 | 406.2 [M + H]⁺ |
| 119 | 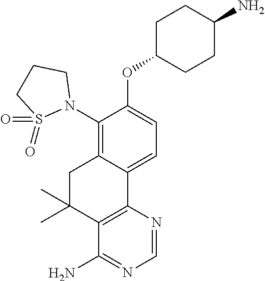 [SMILES: CC1(C)Cc2c(ccc(O[C@H]3CC[C@H](N)CC3)c2N2CCCS2(=O)=O)-c2ncnc(N)c21] | Examples 9, 28 and 42 using CAS 1633-82-5 | DMSO-d₆ + D₂O: 8.55 (s, 1H), 7.94 (d, J = 8.8 Hz, 1H), 7.32 (d, J = 8.8 Hz, 1H), 4.52 (m, 1H), 3.65 (q, J = 8.0 Hz, 1H), 3.49 (overlapped with water peak, 2H), 3.12 (m, 2H), 2.93 (m, 2H), 2.54 (m, 1H), 2.41 (m, 1H), 2.13 (m, 2H), 2.01 (m, 2H), 1.54 (m, 4H), 1.30 (s, 3H), 1.24 (s, 3H) | 2.43 | 458.1 [M + H]⁺ |
| 120 | 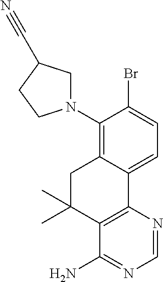 [SMILES: CC1(C)Cc2c(ccc(Br)c2N2CCC(C#N)C2)-c2ncnc(N)c21] | — | DMSO-d₆ + D₂O: 8.28 (s, 1H), 7.84 (d, J = 8.4 Hz, 1H), 7.57 (d, J = 8.4 Hz, 1H), 3.57 (m, 2H), 3.32 (m, 3H), 2.86 (m, 2H), 2.41 (m, 1H), 2.31 (m, 1H), 1.29 (s, 3H), 1.26 (s, 3H) | 4.78 | 398.1, 400.1 [M + H]⁺ |
| 121 | 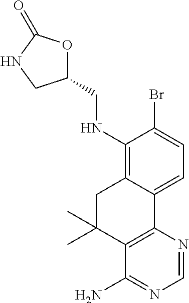 [SMILES: CC1(C)Cc2c(ccc(Br)c2NC[C@@H]2CNC(=O)O2)-c2ncnc(N)c21] | — | DMSO-d₆ + D₂O: 8.27 (s, 1H), 7.62 (d, J = 8.4 Hz, 1H), 7.50 (d, J = 8.4 Hz, 1H), 4.67 (m, 1H), 3.50 (m, 2H), 3.18 (m, 2H), 2.83 (s, 2H), 1.28 (s, 3H), 1.26 (s, 3H) | 3.19 | 418.2, 420.2 [M + H]⁺ |
| 122 | 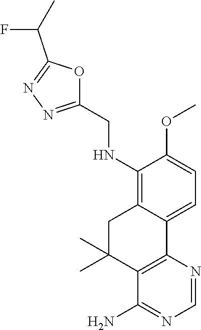 [SMILES: COc1ccc2c(c1NCc1nnc(C(C)F)o1)CC(C)(C)c1c(N)ncnc1-2] | — | DMSO-d₆ + D₂O: 8.21 (s, 1H), 7.69 (d, J = 8.4 Hz, 1H), 6.87 (d, J = 8.4 Hz, 1H), 5.90 (dq, J = 6.4, 47.2 Hz, 1H), 4.43 (s, 2H), 3.73 (s, 3H), 2.72 (s, 2H), 1.63 (dd, J = 6.4, 24.0 Hz, 3H), 1.23 (s, 6H) | 3.47 | 399.3 [M + H]⁺ |
| 123 | 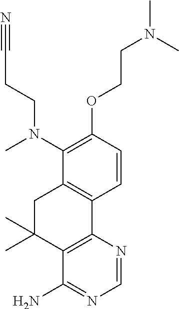 | Examples 5, 19, 22 and 48 using 824- 94-2, 98-74- 8, 2417-90-5, 50-00-0 and 4584-46-7 | DMSO-d₆ + D₂O: 8.22 (s, 1H), 7.89 (d, J = 8.8 Hz, 1H), 6.98 (d, J = 8.8 Hz, 1H), 4.13 (t, J = 5.6 Hz, 2H), 3.26 (m, 2H), 3.14 (m, 1H), 2.77 (m, 3H), 2.66 (m, 4H), 2.46 (m, 1H), 2.29 (s, 6H), 1.30 (s, 3H), 1.22 (s, 3H) | 2.54 | 395.2 [M + H]⁺ |
| 124 | 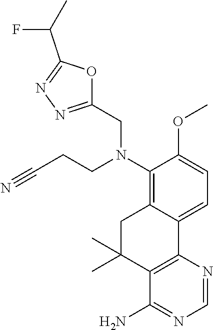 [SMILES: COc1ccc2c(c1N(CCC#N)Cc1nnc(C(C)F)o1)CC(C)(C)c1c(N)ncnc1-2] | Examples 2, 5, 22 and 48 using 98-74- 8 and 6162- 76-1 | DMSO-d₆ + D₂O: 8.30 (s, 1H), 7.93 (d, J = 8.8 Hz, 1H), 7.00 (d, J = 8.8 Hz, 1H), 5.97 (dq, J = 6.4, 47.2 Hz, 1H), 4.39 (m, 2H), 3.72 (d, J = 2.4 Hz, 3H), 3.41 (m, 1H), 3.32 (m, 1H), 3.11 (m, 1H), 2.69 (m, 2H), 2.46 (m, 1H), 1.69 (dd, J = 6.4, 24.4 Hz, 3H), 1.25 (s, 3H), 1.21 (d, J = 6.0 Hz, 3H) | 3.81 | 452.2 [M + H]⁺ |
| 125 | 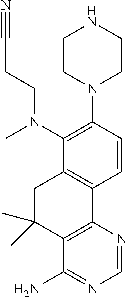 [SMILES: CN(CCC#N)c1c(N2CCNCC2)ccc2c1CC(C)(C)c1c(N)ncnc1-2] | Examples 9 and 118 using 57260- 71-6, 98-74- 8, 2417-90-5 and 50-00-0 | DMSO-d₆ + D₂O: 8.30 (s, 1H), 7.85 (d, J = 8.4 Hz, 1H), 7.06 (d, J = 8.4 Hz, 1H), 3.60 (m, 1H), 3.34 (m, 1H), 3.01 (m, 7H), 2.80 (m, 5H), 2.66 (m, 2H), 2.53 (m, 1H), 1.38 (br, 3H), 1.17 (br, 3H) | 2.44 | 392.3 [M + H]⁺ |
| 126 | 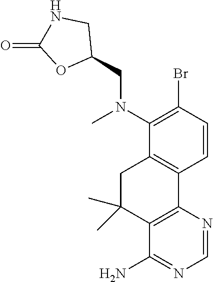 [SMILES: CN(C[C@@H]1CNC(=O)O1)c1c(Br)ccc2c1CC(C)(C)c1c(N)ncnc1-2] | Examples 5, 22, 48 and 120 using 98- 74-8, 50-00-0 and 169048- 83-3 | DMSO-d₆ + D₂O: 8.26 (s, 1H), 7.79 (dd, J = 4.4, 8.4 Hz, 1H), 7.56 (dd, J = 4.0, 8.4 Hz, 1H), 4.65 (m, 1H), 3.53 (m, 1H), 3.31 (m, 2H), 3.04 (m, 2H), 2.81 (m, 4H), 1.28 (m, 6H) | 3.82 | 432.2, 434.2 [M + H]⁺ |
| 127 | 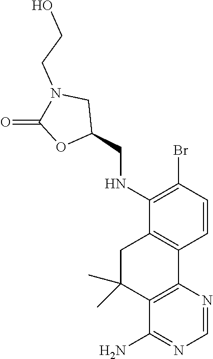 [SMILES: CC1(C)Cc2c(ccc(Br)c2NC[C@@H]2CN(CCO)C(=O)O2)-c2ncnc(N)c21] | Examples 22 and 121 using 105- 36-2 | DMSO-d₆ + D₂O: 8.29 (s, 1H), 7.64 (d, J = 8.4 Hz, 1H), 7.52 (d, J = 8.4 Hz, 1H), 4.62 (m, 1H), 3.70 (t, J = 8.8 Hz, 1H), 3.49 (t, J = 5.6 Hz, 2H), 3.37 (m, 1H), 3.31 (m, 4H), 2.85 (s, 2H), 1.29 (s, 3H), 1.28 (s, 3H) | 3.11 | 462.1, 464.1 [M + H]⁺ |
| 128 | 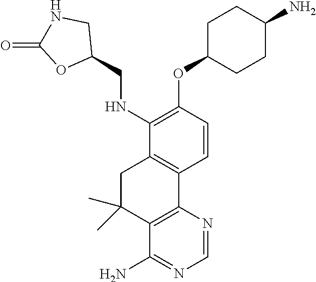 [SMILES: CC1(C)Cc2c(ccc(O[C@H]3CC[C@@H](N)CC3)c2NC[C@@H]2CNC(=O)O2)-c2ncnc(N)c21] | Examples 5, 48 and 54 using 111300-06-2, 98-74-8 and 169048-83-3 | DMSO-d₆ + D₂O: 8.22 (s, 1H), 7.67 (d, J = 8.4 Hz, 1H), 6.91 (d, J = 8.4 Hz, 1H), 4.65 (m, 2H), 3.53 (m, 1H), 3.15 (m, 4H), 2.75 (s, 2H), 2.01 (m, 2H), 1.70 (m, 6H), 1.30 (s, 3H), 1.25 (s, 3H) | 2.35 | 453.2 [M + H]⁺ |
| 129 | 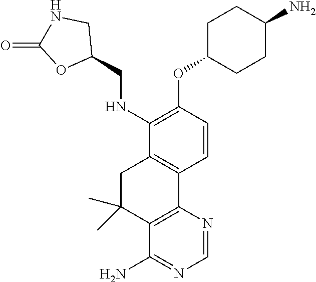 [SMILES: CC1(C)Cc2c(ccc(O[C@H]3CC[C@H](N)CC3)c2NC[C@@H]2CNC(=O)O2)-c2ncnc(N)c21] | Examples 5, 48 and 54 using 167081-25-6, 98-74-8 and 169048-83-3 | DMSO-d₆ + D₂O: 8.22 (s, 1H), 7.66 (d, J = 8.8 Hz, 1H), 6.97 (d, J = 8.8 Hz, 1H), 4.60 (m, 1H), 4.34 (m, 1H), 3.49 (overlapped with water peak, 1H), 3.12 (m, 4H), 2.73 (s, 2H), 2.11 (m, 2H), 1.96 (m, 2H), 1.48 (m, 4H), 1.27 (s, 3H), 1.26 (s, 3H) | 2.19 | 453.3 [M + H]⁺ |
| 130 | 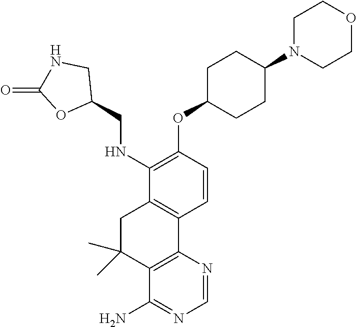 [SMILES: CC1(C)Cc2c(ccc(O[C@H]3CC[C@@H](N4CCOCC4)CC3)c2NC[C@@H]2CNC(=O)O2)-c2ncnc(N)c21] | Examples 22 and 128 using 5414- 19-7 | DMSO-d₆: 23 (s, 1H), 7.68 (d, J = 8.4 Hz, 1H), 7.50 (s, 1H), 6.90 (d, J = 8.4 Hz, 1H), 6.34 (br, 2H), 4.64 (m, 2H), 4.18 (m, 1H), 3.56 (m, 4H), 3.53 (t, J = 8.8 Hz, 1H), 3.24 (m, 1H), 3.18 (m, 2H), 2.74 (s, 2H), 2.47 (overlapped with DMSO peak, 4H), 2.31 (m, 1H), 2.05 (m, 2H), 1.60 (m, 6H), 1.28 (s, 3H), 1.27 (s, 3H) | 2.44 | 523.5 [M + H]⁺ |
| 131 | 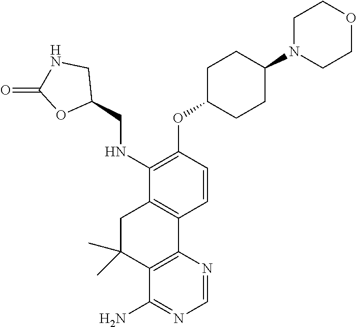 [SMILES: CC1(C)Cc2c(ccc(O[C@H]3CC[C@H](N4CCOCC4)CC3)c2NC[C@@H]2CNC(=O)O2)-c2ncnc(N)c21] | Examples 22 and 129 using 5414- 19-7 | DMSO-d₆: 8.23 (s, 1H), 7.66 (d, J = 8.8 Hz, 1H), 7.48 (s, 1H), 6.94 (d, J = 8.8 Hz, 1H), 6.34 (br, 2H), 4.58 (m, 1H), 4.32 (m, 1H), 4.21 (t, J = 6.4 Hz, 1H), 3.56 (m, 4H), 3.49 (t, J = 8.8 Hz, 1H), 3.24 (m, 1H), 3.15 (m, 2H), 2.73 (s, 2H), 2.46 (m, 4H), 2.25 (m, 1H), 2.10 (m, 2H), 1.87 (m, 2H), 1.41 (m, 4H), 1.27 (s, 3H), 1.26 (s, 3H) | 2.31 | 523.5 [M + H]⁺ |
| 132 | 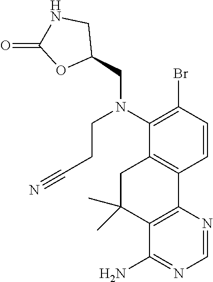 [SMILES: CC1(C)Cc2c(ccc(Br)c2N(CCC#N)C[C@@H]2CNC(=O)O2)-c2ncnc(N)c21] | Examples 2, 5, 8, 22, 48 and 120 using 6162- 76-1 and 169048-83-3 | DMSO-d₆ + D₂O: 8.29 (s, 1H), 7.88 (dd, J = 5.6, 8.4 Hz, 1H), 7.60 (dd, J = 3.2, 8.4 Hz, 1H), 4.60 (m, 1H), 3.36 (m, 5H), 3.08 (m, 1H), 2.98 (m, 2H), 2.63 (m, 2H), 1.31 (m, 6H) | 3.50 | 471.2, 473.2 [M + H]⁺ |

*When a single asterix in the second column is next to a chiral center, the compound indicated is an isomer at this position;if no asterix is indicated at a chiral center and the stereochemistry is not defined, then the compound is a racemic mixture of the enantiomers.
**A hyphen in the third column indicates that the synthesis procedure is described below.

### Preparation of Example 1: 8-methoxy-5,5,7-trimethyl-6H-benzo[h]quinazolin-4-amine

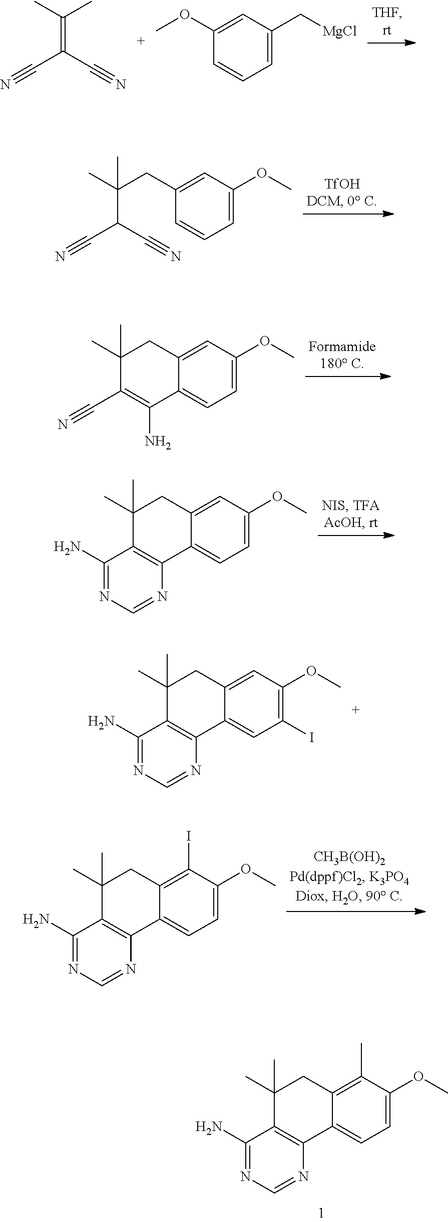

### Step 1: Preparation of 2-[2-(3-methoxyphenyl)-1,1-dimethyl-ethyl]propanedinitrile

3-Methoxybenzylmagnesium chloride (96 mL, 24 mmol, 0.25 M in THF) was added dropwise at 0° C. to a stirred solution of isopropylidenemalononitrile (2 g, 18.5 mmol) in THF (50 mL). The resulting solution was stirred at rt for 3 h. 1N HCl aqueous solution was then added at 0° C. and the resulting mixture was concentrated. The residue was extracted with EA and H₂O. The combined organic layers were dried over Na₂SO₄, filtered, concentrated and purified by column chromatography (silica gel; PE:EA; 5:1; v:v) to afford 2-[2-(3-methoxyphenyl)-1,1-dimethyl-ethyl]propanedinitrile as a yellow oil (1.69 g, 38% yield).

¹H NMR (400 MHz, CDCl₃) δ ppm: 7.27 (m, 1H), 6.86 (m, 1H), 6.79 (d, J=7.6 Hz, 1H), 6.74 (m, 1H), 3.82 (s, 3H), 3.44 (s, 1H), 2.81 (s, 2H), 1.29 (s, 6H). MS m/z (+ESI): 229.1 [M+H]⁺.

### Step 2: Preparation of 1-amino-6-methoxy-3,3-dimethyl-4H-naphthalene-2-carbonitrile

TfOH (1.04 g, 6.24 mmol) was added at 0° C. to a stirred solution of 2-[2-(3-methoxyphenyl)-1,1-dimethyl-ethyl]propanedinitrile (300 mg, 1.25 mmol) in DCM (6 mL) and the resulting mixture was stirred at 0° C. for 2 h. Saturated NaHCO₃ aqueous solution was added, and the mixture was extracted with DCM. The combined organic layers were dried over Na₂SO₄, filtered and concentrated to afford 1-amino-6-methoxy-3,3-dimethyl-4H-naphthalene-2-carbonitrile as a yellow solid (300 mg, 95% yield) which was used in the next step without further purification.

¹H NMR (400 MHz, CDCl₃) δ ppm: 7.31 (d, J=8.8 Hz, 1H), 6.81 (m, 1H), 6.74 (d, J=2.4 Hz, 1H), 4.51 (br, 2H), 3.85 (s, 3H), 2.69 (s, 2H), 1.17 (s, 6H).

MS m/z (+ESI): 229.1 [M+H]⁺.

### Step 3: Preparation of 8-methoxy-5,5-dimethyl-6H-benzo[h]quinazolin-4-amine

A suspension of 1-amino-6-methoxy-3,3-dimethyl-4H-naphthalene-2-carbonitrile (1 g, 3.94 mmol) and formamide (19 mL, 473 mmol) was stirred at 180° C. for 8 h. After being cooled to rt, the reaction mixture was diluted with H₂O and extracted with EA. The combined organic layers were dried over Na₂SO₄, filtered, concentrated and purified by column chromatography (silica gel; PE:EA; 1:1; v:v) to afford 8-methoxy-5,5-dimethyl-6H-benzo[h]quinazolin-4-amine as a light yellow solid (700 mg, 63% yield).

¹H NMR (400 MHz, DMSO-d₆) δ ppm: 8.24 (s, 1H), 7.99 (d, J=8.8 Hz, 1H), 6.86 (m, 1H), 6.79 (d, J=2.8 Hz, 1H), 6.37 (br, 2H), 3.79 (s, 3H), 2.75 (s, 2H), 1.28 (s, 6H).

MS m/z (+ESI): 256.1 [M+H]⁺.

### Step 4: Preparation of 7-iodo-8-methoxy-5,5-dimethyl-6H-benzo[h]quinazolin-4-amine and 9-iodo-8-methoxy-5,5-dimethyl-6H-benzo[h]quinazolin-4-amine

NIS (1.21 g, 5.29 mmol) was added portionwise to a stirred solution of 8-methoxy-5,5-dimethyl-6H-benzo[h]quinazolin-4-amine (300 mg, 1.06 mmol) in AcOH (5 mL) and TFA (0.3 mL). After 20 h, the solvent was removed and the residue was dissolved in H₂O and neutralized with a saturated K₂CO₃ aqueous solution before extraction with EA. The combined organic layers were dried over Na₂SO₄, filtered, concentrated to afford a mixture of 7-iodo-8-methoxy-5,5-dimethyl-6H-benzo[h]quinazolin-4-amine and 9-iodo-8-methoxy-5,5-dimethyl-6H-benzo[h]quinazolin-4-amine (ratio 1:4) as a light yellow solid (403 mg, 79% yield).

MS m/z (+ESI): 276.1, 278.1 [M+H]⁺.

### Step 5: Preparation of 8-methoxy-5,5,7-trimethyl-6H-benzo[h]quinazolin-4-amine

CH₃B(OH)₂ (22 mg, 0.35 mmol) was added to a stirred solution of a mixture of 7-iodo-8-methoxy-5,5-dimethyl-6H-benzo[h]quinazolin-4-amine and 9-iodo-8-methoxy-5,5-dimethyl-6H-benzo[h]quinazolin-4-amine (ratio 1:4) in Diox (5 mL) and H₂O (1 mL), followed by K₃PO₄ (63 mg, 0.30 mmol) and Pd(dppf)Cl₂ (219 mg, 0.30 mmol). After 20 h stirring at 90° C., the reaction mixture was filtered through decalite, concentrated and purified by preparative HPLC to afford 8-methoxy-5,5,7-trimethyl-6H-benzo[h]quinazolin-4-amine as a white solid (79 mg, 6% yield)

¹H NMR (400 MHz, DMSO-d₆+D₂O) δ ppm: 8.28 (s, 1H), 7.89 (d, J=8.8 Hz, 1H), 6.94 (d, J=8.8 Hz, 1H), 3.82 (s, 3H), 2.75 (s, 2H), 2.13 (s, 3H), 1.27 (s, 6H).

MS m/z (+ESI): 270.1 [M+H]⁺.

### Preparation of Example 2: 8-methoxy-5,5-dimethyl-6H-benzo[h]quinazoline-4,7-diamine

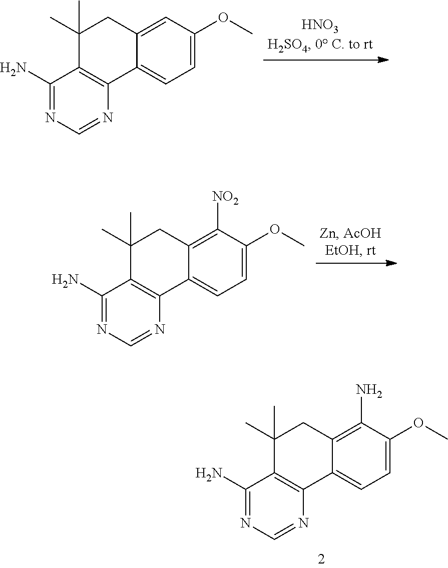 [SMILES: COc1ccc2c(c1)CC(C)(C)c1c(N)ncnc1-2 ; COc1ccc2c(c1[N+](=O)[O-])CC(C)(C)c1c(N)ncnc1-2 ; COc1ccc2c(c1N)CC(C)(C)c1c(N)ncnc1-2]

### Step 1: Preparation of 8-methoxy-5,5-dimethyl-7-nitro-6H-benzo[h]quinazolin-4-amine

HNO₃ (14.6 mL, 228.2 mmol) was added dropwise at 0° C. to a stirred solution of 8-methoxy-5,5-dimethyl-6H-benzo[h]quinazolin-4-amine (4.5 g, 17.55 mmol) in H₂SO₄ (29 mL), followed by H₂O (14.5 mL). After 2 h stirring at rt, the reaction mixture was poured into saturated NaHCO₃ aqueous solution and extracted with EA. The combined organic layers were washed with brine, dried over Na₂SO₄, filtered and concentrated. The residue was purified by column chromatography (silica gel; DCM:EA; 10:1 to 0:1; v:v) to afford 8-methoxy-5,5-dimethyl-7-nitro-6H-benzo[h]quinazoline-4-amine as an off-white solid (640 mg, 11% yield).

¹H NMR (400 MHz, DMSO-d₆+D₂O) δ ppm: 8.51 (s, 1H), 8.11 (d, J=8.8 Hz, 1H), 7.41 (d, J=8.8 Hz, 1H), 3.95 (s, 3H), 2.71 (s, 2H), 1.27 (s, 6H).

MS m/z (+ESI): 301.2 [M+H]⁺.

### Step 2: Preparation of 8-methoxy-5,5-dimethyl-6H-benzo[h]quinazoline-4,7-diamine

Zinc (300 mg, 4.49 mmol) was added to a stirred solution of 8-methoxy-5,5-dimethyl-7-nitro-6H-benzo[h]quinazolin-4-amine (300 mg, 0.15 mmol) in EtOH (10 mL) and AcOH (3 mL). After 4 h stirring, the reaction mixture was filtered, concentrated and purified by preparative HPLC to afford 8-methoxy-5,5-dimethyl-6H-benzo[h]quinazoline-4,7-diamine as a light yellow solid (178 mg, 66% yield). ¹H NMR (400 MHz, DMSO-d₆+D₂O) δ ppm: 8.53 (s, 1H), 7.19 (d, J=8.4 Hz, 1H), 6.96 (d, J=8.4 Hz, 1H), 3.87 (s, 3H), 2.72 (s, 2H), 1.29 (s, 6H).

MS m/z (+ESI): 271.1 [M+H]⁺.

### Preparation of Examples 3 and 4: 6,6-dimethyl-2,3,4,5-tetrahydroquinazolino[7,8-f][1,4]benzoxazin-7-amine and 4,6,6-trimethyl-3,5-dihydro-2H-quinazolino[7,8-f][1,4]benzoxazin-7-amine

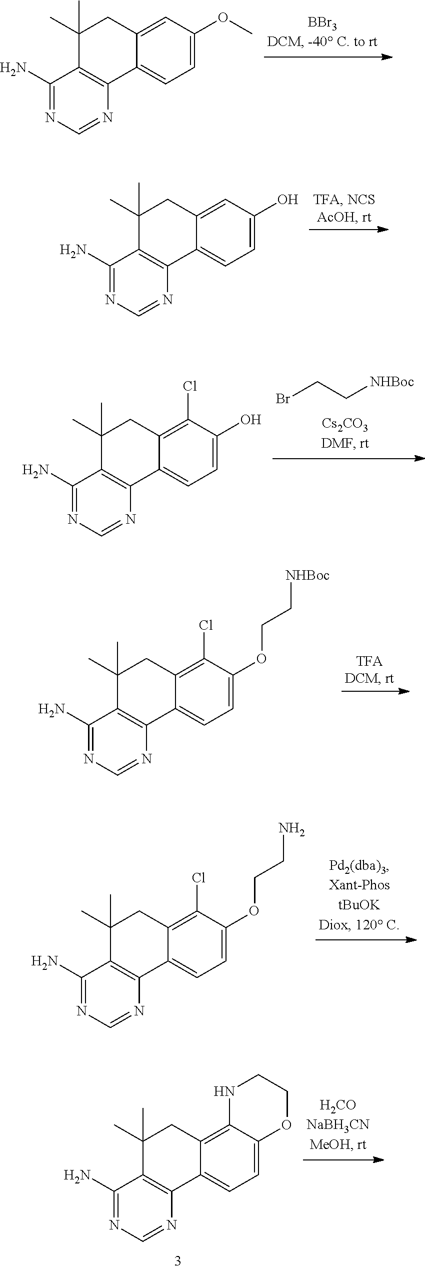

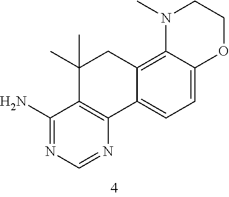 [SMILES: CN1CCOc2ccc3c(c21)CC(C)(C)c1c(N)ncnc1-3]

### Step 1: Preparation of 4-amino-5,5-dimethyl-6H-benzo[h]quinazolin-8-ol

BBr₃ (1M in DCM, 4.8 mL, 4.80 mmol) was added dropwise at −40° C. to a stirred solution of 8-methoxy-5,5-dimethyl-6H-benzo[h]quinazolin-4-amine (450 mg, 1.58 mmol) in DCM (30 mL). The resulting solution was stirred at rt for 18 h. Saturated NaHCO₃ aqueous solution was added and the resulting green solid was collected by filtration, washed with H₂O and dried under reduced pressure to afford 4-amino-5,5-dimethyl-6H-benzo[h]quinazolin-8-ol (350 mg, 82% yield).

¹H NMR (400 MHz, DMSO-d₆) δ ppm: 8.21 (s, 1H), 7.88 (d, J=8.4 Hz, 1H), 6.67 (m, 1H), 6.59 (s, 1H), 6.29 (br, 2H), 2.67 (s, 2H), 1.26 (s, 6H).

MS m/z (+ESI): 242.2 [M+H]⁺.

### Step 2: Preparation of 4 amino-7-chloro-5,5-dimethyl-6H-benzo[h]quinazolin-8-ol

NCS (1.07 g, 7.96 mmol) was added portionwise to a stirred solution of 4-amino-5,5-dimethyl-6H-benzo[h]quinazolin-8-ol (2 g, 6.63 mmol) in AcOH (40 mL) and TFA (10 mL). After 3 h stirring, the solvent was removed and the residue was purified by combiflash to afford 4 amino-7-chloro-5,5-dimethyl-6H-benzo[h]quinazolin-8-ol as a white solid (1.83 g, 15% yield).

MS m/z (+ESI): 276.1, 278.1 [M+H]⁺.

### Step 3: Preparation of tert-butyl N-[2-[(4-amino-7-chloro-5,5-dimethyl-6H-benzo[h]quinazolin-8-yl)oxy]ethyl]carbamate

tert-Butyl N-(2-bromoethyl)carbamate (226 mg, 0.98 mmol) was added to a stirred solution of 4 amino-7-chloro-5,5-dimethyl-6H-benzo[h]quinazolin-8-ol (250 mg, 0.82 mmol) in DMF (5 mL), followed by Cs₂CO₃ (543 mg, 1.63 mmol). After 20 h stirring, the solvent was removed and the residue was purified by combiflash to afford tert-butyl N-[2-[(4-amino-7-chloro-5,5-dimethyl-6H-benzo[h]quinazolin-8-yl)oxy]ethyl]carbamate as a white solid (342 mg, 79% yield).

¹H NMR (400 MHz, DMSO-d₆) δ ppm: 8.27 (s, 1H), 8.02 (d, J=8.4 Hz, 1H), 7.11 (d, J=8.4 Hz, 1H), 6.98 (br, 1H), 6.50 (br, 2H), 4.09 (t, J=5.2 Hz, 2H), 3.33 (t, J=5.2 Hz, 2H), 2.91 (s, 2H), 1.38 (s, 9H), 1.30 (s, 6H).

MS m/z (+ESI): 419.2, 421.3 [M+H]⁺.

### Step 4: Preparation of 8-(2-aminoethoxy)-7-chloro-5,5-dimethyl-6H-benzo[h]quinazolin-4-amine

A solution of tert-butyl N-[2-[(4-amino-7-chloro-5,5-dimethyl-6H-benzo[h]quinazolin-8-yl)oxy]ethyl]carbamate (300 mg, 0.64 mmol) in TFA (3 mL) was stirred for 1 h before concentration and purification by preparative HPLC to afford 8-(2-aminoethoxy)-7-chloro-5,5-dimethyl-6H-benzo[h]quinazolin-4-amine as a white solid (300 mg, 98% yield).

MS m/z (+ESI): 319.2, 321.2 [M+H]⁺.

### Step 5: Preparation of 6,6-dimethyl-2,3,4,5-tetrahydroquinazolino[7,8-f][1,4]benzoxazin-7-amine

t-BuOK (285 mg, 2.94 mmol) was added to a stirred solution of 8-(2-aminoethoxy)-7-chloro-5,5-dimethyl-6H-benzo[h]quinazolin-4-amine (280 mg, 0.59 mmol) in Diox (10 mL), followed by Pd₂(dba)₃ (55 mg, 0.06 mmol) and Xant-Phos (70 mg, 0.12 mmol). After 18 h stirring at 120° C., the reaction mixture was filtered through decalite, concentrated and purified by preparative HPLC to afford 6,6-dimethyl-2,3,4,5-tetrahydroquinazolino[7,8-f][1,4]benzoxazin-7-amine as a light yellow solid (166 mg, 36% yield)

¹H NMR (400 MHz, DMSO-d₆+D₂O) δ ppm: 8.52 (s, 1H), 7.10 (d, J=8.4 Hz, 1H), 6.79 (d, J=8.4 Hz, 1H), 4.18 (t, J=4.4 Hz, 2H), 3.33 (t, J=4.4 Hz, 2H), 2.66 (s, 2H), 1.29 (s, 6H).

MS m/z (+ESI): 283.2 [M+H]⁺.

### Step 6: Preparation of 4,6,6-trimethyl-3,5-dihydro-2H-quinazolino[7,8-f][1,4]benzoxazin-7-amine

H₂CO (11 mg, 0.13 mmol) was added to a stirred solution of 6,6-dimethyl-2,3,4,5-tetrahydroquinazolino[7,8-f][1,4]benzoxazin-7-amine (38 mg, 0.13 mmol) in MeOH (5 mL), followed by NaBH₃CN (9 mg, 0.13 mmol). After 1 h stirring, the reaction mixture was concentrated and purified by preparative HPLC to afford 4,6,6-trimethyl-3,5-dihydro-2H-quinazolino[7,8-f][1,4]benzoxazin-7-amine as a white solid (29 mg, 72% yield).

¹H NMR (400 MHz, DMSO-d₆+D₂O) δ ppm: 8.54 (s, 1H), 7.42 (d, J=8.4 Hz, 1H), 6.92 (d, J=8.4 Hz, 1H), 4.20 (t, J=4.4 Hz, 2H), 3.09 (t, J=4.4 Hz, 2H), 2.84 (s, 2H), 2.63 (s, 3H), 1.28 (s, 6H).

MS m/z (+ESI): 297.2 [M+H]⁺.

### Preparation of Example 5: 8-(trans-4-aminocyclohexoxy)-5,5-dimethyl-6H-benzo[h]quinazoline-4,7-diamine

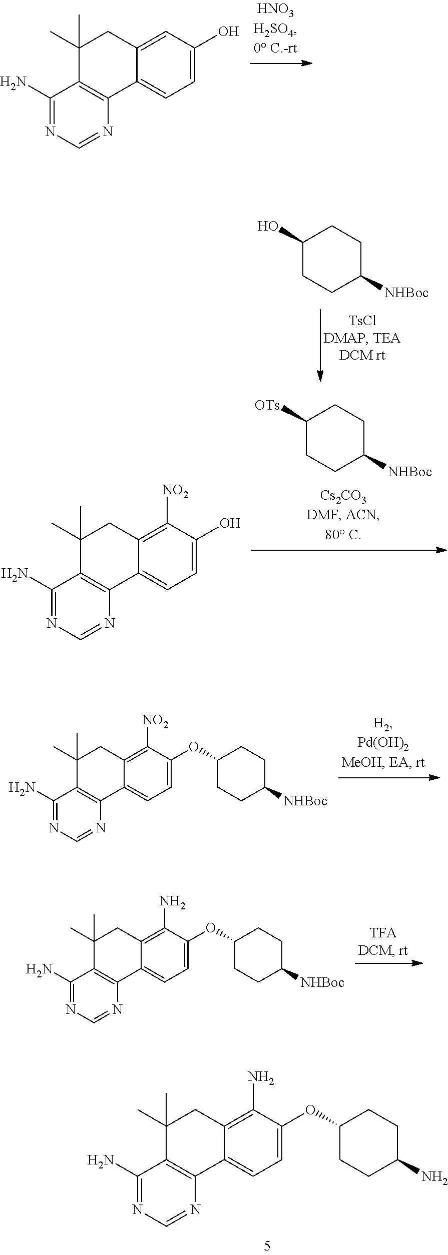

### Step 1: Preparation of [cis-4-(tert-butoxycarbonylamino)cyclohexyl]4-methylbenzenesulfonate

TsCl (139 g, 721 mmol) was added portionwise to a stirred solution of tert-butyl N-(cis-4-hydroxycyclohexyl)carbamate (80 g, 360 mmol) in DCM (300 mL), followed by TEA (152 mL, 1081 mmol) and DMAP (4.5 g, 36 mmol). After 24 h stirring, the reaction mixture was filtered. The filtrate was concentrated and the residue was purified by column chromatography (silica gel; PE:EA; 10:1; v:v) to afford [cis-4-(tert-butoxycarbonylamino)cyclohexyl]4-methylbenzenesulfonate as a white solid (105 g, 71% yield).

¹H NMR (400 MHz, DMSO-d₆) δ ppm: 7.78 (d, J=8.0 Hz, 2H), 7.47 (d, J=8.0 Hz, 2H), 6.81 (d, J=7.2 Hz, 1H), 4.58 (m, 1H), 3.24 (m, 1H), 2.41 (s, 3H), 1.66 (m, 2H), 1.51 (m, 4H), 1.41 (m, 2H), 1.36 (s, 9H).

MS m/z (+ESI): 370.1 [M+H]⁺.

### Step 2: Preparation of 4-amino-5,5-dimethyl-7-nitro-6H-benzo[h]quinazolin-8-ol

The title compound was prepared as a light yellow solid (260 mg, 35% yield) following Scheme 1 and in analogy to Example 2 (step 1) using 4-amino-5,5-dimethyl-6H-benzo[h]quinazolin-8-ol (700 mg, 2.32 mmol) as starting material.

¹H NMR (400 MHz, DMSO-d₆) δ ppm: 12.76 (br, 1H), 8.27 (s, 1H), 8.09 (d, J=8.4 Hz, 1H), 7.01 (d, J=8.4 Hz, 1H), 6.54 (br, 2H), 2.62 (s, 2H), 1.27 (s, 6H).

MS m/z (+ESI): 287.2 [M+H]⁺.

### Step 3: Preparation of tert-butyl N-[trans-4-[(4-amino-5,5-dimethyl-7-nitro-6H-benzo[h]quinazolin-8-yl)oxy]cyclohexyl]carbamate

Cs₂CO₃ (820 mg, 2.45 mmol) was added to a stirred solution of 4-amino-5,5-dimethyl-7-nitro-6H-benzo[h]quinazolin-8-ol (260 mg, 0.82 mmol) and [cis-4-(tert-butoxycarbonylamino)cyclohexyl]4-methylbenzenesulfonate (2.0 g, 4.90 mmol) in DMF (8 mL) and ACN (8 mL). After 40 h stirring at 80° C., the reaction mixture was filtered. The filtrate was concentrated and the residue was purified by combiflash to afford tert-butyl N-[trans-4-[(4-amino-5,5-dimethyl-7-nitro-6H-benzo[h]quinazolin-8-yl)oxy]cyclohexyl]carbamate as a light yellow solid (400 mg, 91% yield).

MS m/z (+ESI): 484.4 [M+H]⁺.

### Step 4: Preparation of tert-butyl N-[trans-4-[(4,7-diamino-5,5-dimethyl-6H-benzo[h]quinazolin-8-yl)oxy]cyclohexyl]carbamate

Pd(OH)₂ (20% on carbon, 52 mg, 0.07 mmol) was added to a stirred solution of tert-butyl N-[trans-4-[(4-amino-5,5-dimethyl-7-nitro-6H-benzo[h]quinazolin-8-yl)oxy]cyclohexyl]carbamate (400 mg, 0.74 mmol) in MeOH (10 mL) and EA (10 mL). After 20 h stirring under hydrogen flow, the catalyst was removed by filtration and the solution was concentrated to afford tert-butyl N-[trans-4-[(4,7-diamino-5,5-dimethyl-6H-benzo[h]quinazolin-8-yl)oxy]cyclohexyl]carbamate as a light yellow solid (340 mg, 96% yield) which was used in the next step without further purification.

MS m/z (+ESI): 454.3 [M+H]⁺.

### Step 5: Preparation of 8-(trans-4-aminocyclohexoxy)-5,5-dimethyl-6H-benzo[h]quinazoline-4,7-diamine

The title compound was prepared as a light yellow solid (246 mg, 97% yield) following Scheme 1 and in analogy to Example 4 (step 4) using tert-butyl N-[trans-4-[(4,7-diamino-5,5-dimethyl-6H-benzo[h]quinazolin-8-yl)oxy]cyclohexyl]carbamate (340 mg, 0.71 mmol) as starting material. ¹H NMR (400 MHz, DMSO-d₆+D₂O) δ ppm: 8.54 (s, 1H), 7.18 (d, J=8.8 Hz, 1H), 7.04 (d, J=8.8 Hz, 1H), 4.39 (m, 1H), 3.09 (m, 1H), 2.72 (s, 2H), 2.12 (m, 2H), 2.00 (m, 2H), 1.50 (m, 4H), 1.29 (s, 6H).

MS m/z (+ESI): 354.3 [M+H]⁺.

### Preparation of Example 6: N-[4-amino-8-(trans-4-aminocyclohexoxy)-5,5-dimethyl-6H-benzo[h]quinazolin-7-yl]acetamide

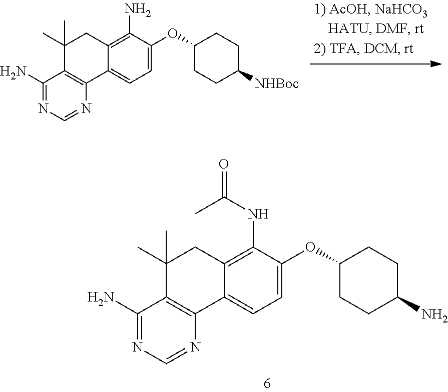 [SMILES: CC(C)(C)OC(=O)N[C@H]1CC[C@H](Oc2ccc3c(c2N)CC(C)(C)c2c(N)ncnc2-3)CC1 ; CC(=O)Nc1c(O[C@H]2CC[C@H](N)CC2)ccc2c1CC(C)(C)c1c(N)ncnc1-2]

HATU (75 mg, 0.19 mmol) was added to a stirred solution of tert-butyl N-[trans-4-[(4,7-diamino-5,5-dimethyl-6H-benzo[h]quinazolin-8-yl)oxy]cyclohexyl]carbamate (80 mg, 0.16 mmol) in DMF (2 mL), followed by NaHCO₃ (26.9 mg, 0.32 mmol) and AcOH (19.3 mg, 0.32 mmol). After 1 h stirring, the reaction mixture was extracted with EA and H₂O. The combined organic layers were dried over Na₂SO₄, filtered, concentrated. The residue was dissolved in DCM (1 mL) and TFA (1 mL). After 1 h stirring the reaction mixture was concentrated and purified by preparative HPLC to afford N-[4-amino-8-(trans-4-aminocyclohexoxy)-5,5-dimethyl-6H-benzo[h]quinazolin-7-yl]acetamide as a white solid (29 mg, 44% yield).

¹H NMR (400 MHz, DMSO-d₆+D₂O) δ ppm: 8.52 (s, 1H), 7.77 (d, J=8.8 Hz, 1H), 7.21 (d, J=8.8 Hz, 1H), 4.39 (m, 1H), 3.09 (m, 1H), 2.62 (s, 2H), 2.07 (m, 2H), 2.04 (s, 3H), 1.97 (m, 2H), 1.48 (m, 4H), 1.26 (s, 6H).

MS m/z (+ESI): 396.2 [M+H]⁺.

### Preparation of Example 7: 8-(trans-4-aminocyclohexoxy)-N7,N7-diethyl-5,5-dimethyl-6H-benzo[h]quinazoline-4,7-diamine

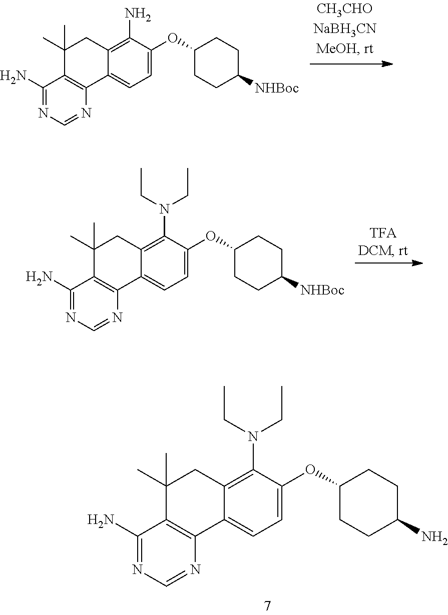 [SMILES: CC(C)(C)OC(=O)N[C@H]1CC[C@H](Oc2ccc3c(c2N)CC(C)(C)c2c(N)ncnc2-3)CC1 ; CCN(CC)c1c(O[C@H]2CC[C@H](NC(=O)OC(C)(C)C)CC2)ccc2c1CC(C)(C)c1c(N)ncnc1-2 ; CCN(CC)c1c(O[C@H]2CC[C@H](N)CC2)ccc2c1CC(C)(C)c1c(N)ncnc1-2]

### Step 1: Preparation of tert-butyl N-[trans-4-[[4-amino-7-(diethylamino)-5,5-dimethyl-6H-benzo[h]quinazolin-8-yl]oxy]cyclohexyl]carbamate

NaBH₃CN (8 mg, 0.12 mmol) was added to a stirred solution of acetaldehyde (27 μL, 0.48 mmol) and tert-butyl N-[trans-4-[(4,7-diamino-5,5-dimethyl-6H-benzo[h]quinazolin-8-yl)oxy]cyclohexyl]carbamate (60 mg, 0.12 mmol) in MeOH (5 mL). After 2 h stirring, the solvent was removed and the crude was extracted with EA and H₂O. The combined organic layers were dried over Na₂SO₄, filtered, concentrated to afford tert-butyl N-[trans-4-[[4-amino-7-(diethylamino)-5,5-dimethyl-6H-benzo[h]quinazolin-8-yl]oxy]cyclohexyl]carbamate as a white solid (60 mg, 89% yield) which was used in the next step without further purification.

MS m/z (+ESI): 510.3 [M+H]⁺.

### Step 2: Preparation of 8-(trans-4-aminocyclohexoxy)-N7,N7-diethyl-5,5-dimethyl-6H-benzo[h]quinazoline-4,7-diamine

The title compound was prepared as a light yellow solid (44 mg, 99% yield) following Scheme 1 and in analogy to Example 4 (step 4) using tert-butyl N-[trans-4-[[4-amino-7-(diethylamino)-5,5-dimethyl-6H-benzo[h]quinazolin-8-yl]oxy]cyclohexyl]carbamate (60 mg, 0.11 mmol) as starting material.

¹H NMR (400 MHz, DMSO-d₆+D₂O) δ ppm: 8.54 (s, 1H), 7.71 (d, J=8.8 Hz, 1H), 7.20 (d, J=8.8 Hz, 1H), 4.49 (m, 1H), 3.20 (m, 2H), 3.11 (m, 1H), 2.98 (m, 4H), 2.14 (m, 2H), 1.98 (m, 2H), 1.50 (m, 4H), 1.26 (s, 6H), 0.84 (t, J=7.2 Hz, 6H).

MS m/z (+ESI): 410.3 [M+H]⁺.

### Preparation of Example 8: 8-(trans-4-aminocyclohexoxy)-N7-ethyl-5,5-dimethyl-6H-benzo[h]quinazoline-4,7-diamine

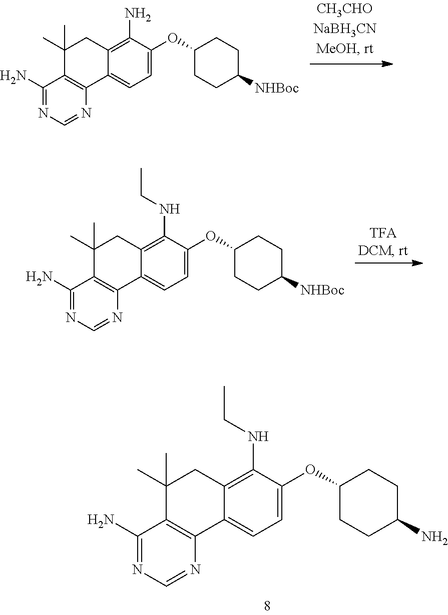 [SMILES: CC(C)(C)OC(=O)N[C@H]1CC[C@H](Oc2ccc3c(c2N)CC(C)(C)c2c(N)ncnc2-3)CC1 ; CCNc1c(O[C@H]2CC[C@H](NC(=O)OC(C)(C)C)CC2)ccc2c1CC(C)(C)c1c(N)ncnc1-2 ; CCNc1c(O[C@H]2CC[C@H](N)CC2)ccc2c1CC(C)(C)c1c(N)ncnc1-2]

### Step 1: Preparation of tert-butyl N-[trans-4-[[4-amino-7-(ethylamino)-5,5-dimethyl-6H-benzo[h]quinazolin-8-yl]oxy]cyclohexyl]carbamate

Acetaldehyde (5 mg, 0.12 mmol) was added to a stirred solution of tert-butyl N-[trans-4-[(4,7-diamino-5,5-dimethyl-6H-benzo[h]quinazolin-8-yl)oxy]cyclohexyl]carbamate (60 mg, 0.12 mmol) in MeOH (5 mL). After 2 h stirring, NaBH₃CN (8 mg, 0.12 mmol) was added. After 1 h stirring, the solvent was removed and the crude was extracted with EA and H₂O. The combined organic layers were dried over Na₂SO₄, filtered and concentrated to afford tert-butyl N-[trans-4-[[4-amino-7-(ethylamino)-5,5-dimethyl-6H-benzo[h]quinazolin-8-yl]oxy]cyclohexyl]carbamate as a white solid (60 mg, 84% yield).

MS m/z (+ESI): 482.2 [M+H]⁺.

### Step 2: Preparation of 8-(trans-4-aminocyclohexoxy)-N7-ethyl-5,5-dimethyl-6H-benzo[h]quinazoline-4,7-diamine

The title compound was prepared as an off-white solid (38 mg, 97% yield) following Scheme 1 and in analogy to Example 4 (step 4) using tert-butyl N-[trans-4-[[4-amino-7-(ethylamino)-5,5-dimethyl-6H-benzo[h]quinazolin-8-yl]oxy]cyclohexyl]carbamate (60 mg, 0.10 mmol) as starting material.

¹H NMR (400 MHz, DMSO-d₆+D₂O) δ ppm: 8.50 (s, 1H), 7.68 (d, J=8.8 Hz, 1H), 7.25 (d, J=8.8 Hz, 1H), 4.49 (m, 1H), 3.12 (m, 3H), 2.86 (s, 2H), 2.13 (m, 2H), 2.00 (m, 2H), 1.52 (m, 4H), 1.28 (s, 6H), 1.10 (t, J=7.2 Hz, 3H).

MS m/z (+ESI): 382.2 [M+H]⁺.

### Preparation of Example 9: 8-(trans-4-aminocyclohexoxy)-5,5-dimethyl-7-(1-piperidyl)-6H-benzo[h]quinazolin-4-amine

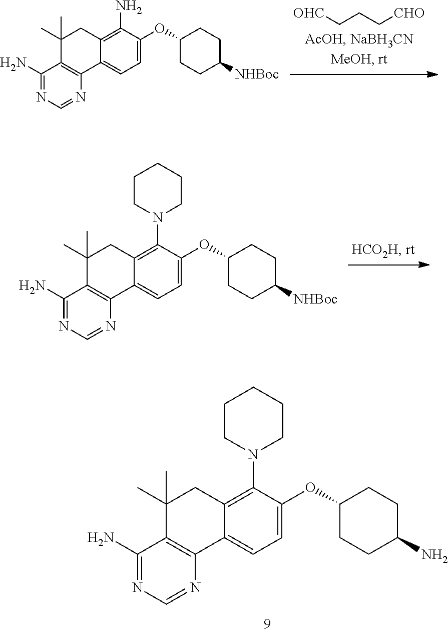 [SMILES: CC(C)(C)OC(=O)N[C@H]1CC[C@H](Oc2ccc3c(c2N)CC(C)(C)c2c(N)ncnc2-3)CC1 ; O=CCCCC=O ; CC(C)(C)OC(=O)N[C@H]1CC[C@H](Oc2ccc3c(c2N2CCCCC2)CC(C)(C)c2c(N)ncnc2-3)CC1 ; CC1(C)Cc2c(ccc(O[C@H]3CC[C@H](N)CC3)c2N2CCCCC2)-c2ncnc(N)c21]

### Step 1: Preparation of tert-butyl N-[trans-4-[[4-amino-5,5-dimethyl-7-(1-piperidyl)-6H-benzo[h]quinazolin-8-yl]oxy]cyclohexyl]carbamate

Glutaraldehyde (90 μL, 0.16 mmol) was added to a stirred solution of tert-butyl N-[trans-4-[(4,7-diamino-5,5-dimethyl-6H-benzo[h]quinazolin-8-yl)oxy]cyclohexyl]carbamate (80 mg, 0.16 mmol) in MeOH (10 mL), followed by a drop of AcOH and NaBH₃CN (31 mg, 0.16 mmol). After 2 h stirring, the reaction mixture was extracted with DCM and H₂O. The combined organic layers were washed with brine, dried over Na₂SO₄, filtered, concentrated. The residue was purified by column chromatography (silica gel; EA:DCM; 1:2 to 3:1; v:v) to afford tert-butyl N-[trans-4-[[4-amino-5,5-dimethyl-7-(1-piperidyl)-6H-benzo[h]quinazolin-8-yl]oxy]cyclohexyl]carbamate as a colorless oil (73 mg, 79% yield).

MS m/z (+ESI): 522.3 [M+H]⁺.

### Step 2: Preparation of 8-(trans-4-aminocyclohexoxy)-5,5-dimethyl-7-(1-piperidyl)-6H-benzo[h]quinazolin-4-amine

A solution of tert-butyl N-[trans-4-[[4-amino-5,5-dimethyl-7-(1-piperidyl)-6H-benzo[h]quinazolin-8-yl]oxy]cyclohexyl]carbamate (85 mg, 0.15 mmol) in HCO₂H (2 mL) was stirred for 16 h before concentration and purification by preparative HPLC to afford 8-(trans-4-aminocyclohexoxy)-5,5-dimethyl-7-(1-piperidyl)-6H-benzo[h]quinazolin-4-amine as an off-white solid (33 mg, 52% yield).

¹H NMR (400 MHz, DMSO-d₆+D₂O) δ ppm: 8.20 (s, 1H), 7.79 (d, J=8.8 Hz, 1H), 6.96 (d, J=8.8 Hz, 1H), 4.34 (m, 1H), 3.20 (m, 2H), 3.10 (m, 1H), 2.87 (s, 2H), 2.67 (m, 2H), 2.13 (m, 2H), 1.99 (m, 2H), 1.75 (m, 1H), 1.52 (m, 9H), 1.24 (s, 6H).

MS m/z (+ESI): 422.3 [M+H]⁺.

### Preparation of Example 11: 2-[[4-amino-8-(trans-4-aminocyclohexoxy)-5,5-dimethyl-6H-benzo[h]quinazolin-7-yl]-(2-hydroxyethyl)amino]ethanol

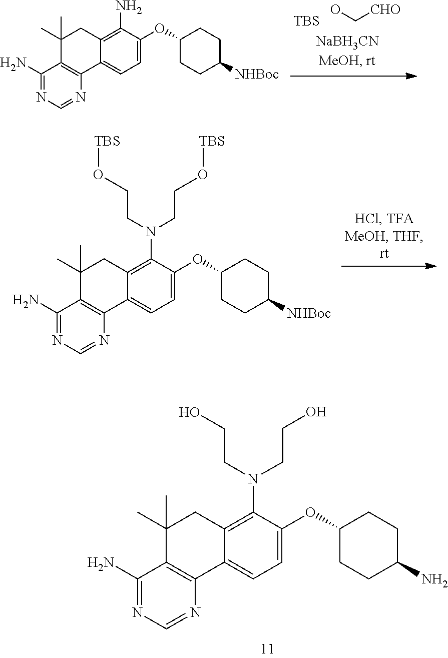

### Step 1: Preparation of tert-butyl N-[trans-4-[[4-amino-7-[bis[2-[tert-butyl(dimethyl)silyl]oxyethyl]amino]-5,5-dimethyl-6H-benzo[h]quinazolin-8-yl]oxy]cyclohexyl]carbamate

The title compound was prepared as a colorless oil (120 mg, 90% yield) following Scheme 1 and in analogy to Example 7 (step 1) using tert-butyl N-[trans-4-[(4,7-diamino-5,5-dimethyl-6H-benzo[h]quinazolin-8-yl)oxy]cyclohexyl]carbamate (70 mg, 0.14 mmol) and (tert-butyldimethylsilyloxy)acetaldehyde as starting materials.

MS m/z (+ESI): 714.5 [M+H]⁺.

### Step 2: Preparation of 2-[[4-amino-8-(trans-4-aminocyclohexoxy)-5,5-dimethyl-6H-benzo[h]quinazolin-7-yl]-(2-hydroxyethyl)amino]ethanol

TFA (234 μL, 3.12 mmol) and a 4M HCl aqueous solution (1.56 mL, 6.23 mmol) were added to a stirred solution of tert-butyl N-[trans-4-[[4-amino-7-[bis[2-[tert-butyl(dimethyl)silyl]oxyethyl]amino]-5,5-dimethyl-6H-benzo[h]quinazolin-8-yl]oxy]cyclohexyl]carbamate (150 mg, 0.16 mmol) in THF (6 mL) and MeOH (100 μL). After 2 h stirring, the reaction mixture was concentrated and the residue was purified by preparative HPLC to afford 2-[[4-amino-8-(trans-4-aminocyclohexoxy)-5,5-dimethyl-6H-benzo[h]quinazolin-7-yl]-(2-hydroxyethyl)amino]ethanol as a white solid (49 mg, 72% yield).

¹H NMR (400 MHz, DMSO-d₆+D₂O) δ ppm: 8.55 (s, 1H), 7.73 (d, J=8.8 Hz, 1H), 7.20 (d, J=8.8 Hz, 1H), 4.49 (m, 1H), 3.32 (m, 4H), 3.09 (m, 5H), 3.02 (s, 2H), 2.16 (m, 2H), 2.00 (m, 2H), 1.53 (m, 4H), 1.27 (s, 6H).

MS m/z (+ESI): 442.4 [M+H]⁺.

### Preparation of Examples 15, 16 and 17: 1-[4-amino-8-(trans-4-aminocyclohexoxy)-5,5-dimethyl-6H-benzo[h]quinazolin-7-yl]pyrrolidin-3-ol—isomer 1, 1-[4-amino-8-(trans-4-aminocyclohexoxy)-5,5-dimethyl-6H-benzo[h]quinazolin-7-yl]pyrrolidin-3-ol—isomer 2 and 8-(trans-4-aminocyclohexoxy)-5,5-dimethyl-7-pyrrol-1-yl-6H-benzo[h]quinazolin-4-amine

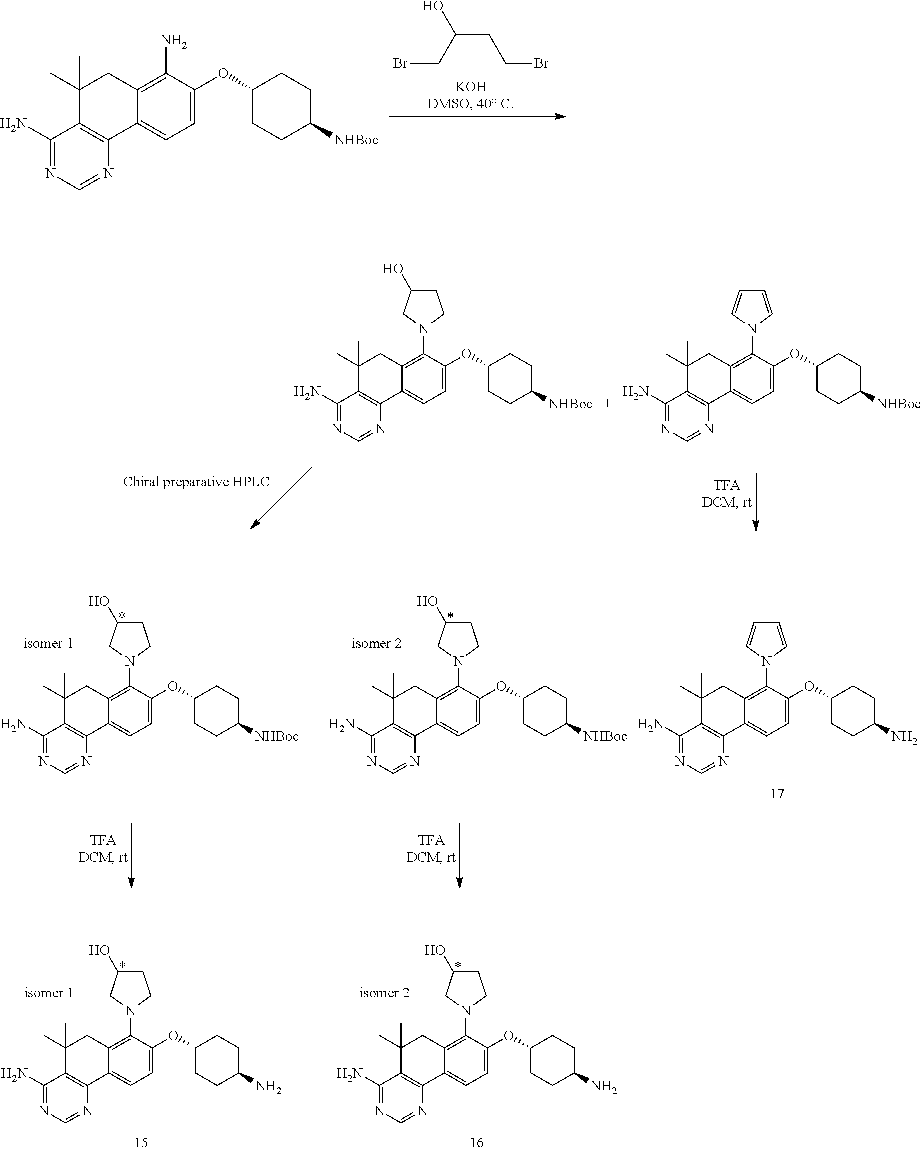

### Step 1: Preparation of tert-butyl N-[trans-4-[[4-amino-7-(3-hydroxypyrrolidin-1-yl)-5,5-dimethyl-6H-benzo[h]quinazolin-8-yl]oxy]cyclohexyl]carbamate and tert-butyl N-[trans-4-[(4-amino-5,5-dimethyl-7-pyrrol-1-yl-6H-benzo[h]quinazolin-8-yl)oxy]cyclohexyl]carbamate

1,4-Dibromo-2-butanol (727 mg, 2.98 mmol) was added to a stirred solution of tert-butyl N-[trans-4-[(4,7-diamino-5,5-dimethyl-6H-benzo[h]quinazolin-8-yl)oxy]cyclohexyl]carbamate (150 g, 0.30 mmol) in DMSO (6 mL), followed by KOH (136 g, 2.38 mmol). After 16 h stirring at 40° C., the reaction mixture was concentrated and the residue was purified by preparative HPLC to afford tert-butyl N-[trans-4-[[4-amino-7-(3-hydroxypyrrolidin-1-yl)-5,5-dimethyl-6H-benzo[h]quinazolin-8-yl]oxy]cyclohexyl]carbamate as a yellow oil (90 mg, 46% yield) and tert-butyl N-[trans-4-[(4-amino-5,5-dimethyl-7-pyrrol-1-yl-6H-benzo[h]quinazolin-8-yl)oxy]cyclohexyl]carbamate as a yellow oil (50 mg, 30% yield).

MS m/z (+ESI): 524.4 [M+H]⁺ and 504.4 [M+H]⁺.

### Step 2: Preparation of tert-butyl N-[trans-4-[[4-amino-7-(3-hydroxypyrrolidin-1-yl)-5,5-dimethyl-6H-benzo[h]quinazolin-8-yl]oxy]cyclohexyl]carbamate—isomer 1 and tert-butyl N-[trans-4-[[4-amino-7-(3-hydroxypyrrolidin-1-yl)-5,5-dimethyl-6H-benzo[h]quinazolin-8-yl]oxy]cyclohexyl]carbamate—isomer 2

tert-Butyl N-[trans-4-[[4-amino-7-(3-hydroxypyrrolidin-1-yl)-5,5-dimethyl-6H-benzo[h]quinazolin-8-yl]oxy]cyclohexyl]carbamate (430 g, 0.66 mmol) was purified by chiral preparative HPLC to afford tert-butyl N-[trans-4-[[4-amino-7-(3-hydroxypyrrolidin-1-yl)-5,5-dimethyl-6H-benzo[h]quinazolin-8-yl]oxy]cyclohexyl]carbamate—isomer 1 as a yellow viscous oil (226 mg, 39% yield) and tert-butyl N-[trans-4-[[4-amino-7-(3-hydroxypyrrolidin-1-yl)-5,5-dimethyl-6H-benzo[h]quinazolin-8-yl]oxy]cyclohexyl]carbamate—isomer 2 as a yellow semisolid (91 mg, 21% yield).

MS m/z (+ESI): 524.3 [M+H]⁺.

### Step 3: Preparation of 1-[4-amino-8-(trans-4-aminocyclohexoxy)-5,5-dimethyl-6H-benzo[h]quinazolin-7-yl]pyrrolidin-3-ol—isomer 1

The title compound was prepared as a white solid (37 mg, 34% yield) following Scheme 1 and in analogy to Example 4 (step 4) using tert-butyl N-[trans-4-[[4-amino-7-(3-hydroxypyrrolidin-1-yl)-5,5-dimethyl-6H-benzo[h]quinazolin-8-yl]oxy]cyclohexyl]carbamate—isomer 1 (210 mg, 0.24 mmol) as starting material.

¹H NMR (400 MHz, DMSO-d₆+D₂O) δ ppm: 8.55 (s, 1H), 7.72 (d, J=8.8 Hz, 1H), 7.22 (d, J=8.8 Hz, 1H), 4.48 (m, 1H), 4.40 (m, 1H), 3.26 (m, 2H), 3.09 (m, 2H), 2.96 (m, 3H), 2.12 (m, 3H), 1.99 (m, 2H), 1.82 (m, 1H), 1.52 (m, 4H), 1.26 (s, 6H).

MS m/z (+ESI): 424.4 [M+H]⁺.

[α]_{D}²⁶=−6° (c=0.1, MeOH).

### Step 4: Preparation of 1-[4-amino-8-(trans-4-aminocyclohexoxy)-5,5-dimethyl-6H-benzo[h]quinazolin-7-yl]pyrrolidin-3-ol—isomer 2

The title compound was prepared as a white solid (27 mg, 44% yield) following Scheme 1 and in analogy to Example 4 (step 4) using tert-butyl N-[trans-4-[[4-amino-7-(3-hydroxypyrrolidin-1-yl)-5,5-dimethyl-6H-benzo[h]quinazolin-8-yl]oxy]cyclohexyl]carbamate—isomer 2 (90 mg, 0.14 mmol) as starting material.

¹H NMR (400 MHz, DMSO-d₆+D₂O) δ ppm: 8.56 (s, 1H), 7.74 (d, J=8.8 Hz, 1H), 7.23 (d, J=8.8 Hz, 1H), 4.48 (m, 1H), 4.39 (m, 1H), 3.26 (m, 2H), 3.08 (m, 2H), 2.96 (m, 3H), 2.12 (m, 3H), 1.99 (m, 2H), 1.82 (m, 1H), 1.53 (m, 4H), 1.26 (s, 6H).

MS m/z (+ESI): 424.4 [M+H]⁺.

[α]_{D}²⁶=+7.5° (c=0.1, MeOH).

### Step 5: Preparation of 8-(trans-4-aminocyclohexoxy)-5,5-dimethyl-7-pyrrol-1-yl-6H-benzo[h]quinazolin-4-amine

The title compound was prepared as a white solid (36 mg, 37% yield) following Scheme 1 and in analogy to Example 4 (step 4) using tert-butyl N-[trans-4-[(4-amino-5,5-dimethyl-7-pyrrol-1-yl-6H-benzo[h]quinazolin-8-yl)oxy]cyclohexyl]carbamate (130 mg, 0.23 mmol) as starting material.

¹H NMR (400 MHz, DMSO-d₆+D₂O) δ ppm: 8.26 (s, 1H), 8.07 (d, J=8.8 Hz, 1H), 7.15 (d, J=8.8 Hz, 1H), 6.68 (t, J=2.0 Hz, 2H), 6.19 (t, J=2.0 Hz, 2H), 4.22 (m, 1H), 2.55 (m, 1H), 2.34 (s, 2H), 1.90 (m, 2H), 1.70 (m, 2H), 1.16 (m, 10H).

MS m/z (+ESI): 404.3 [M+H]⁺.

### Preparation of Example 19: 8-(trans-4-aminocyclohexoxy)-5,5-dimethyl-7-propoxy-6H-benzo[h]quinazolin-4-amine

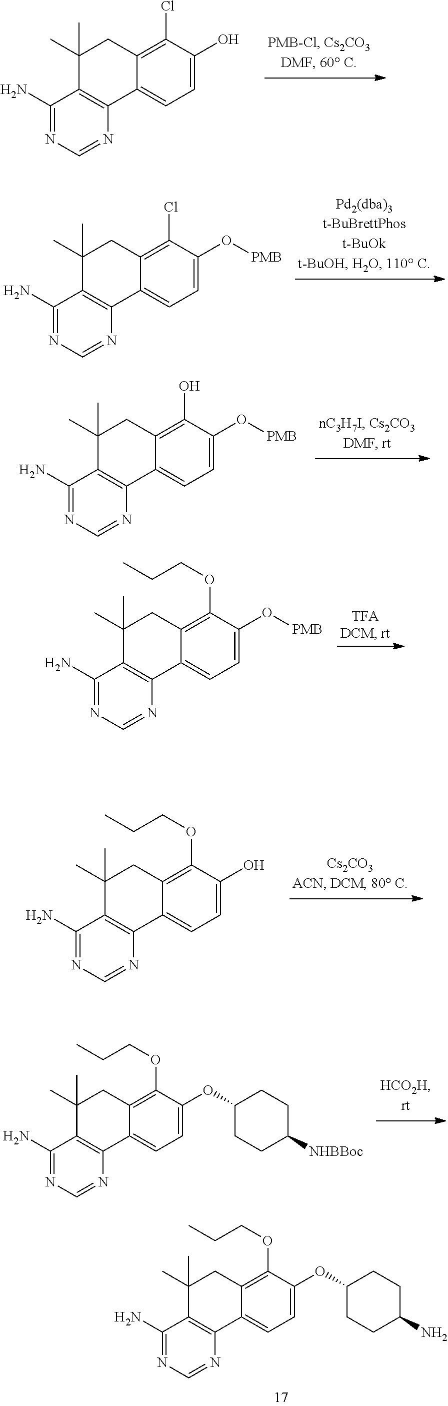

### Step 1: Preparation of 7-chloro-8-[(4-methoxyphenyl)methoxy]-5,5-dimethyl-6H-benzo[h]quinazolin-4-amine

PMB-Cl (492 mg, 3.05 mmol) was added to a stirred solution of 4-amino-7-chloro-5,5-dimethyl-6H-benzo[h]quinazolin-8-ol (700 mg, 2.03 mmol) in DMF (10 mL), followed by Cs₂CO₃ (1.35 g, 4.06 mmol). After 20 h stirring at 60° C., the reaction mixture was concentrated and extracted with DCM and H₂O. The combined organic layers were dried over Na₂SO₄, filtered and concentrated. The residue was purified by combiflash to afford 7-chloro-8-[(4-methoxyphenyl)methoxy]-5,5-dimethyl-6H-benzo[h]quinazolin-4-amine as a white solid (520 mg, 58% yield).

¹H NMR (400 MHz, DMSO-d₆) δ ppm: 8.27 (s, 1H), 8.03 (d, J=8.8 Hz, 1H), 7.42 (d, J=8.8 Hz, 2H), 7.22 (d, J=8.8 Hz, 1H), 6.97 (d, J=8.8 Hz, 2H), 6.49 (s, 2H), 5.16 (s, 2H), 3.76 (s, 3H), 2.90 (s, 2H), 1.30 (s, 6H).

MS m/z (+ESI): 396.2 [M+H]⁺.

### Step 2: Preparation of 4-amino-8-[(4-methoxyphenyl)methoxy]-5,5-dimethyl-6H-benzo[h]quinazolin-7-ol

t-BuOK (410 mg, 3.55 mmol) was added to a stirred solution of 7-chloro-8-[(4-methoxyphenyl)methoxy]-5,5-dimethyl-6H-benzo[h]quinazolin-4-amine (520 mg, 1.18 mmol) in t-BuOH (10 mL) and H₂O (0.5 mL), followed by Pd₂(dba)₃ (110 mg, 0.12 mmol) and t-BuBrettPhos (118 mg, 0.24 mmol). After 2 h stirring at 110° C., the reaction mixture was filtered through decalite, concentrated and purified by preparative HPLC to afford 4-amino-8-[(4-methoxyphenyl)methoxy]-5,5-dimethyl-6H-benzo[h]quinazolin-7-ol as a yellow solid (184 mg, 37% yield).

MS m/z (+ESI): 378.2 [M+H]⁺.

### Step 3: Preparation of 8-[(4-methoxyphenyl)methoxy]-5,5-dimethyl-7-propoxy-6H-benzo[h]quinazolin-4-amine

The title compound was prepared as a light yellow solid (150 mg, 73% yield) following Scheme 1 and in analogy to Example 4 (step 4) using 4-amino-8-[(4-methoxyphenyl)methoxy]-5,5-dimethyl-6H-benzo[h]quinazolin-7-ol (184 mg, 0.44 mmol) and n-propyl iodide as starting materials. MS m/z (+ESI): 420.3 [M+H]⁺.

### Step 4: Preparation of 4-amino-5,5-dimethyl-7-propoxy-6H-benzo[h]quinazolin-8-ol

A solution of 8-[(4-methoxyphenyl)methoxy]-5,5-dimethyl-7-propoxy-6H-benzo[h]quinazolin-4-amine (30 mg, 0.06 mmol) in TFA (1 mL) and DCM (1 mL) was stirred for 1 h before concentration and purification by preparative HPLC to afford 4-amino-5,5-dimethyl-7-propoxy-6H-benzo[h]quinazolin-8-ol as a white solid (9 mg, 44% yield).

¹H NMR (400 MHz, DMSO-d₆+D₂O) δ ppm: 8.20 (s, 1H), 7.57 (d, J=8.4 Hz, 1H), 6.80 (d, J=8.4 Hz, 1H), 3.79 (t, J=6.8 Hz, 2H), 2.74 (s, 2H), 1.67 (qt, J₁=7.2 Hz, J₂=6.8 Hz, 2H), 1.23 (s, 6H), 0.95 (t, J=7.2 Hz, 3H).

MS m/z (+ESI): 300.2 [M+H]⁺.

### Step 5: Preparation of 8-(trans-4-aminocyclohexoxy)-5,5-dimethyl-7-propoxy-6H-benzo[h]quinazolin-4-amine

The title compound was prepared as a white solid following Scheme 1 and in analogy to Example 5 (steps 1 and 3) and Example 9 (step 2) using 4-amino-5,5-dimethyl-7-propoxy-6H-benzo[h]quinazolin-8-ol and using tert-butyl N-(cis-4-hydroxycyclohexyl)carbamate as starting materials.

¹H NMR (400 MHz, DMSO-d₆+D₂O) δ ppm: 8.18 (s, 1H), 7.15 (d, J=8.8 Hz, 1H), 7.00 (d, J=8.8 Hz, 1H), 4.32 (m, 1H), 3.82 (t, J=6.8 Hz, 2H), 3.06 (m, 1H), 2.73 (s, 2H), 2.11 (m, 2H), 1.97 (m, 2H), 1.66 (qt, J₁=7.2 Hz, J₂=6.8 Hz, 2H), 1.46 (m, 4H), 1.24 (s, 6H), 0.96 (t, J=7.2 Hz 3H).

MS m/z (+ESI): 397.3 [M+H]⁺.

### Preparation of Example 21: 1-(4-amino-8-methoxy-5,5-dimethyl-6H-benzo[h]quinazolin-9-yl)piperidin-4-ol

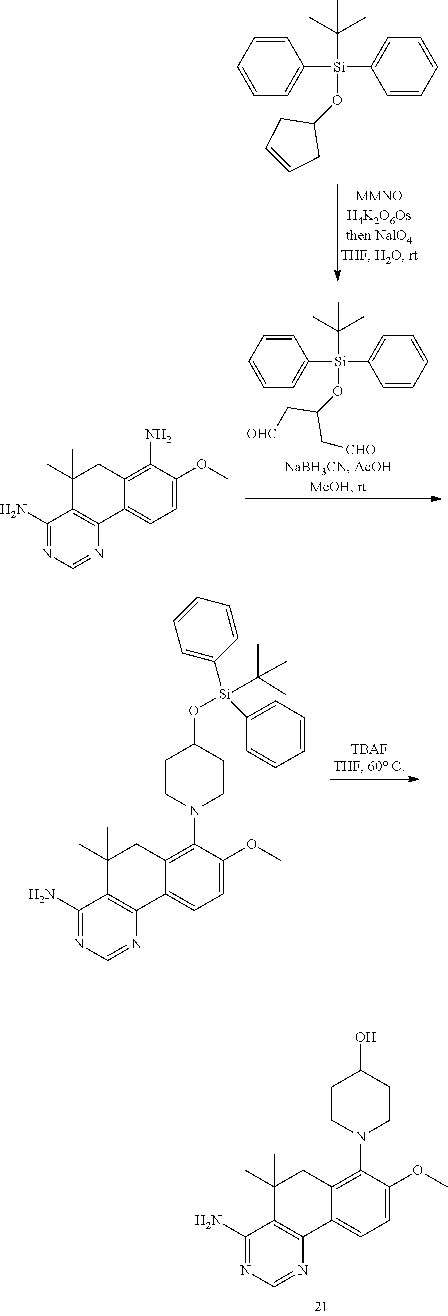 [SMILES: CC(C)(C)[Si](OC1CC=CC1)(c1ccccc1)c1ccccc1 ; COc1ccc2c(c1N)CC(C)(C)c1c(N)ncnc1-2 ; CC(C)(C)[Si](OC(CC=O)CC=O)(c1ccccc1)c1ccccc1 ; COc1ccc2c(c1N1CCC(O[Si](c3ccccc3)(c3ccccc3)C(C)(C)C)CC1)CC(C)(C)c1c(N)ncnc1-2 ; COc1ccc2c(c1N1CCC(O)CC1)CC(C)(C)c1c(N)ncnc1-2]

### Step 1: Preparation of 3-[tert-butyl(diphenyl)silyl]oxypentanedial

MMNO (3.2 g, 26.51 mmol) was added to a stirred solution of tert-butyl-cyclopent-3-en-1-yloxy-diphenyl-silane (3.8 g, 10.60 mmol) in THF (40 mL) and H₂O (10 mL), followed by potassium osmate(VI) dihydrate (40 mg, 0.11 mmol). After 20 h stirring, NaIO₄ (2.74 g, 12.73 mmol) was added and the resulting suspension was stirred for 3 h before filtration. The filtrate was concentrated and the residue was purified by column chromatography (silica gel; EA:PE; 1:3; v:v) to afford 3-[tert-butyl(diphenyl)silyl]oxypentanedial as an off-white viscous oil (2 g, 27% yield).

¹H NMR (400 MHz, CDCl₃) δ ppm: 9.65 (t, J=1.6 Hz, 2H), 7.66 (m, 4H), 7.42 (m, 6H), 4.72 (m, 1H), 2.66 (m, 4H), 1.05 (s, 9H).

### Step 2: Preparation of 7-[4-[tert-butyl(diphenyl)silyl]oxy-1-piperidyl]-8-methoxy-5,5-dimethyl-6H-benzo[h]quinazolin-4-amine

3-[tert-Butyl(diphenyl)silyl]oxypentanedial (1.49 g, 2.11 mmol) was added to a stirred solution of 8-methoxy-5,5-dimethyl-6H-benzo[h]quinazoline-4,7-diamine (200 mg, 0.70 mmol) in MeOH (3 mL), followed by NaBH₃CN (139 mg, 2.11 mmol) and a drop of AcOH. After 2 h stirring, the reaction mixture was quenched with NH₄Cl aqueous solution and extracted with DCM. The combined organic layers were dried over Na₂SO₄, filtered and concentrated. The residue was purified by combiflash to afford 7-[4-[tert-butyl(diphenyl)silyl]oxy-1-piperidyl]-8-methoxy-5,5-dimethyl-6H-benzo[h]quinazolin-4-amine as a white solid (350 mg, 76% yield).

MS m/z (+ESI): 593.5 [M+H]⁺.

### Step 3: Preparation of 1-(4-amino-8-methoxy-5,5-dimethyl-6H-benzo[h]quinazolin-9-yl)piperidin-4-ol

TBAF (218 mg, 0.75 mmol) was added to a stirred solution of 7-[4-[tert-butyl(diphenyl)silyl]oxy-1-piperidyl]-8-methoxy-5,5-dimethyl-6H-benzo[h]quinazolin-4-amine (330 mg, 0.50 mmol) in THF (3 mL). After 20 h stirring at 60° C., the reaction mixture was concentrated and the residue was purified by preparative HPLC to afford 1-(4-amino-8-methoxy-5,5-dimethyl-6H-benzo[h]quinazolin-9-yl)piperidin-4-ol as a white solid (80 mg, 45% yield).

¹H NMR (400 MHz, DMSO-d₆+D₂O) δ ppm: 8.22 (s, 1H), 7.84 (d, J=8.4 Hz, 1H), 6.90 (d, J=8.8 Hz, 1H), 3.79 (s, 3H), 3.46 (m, 1H), 3.23 (m, 2H), 2.88 (s, 2H), 2.68 (m, 2H), 1.8 (m, 2H), 1.49 (m, 2H), 1.25 (s, 6H).

MS m/z (+ESI): 355.3 [M+H]⁺.

### Preparation of Example 22: 2-[[4-amino-8-(4-cis-aminocyclohexoxy)-5,5-dimethyl-6H-benzo[h]quinazolin-7-yl]-ethyl-amino]ethanol

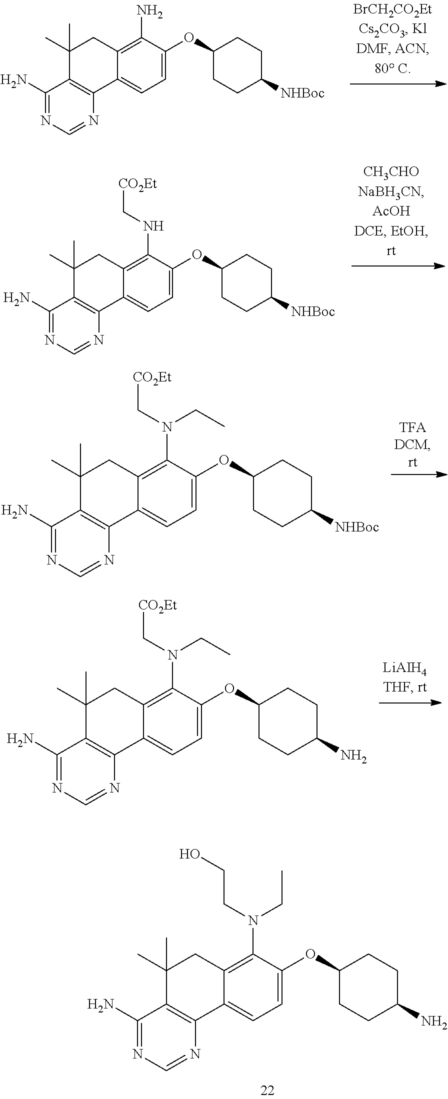 [SMILES: CC(C)(C)OC(=O)N[C@H]1CC[C@@H](Oc2ccc3c(c2N)CC(C)(C)c2c(N)ncnc2-3)CC1 ; CCOC(=O)CNc1c(O[C@H]2CC[C@@H](NC(=O)OC(C)(C)C)CC2)ccc2c1CC(C)(C)c1c(N)ncnc1-2 ; CCOC(=O)CN(CC)c1c(O[C@H]2CC[C@@H](NC(=O)OC(C)(C)C)CC2)ccc2c1CC(C)(C)c1c(N)ncnc1-2 ; CCOC(=O)CN(CC)c1c(O[C@H]2CC[C@@H](N)CC2)ccc2c1CC(C)(C)c1c(N)ncnc1-2 ; CCN(CCO)c1c(O[C@H]2CC[C@@H](N)CC2)ccc2c1CC(C)(C)c1c(N)ncnc1-2]

The starting material was prepared according to procedure described for Example 5 (steps 1 to 4) from 8-methoxy-5,5-dimethyl-6H-benzo[h]quinazoline-4,7-diamine and tert-butyl N-(trans-4-hydroxycyclohexyl)carbamate.

### Step 1: Preparation of ethyl 2-[[4-amino-8-[cis-4-(tert-butoxycarbonylamino)cyclohexoxy]-5,5-dimethyl-6H-benzo[h]quinazolin-7-yl]amino]acetate

Ethyl 2-bromoacetate (504 μL, 4.41 mmol) was added to a stirred solution of tert-butyl N-[cis-4-[(4,7-diamino-5,5-dimethyl-6H-benzo[h]quinazolin-8-yl)oxy]cyclohexyl]carbamate (500 mg, 0.88 mmol) in DMF (5 mL), followed by Cs₂CO₃ (880 mg, 2.65 mmol) and KI (74 mg, 0.44 mmol). After 4 h stirring, the reaction mixture was concentrated and extracted with EA and H₂O. The combined organic layers were dried over Na₂SO₄, filtered and concentrated. The residue was purified by combiflash to afford ethyl 2-[[4-amino-8-[cis-4-(tert-butoxycarbonylamino)cyclohexoxy]-5,5-dimethyl-6H-benzo[h]quinazolin-7-yl]amino]acetate as a yellow solid (390 mg, 70% yield).

MS m/z (+ESI): 540.3 [M+H]⁺.

### Step 2: Preparation of ethyl 2-[[4-amino-8-[cis-4-(tert-butoxycarbonylamino)cyclohexoxy]-5,5-dimethyl-6H-benzo[h]quinazolin-7-yl]-ethyl-amino]acetate

Acetaldehyde (31 μL, 5.51 mmol) was added to a stirred solution of ethyl 2-[[4-amino-8-[cis-4-(tert-butoxycarbonylamino)cyclohexoxy]-5,5-dimethyl-6H-benzo[h]quinazolin-7-yl]amino]acetate (350 mg, 0.55 mmol) in DCE (6 mL) and EtOH (2 mL), followed by NaBH₃CN (182 mg, 2.75 mmol) and a drop of AcOH. After 3 h stirring, the reaction mixture was quenched with NH₄Cl aqueous solution and extracted with DCM. The combined organic layers were dried over Na₂SO₄, filtered and concentrated. The residue was purified by combiflash to afford ethyl 2-[[4-amino-8-[cis-4-(tert-butoxycarbonylamino)cyclohexoxy]-5,5-dimethyl-6H-benzo[h]quinazolin-7-yl]-ethyl-amino]acetate as a yellow solid (2100 mg, 57% yield).

MS m/z (+ESI): 563.8 [M+H]⁺.

### Step 3: Preparation of ethyl 2-[[4-amino-8-(cis-4-aminocyclohexoxy)-5,5-dimethyl-6H-benzo[h]quinazolin-7-yl]-ethyl-amino]acetate

The title compound was prepared as a yellow solid (150 mg, 86% yield) following Scheme 1 and in analogy to Example 4 (step 4) using ethyl 2-[[4-amino-8-[cis-4-(tert-butoxycarbonylamino)cyclohexoxy]-5,5-dimethyl-6H-benzo[h]quinazolin-7-yl]-ethyl-amino]acetate (200 mg, 0.30 mmol) as starting material.

MS m/z (+ESI): 468.3 [M+H]⁺.

### Step 4: Preparation of 2-[[4-amino-8-(cis-4-aminocyclohexoxy)-5,5-dimethyl-6H-benzo[h]quinazolin-7-yl]-ethyl-amino]ethanol

LiAlH₄ (28 mg, 0.72 mmol) was added to a stirred solution of ethyl 2-[[4-amino-8-(cis-4-aminocyclohexoxy)-5,5-dimethyl-6H-benzo[h]quinazolin-7-yl]-ethyl-amino]acetate (140 mg, 0.24 mmol) in THF (5 mL). After 1 h stirring, the reaction mixture was quenched with H₂O and the resulting suspension was filtered. The filtrate was concentrated and the residue was purified by preparative HPLC to afford 2-[[4-amino-8-(cis-4-aminocyclohexoxy)-5,5-dimethyl-6H-benzo[h]quinazolin-7-yl]-ethyl-amino]ethanol as an off-white solid (52 mg, 50% yield).

¹H NMR (400 MHz, DMSO-d₆+D₂O) δ ppm: 8.19 (s, 1H), 7.84 (d, J=8.8 Hz, 1H), 6.89 (d, J=8.8 Hz, 1H), 4.62 (m, 1H), 3.35 (m, 2H), 3.04 (m, 7H), 1.98 (m, 2H), 1.70 (m, 6H), 1.24 (s, 6H), 0.84 (t, J=7.2 Hz, 3H).

MS m/z (+ESI): 426.5 [M+H]⁺.

### Preparation of Example 25: 1-(4-amino-8-methoxy-5,5-dimethyl-6H-benzo[h]quinazolin-7-yl)azetidin-3-ol

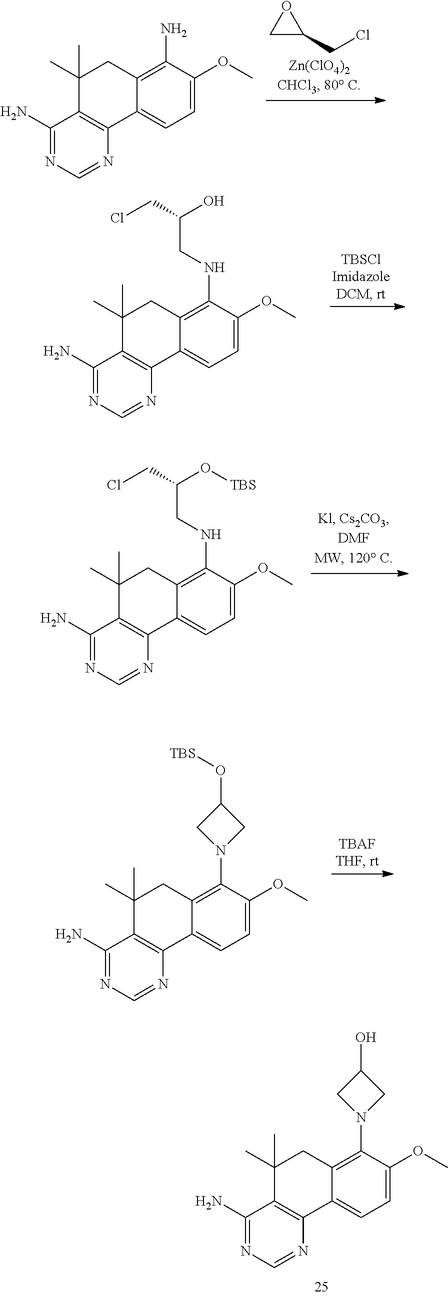

### Step 1: Preparation of (2R)-1-[(4-amino-8-methoxy-5,5-dimethyl-6H-benzo[h]quinazolin-7-yl)amino]-3-chloro-propan-2-ol

(R)-(−)-Epichlorohydrin (133 μL, 1.66 mmol) was added to a stirred solution of 8-methoxy-5,5-dimethyl-6H-benzo[h]quinazoline-4,7-diamine (500 mg, 1.66 mmol) in CHCl₃ (3 mL), followed by Zn(ClO₄)₂(126 mg, 0.33 mmol). After 18 h stirring at 80° C., the reaction mixture was extracted with DCM and H₂O. The combined organic layers were dried over Na₂SO₄, filtered and concentrated to afford (2R)-1-[(4-amino-8-methoxy-5,5-dimethyl-6H-benzo[h]quinazolin-7-yl)amino]-3-chloro-propan-2-ol as a light yellow solid (490 mg, 32% yield) that was used in the next step without further purification.

MS m/z (+ESI): 363.2, 365.2 [M+H]⁺.

### Step 2: Preparation of 8-methoxy-5,5-dimethyl-N7-[(2R)-2-[tert-butyl(dimethyl)silyl]oxy-3-chloro-propyl]-6H-benzo[h]quinazoline-4,7-diamine

Imidazole (74 mg, 1.08 mmol) was added to a stirred solution of (2R)-1-[(4-amino-8-methoxy-5,5-dimethyl-6H-benzo[h]quinazolin-7-yl)amino]-3-chloro-propan-2-ol (490 mg, 0.54 mmol) in DCM (3 mL), followed by TBSCl (168 mg, 1.08 mmol). After 8 h stirring, the reaction mixture was extracted with DCM and H₂O. The combined organic layers were dried over Na₂SO₄, filtered and concentrated. The residue was purified by combiflash to afford 8-methoxy-5,5-dimethyl-N7-[(2R)-2-[tert-butyl(dimethyl)silyl]oxy-3-chloro-propyl]-6H-benzo[h]quinazoline-4,7-diamine as a colorless oil (170 mg, 59% yield).

MS m/z (+ESI): 477.2, 479.2 [M+H]⁺.

### Step 3: Preparation of 7-[3-[tert-butyl(dimethyl)silyl]oxyazetidin-1-yl]-8-methoxy-5,5-dimethyl-6H-benzo[h]quinazolin-4-amine

Cs₂CO₃ (188 mg, 0.56 mmol) was added to a stirred solution of 8-methoxy-5,5-dimethyl-N7-[(2R)-2-[tert-butyl(dimethyl)silyl]oxy-3-chloro-propyl]-6H-benzo[h]quinazoline-4,7-diamine (150 mg, 0.28 mmol) in DMF (2 mL), followed by KI (47 mg, 0.28 mmol). After 3 h stirring at 120° C. under microwave irradiation, the reaction mixture was concentrated and extracted with EA and H₂O. The combined organic layers were dried over Na₂SO₄, filtered and concentrated to afford 7-[3-[tert-butyl(dimethyl)silyl]oxyazetidin-1-yl]-8-methoxy-5,5-dimethyl-6H-benzo[h]quinazolin-4-amine as a yellow solid (140 mg, 78% yield) which was used in the next step without further purification.

MS m/z (+ESI): 441.2 [M+H]⁺.

### Step 4: Preparation of 1-(4-amino-8-methoxy-5,5-dimethyl-6H-benzo[h]quinazolin-7-yl)azetidin-3-ol

TBAF (95 mg, 0.31 mmol) was added to a stirred solution of 7-[3-[tert-butyl(dimethyl)silyl]oxyazetidin-1-yl]-8-methoxy-5,5-dimethyl-6H-benzo[h]quinazolin-4-amine (130 mg, 0.21 mmol) in THF (1.5 mL). After 2 h stirring, the reaction mixture was concentrated and the residue was purified by preparative HPLC to afford 1-(4-amino-8-methoxy-5,5-dimethyl-6H-benzo[h]quinazolin-7-yl)azetidin-3-ol as a yellow solid (33 mg, 46% yield).

¹H NMR (400 MHz, DMSO-d₆+D₂O) δ ppm: 8.21 (s, 1H), 7.55 (d, J=8.4 Hz, 1H), 6.85 (d, J=8.8 Hz, 1H), 4.32 (m, 1H), 4.26 (m, 2H), 3.77 (m, 2H), 3.74 (s, 3H), 2.66 (s, 2H), 1.22 (s, 6H).

MS m/z (+ESI): 327.3 [M+H]⁺.

### Preparation of Example 26: 2-[[4-amino-8-(cis-4-aminocyclohexoxy)-5,5-dimethyl-6H-benzo[h]quinazolin-7-yl]-methyl-amino]-N-methyl-acetamide

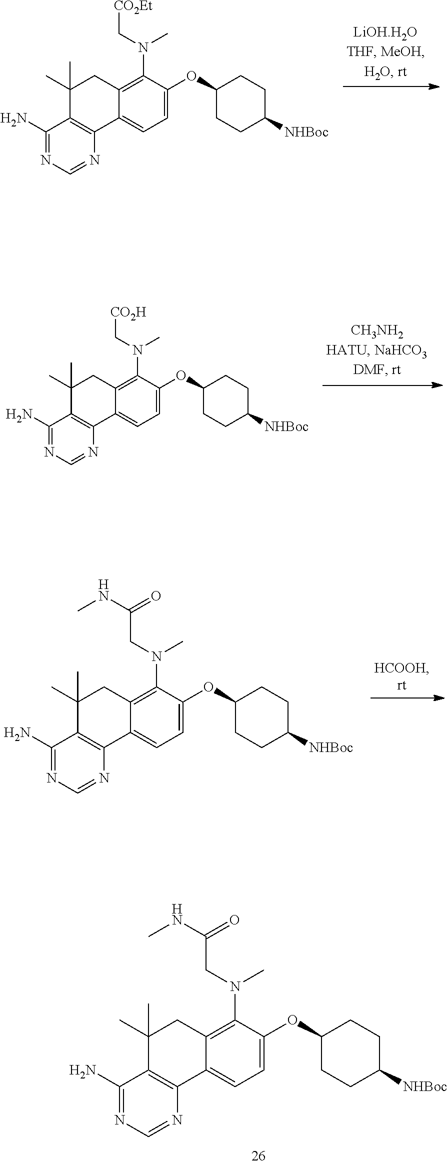 [SMILES: CCOC(=O)CN(C)c1c(O[C@H]2CC[C@@H](NC(=O)OC(C)(C)C)CC2)ccc2c1CC(C)(C)c1c(N)ncnc1-2 ; CN(CC(=O)O)c1c(O[C@H]2CC[C@@H](NC(=O)OC(C)(C)C)CC2)ccc2c1CC(C)(C)c1c(N)ncnc1-2 ; CNC(=O)CN(C)c1c(O[C@H]2CC[C@@H](NC(=O)OC(C)(C)C)CC2)ccc2c1CC(C)(C)c1c(N)ncnc1-2 ; CNC(=O)CN(C)c1c(O[C@H]2CC[C@@H](NC(=O)OC(C)(C)C)CC2)ccc2c1CC(C)(C)c1c(N)ncnc1-2]

The starting material was prepared according to procedure described for Example 23 from 4-amino-5,5-dimethyl-6H-benzo[h]quinazolin-8-ol and using tert-butyl N-(trans-4-hydroxycyclohexyl)carbamate, ethyl bromoacetate and formaldehyde.

### Step 1: Preparation of 2-[[4-amino-8-[cis-4-(tert-butoxycarbonylamino)cyclohexoxy]-5,5-dimethyl-6H-benzo[h]quinazolin-7-yl]-methyl-amino]acetic acid

LiOH·H₂O (11 mg, 0.46 mmol) was added to a stirred solution of ethyl 2-[[4-amino-8-[cis-4-(tert-butoxycarbonylamino)cyclohexoxy]-5,5-dimethyl-6H-benzo[h]quinazolin-7-yl]-methyl-amino]acetate (150 mg, 0.23 mmol) in THF (6 mL), MeOH (2 mL) and H₂O (2 mL). After 1 h stirring, the reaction mixture was concentrated. The residue was diluted with H₂O and the pH was adjusted to 5 with a 3M HCl aqueous solution. The resulting suspension was filtered, the cake was washed with water and dried under vacuum to afford 2-[[4-amino-8-[cis-4-(tert-butoxycarbonylamino)cyclohexoxy]-5,5-dimethyl-6H-benzo[h]quinazolin-7-yl]-methyl-amino]acetic acid as a yellow solid (120 mg, 84% yield) which was used in the next step without further purification.

MS m/z (+ESI): 526.3 [M+H]⁺.

### Step 2: Preparation of tert-butyl N-[cis-4-[[4-amino-5,5-dimethyl-7-[methyl-[2-(methylamino)-2-oxo-ethyl]amino]-6H-benzo[h]quinazolin-8-yl]oxy]cyclohexyl]carbamate

Methylamine (2M in THF, 445 μL, 0.89 mmol) was added to a stirred solution of 2-[[4-amino-8-[cis-4-(tert-butoxycarbonylamino)cyclohexoxy]-5,5-dimethyl-6H-benzo[h]quinazolin-7-yl]-methyl-amino]acetic acid (110 mg, 0.18 mmol) in DMF (5 mL), followed by HATU (103 mg, 0.27 mmol) and NaHCO₃ (76 mg, 0.89 mmol). After 1 h stirring, the reaction mixture was concentrated and extracted with EA and H₂O. The combined organic layers were dried over Na₂SO₄, filtered and concentrated to afford tert-butyl N-[cis-4-[[4-amino-5,5-dimethyl-7-[methyl-[2-(methylamino)-2-oxo-ethyl]amino]-6H-benzo[h]quinazolin-8-yl]oxy]cyclohexyl]carbamate as a yellow solid (95 mg, 84% yield) which was used in the next step without further purification.

MS m/z (+ESI): 539.3 [M+H]⁺.

### Step 3: Preparation of 2-[[4-amino-8-(cis-4-aminocyclohexoxy)-5,5-dimethyl-6H-benzo[h]quinazolin-7-yl]-methyl-amino]-N-methyl-acetamide

The title compound was prepared as a yellow solid (34 mg, 50% yield) following Scheme 1 and in analogy to Example 9 (step 2) using tert-butyl N-[cis-4-[[4-amino-5,5-dimethyl-7-[methyl-[2-(methylamino)-2-oxo-ethyl]amino]-6H-benzo[h]quinazolin-8-yl]oxy]cyclohexyl]carbamate (90 mg, 0.14 mmol) as starting material.

¹H NMR (400 MHz, DMSO-d₆+D₂O) δ ppm: 8.21 (s, 1H), 7.86 (d, J=8.8 Hz, 1H), 6.93 (d, J=8.8 Hz, 1H), 4.66 (m, 1H), 3.64 (m, 1H), 3.45 (m, 1H), 3.09 (m, 1H), 2.94 (s, 2H), 2.73 (s, 3H), 2.68 (s, 3H), 2.03 (m, 2H), 1.72 (m, 6H), 1.29 (s, 3H), 1.27 (s, 3H).

MS m/z (+ESI): 439.4 [M+H]⁺.

### Preparation of Example 28: [1-(4-amino-8-methoxy-5,5-dimethyl-6H-benzo[h]quinazolin-7-yl)azetidin-3-yl]methanol

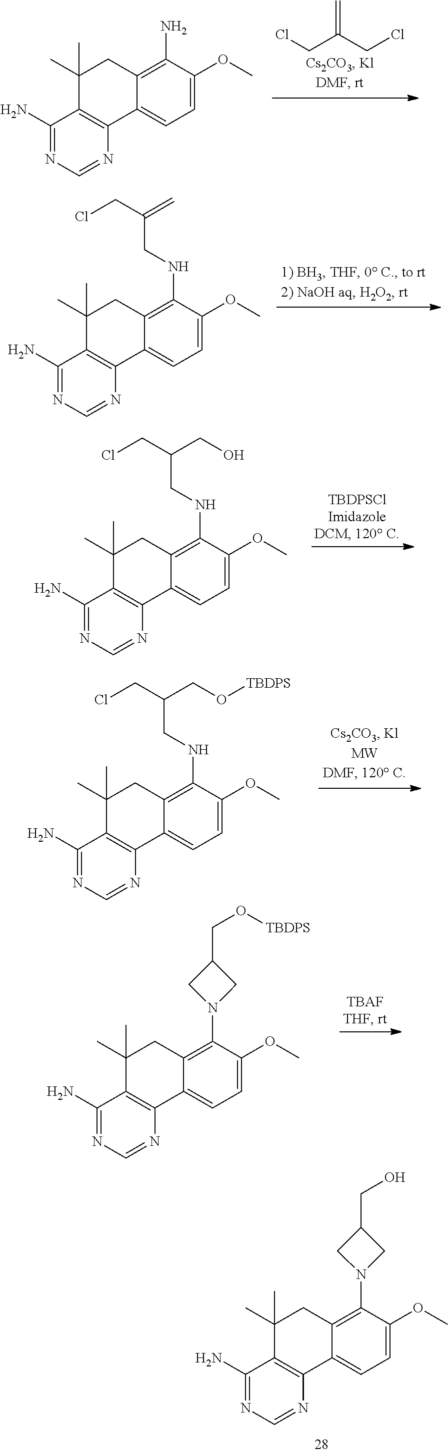

### Step 1: Preparation of N7-[2-(chloromethyl)allyl]-8-methoxy-5,5-dimethyl-6H-benzo[h]quinazoline-4,7-diamine

3-Chloro-2-chloromethyl-1-propene (1.05 mL, 8.78 mmol) was added to a stirred solution of 8-methoxy-5,5-dimethyl-6H-benzo[h]quinazoline-4,7-diamine (500 mg, 1.76 mmol) in DMF (4 mL), followed by Cs₂CO₃ (1.17 g, 3.51 mmol) and KI (147 mg, 0.88 mmol). After 6 h stirring, the reaction mixture was extracted with DCM and H₂O. The combined organic layers were dried over Na₂SO₄, filtered and concentrated. The residue was purified by combiflash to afford N7-[2-(chloromethyl)allyl]-8-methoxy-5,5-dimethyl-6H-benzo[h]quinazoline-4,7-diamine as a yellow solid (320 mg, 46% yield)

¹H NMR (400 MHz, DMSO-d₆) δ ppm: 8.23 (s, 1H), 7.67 (d, J=8.8 Hz, 1H), 6.89 (d, J=8.8 Hz, 1H), 6.34 (br, 2H), 5.22 (s, 1H), 5.14 (s, 1H), 4.28 (s, 2H), 4.25 (m, 1H), 3.82 (s, 3H), 3.68 (d, J=6.8 Hz, 2H), 2.75 (s, 2H), 1.26 (s, 6H).

MS m/z (+ESI): 359.2, 361.3 [M+H]⁺.

### Step 2: Preparation of 2-[[(4-amino-8-methoxy-5,5-dimethyl-6H-benzo[h]quinazolin-7-yl)amino]methyl]-3-chloro-propan-1-ol

1M Borane THF complex solution (1.6 mL, 1.6 mmol) was added at 0° C. to a stirred solution of N7-[2-(chloromethyl)allyl]-8-methoxy-5,5-dimethyl-6H-benzo[h]quinazoline-4,7-diamine (320 mg, 0.80 mmol) in THF (1 mL). After 2 h stirring at rt, 2.5M NaOH aqueous solution (3.2 mL, 8.02 mmol) was added dropwise to the reaction mixture, followed H₂O₂(0.82 mL, 8.02 mmol). After 2 h stirring at rt, the resulting solution was extracted with DCM and H₂O. The combined organic layers were dried over Na₂SO₄, filtered and concentrated. The residue was purified by combiflash to afford 2-[[(4-amino-8-methoxy-5,5-dimethyl-6H-benzo[h]quinazolin-7-yl)amino]methyl]-3-chloro-propan-1-ol as a light yellow solid (180 mg, 53% yield).

MS m/z (+ESI): 377.2, 379.3 [M+H]⁺.

### Step 3: Preparation of N7-[2-[[tert-butyl(diphenyl)silyl]oxymethyl]-3-chloro-propyl]-8-methoxy-5,5-dimethyl-6H-benzo[h]quinazoline-4,7-diamine

TBDPSCl (228 μL, 0.86 mmol) was added to a stirred solution of 2-[[(4-amino-8-methoxy-5,5-dimethyl-6H-benzo[h]quinazolin-7-yl)amino]methyl]-3-chloro-propan-1-ol (180 mg, 0.43 mmol, in DCM (2 mL), followed by imidazole (148 mg, 2.15 mmol). After 2 h stirring at 120° C., the reaction mixture was extracted with DCM and H₂O. The combined organic layers were dried over Na₂SO₄, filtered and concentrated to afford N7-[2-[[tert-butyl(diphenyl)silyl]oxymethyl]-3-chloro-propyl]-8-methoxy-5,5-dimethyl-6H-benzo[h]quinazoline-4,7-diamine as a light yellow oil (600 mg, 34% yield) which was used in the next step without further purification.

MS m/z (+ESI): no mass signal.

### Step 4: Preparation of 7-[3-[[tert-butyl(diphenyl)silyl]oxymethyl]azetidin-1-yl]-8-methoxy-5,5-dimethyl-6H-benzo[h]quinazolin-4-amine

The title compound was prepared as a yellow oil (560 mg, 66% yield) following Scheme 1 and in analogy to Example 25 (step 3) using N7-[2-[[tert-butyl(diphenyl)silyl]oxymethyl]-3-chloro-propyl]-8-methoxy-5,5-dimethyl-6H-benzo[h]quinazoline-4,7-diamine (600 mg, 0.15 mmol) as starting material.

MS m/z (+ESI): no mass signal.

### Step 5: Preparation of [1-(4-amino-8-methoxy-5,5-dimethyl-6H-benzo[h]quinazolin-7-yl)azetidin-3-yl]methanol

TBAF (63 mg, 0.19 mmol) was added to a stirred solution of 7-[3-[[tert-butyl(diphenyl)silyl]oxymethyl]azetidin-1-yl]-8-methoxy-5,5-dimethyl-6H-benzo[h]quinazolin-4-amine (560 mg, 0.09 mmol) in THF (1.5 mL). After 2 h stirring, the reaction mixture was concentrated. The residue was purified by preparative HPLC to afford [1-(4-amino-8-methoxy-5,5-dimethyl-6H-benzo[h]quinazolin-7-yl)azetidin-3-yl]methanol as a white solid (12 mg, 34% yield).

¹H NMR (400 MHz, DMSO-d₆+D₂O) δ ppm: 8.20 (s, 1H), 7.53 (d, J=8.8 Hz, 1H), 6.84 (d, J=8.4 Hz, 1H), 4.08 (m, 2H), 3.78 (m, 2H), 3.73 (s, 3H), 3.55 (m, 2H), 2.69 (s, 2H), 2.57 (m, 1H), 1.21 (s, 6H).

MS m/z (+ESI): 341.2 [M+H]⁺.

### Preparation of Example 32: 8-(cis-4-aminocyclohexoxy)-N7,5,5-trimethyl-N7-(2-methylsulfonylethyl)-6H-benzo[h]quinazoline-4,7-diamine

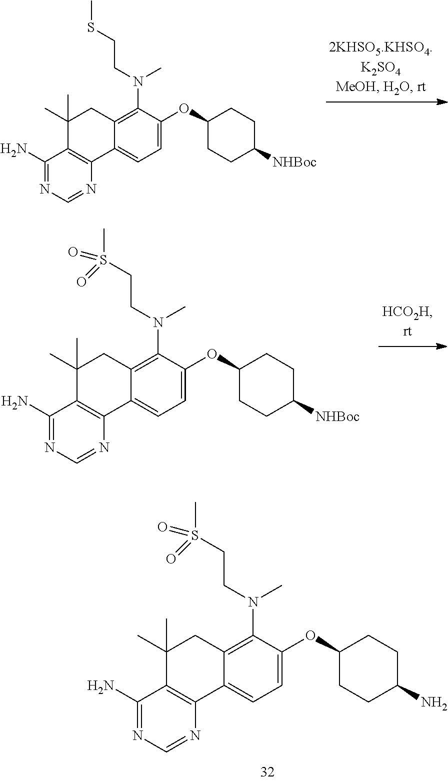

The starting material was prepared according to procedures described for Examples 5 and 8 from 4-amino-5,5-dimethyl-6H-benzo[h]quinazolin-8-ol and using tert-butyl N-(trans-4-hydroxycyclohexyl)carbamate, (methylthio)acetaldehyde and formaldehyde.

### Step 1: Preparation of tert-butyl N-[cis-4-[[4-amino-5,5-dimethyl-7-[methyl(2-methylsulfonylethyl)amino]-6H-benzo[h]quinazolin-8-yl]oxy]cyclohexyl]carbamate

Potassium peroxomonosulfate (4.5% active oxygen, 521 mg, 0.83 mmol) was added to a stirred solution of tert-butyl N-[cis-4-[[4-amino-5,5-dimethyl-7-[methyl(2-methylsulfanylethyl)amino]-6H-benzo[h]quinazolin-8-yl]oxy]cyclohexyl]carbamate (250 mg, 0.41 mmol) in MeOH (8 mL) and H₂O (2 mL). After 1 h stirring, the reaction suspension was filtered, the filtrate was concentrated and the crude was purified by combiflash to afford tert-butyl N-[cis-4-[[4-amino-5,5-dimethyl-7-[methyl(2-methylsulfonylethyl)amino]-6H-benzo[h]quinazolin-8-yl]oxy]cyclohexyl]carbamate as a yellow solid (230 mg, 87% yield).

MS m/z (+ESI): 574.3 [M+H]⁺.

### Step 2: Preparation of 8-(cis-4-aminocyclohexoxy)-N7,5,5-trimethyl-N7-(2-methylsulfonylethyl)-6H-benzo[h]quinazoline-4,7-diamine

The title compound was prepared as a yellow solid (83 mg, 73% yield) following Scheme 1 and in analogy to Example 9 (step 2) using tert-butyl N-[cis-4-[[4-amino-55-dimethyl-7-[methyl(2-methylsulfonylethyl)amino]-6H-benzo[h]quinazolin-8-yl]oxy]cyclohexyl]carbamate (150 mg, 0.23 mmol) as starting material.

¹H NMR (400 MHz, DMSO-d₆+D₂O) δ ppm: 8.22 (s, 1H), 7.88 (d, J=8.8 Hz, 1H), 6.94 (d, J=8.8 Hz, 1H), 4.65 (m, 1H), 3.53 (m, 2H), 3.15 (m, 4H), 2.98 (s, 3H), 2.78 (s, 1H), 2.75 (s, 3H), 2.01 (m, 2H), 1.72 (m, 6H), 1.28 (s, 3H), 1.25 (s, 3H).

MS m/z (+ESI): 474.3 [M+H]⁺.

### Preparation of Example 34: 4,7-diamino-8-(trans-4-aminocyclohexoxy)-5,5-dimethyl-benzo[h]quinazolin-6-one

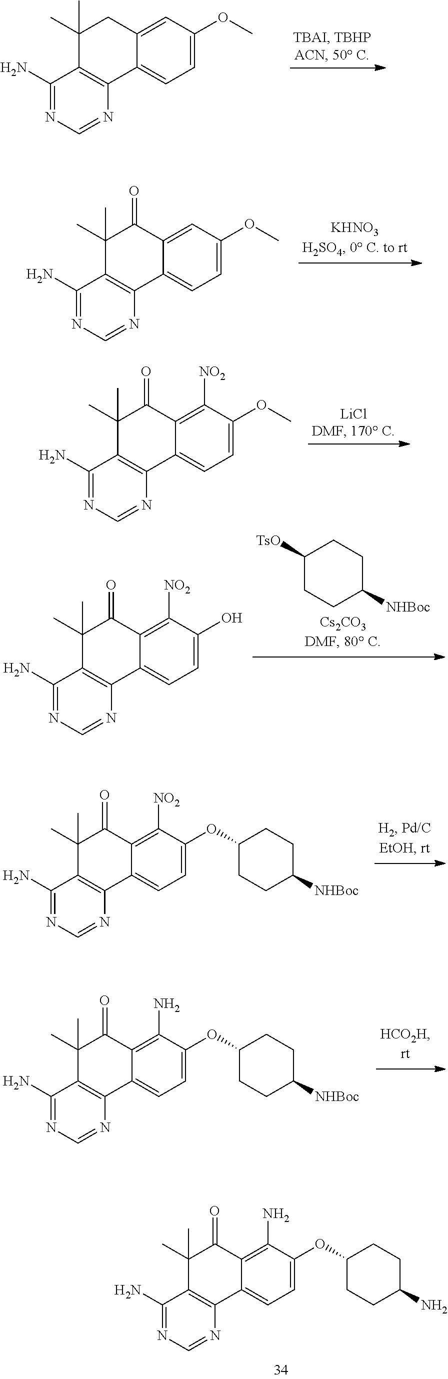

### Step 1: Preparation of 4-amino-8-methoxy-5,5-dimethyl-benzo[h]quinazolin-6-one

TBAI (7.4 g, 19.5 mmol) was added to a stirred suspension of 8-methoxy-5,5-dimethyl-6H-benzo[h]quinazolin-4-amine (25 g, 97.8 mmol) in ACN (150 mL). The resulting suspension was heated to 45° C. before the dropwise addition of TBHP solution (100 g, 782 mmol) over 3 h. After 16 h stirring at 50° C. the reaction mixture was cooled down to rt before the dropwise addition of H₂O (150 mL) over 30 min. After 16 h stirring at 0° C., the resulting precipitate was collected by filtration, washed with a mixture of ACN (25 mL) and H₂O (75 mL) and dried under vacuum to afford 4-amino-8-methoxy-5,5-dimethyl-benzo[h]quinazolin-6-one as a light grey solid (26.3 g, 69% yield) which was used in the next step without further purification.

¹H NMR (400 MHz, DMSO-d₆) δ ppm: 8.56 (d, J=8.8 Hz, 1H), 8.38 (s, 1H), 7.48 (d, J=2.8 Hz, 1H), 7.42 (dd, J=8.8 Hz, 2.8 Hz, 1H), 6.87 (s, 2H), 3.89 (s, 3H), 1.51 (s, 6H)

MS m/z (+ESI): 270.2 [M+H]⁺.

### Step 2: Preparation of 4-amino-8-methoxy-5,5-dimethyl-7-nitro-benzo[h]quinazolin-6-one

KHNO₃ (2.53 g, 24.5 mmol) was added portionwise at 0° C. to a stirred solution of 4-amino-8-methoxy-5,5-dimethyl-benzo[h]quinazolin-6-one (5.1 g, 18.8 mmol) in H₂SO₄ (10 mL). After 1 h stirring at rt, the reaction mixture was poured into ice water and basified to pH 9 with a 2N NaOH aqueous solution. The resulting precipitate was collected by filtration to afford 4-amino-8-methoxy-5,5-dimethyl-7-nitro-benzo[h]quinazolin-6-one as a light yellow solid (5.9 g, 65% yield) which was used in the next step without further purification.

MS m/z (+ESI): 315.1 [M+H]⁺.

### Step 3: Preparation of 4-amino-8-hydroxy-5,5-dimethyl-7-nitro-benzo[h]quinazolin-6-one

LiCl (1.61 g, 36.7 mmol) was added to a stirred suspension of 4-amino-8-methoxy-5,5-dimethyl-7-nitro-benzo[h]quinazolin-6-one (5.5 g, 12.25 mmol) in DMF (30 mL). After 2 h stirring at 170° C., the solvent was removed to afford 4-amino-8-hydroxy-5,5-dimethyl-7-nitro-benzo[h]quinazolin-6-one as a yellow liquid (5.2 g, 98% yield) which was used in the next step without further purification.

MS m/z (+ESI): 301.1 [M+H]⁺.

### Step 4: Preparation of tert-butyl N-[trans-4-(4-amino-5,5-dimethyl-7-nitro-6-oxo-benzo[h]quinazolin-8-yl)oxycyclohexyl]carbamate

The title compound was prepared as a yellow solid (6 g, 59% yield) following Scheme 1 and in analogy to Example 5 (step 3) using [cis-4-(tert-butoxycarbonylamino)cyclohexyl]4-methylbenzenesulfonate (14.9 g, 36.3 mmol) as starting material.

¹H NMR (400 MHz, DMSO-d₆) δ ppm: 8.72 (d, J=9.2 Hz, 1H), 8.40 (s, 1H), 7.96 (d, J=9.2 Hz, 1H), 7.00 (s, 2H), 6.85 (d, J=7.6 Hz, 1H), 4.61 (m, 1H), 3.35 (m, 1H), 2.02 (m, 2H), 1.79 (m, 2H), 1.49 (s, 6H), 1.38 (m, 13H).

MS m/z (+ESI): 498.2 [M+H]⁺.

### Step 5: Preparation of tert-butyl N-[trans-4-(4,7-diamino-5,5-dimethyl-6-oxo-benzo[h]quinazolin-8-yl)oxycyclohexyl]carbamate

10% Pd/C (19 mg, 0.02 mmol) was added to a stirred solution of tert-butyl N-[trans-4-(4-amino-5,5-dimethyl-7-nitro-6-oxo-benzo[h]quinazolin-8-yl)oxycyclohexyl]carbamate (100 mg, 0.18 mmol) in EtOH (10 mL). After 20 h stirring under hydrogen flow, the catalyst was removed by filtration and the solution was concentrated to afford tert-butyl N-[trans-4-(4,7-diamino-5,5-dimethyl-6-oxo-benzo[h]quinazolin-8-yl)oxycyclohexyl]carbamate as a yellow solid (90 mg, 95% yield) which was used in the next step without further purification.

MS m/z (+ESI): 468.3 [M+H]⁺.

### Step 6: Preparation of 4,7-diamino-8-(trans-4-aminocyclohexoxy)-5,5-dimethyl-benzo[h]quinazolin-6-one

The title compound was prepared as a yellow solid (42 mg, 62% yield) following Scheme 1 and in analogy to Example 9 (step 2) using tert-butyl N-[trans-4-(4,7-diamino-5,5-dimethyl-6-oxo-benzo[h]quinazolin-8-yl)oxycyclohexyl]carbamate (90 mg, 0.17 mmol) as starting material.

¹H NMR (400 MHz, DMSO-d₆+D₂O) δ ppm: 8.30 (s, 1H), 7.82 (d, J=8.4 Hz, 1H), 7.26 (d, J=8.4 Hz, 1H), 4.37 (m, 1H), 3.01 (m, 1H), 2.11 (m, 2H), 1.97 (m, 2H), 1.48 (m, 10H).

MS m/z (+ESI): 368.3 [M+H]⁺.

### Preparation of Examples 38 and 39: N-[2-[[4-amino-8-(trans-4-aminocyclohexoxy)-5,5-dimethyl-6H-benzo[h]quinazolin-7-yl]-methyl-amino]ethyl]acetamide and N-[2-[[4-amino-8-(trans-4-aminocyclohexoxy)-5,5-dimethyl-6H-benzo[h]quinazolin-7-yl]-methyl-amino]ethyl]methanesulfonamide

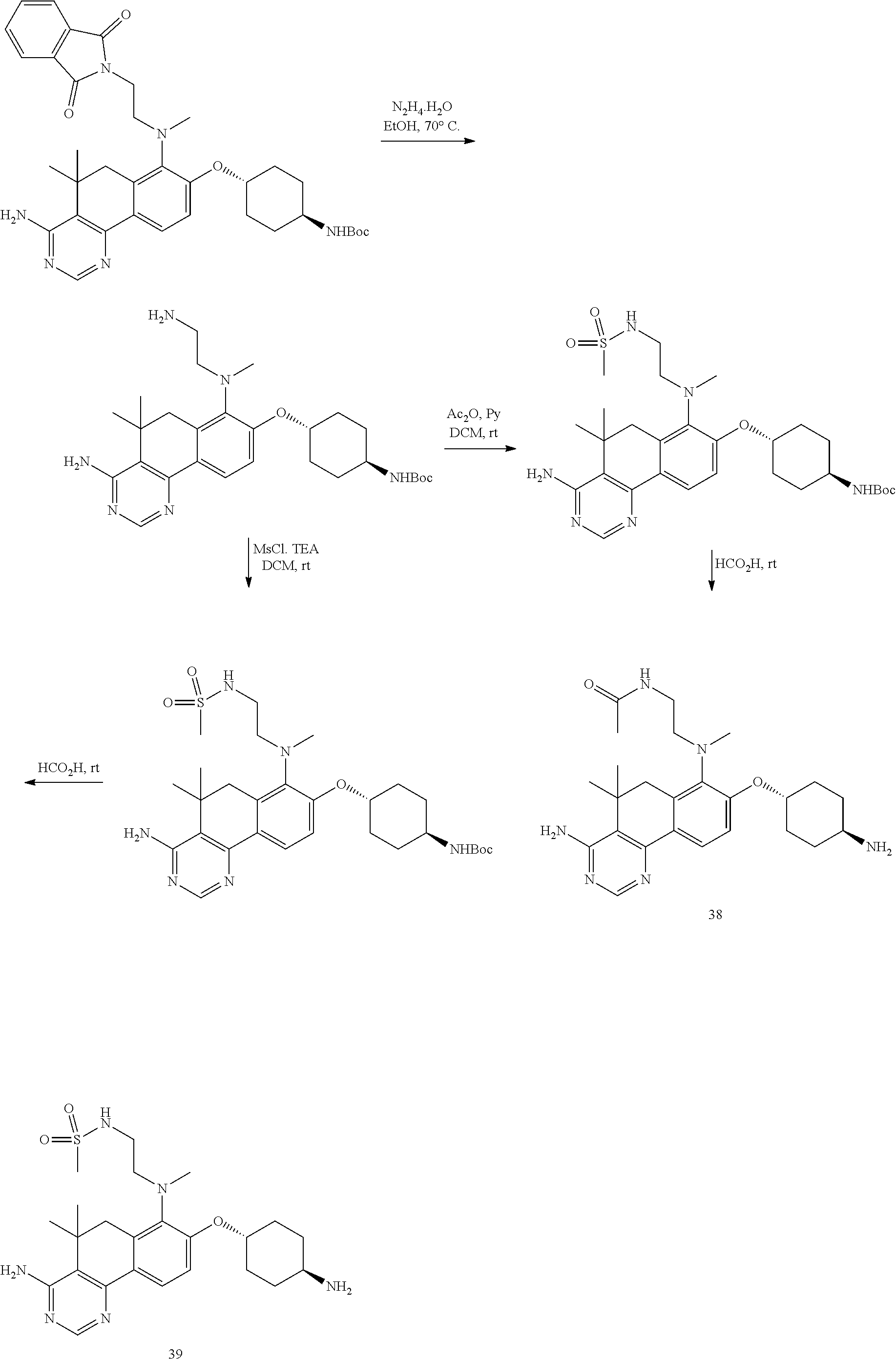 [SMILES: CN(CCN1C(=O)c2ccccc2C1=O)c1c(O[C@H]2CC[C@H](NC(=O)OC(C)(C)C)CC2)ccc2c1CC(C)(C)c1c(N)ncnc1-2 ; CN(CCN)c1c(O[C@H]2CC[C@H](NC(=O)OC(C)(C)C)CC2)ccc2c1CC(C)(C)c1c(N)ncnc1-2 ; CN(CCNS(C)(=O)=O)c1c(O[C@H]2CC[C@H](NC(=O)OC(C)(C)C)CC2)ccc2c1CC(C)(C)c1c(N)ncnc1-2 ; CN(CCNS(C)(=O)=O)c1c(O[C@H]2CC[C@H](NC(=O)OC(C)(C)C)CC2)ccc2c1CC(C)(C)c1c(N)ncnc1-2 ; CC(=O)NCCN(C)c1c(O[C@H]2CC[C@H](N)CC2)ccc2c1CC(C)(C)c1c(N)ncnc1-2 ; CN(CCNS(C)(=O)=O)c1c(O[C@H]2CC[C@H](N)CC2)ccc2c1CC(C)(C)c1c(N)ncnc1-2]

The starting material was prepared according to procedures described for Examples 5 and 8 from 4-amino-5,5-dimethyl-6H-benzo[h]quinazolin-8-ol and using tert-butyl N-(cis-4-hydroxycyclohexyl)carbamate, 1,3-dihydro-1,3-dioxo-2H-isoindole-2-acetaldehyde and formaldehyde.

### Step 1: Preparation of tert-butyl N-[trans-4-[[4-amino-7-[2-aminoethyl(methyl)amino]-5,5-dimethyl-6H-benzo[h]quinazolin-8-yl]oxy]cyclohexyl]carbamate

N₂H₄·H₂O (47 mg, 0.93 mmol) was added to a stirred solution of tert-butyl N-[trans-4-[[4-amino-7-[2-(1,3-dioxoisoindolin-2-yl)ethyl-methyl-amino]-5,5-dimethyl-6H-benzo[h]quinazolin-8-yl]oxy]cyclohexyl]carbamate (220 mg, 0.31 mmol) in EtOH (2 mL). After 2 h stirring at 70° C., the solvent was removed and the crude was purified by combiflash to afford tert-butyl N-[trans-4-[[4-amino-7-[2-aminoethyl(methyl)amino]-5,5-dimethyl-6H-benzo[h]quinazolin-8-yl]oxy]cyclohexyl]carbamate as a yellow solid (158 mg, 54% yield).

MS m/z (+ESI): 511.4 [M+H]⁺.

### Step 2: Preparation of tert-butyl N-[trans-4-[[7-[2-acetamidoethyl(methyl)amino]-4-amino-5,5-dimethyl-6H-benzo[h]quinazolin-8-yl]oxy]cyclohexyl]carbamate

Ac₂O (15 μL, 0.16 mmol) was added to a stirred solution of tert-butyl N-[trans-4-[[4-amino-7-[2-aminoethyl(methyl)amino]-5,5-dimethyl-6H-benzo[h]quinazolin-8-yl]oxy]cyclohexyl]carbamate (60 mg, 0.11 mmol) in DCM (1 mL), followed by pyridine (13 μL, 0.16 mmol). After 2 h stirring, the reaction mixture was extracted with DCM and H₂O. The combined organic layers were dried over Na₂SO₄, filtered and concentrated to afford tert-butyl N-[trans-4-[[7-[2-acetamidoethyl(methyl)amino]-4-amino-5,5-dimethyl-6H-benzo[h]quinazolin-8-yl]oxy]cyclohexyl]carbamate as a yellow solid (60 mg, 92% yield) which was used in the next step without further purification.

MS m/z (+ESI): 553.3 [M+H]⁺.

### Step 3: Preparation of N-[2-[[4-amino-8-(trans-4-aminocyclohexoxy)-5,5-dimethyl-6H-benzo[h]quinazolin-7-yl]-methyl-amino]ethyl]acetamide

The title compound was prepared as a white solid (36 mg, 80% yield) following Scheme 1 and in analogy to Example 9 (step 2) using tert-butyl N-[trans-4-[[7-[2-acetamidoethyl(methyl)amino]-4-amino-5,5-dimethyl-6H-benzo[h]quinazolin-8-yl]oxy]cyclohexyl]carbamate (55 mg, 0.90 mmol) as starting material.

¹H NMR (400 MHz, DMSO-d₆+D₂O) δ ppm: 8.20 (s, 1H), 7.84 (d, J=8.8 Hz, 1H), 6.99 (d, J=8.8 Hz, 1H), 4.36 (m, 1H), 3.02 (m, 5H), 2.86 (m, 2H), 2.63 (s, 3H), 2.14 (m, 2H), 1.97 (m, 2H), 1.77 (s, 3H), 1.48 (m, 4H), 1.25 (s, 3H), 1.23 (s, 3H).

MS m/z (+ESI): 453.5 [M+H]⁺.

### Step 4: Preparation of tert-butyl N-[trans-4-[[4-amino-7-[2-(methanesulfonamido)ethyl-methyl-amino]-5,5-dimethyl-6H-benzo[h]quinazolin-8-yl]oxy]cyclohexYl]carbamate

MsCl (18 μL, 0.22 mmol) was added to a stirred solution of tert-butyl N-[trans-4-[[4-amino-7-[2-aminoethyl(methyl)amino]-5,5-dimethyl-6H-benzo[h]quinazolin-8-yl]oxy]cyclohexyl]carbamate (85 mg, 0.15 mmol) in DCM (1 mL), followed by TEA (42 μL, 0.30 mmol). After 2 h stirring, the reaction mixture was extracted with DCM and H₂O. The combined organic layers were dried over Na₂SO₄, filtered and concentrated to afford tert-butyl N-[trans-4-[[4-amino-7-[2-(methanesulfonamido)ethyl-methyl-amino]-5,5-dimethyl-6H-benzo[h]quinazolin-8-yl]oxy]cyclohexyl]carbamate as a yellow solid (70 mg, 710% yield) which was used in the next step without further purification.

MS m/z (+ESI): 589.3 [M+H]⁺.

### Step 5: Preparation of N-[2-[[4-amino-8-(trans-4-aminocyclohexoxy)-5,5-dimethyl-6H-benzo[h]quinazolin-7-yl]-methyl-amino]ethyl]methanesulfonamide

The title compound was prepared as a white solid (11 mg, 20% yield) following Scheme 1 and in analogy to Example 9 (step 2) using tert-butyl N-[trans-4-[[4-amino-7-[2-(methanesulfonamido)ethyl-methyl-amino]-5,5-dimethyl-6H-benzo[h]quinazolin-8-yl]oxy]cyclohexyl]carbamate (70 mg, 0.10 mmol) as starting material.

¹H NMR (400 MHz, DMSO-d₆+D₂O) δ ppm: 8.22 (s, 1H), 7.86 (d, J=8.8 Hz, 1H), 7.00 (d, J=8.8 Hz, 1H), 4.38 (m, 1H), 2.99 (m, 5H), 2.79 (m, 2H), 2.85 (s, 3H), 2.65 (s, 3H), 2.15 (m, 2H), 1.97 (m, 2H), 1.49 (m, 4H), 1.28 (s, 3H), 1.24 (s, 3H)

MS m/z (+ESI): 489.3 [M+H]⁺.

### Preparation of Example 42: N-[4-amino-8-(trans-4-aminocyclohexoxy)-5,5-dimethyl-6H-benzo[h]quinazolin-7-yl]-2-hydroxy-ethanesulfonamide

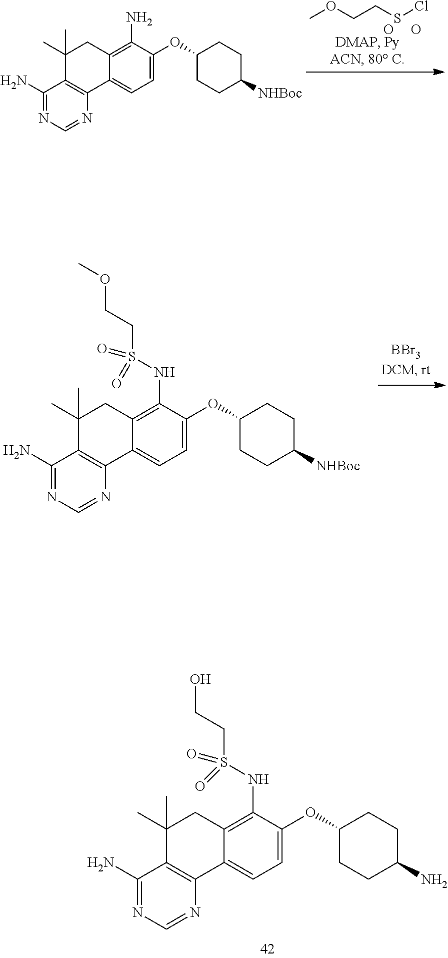

### Step 1: Preparation of tert-butyl N-[trans-4-[[4-amino-7-(2-methoxyethylsulfonylamino)-5,5-dimethyl-6H-benzo[h]quinazolin-8-yl]oxy]cyclohexyl]carbamate

2-methoxyethanesulfonyl chloride (451 mg, 2.80 mmol) was added to a stirred solution of tert-butyl N-[trans-4-[(4,7-diamino-5,5-dimethyl-6H-benzo[h]quinazolin-8-yl)oxy]cyclohexyl]carbamate (300 mg, 0.56 mmol) in ACN (10 mL), followed by DMAP (69 mg, 0.56 mmol) and pyridine (458 μL, 5.60 mmol). After 16 h stirring at 80° C., the solvent was removed and the residue was extracted with EA and H₂O. The combined organic layers were dried over Na₂SO₄, filtered and concentrated to afford tert-butyl N-[trans-4-[[4-amino-7-(2-methoxyethylsulfonylamino)-5,5-dimethyl-6H-benzo[h]quinazolin-8-yl]oxy]cyclohexyl]carbamate as a yellow solid (140 mg, 28% yield) which was used in the next step without further purification.

MS m/z (+ESI): 576.3 [M+H]⁺.

### Step 2: Preparation of N-[4-amino-8-(trans-4-aminocyclohexoxy)-5,5-dimethyl-6H-benzo[h]quinazolin-7-yl]-2-hydroxy-ethanesulfonamide

The title compound was prepared as a white solid (33 mg, 36% yield) following Scheme 1 and in analogy to Example 4 (step 1) using tert-butyl N-[trans-4-[[4-amino-7-(2-methoxyethylsulfonylamino)-5,5-dimethyl-6H-benzo[h]quinazolin-8-yl]oxy]cyclohexyl]carbamate (130 mg, 0.15 mmol) as starting material.

¹H NMR (400 MHz, DMSO-d₆+D₂O) δ ppm: 8.24 (s, 1H), 8.00 (d, J=8.8 Hz, 1H), 7.11 (d, J=8.8 Hz, 1H), 4.42 (m, 1H), 3.82 (t, J=7.2 Hz, 2H), 3.26 (t, J=7.2 Hz, 2H), 3.02 (m, 1H), 2.85 (s, 2H), 2.14 (m, 2H), 2.00 (m, 2H), 1.51 (m, 4H), 1.25 (s, 6H).

MS m/z (+ESI): 462.3 [M+H]⁺.

### Preparation of Example 48: 1-[[[4-amino-8-(trans-4-aminocyclohexoxy)-5,5-dimethyl-6H-benzo[h]quinazolin-7-yl]-methyl-amino]methyl]cyclopropanecarbonitrile

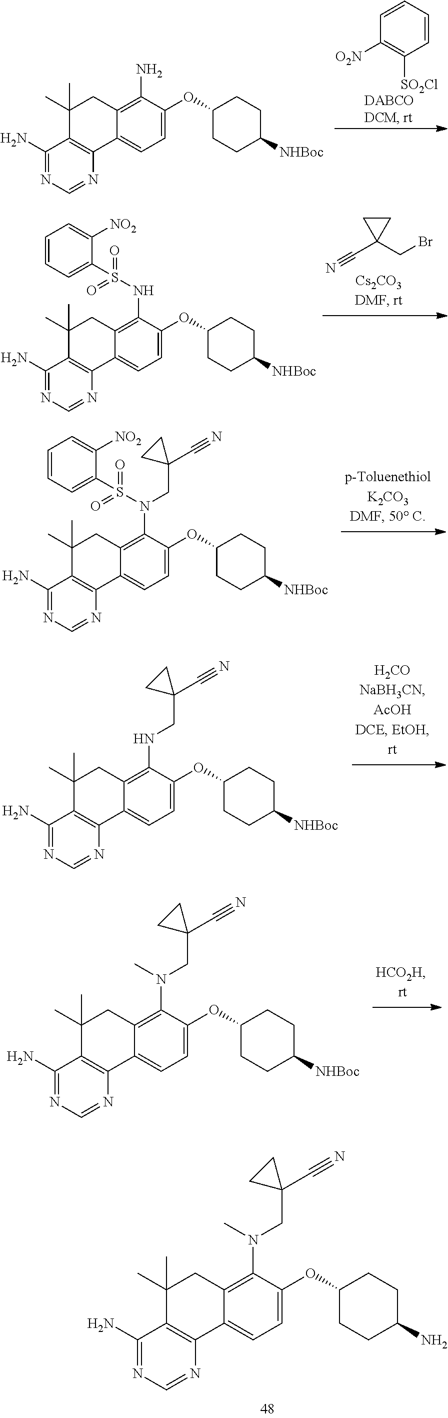

### Step 1: Preparation of tert-butyl N-[trans-4-[[4-amino-5,5-dimethyl-7-[(2-nitrophenyl)sulfonylamino]-6H-benzo[h]quinazolin-8-yl]oxy]cyclohexyl]carbamate

2-Nitrobenzenesulfonyl chloride (2.72 g, 11.90 mmol) was added to a stirred solution of tert-butyl N-[trans-4-[(4,7-diamino-5,5-dimethyl-6H-benzo[h]quinazolin-8-yl)oxy]cyclohexyl]carbamate (2 g, 3.97 mmol) in DCM (200 mL), followed by DABCO (2.25 g, 19.84 mmol). After 16 h stirring, the reaction mixture was extracted with DCM and H₂O. The combined organic layers were dried over Na₂SO₄, filtered, concentrated and purified by combiflash to afford tert-butyl N-[trans-4-[[4-amino-5,5-dimethyl-7-[(2-nitrophenyl)sulfonylamino]-6H-benzo[h]quinazolin-8-yl]oxy]cyclohexyl]carbamate as a yellow solid (2.53 g, 53% yield).

MS m/z (+ESI): 639.3 [M+H]⁺.

### Step 2: Preparation of tert-butyl N-[trans-4-[[4-amino-7-[(1-cyanocyclopropyl)methyl-(2-nitrophenyl)sulfonyl-amino]-5,5-dimethyl-6H-benzo[h]quinazolin-8-yl]oxy]cyclohexyl]carbamate

The title compound was prepared as a light yellow solid (255 mg, 76% yield) following Scheme 1 and in analogy to Example 4 (step 3) using tert-butyl N-[trans-4-[[4-amino-5,5-dimethyl-7-[(2-nitrophenyl)sulfonylamino]-6H-benzo[h]quinazolin-8-yl]oxy]cyclohexyl]carbamate (300 mg, 0.42 mmol) and 1-(bromomethyl)cyclopropanecarbonitrile (150 mg, 0.85 mmol) as starting materials.

MS m/z (+ESI): 718.3 [M+H]⁺.

### Step 3: Preparation of tert-butyl N-[trans-4-[[4-amino-7-[(1-cyanocyclopropyl)methylamino]-5,5-dimethyl-6H-benzo[h]quinazolin-8-yl]oxy]cyclohexyl]carbamate

p-Toluenethiol (108 mg, 0.86 mmol) was added to a stirred solution of tert-butyl N-[trans-4-[[4-amino-5,5-dimethyl-7-[(2-nitrophenyl)sulfonylamino]-6H-benzo[h]quinazolin-8-yl]oxy]cyclohexyl]carbamate (275 mg, 0.34 mmol) in DMF (2 mL), followed by K₂CO₃ (120 mg, 0.86 mmol). After 3 h stirring at 50° C., the reaction mixture was extracted with DCM and H₂O. The combined organic layers were dried over Na₂SO₄, filtered, concentrated and purified by combiflash to afford tert-butyl N-[trans-4-[[4-amino-7-[(1-cyanocyclopropyl)methylamino]-5,5-dimethyl-6H-benzo[h]quinazolin-8-yl]oxy]cyclohexyl]carbamate as a white solid (184 mg, 66% yield).

MS m/z (+ESI): 533.3 [M+H]⁺.

### Step 4: Preparation of 1-[[[4-amino-8-(trans-4-aminocyclohexoxy)-5,5-dimethyl-6H-benzo[h]quinazolin-7-yl]-methyl-amino]methyl]cyclopropanecarbonitrile

The title compound was prepared as a white solid (34 mg, 58% yield) following Scheme 1 and in analogy to Example 22 (step 2) and Example 9 (step 2) using tert-butyl N-[trans-4-[[4-amino-7-[(1-cyanocyclopropyl)methylamino]-5,5-dimethyl-6H-benzo[h]quinazolin-8-yl]oxy]cyclohexyl]carbamate (80 mg, 0.14 mmol) as starting material.

¹H NMR (400 MHz, DMSO-d₆+D₂O) δ ppm: 8.22 (s, 1H), 7.87 (d, J=8.8 Hz, 1H), 7.00 (d, J=8.8 Hz, 1H), 4.35 (m, 1H), 3.24 (m, 2H), 3.06 (m, 1H), 2.86 (m, 2H), 2.69 (s, 3H), 2.12 (m, 2H), 1.96 (m, 2H), 1.48 (m, 4H), 1.31 (s, 3H), 1.25 (m, 1H), 1.23 (s, 3H), 1.03 (m, 1H), 0.94 (m, 1H), 0.66 (m, 1H).

MS m/z (+ESI): 477.2 [M+H]⁺.

### Preparation of Examples 49 and 50: 1-[4-amino-8-(trans-4-aminocyclohexoxy)-5,5-dimethyl-6H-benzo[h]quinazolin-7-yl]pyrrolidine-3-carbonitrile—isomer 1 and 1-[4-amino-8-(trans-4-aminocyclohexoxy)-5,5-dimethyl-6H-benzo[h]quinazolin-7-yl]pyrrolidine-3-carbonitrile—isomer 2

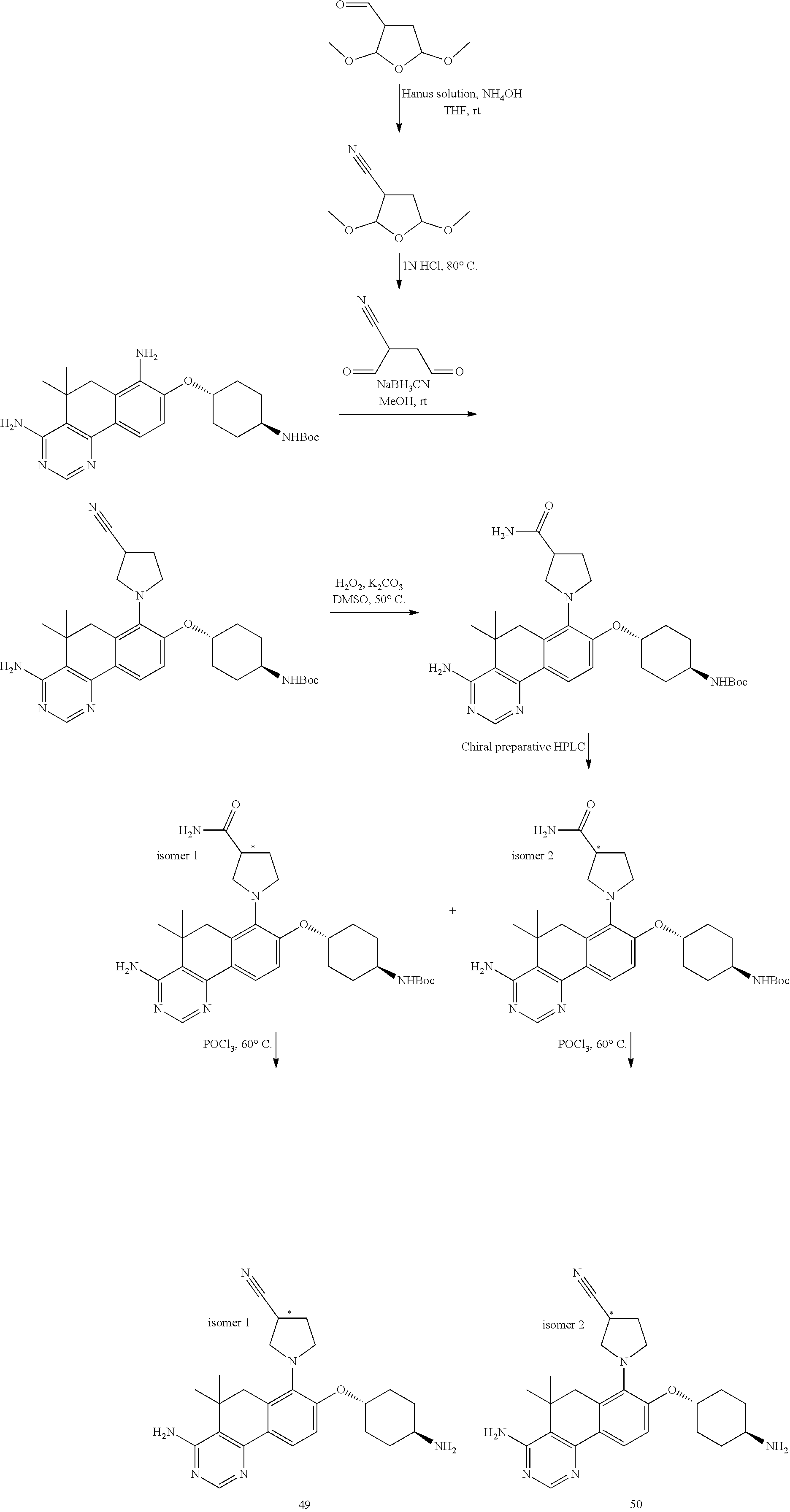

### Step 1: Preparation of 2,5-dimethoxytetrahydrofuran-3-carbonitrile

NH₄OH (866 μL, 2.25 mmol) was added to a stirred solution of 2,5-dimethoxytetrahydrofuran-3-carbaldehyde (50 mg, 0.28 mmol) in THF (0.4 mL), followed by Hanus solution (109 μL, 0.42 mmol). After 16 h stirring, the reaction mixture was quenched with a saturated Na₂SO₃ aqueous solution and then extracted with EA and H₂O. The combined organic layers were dried over Na₂SO₄, filtered, concentrated and purified by column chromatography (silica gel; PE:EA; 5:1; v:v) to afford 2,5-dimethoxytetrahydrofuran-3-carbonitrile as a yellow oil (44 mg, 36% yield).

¹H NMR (400 MHz, CDCl₃) δ ppm: 5.27 (d, J=3.2 Hz, 1H), 5.21 (dd, J=5.2 Hz, 2.4 Hz, 1H), 3.46 (s, 3H), 3.41 (s, 3H), 3.24 (m, 1H), 2.39 (m, 2H).

### Step 2: Preparation of 2-formyl-4-oxo-butanenitrile

A solution of 2,5-dimethoxytetrahydrofuran-3-carbonitrile (500 mg, 2.54 mmol) in 1N HCl aqueous solution (3 mL) was stirred for 1 h at 80° C. before being directly used in the next step.

### Step 3: Preparation of tert-butyl N-[trans-4-[[4-amino-7-(3-cyanopyrrolidin-1-yl)-5,5-dimethyl-6H-benzo[h]quinazolin-8-yl]oxy]cyclohexyl]carbamate

The title compound was prepared as a yellow solid (575 mg, 51% yield) following Scheme 1 and in analogy to Example 7 (step 1) using tert-butyl N-[trans-4-[(4,7-diamino-5,5-dimethyl-6H-benzo[h]quinazolin-8-yl)oxy]cyclohexyl]carbamate (150 mg, 1.08 mmol) as starting material.

¹H NMR (400 MHz, DMSO-d₆) δ ppm: 8.23 (s, 1H), 7.87 (d, J=8.8 Hz, 1H), 7.00 (d, J=8.8 Hz, 1H), 6.83 (d, J=7.6 Hz, 1H), 6.34 (s, 2H), 4.36 (m, 1H), 3.42 (m, 2H), 3.19 (m, 4H), 2.85 (m, 2H), 2.32 (m, 1H), 2.12 (m, 3H), 1.80 (m, 2H), 1.43 (m, 13H), 1.27 (s, 3H), 1.23 (s, 3H).

MS m/z (+ESI): 533.4 [M+H]⁺.

### Step 4: Preparation of tert-butyl N-[trans-4-[[4-amino-7-(3-carbamoylpyrrolidin-1-yl)-5,5-dimethyl-6H-benzo[h]quinazolin-8-yl]oxy]cyclohexyl]carbamate

H₂O₂(132 μL, 1.67 mmol) was added to a stirred solution of tert-butyl N-[trans-4-[[4-amino-7-(3-cyanopyrrolidin-1-yl)-5,5-dimethyl-6H-benzo[h]quinazolin-8-yl]oxy]cyclohexyl]carbamate (100 mg, 0.17 mmol) in DMSO, followed by K₂CO₃ (119 mg, 0.84 mmol). After 16 h stirring at 50° C., the reaction mixture was concentrated and purified by combiflash to afford tert-butyl N-[trans-4-[[4-amino-7-(3-carbamoylpyrrolidin-1-yl)-5,5-dimethyl-6H-benzo[h]quinazolin-8-yl]oxy]cyclohexyl]carbamate as a white solid (74 mg, 72% yield).

¹H NMR (400 MHz, DMSO-d₆) δ ppm: 8.22 (s, 1H), 7.84 (d, J=8.8 Hz, 1H), 7.33 (s, 1H), 6.97 (d, J=8.8 Hz, 1H), 6.82 (m, 2H), 6.32 (s, 2H), 4.34 (m, 1H), 3.21 (m, 2H), 3.13 (m, 2H), 3.01 (m, 2H), 2.83 (m, 2H), 2.08 (m, 4H), 1.83 (m, 2H), 1.38 (m, 13H), 1.26 (s, 3H), 1.23 (s, 3H).

MS m/z (+ESI): 551.3 [M+H]⁺.

### Step 5: Preparation of tert-butyl N-[4-[[4-amino-7-(3-carbamoylpyrrolidin-1-yl)-5,5-dimethyl-6H-benzo[h]quinazolin-8-yl]oxy]cyclohexyl]carbamate—isomer 1 and tert-butyl N-[4-[[4-amino-7-(3-carbamoylpyrrolidin-1-yl)-5,5-dimethyl-6H-benzo[h]quinazolin-8-yl]oxy]cyclohexyl]carbamate—isomer 2

tert-butyl N-[4-[[4-amino-7-(3-carbamoylpyrrolidin-1-yl)-5,5-dimethyl-6H-benzo[h]quinazolin-8-yl]oxy]cyclohexyl]carbamate—isomer 1 and tert-butyl N-[4-[[4-amino-7-(3-carbamoylpyrrolidin-1-yl)-5,5-dimethyl-6H-benzo[h]quinazolin-8-yl]oxy]cyclohexyl]carbamate—isomer 2 were prepared as yellow solids (137 mg, 43% yield each) following Scheme 1 and in analogy to Example 15 (step 2).

MS m/z (+ESI): 551.3 [M+H]⁺.

### Step 6: Preparation of 1-[4-amino-8-(trans-4-aminocyclohexoxy)-5,5-dimethyl-6H-benzo[h]quinazolin-7-yl]pyrrolidine-3-carbonitrile—isomer 1

A suspension of tert-butyl N-[4-[[4-amino-7-(3-carbamoylpyrrolidin-1-yl)-5,5-dimethyl-6H-benzo[h]quinazolin-8-yl]oxy]cyclohexyl]carbamate—isomer 1 (135 mg, 0.22 mmol) in POCl₃ (8 mL) was stirred at 60° C. for 2 h before concentration. The residue was purified by preparative HPLC to afford 1-[4-amino-8-(trans-4-aminocyclohexoxy)-5,5-dimethyl-6H-benzo[h]quinazolin-7-yl]pyrrolidine-3-carbonitrile—isomer 1 as a white solid (49 mg, 51% yield).

¹H NMR (400 MHz, DMSO-d₆+D₂O) δ ppm: 8.27 (s, 1H), 7.86 (d, J=8.8 Hz, 1H), 7.07 (d, J=8.8 Hz, 1H), 4.41 (m, 1H), 3.41 (overlapped with water peak, 2H), 3.21 (m, 2H), 3.11 (m, 2H), 2.86 (m, 2H), 2.32 (m, 1H), 2.15 (m, 3H), 1.98 (m, 2H), 1.51 (m, 4H), 1.26 (s, 3H), 1.23 (s, 3H).

MS m/z (+ESI): 433.2 [M+H]⁺.

[α]_{D}²⁵=−20.0° (c=0.07, MeOH).

### Step 7: Preparation of 1-[4-amino-8-(trans-4-aminocyclohexoxy)-5,5-dimethyl-6H-benzo[h]quinazolin-7-yl]pyrrolidine-3-carbonitrile—isomer 2

The title compound was prepared as an off-white solid (46 mg, 47% yield) following Scheme 1 and in analogy to Example 49 (step 6) using tert-butyl N-[4-[[4-amino-7-(3-carbamoylpyrrolidin-1-yl)-5,5-dimethyl-6H-benzo[h]quinazolin-8-yl]oxy]cyclohexyl]carbamate—isomer 2 (135 mg, 0.22 mmol) as starting material.

¹H NMR (400 MHz, DMSO-d₆+D₂O) δ ppm: 8.53 (s, 1H), 7.77 (d, J=8.8 Hz, 1H), 7.23 (d, J=8.8 Hz, 1H), 4.48 (m, 1H), 3.41 (overlapped with water peak, 2H), 3.20 (m, 2H), 3.11 (m, 2H), 2.94 (m, 2H), 2.33 (m, 1H), 2.16 (m, 3H), 1.99 (m, 2H), 1.53 (m, 4H), 1.28 (s, 3H), 1.25 (s, 3H).

MS m/z (+ESI): 433.2 [M+H]⁺.

[α]_{D}2⁵¹=22.8⁰ (c=0.07, MeOH).

### Preparation of Example 51: 3-[[4-amino-8-(trans-4-aminocyclohexoxy)-5,5-dimethyl-6H-benzo[h]quinazolin-7-yl]-cyclopropyl-amino]propanenitrile

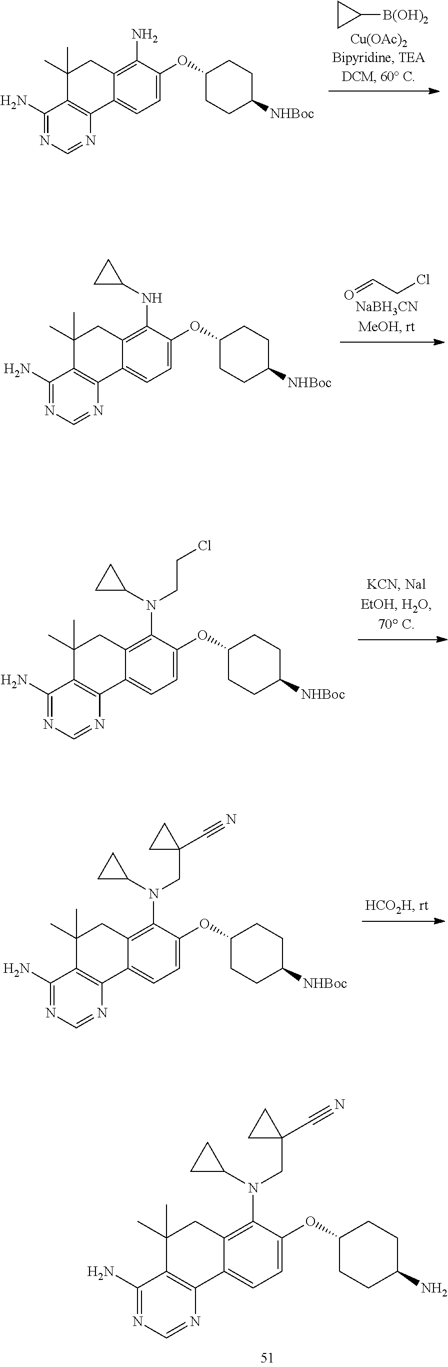

### Step 1: Preparation of tert-butyl N-[trans-4-[[4-amino-7-(cyclopropylamino)-5,5-dimethyl-6H-benzo[h]quinazolin-8-yl]oxy]cyclohexyl]carbamate

Cyclopropylboronic acid (444 mg, 4.96 mmol) was added to a stirred solution of tert-butyl N-[trans-4-[(4,7-diamino-5,5-dimethyl-6H-benzo[h]quinazolin-8-yl)oxy]cyclohexyl]carbamate (500 mg, 0.99 mmol) in DCM (10 mL), followed by Cu(OAc)₂ (421 mg, 1.98 mmol), 2,2′-bipyridine (316 mg, 1.98 mmol) and TEA (419 μL, 2.98 mmol). After 3 h stirring at 60° C., the reaction mixture was filtered. The filtrate was concentrated and purified by combiflash to afford tert-butyl N-[trans-4-[[4-amino-7-(cyclopropylamino)-5,5-dimethyl-6H-benzo[h]quinazolin-8-yl]oxy]cyclohexyl]carbamate as a light yellow solid (230 mg, 42% yield).

¹H NMR (400 MHz, DMSO-d₆) δ ppm: 8.23 (s, 1H), 7.62 (d, J=8.8 Hz, 1H), 6.90 (d, J=8.8 Hz, 1H), 6.81 (d, J=7.2 Hz, 1H), 6.33 (br, 2H), 4.26 (m, 1H), 4.07 (m, 1H), 3.29 (m, 1H), 2.85 (s, 2H), 2.60 (m, 1H), 2.05 (m, 2H), 1.81 (m, 2H), 1.41 (m, 2H), 1.38 (s, 9H), 1.32 (m, 2H), 1.27 (s, 6H), 0.54 (m, 2H), 0.40 (m, 2H).

MS m/z (+ESI): 494.4 [M+H]⁺.

### Step 2: Preparation of tert-butyl N-[trans-4-[[4-amino-7-[2-chloroethyl(cyclopropyl)amino]-5,5-dimethyl-6H-benzo[h]quinazolin-8-yl]oxy]cyclohexyl]carbamate

The title compound was prepared as a white solid (182 mg, 91% yield) following Scheme 1 and in analogy to Example 8 (step 1) using tert-butyl N-[trans-4-[[4-amino-7-(cyclopropylamino)-5,5-dimethyl-6H-benzo[h]quinazolin-8-yl]oxy]cyclohexyl]carbamate (180 mg, 0.33 mmol) as starting material.

MS m/z (+ESI): 556.3, 558.3 [M+H]⁺.

### Step 3: Preparation of tert-butyl N-[trans-4-[[4-amino-7-[2-cyanoethyl(cyclopropyl)amino]-5,5-dimethyl-6H-benzo[h]quinazolin-8-yl]oxy]cyclohexyl]carbamate

KCN (56 mg, 0.85 mmol) was added to a stirred solution of tert-butyl N-[trans-4-[[4-amino-7-[2-chloroethyl(cyclopropyl)amino]-5,5-dimethyl-6H-benzo[h]quinazolin-8-yl]oxy]cyclohexyl]carbamate (175 mg, 0.28 mmol) in EtOH (1.5 mL) and H₂O (0.3 mL), followed by NaI (43 mg, 0.28 mmol). After 8 h stirring at 70° C., the reaction mixture was extracted with DCM and H₂O. The combined organic layers were dried over Na₂SO₄, filtered, concentrated and purified by combiflash to afford tert-butyl N-[trans-4-[[4-amino-7-[2-cyanoethyl(cyclopropyl)amino]-5,5-dimethyl-6H-benzo[h]quinazolin-8-yl]oxy]cyclohexyl]carbamate as a white solid (120 mg, 70% yield).

MS m/z (+ESI): 547.4 [M+H]⁺.

### Step 4: Preparation of 3-[[4-amino-8-(trans-4-aminocyclohexoxy)-5,5-dimethyl-6H-benzo[h]quinazolin-7-yl]-cyclopropyl-amino]propanenitrile

The title compound was prepared as a white solid (39 mg, 48% yield) following Scheme 1 and in analogy to Example 9 (step 2) using tert-butyl N-[trans-4-[[4-amino-7-[2-cyanoethyl(cyclopropyl)amino]-5,5-dimethyl-6H-benzo[h]quinazolin-8-yl]oxy]cyclohexyl]carbamate (110 mg, 0.18 mmol) as starting material.

¹H NMR (400 MHz, DMSO-d₆+D₂O) δ ppm: 8.22 (s, 1H), 7.91 (d, J=8.8 Hz, 1H), 7.04 (d, J=8.8 Hz, 1H), 4.42 (m, 1H), 3.29 (t, J=6.0 Hz, 2H), 3.01 (m, 1H), 2.88 (m, 2H), 2.66 (m, 2H), 2.40 (m, 1H), 2.16 (m, 2H), 1.95 (m, 2H), 1.48 (m, 4H), 1.23 (s, 3H), 1.21 (s, 3H), 0.56 (m, 2H), 0.26 (m, 1H), 0.14 (m, 1H).

MS m/z (+ESI): 447.3 [M+H]⁺.

### Preparation of Example 52: 3-[[4-amino-5,5-dimethyl-8-(trans-4-morpholinocyclohexoxy)-6H-benzo[h]quinazolin-7-yl]-methyl-amino]propanenitrile

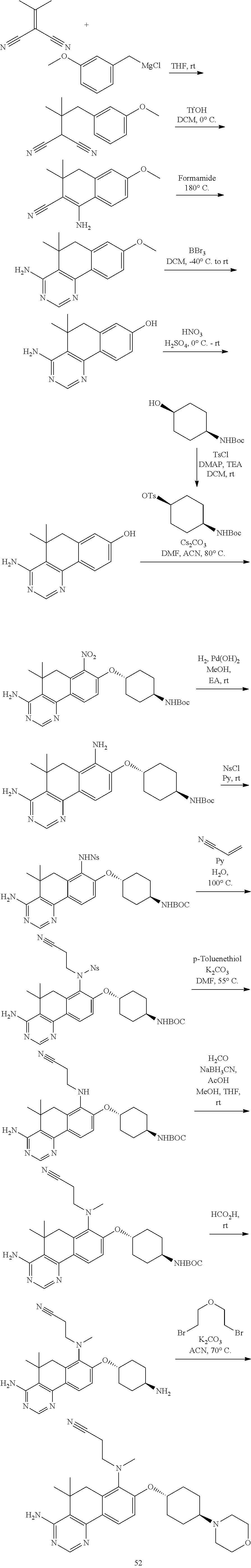

### Step 1: Preparation of 2-[2-(3-methoxyphenyl)-1,1-dimethyl-ethyl]propanedinitrile

3-Methoxybenzylmagnesium chloride (96 mL, 24 mmol, 0.25 M in THF) was added dropwise at 0° C. to a stirred solution of isopropylidenemalononitrile (2 g, 18.5 mmol) in THF (50 mL). The resulting solution was stirred at rt for 3 h. iN HCl aqueous solution was then added at 0° C. and the resulting mixture was concentrated. The residue was extracted with EA and H₂O. The combined organic layers were dried over Na₂SO₄, filtered, concentrated and purified by column chromatography (silica gel; PE:EA; 5:1; v:v) to afford 2-[2-(3-methoxyphenyl)-1,1-dimethyl-ethyl]propanedinitrile as a yellow oil (1.69 g, 38% yield).

¹H NMR (400 MHz, CDCl₃) δ ppm: 7.27 (m, 1H), 6.86 (m, 1H), 6.79 (d, J=7.6 Hz, 1H), 6.74 (m, 1H), 3.82 (s, 3H), 3.44 (s, 1H), 2.81 (s, 2H), 1.29 (s, 6H).

MS m/z (+ESI): 229.1 [M+H]⁺.

### Step 2: Preparation of 1-amino-6-methoxy-3,3-dimethyl-4H-naphthalene-2-carbonitrile

TfOH (1.04 g, 6.24 mmol) was added at 0° C. to a stirred solution of 2-[2-(3-methoxyphenyl)-1,1-dimethyl-ethyl]propanedinitrile (300 mg, 1.25 mmol) in DCM (6 mL) and the resulting mixture was stirred at 0° C. for 2 h. Saturated NaHCO₃ aqueous solution was added, and the mixture was extracted with DCM. The combined organic layers were dried over Na₂SO₄, filtered and concentrated to afford 1-amino-6-methoxy-3,3-dimethyl-4H-naphthalene-2-carbonitrile as a yellow solid (300 mg, 95% yield) which was used in the next step without further purification.

¹H NMR (400 MHz, CDCl₃) δ ppm: 7.31 (d, J=8.8 Hz, 1H), 6.81 (m, 1H), 6.74 (d, J=2.4 Hz, 1H), 4.51 (br, 2H), 3.85 (s, 3H), 2.69 (s, 2H), 1.17 (s, 6H).

MS m/z (+ESI): 229.1 [M+H]⁺.

### Step 3: Preparation of 8-methoxy-5,5-dimethyl-6H-benzo[h]quinazolin-4-amine

A suspension of 1-amino-6-methoxy-3,3-dimethyl-4H-naphthalene-2-carbonitrile (1 g, 3.94 mmol) and formamide (19 mL, 473 mmol) was stirred at 180° C. for 8 h. After being cooled to rt, the reaction mixture was diluted with H₂O and extracted with EA. The combined organic layers were dried over Na₂SO₄, filtered, concentrated and purified by column chromatography (silica gel; PE:EA; 1:1; v:v) to afford 8-methoxy-5,5-dimethyl-6H-benzo[h]quinazolin-4-amine as a light yellow solid (700 mg, 63% yield).

¹H NMR (400 MHz, DMSO-d₆) δ ppm: 8.24 (s, 1H), 7.99 (d, J=8.8 Hz, 1H), 6.86 (m, 1H), 6.79 (d, J=2.8 Hz, 1H), 6.37 (br, 2H), 3.79 (s, 3H), 2.75 (s, 2H), 1.28 (s, 6H).

MS m/z (+ESI): 256.1 [M+H]⁺.

### Step 4: Preparation of 4-amino-5,5-dimethyl-6H-benzo[h]quinazolin-8-ol

BBr₃ (1M in DCM, 4.8 mL, 4.80 mmol) was added dropwise at −40° C. to a stirred solution of 8-methoxy-5,5-dimethyl-6H-benzo[h]quinazolin-4-amine (450 mg, 1.58 mmol) in DCM (30 mL). The resulting solution was stirred at rt for 18 h. Saturated NaHCO₃ aqueous solution was added and the resulting green solid was collected by filtration, washed with H₂O and dried under reduced pressure to afford 4-amino-5,5-dimethyl-6H-benzo[h]quinazolin-8-ol (350 mg, 82% yield).

¹H NMR (400 MHz, DMSO-d₆) δ ppm: 8.21 (s, 1H), 7.88 (d, J=8.4 Hz, 1H), 6.67 (m, 1H), 6.59 (s, 1H), 6.29 (br, 2H), 2.67 (s, 2H), 1.26 (s, 6H).

MS m/z (+ESI): 242.2 [M+H]⁺.

### Step 5: Preparation of 4-amino-5,5-dimethyl-7-nitro-6H-benzo[h]quinazolin-8-ol

HNO₃ (7.5 mL, 117.2 mmol) was added dropwise at 0° C. to a stirred solution of 4-amino-5,5-dimethyl-6H-benzo[h]quinazolin-8-ol (700 mg, 2.32 mmol) in H₂SO₄ (7 mL). After 48 h stirring the reaction mixture was poured into 200 mL ice water and neutralized with NaHCO₃. The resulting mixture was extracted with EA. The combined organic layers were washed with brine, dried over Na₂SO₄, filtered and concentrated. The residue was purified by combiflash to afford 4-amino-5,5-dimethyl-7-nitro-6H-benzo[h]quinazolin-8-ol as a yellow solid (260 mg, 35% yield).

¹H NMR (400 MHz, DMSO-d₆) δ ppm: 12.76 (br, 1H), 8.27 (s, 1H), 8.09 (d, J=8.4 Hz, 1H), 7.01 (d, J=8.4 Hz, 1H), 6.54 (br, 2H), 2.62 (s, 2H), 1.27 (s, 6H).

MS m/z (+ESI): 287.2 [M+H]⁺.

### Step 6: Preparation of [cis-4-(tert-butoxycarbonylamino)cyclohexyl]4-methylbenzenesulfonate

TsCl (139 g, 721 mmol) was added portionwise to a stirred solution of tert-butyl N-(cis-4-hydroxycyclohexyl)carbamate (80 g, 360 mmol) in DCM (300 mL), followed by TEA (152 mL, 1081 mmol) and DMAP (4.5 g, 36 mmol). After 24 h stirring, the reaction mixture was filtered. The filtrate was concentrated and the residue was purified by column chromatography (silica gel; PE:EA; 10:1; v:v) to afford [cis-4-(tert-butoxycarbonylamino)cyclohexyl]4-methylbenzenesulfonate as a white solid (105 g, 71% yield).

¹H NMR (400 MHz, DMSO-d₆) δ ppm: 7.78 (d, J=8.0 Hz, 2H), 7.47 (d, J=8.0 Hz, 2H), 6.81 (d, J=7.2 Hz, 1H), 4.58 (m, 1H), 3.24 (m, 1H), 2.41 (s, 3H), 1.66 (m, 2H), 1.51 (m, 4H), 1.41 (m, 2H), 1.36 (s, 9H).

MS m/z (+ESI): 370.1 [M+H]⁺.

### Step 7: Preparation of tert-butyl N-[trans-4-[(4-amino-5,5-dimethyl-7-nitro-6H-benzo[h]quinazolin-8-yl)oxy]cyclohexyl]carbamate

Cs₂CO₃ (820 mg, 2.45 mmol) was added to a stirred solution of 4-amino-5,5-dimethyl-7-nitro-6H-benzo[h]quinazolin-8-ol (260 mg, 0.82 mmol) and [cis-4-(tert-butoxycarbonylamino)cyclohexyl]4-methylbenzenesulfonate (2.0 g, 4.90 mmol) in DMF (8 mL) and ACN (8 mL). After 40 h stirring at 80° C., the reaction mixture was filtered. The filtrate was concentrated and the residue was purified by combiflash to afford tert-butyl N-[trans-4-[(4-amino-5,5-dimethyl-7-nitro-6H-benzo[h]quinazolin-8-yl)oxy]cyclohexyl]carbamate as a light yellow solid (400 mg, 91% yield).

¹H NMR (400 MHz, DMSO-d₆) δ ppm: 8.29 (s, 1H), 8.19 (d, J=8.8 Hz, 1H), 7.38 (d, J=8.8 Hz, 1H), 6.84 (d, J=7.2 Hz, 1H), 6.57 (s, 2H), 4.52 (m, 1H), 3.28 (m, 1H), 2.62 (s, 2H), 2.03 (m, 2H), 1.79 (m, 2H), 1.38 (m, 13H), 1.27 (s, 6H).

MS m/z (+ESI): 484.4 [M+H]⁺.

### Step 8: Preparation of tert-butyl N-[trans-4-[(4,7-diamino-5,5-dimethyl-6H-benzo[h]quinazolin-8-yl)oxy]cyclohexyl]carbamate

Pd(OH)₂ (20% on carbon, 52 mg, 0.07 mmol) was added to a stirred solution of tert-butyl N-[trans-4-[(4-amino-5,5-dimethyl-7-nitro-6H-benzo[h]quinazolin-8-yl)oxy]cyclohexyl]carbamate (400 mg, 0.74 mmol) in MeOH (10 mL) and EA (10 mL). After 20 h stirring under hydrogen flow, the catalyst was removed by filtration and the solution was concentrated to afford tert-butyl N-[trans-4-[(4,7-diamino-5,5-dimethyl-6H-benzo[h]quinazolin-8-yl)oxy]cyclohexyl]carbamate as a light yellow solid (340 mg, 96% yield) which was used in the next step without further purification.

¹H NMR (400 MHz, DMSO-d₆) δ ppm: 8.21 (s, 1H), 7.41 (d, J=8.4 Hz, 1H), 6.82 (d, J=8.4 Hz, 1H), 6.79 (d, J=7.2 Hz, 1H), 6.28 (br, 2H), 4.51 (s, 2H), 4.22 (m, 1H), 3.29 (m, 1H), 2.61 (s, 2H), 2.06 (m, 2H), 1.83 (m, 2H), 1.46 (m, 2H), 1.38 (s, 9H), 1.36 (m, 2H), 1.28 (s, 6H).

MS m/z (+ESI): 454.3 [M+H]⁺.

### Step 9: Preparation of tert-butyl N-[trans-4-[[4-amino-5,5-dimethyl-7-[(4-nitrophenyl)sulfonylamino]-6H-benzo[h]quinazolin-8-yl]oxy]cyclohexyl]carbamate

NsCl (7.8 g, 34.13 mmol) was added to a stirred solution of tert-butyl N-[trans-4-[(4,7-diamino-5,5-dimethyl-6H-benzo[h]quinazolin-8-yl)oxy]cyclohexyl]carbamate (4.3 g, 8.53 mmol) in Py (40 mL). After 30 min stirring, the reaction mixture was poured into H₂O. The resulting suspension was filtered and the filtered cake was washed with water and dried under high vacuum to afford tert-butyl N-[trans-4-[[4-amino-5,5-dimethyl-7-[(4-nitrophenyl)sulfonylamino]-6H-benzo[h]quinazolin-8-yl]oxy]cyclohexyl]carbamate as a light yellow solid (6 g, 94% yield).

¹H NMR (400 MHz, DMSO-d₆) δ ppm: 9.62 (s, 1H), 8.39 (d, J=8.8 Hz, 2H), 8.25 (s, 1H), 7.94 (m, 3H), 6.91 (d, J=8.8 Hz, 1H), 6.72 (d, J=7.2 Hz, 1H), 6.41 (s, 2H), 4.00 (m, 1H), 3.03 (m, 1H), 2.87 (s, 2H), 1.63 (m, 4H), 1.37 (s, 9H), 1.27 (s, 6H), 1.13 (m, 4H).

MS m/z (+ESI): 639.0 [M+H]⁺.

### Step 10: Preparation of tert-butyl N-[trans-4-[[4-amino-7-[2-cyanoethyl-(4-nitrophenyl)sulfonyl-amino]-5,5-dimethyl-6H-benzo[h]quinazolin-8-yl]oxy]cyclohexyl]carbamate

Acrylonitrile (18.97 mL, 285.2 mmol) was added to a stirred solution of tert-butyl N-[trans-4-[[4-amino-5,5-dimethyl-7-[(4-nitrophenyl)sulfonylamino]-6H-benzo[h]quinazolin-8-yl]oxy]cyclohexyl]carbamate (5.5 g, 7.32 mmol) in Py (33 mL) and H₂O (6.6 mL), followed by DMAP (452 mg, 3.66 mmol). After 3 h stirring at 100° C., the reaction mixture was cooled down to rt before the addition of H₂O (500 mL). The resulting suspension was filtered and the filtered cake was washed with water and dried under high vacuum to afford tert-butyl N-[trans-4-[[4-amino-7-[2-cyanoethyl-(4-nitrophenyl)sulfonyl-amino]-5,5-dimethyl-6H-benzo[h]quinazolin-8-yl]oxy]cyclohexyl]carbamate as a light yellow solid (5.5 g, 92% yield).

¹H NMR (400 MHz, DMSO-d₆) δ ppm: 8.41 (d, J=8.8 Hz, 2H), 8.27 (s, 1H), 8.10 (d, J=8.8 Hz, 1H), 8.02 (d, J=8.8 Hz, 2H), 7.03 (d, J=8.8 Hz, 1H), 6.73 (d, J=7.2 Hz, 1H), 6.47 (s, 2H), 4.15 (m, 1H), 4.05 (m, 1H), 3.65 (m, 1H), 2.95 (m, 3H), 2.72 (m, 1H), 2.62 (m, 1H), 1.80 (m, 1H), 1.63 (m, 2H), 1.47 (m, 1H), 1.41 (s, 3H), 1.36 (s, 9H), 1.25 (s, 3H), 1.15 (m, 2H), 0.86 (m, 1H), 0.58 (m, 1H).

MS m/z (+ESI): 692.0 [M+H]⁺.

### Step 11: Preparation of tert-butyl N-[trans-4-[[4-amino-7-(2-cyanoethylamino)-5,5-dimethyl-6H-benzo[h]quinazolin-8-yl]oxy]cyclohexyl]carbamate

p-Toluenethiol (1.39 g, 11.06 mmol) was added to a stirred solution of tert-butyl N-[trans-4-[[4-amino-7-[2-cyanoethyl-(4-nitrophenyl)sulfonyl-amino]-5,5-dimethyl-6H-benzo[h]quinazolin-8-yl]oxy]cyclohexyl]carbamate (5.5 g, 6.76 mmol) in DMF (40 mL), followed by K₂CO₃ (2.57 g, 18.43 mmol). After 1.5 h stirring at 55° C., solvent was removed and the crude was extracted with EA and H₂O.

The combined organic layers were dried over Na₂SO₄, filtered, concentrated and the residue was purified by combiflash to afford tert-butyl N-[trans-4-[[4-amino-7-(2-cyanoethylamino)-5,5-dimethyl-6H-benzo[h]quinazolin-8-yl]oxy]cyclohexyl]carbamate as a light yellow solid (2.95 g, 71% yield).

¹H NMR (400 MHz, DMSO-d₆) δ ppm: 8.23 (s, 1H), 7.67 (d, J=8.8 Hz, 1H), 6.94 (d, J=8.8 Hz, 1H), 6.82 (d, J=7.6 Hz, 1H), 6.33 (s, 2H), 4.27 (m, 2H), 3.28 (m, 1H), 3.20 (m, 2H), 2.75 (s, 2H), 2.60 (t, J=6.4 Hz, 2H), 2.07 (m, 2H), 1.83 (m, 2H), 1.48 (m, 2H), 1.38 (s, 9H), 1.33 (m, 2H), 1.27 (s, 6H).

MS m/z (+ESI): 507.4 [M+H]⁺.

### Step 12: Preparation of tert-butyl N-[trans-4-[[4-amino-7-[2-cyanoethyl(methyl)amino]-5,5-dimethyl-6H-benzo[h]quinazolin-8-yl]oxy]cyclohexyl]carbamate

H₂CO (2.13 g, 26.20 mmol) was added to a stirred solution of tert-butyl N-[trans-4-[[4-amino-7-(2-cyanoethylamino)-5,5-dimethyl-6H-benzo[h]quinazolin-8-yl]oxy]cyclohexyl]carbamate (2.95 g, 5.24 mmol) in MeOH (25 mL) and THF (5 mL), followed by NaBH₃CN (693 mg, 10.48 mmol) and AcOH (304 μL, 5.24 mmol). After 2 h stirring, the reaction mixture was concentrated and the crude was extracted with DCM and H₂O. The combined organic layers were dried over Na₂SO₄, filtered, concentrated to afford tert-butyl N-[trans-4-[[4-amino-7-[2-cyanoethyl(methyl)amino]-5,5-dimethyl-6H-benzo[h]quinazolin-8-yl]oxy]cyclohexyl]carbamate as an off-white solid (2.98 g, 98% yield) which was used in the next step without further purification.

MS m/z (+ESI): 521.4 [M+H]⁺.

### Step 13: Preparation of 3-[[4-amino-8-(trans-4-aminocyclohexoxy)-5,5-dimethyl-6H-benzo[h]quinazolin-7-yl]-methyl-amino]propanenitrile

A solution of tert-butyl N-[trans-4-[[4-amino-7-[2-cyanoethyl(methyl)amino]-5,5-dimethyl-6H-benzo[h]quinazolin-8-yl]oxy]cyclohexyl]carbamate (2.98 g, 5.15 mmol) in HCO₂H (25 mL) was stirred for 2.5 h. Solvent was then removed and the residue was purified by combiflash to afford 3-[[4-amino-8-(trans-4-aminocyclohexoxy)-5,5-dimethyl-6H-benzo[h]quinazolin-7-yl]-methyl-amino]propanenitrile as a white solid (2.15 g, 97% yield).

¹H NMR (400 MHz, DMSO-d₆+D₂O) δ ppm: 8.21 (s, 1H), 7.87 (d, J=8.8 Hz, 1H), 7.02 (d, J=8.8 Hz, 1H), 4.38 (m, 1H), 3.21 (m, 2H), 3.09 (m, 3H), 2.75 (m, 1H), 2.66 (m, 1H), 2.64 (s, 3H), 2.15 (m, 2H), 1.97 (m, 2H), 1.49 (m, 4H), 1.30 (s, 3H), 1.21 (s, 3H)

MS m/z (+ESI): 421.3 [M+H]⁺.

### Step 14: Preparation of 3-[[4-amino-5,5-dimethyl-8-(trans-4-morpholinocyclohexoxy)-6H-benzo[h]quinazolin-7-yl]-methyl-amino]propanenitrile

2-Bromoethyl ether (288 mg, 1.22 mmol) was added to a stirred solution of 3-[[4-amino-8-(trans-4-aminocyclohexoxy)-5,5-dimethyl-6H-benzo[h]quinazolin-7-yl]-methyl-amino]propanenitrile (265 mg, 0.57 mmol) in ACN (5 mL), followed by K₂CO₃ (340 mg, 2.43 mmol). After 18 h stirring at 70° C., the suspension was extracted with DCM and H₂O. The combined organic layers were dried over Na₂SO₄, filtered, concentrated and purified by preparative HPLC to afford 3-[[4-amino-5,5-dimethyl-8-(trans-4-morpholinocyclohexoxy)-6H-benzo[h]quinazolin-7-yl]-methyl-amino]propanenitrile as a white solid (491 mg, 60% yield).

¹H NMR (400 MHz, DMSO-d₆+D₂O) δ ppm: 8.22 (s, 1H), 7.88 (d, J=8.8 Hz, 1H), 6.98 (d, J=8.8 Hz, 1H), 4.37 (m, 1H), 3.56 (m, 4H), 3.23 (t, J=6.4 Hz, 2H), 2.97 (m, 2H), 2.65 (s, 3H), 2.46 (m, 6H), 2.26 (m, 1H), 2.14 (m, 2H), 1.85 (m, 2H), 1.42 (m, 4H), 1.31 (s, 3H), 1.23 (s, 3H).

MS m/z (+ESI): 491.3 [M+H]⁺.

### Preparation of Example 54: 8-(trans-4-aminocyclohexoxy)-N7-but-3-ynyl-N7,5,5-trimethyl-6H-benzo[h]quinazoline-4,7-diamine

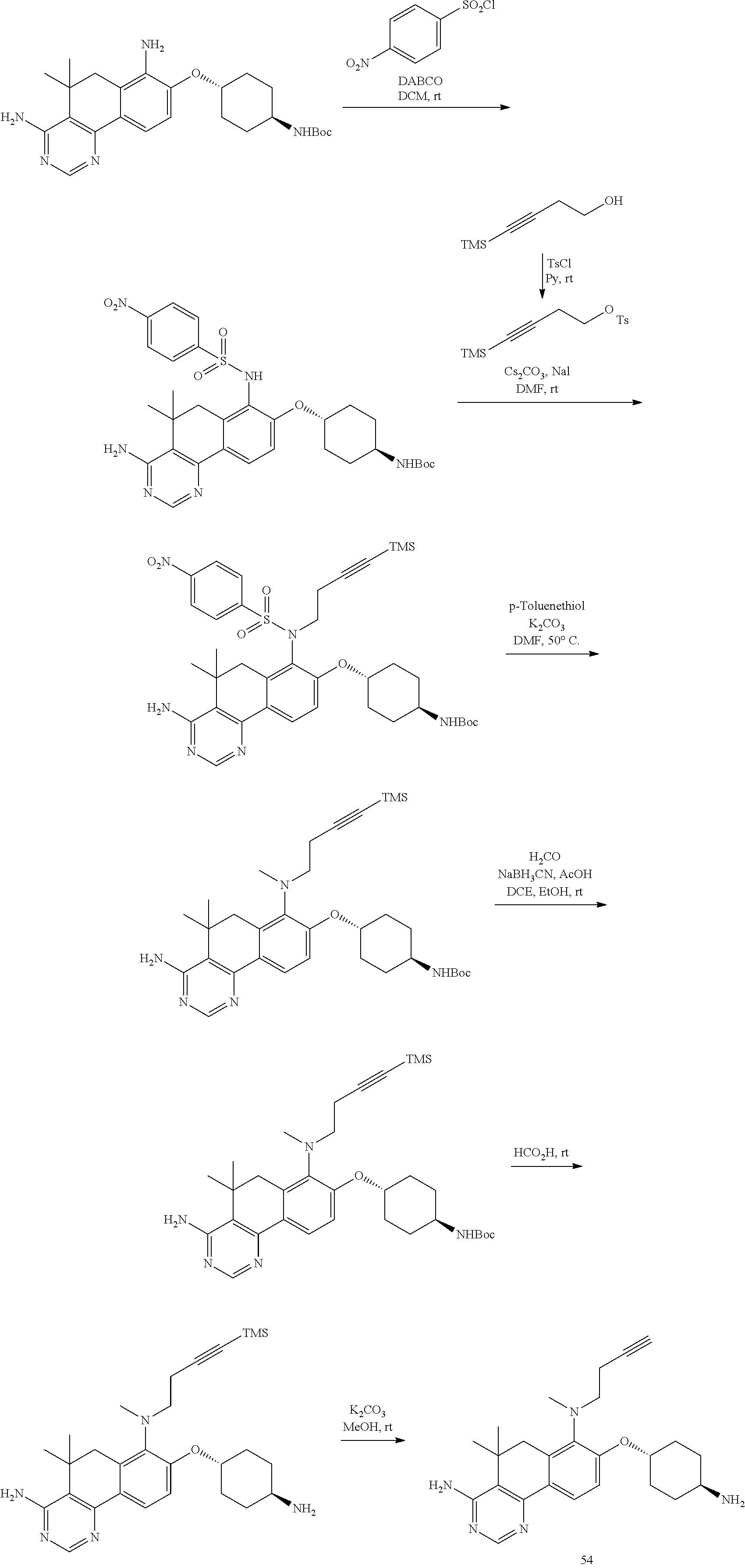

### Step 1: Preparation of 8-(trans-4-aminocyclohexoxy)-N7,5,5-trimethyl-N7-(4-trimethylsilylbut-3-ynyl)-6H-benzo[h]quinazoline-4,7-diamine

The title compound was prepared as a light yellow solid following Scheme 1 and in analogy to examples 5, 9, 21, 48 using tert-butyl N-[trans-4-[(4,7-diamino-5,5-dimethyl-6H-benzo[h]quinazolin-8-yl)oxy]cyclohexyl]carbamate, 4-trimethylsilylbut-3-yn-1-ol, 4-nitrobenzenesulfonyl chloride and formaldehyde as starting materials.

MS m/z (+ESI): 492.1 [M+H]⁺.

### Step 2: Preparation of 8-(trans-4-aminocyclohexoxy)-N7-but-3-ynyl-N7,5,5-trimethyl-6H-benzo[h]quinazoline-4,7-diamine

K₂CO₃ (108 mg, 0.78 mmol) was added to a stirred solution of 8-(trans-4-aminocyclohexoxy)-N7,5,5-trimethyl-N7-(4-trimethylsilylbut-3-ynyl)-6H-benzo[h]quinazoline-4,7-diamine (150 mg, 0.26 mmol) in MeOH (0.5 mL). After 2 h stirring, the suspension was filtered. The filtrate was concentrated and purified by preparative HPLC to afford 8-(trans-4-aminocyclohexoxy)-N7-but-3-ynyl-N7,5,5-trimethyl-6H-benzo[h]quinazoline-4,7-diamine as a white solid (90 mg, 80% yield).

¹H NMR (400 MHz, DMSO-d₆+D₂O) δ ppm: 8.21 (s, 1H), 7.85 (d, J=8.8 Hz, 1H), 6.99 (d, J=8.8 Hz, 1H), 4.37 (m, 1H), 3.15 (m, 1H), 3.06 (m, 3H), 2.78 (m, 1H), 2.71 (t, J=2.4 Hz, 1H), 2.64 (s, 3H), 2.17 (m, 4H), 1.97 (m, 2H), 1.48 (m, 4H), 1.27 (s, 3H), 1.21 (s, 3H).

MS m/z (+ESI): 420.2 [M+H]⁺.

### Preparation of Example 56: 1-[4-amino-8-(trans-4-aminocyclohexoxy)-5,5-dimethyl-6H-benzo[h]quinazolin-7-yl]-2H-pyrrol-5-one

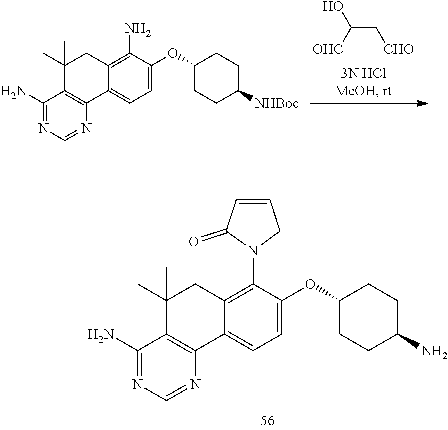 [SMILES: CC(C)(C)OC(=O)N[C@H]1CC[C@H](Oc2ccc3c(c2N)CC(C)(C)c2c(N)ncnc2-3)CC1 ; O=CCC(O)C=O ; CC1(C)Cc2c(ccc(O[C@H]3CC[C@H](N)CC3)c2N2CC=CC2=O)-c2ncnc(N)c21]

2-Hydroxybutanedial (126 mg, 1.11 mmol) was added to a stirred solution of tert-butyl N-[trans-4-[(4,7-diamino-5,5-dimethyl-6H-benzo[h]quinazolin-8-yl)oxy]cyclohexyl]carbamate (200 mg, 0.37 mmol) in MeOH (6 mL) and a 3N HCl aqueous solution (3 mL). After 4 h stirring, the reaction mixture was concentrated and the residue was purified by preparative HPLC to afford 1-[4-amino-8-(trans-4-aminocyclohexoxy)-5,5-dimethyl-6H-benzo[h]quinazolin-7-yl]-2H-pyrrol-5-one as a light yellow solid (10 mg, 6% yield).

¹H NMR (400 MHz, DMSO-d₆+D₂O) δ ppm: 8.50 (s, 1H), 7.92 (d, J=8.8 Hz, 1H), 7.52 (d, J=6.0 Hz, 1H), 7.29 (d, J=8.8 Hz, 1H), 6.27 (dt, J=6.0 Hz, 1.6 Hz, 1H), 4.39 (m, 2H), 4.13 (d, J=16.0 Hz, 1H), 3.07 (m, 1H), 2.57 (d, J=7.2 Hz, 2H), 2.02 (m, 4H), 1.44 (m, 4H), 1.31 (s, 3H), 1.18 (s, 3H).

MS m/z (+ESI): 420.4 [M+H]⁺.

### Preparation of Example 57: 8-(trans-4-aminocyclohexoxy)-7-(3-methoxypyrrol-1-yl)-5,5-dimethyl-6H-benzo[h]quinazolin-4-amine

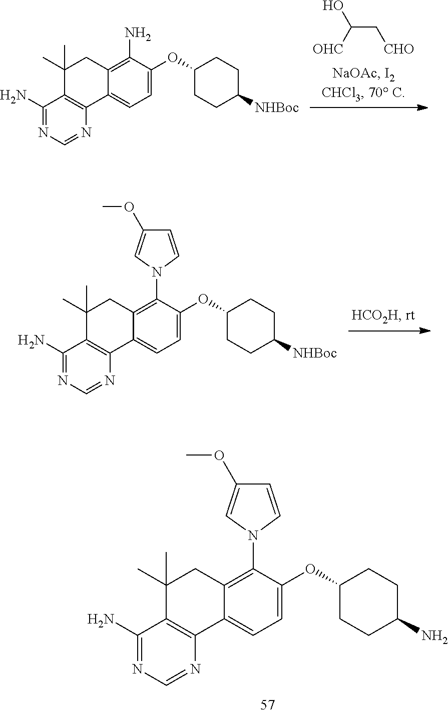 [SMILES: CC(C)(C)OC(=O)N[C@H]1CC[C@H](Oc2ccc3c(c2N)CC(C)(C)c2c(N)ncnc2-3)CC1 ; O=CCC(O)C=O ; COc1ccn(-c2c(O[C@H]3CC[C@H](NC(=O)OC(C)(C)C)CC3)ccc3c2CC(C)(C)c2c(N)ncnc2-3)c1 ; COc1ccn(-c2c(O[C@H]3CC[C@H](N)CC3)ccc3c2CC(C)(C)c2c(N)ncnc2-3)c1]

### Step 1: Preparation of tert-butyl N-[trans-4-[[4-amino-7-(3-methoxypyrrol-1-yl)-5,5-dimethyl-6H-benzo[h]quinazolin-8-yl]oxy]cyclohexyl]carbamate

NaOAc (87 mg, 1.03 mmol) was added to a stirred solution of tert-butyl N-[trans-4-[(4,7-diamino-5,5-dimethyl-6H-benzo[h]quinazolin-8-yl)oxy]cyclohexyl]carbamate (93 mg, 0.17 mmol) and 2-methoxybutanedial (25 mg, 0.17 mmol) in CHCl₃ (3 mL), followed by I₂ (0.4 mg, 0.02 mmol). After 8 h stirring at 70° C., the reaction mixture was concentrated and the residue was purified by column chromatography (silica gel; PE:EA; 1:1; v:v) to afford tert-butyl N-[trans-4-[[4-amino-7-(3-methoxypyrrol-1-yl)-5,5-dimethyl-6H-benzo[h]quinazolin-8-yl]oxy]cyclohexyl]carbamate as a yellow solid (50 mg, 49% yield).

¹H NMR (400 MHz, DMSO-d₆) δ ppm: 8.27 (s, 1H), 8.06 (d, J=8.8 Hz, 1H), 7.15 (d, J=8.8 Hz, 1H), 6.79 (d, J=8.0 Hz, 1H), 6.48 (t, J=2.4 Hz, 1H), 6.43 (s, 2H), 6.32 (t, J=2.0 Hz, 1H), 5.90 (dd, J=2.8 Hz, 2.0 Hz 1H), 4.22 (m, 1H), 3.63 (s, 3H), 3.22 (m, 1H), 2.42 (s, 2H), 1.94 (m, 2H), 1.73 (m, 2H), 1.38 (m, 4H), 1.23 (m, 15H).

MS m/z (+ESI): 534.3 [M+H]⁺.

### Step 2: Preparation of 8-(trans-4-aminocyclohexoxy)-7-(3-methoxypyrrol-1-yl)-5,5-dimethyl-6H-benzo[h]quinazolin-4-amine

The title compound was prepared as a black solid (22 mg, 22% yield) following Scheme 1 and in analogy to Example 9 (step 2) using tert-butyl N-[trans-4-[[4-amino-7-(3-methoxypyrrol-1-yl)-5,5-dimethyl-6H-benzo[h]quinazolin-8-yl]oxy]cyclohexyl]carbamate (130 mg, 0.22 mmol) as starting material.

¹H NMR (400 MHz, DMSO-d₆+D₂O) δ ppm: 8.26 (s, 1H), 8.06 (d, J=8.8 Hz, 1H), 7.18 (d, J=8.8 Hz, 1H), 6.48 (t, J=2.4 Hz, 1H), 6.31 (t, J=2.0 Hz, 1H), 5.90 (t, J=2.0 Hz, 1H), 4.23 (m, 1H), 3.62 (s, 3H), 2.96 (m, 1H), 2.41 (s, 2H), 1.99 (m, 2H), 1.87 (m, 2H), 1.38 (m, 4H), 1.18 (s, 6H).

MS m/z (+ESI): 434.3 [M+H]⁺.

### Preparation of Example 61: 8-methoxy-7-(3-methoxypropyl)-5,5-dimethyl-6H-benzo[h]quinazolin-4-amine

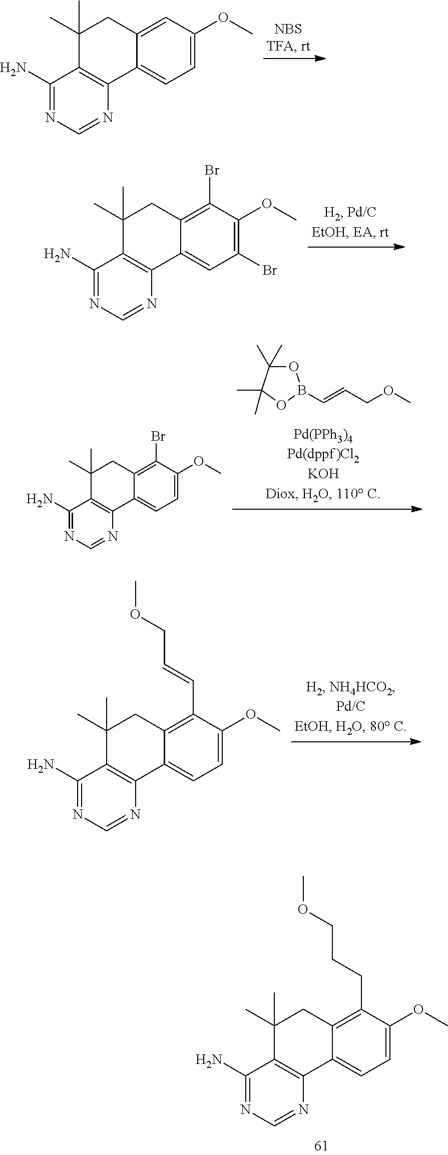

### Step 1: Preparation of 7,9-dibromo-8-methoxy-5,5-dimethyl-6H-benzo[h]quinazolin-4-amine

NBS (4.26 g, 23.48 mmol) was added to a stirred solution of 8-methoxy-5,5-dimethyl-6H-benzo[h]quinazolin-4-amine (3 g, 11.74 mmol) in TFA (30 mL). After 16 h stirring, the reaction mixture was concentrated. Saturated NaHCO₃ aqueous solution (200 mL) was added to the residue and the resulting suspension was filtered. The cake was washed with H₂O and dried under high vacuum to afford 7,9-dibromo-8-methoxy-5,5-dimethyl-6H-benzo[h]quinazolin-4-amine as a yellow solid (5.2 g, 96% yield).

¹H NMR (400 MHz, DMSO-d₆) δ ppm: 8.39 (s, 1H), 8.26 (s, 1H), 7.15 (br, 2H), 3.85 (s, 3H), 2.91 (s, 2H), 1.31 (s, 6H).

MS m/z (+ESI): 412.0, 414.0 [M+H]⁺.

### Step 2: Preparation of 7-bromo-8-methoxy-5,5-dimethyl-6H-benzo[h]quinazolin-4-amine

10% Pd/C (300 mg, 0.28 mmol) was added to a stirred solution of 7,9-dibromo-8-methoxy-5,5-dimethyl-6H-benzo[h]quinazolin-4-amine (1 g, 1.94 mmol) in EtOH (60 mL) and EA (20 mL). After 24 h stirring under hydrogen flow, the catalyst was removed by filtration and the solution was concentrated. The residue was purified by column chromatography (silica gel; DCM:MeOH; 8:1; v:v) to afford 7-bromo-8-methoxy-5,5-dimethyl-6H-benzo[h]quinazolin-4-amine as a white solid (400 mg, 52% yield).

MS m/z (+ESI): 334.1, 336.1 [M+H]⁺.

### Step 3: Preparation of 8-methoxy-7-[(E)-3-methoxyprop-1-enyl]-5,5-dimethyl-6H-benzo[h]quinazolin-4-amine

2-[(E)-3-methoxyprop-1-enyl]-4,4,5,5-tetramethyl-1,3,2-dioxaborolane (764 mg, 3.66 mmol) was added to a stirred solution of 7-bromo-8-methoxy-5,5-dimethyl-6H-benzo[h]quinazolin-4-amine (180 mg, 0.46 mmol) in Diox (10 mL) and H₂O (2 mL), followed by Pd(PPh₃)₄(54 mg, 0.04 mmol), Pd(dppf)Cl₂ (34 mg, 0.04 mmol) and KOH (131 mg, 2.29 mmol). After 2 h stirring at 110° C., the reaction mixture was filtered through decalite, concentrated and purified by combiflash to afford 8-methoxy-7-[(E)-3-methoxyprop-1-enyl]-5,5-dimethyl-6H-benzo[h]quinazolin-4-amine as a white solid (120 mg, 80% yield).

¹H NMR (400 MHz, DMSO-d₆+D₂O) δ ppm: 8.24 (s, 1H), 8.00 (d, J=8.8 Hz, 1H), 6.97 (d, J=8.8 Hz, 1H), 6.61 (d, J=16.4 Hz, 1H), 5.93 (dt, J=16.4 Hz, 5.6 Hz, 1H), 4.07 (d, J=5.6 Hz, 2H), 3.81 (s, 3H), 3.30 (s, 3H), 2.84 (s, 2H), 1.24 (s, 6H).

MS m/z (+ESI): 326.3 [M+H]⁺.

### Step 4: Preparation of 8-methoxy-7-(3-methoxypropyl)-5,5-dimethyl-6H-benzo[h]quinazolin-4-amine

10% Pd/C (130 mg, 0.13 mmol) was added to a stirred solution of 8-methoxy-7-[(E)-3-methoxyprop-1-enyl]-5,5-dimethyl-6H-benzo[h]quinazolin-4-amine (80 mg, 0.24 mmol) in EtOH (10 mL) and H₂O (0.5 mL), following NH₄HCO₂ (78 mg, 1.22 mmol). After 2 h stirring at 80° C. under hydrogen flow, the catalyst was removed by filtration and the solution was concentrated. The residue was purified by preparative HPLC to afford 8-methoxy-7-(3-methoxypropyl)-5,5-dimethyl-6H-benzo[h]quinazolin-4-amine as a white solid (65 mg, 81% yield).

¹H NMR (400 MHz, DMSO-d₆+D₂O) δ ppm: 8.23 (s, 1H), 7.95 (d, J=8.8 Hz, 1H), 6.92 (d, J=8.8 Hz, 1H), 3.81 (s, 3H), 3.35 (t, J=6.4 Hz, 2H), 3.24 (s, 3H), 2.74 (s, 2H), 2.67 (m, 2H), 1.63 (m, 2H), 1.27 (s, 6H).

MS m/z (+ESI): 328.4 [M+H]⁺.

### Preparation of Example 89: (5R)-5-[2-[(4-amino-8-methoxy-5,5-dimethyl-6H-benzo[h]quinazolin-7-yl)amino]ethyl]oxazolidin-2-one

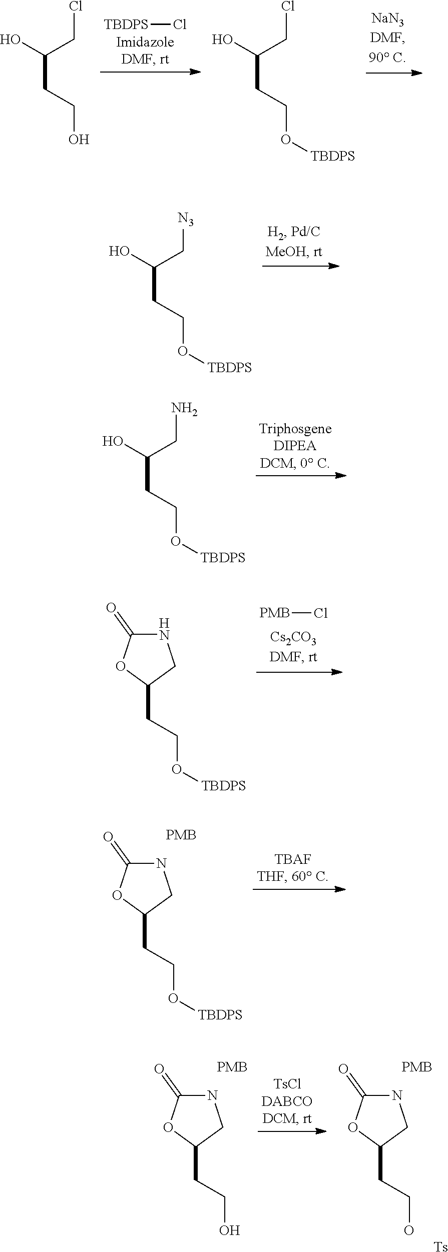

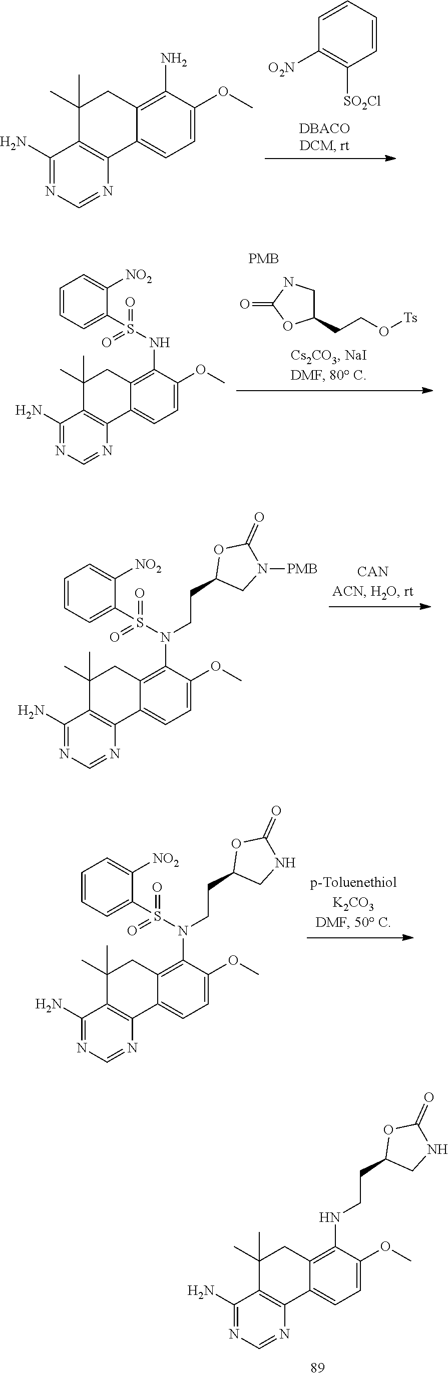

### Step 1: Preparation of(2R)-4-[tert-butyl(diphenyl)silyl]oxy-1-chloro-butan-2-ol

The title compound was prepared as a colorless oil (3.3 g, 80% yield) following Scheme 1 and in analogy to Example 28 (step 3) using (3R)-4-chlorobutane-1,3-diol (1.5 g, 10.84 mmol) as starting material.

¹H NMR (400 MHz, DMSO-d₆) δ ppm: 7.65 (m, 4H), 7.45 (m, 6H), 5.06 (d, J=5.2 Hz, 1H), 3.87 (m, 1H), 3.76 (m, 2H), 3.59 (dd, J=10.8 Hz, 4.8 Hz, 1H), 3.52 (dd, J=10.8 Hz, 5.6 Hz, 1H), 1.82 (m, 1H), 1.61 (m, 1H), 0.98 (s, 9H).

### Step 2: Preparation of (2R)-1-azido-4-[tert-butyl(diphenyl)silyl]oxy-butan-2-ol

NaN₃ (87 mg, 1.31 mmol) was added to a stirred solution of (2R)-4-[tert-butyl(diphenyl)silyl]oxy-1-chloro-butan-2-ol (100 mg, 0.26 mmol) in DMF (1 mL). After 16 h stirring at 90° C., the reaction mixture was extracted with EA and H₂O. The combined organic layers were dried over Na₂SO₄, filtered, concentrated and purified by column chromatography (silica gel; PE:EA; 10:1; v:v) to afford (2R)-1-azido-4-[tert-butyl(diphenyl)silyl]oxy-butan-2-ol as a colorless oil (86 mg, 80% yield).

¹H NMR (400 MHz, DMSO-d₆) δ ppm: 7.63 (m, 4H), 7.45 (m, 6H), 5.04 (d, J=5.6 Hz, 1H), 3.87 (m, 1H), 3.73 (m, 2H), 3.23 (dd, J=12.4 Hz, 4.0 Hz, 1H), 3.17 (dd, J=12.8 Hz, 6.8 Hz, 1H), 1.64 (m, 2H), 0.98 (s, 9H).

### Step 3: Preparation of (2R)-1-amino-4-[tert-buty(diphenyl)silyl]oxy-butan-2-ol

10% Pd/C (22 mg, 0.02 mmol) was added to a stirred solution of (2R)-1-azido-4-[tert-butyl(diphenyl)silyl]oxy-butan-2-ol (86 mg, 0.18 mmol) in MeOH (1 mL). After 2 h stirring under hydrogen flow, the catalyst was removed by filtration and the solution was concentrated to afford (2R)-1-amino-4-[tert-butyl(diphenyl)silyl]oxy-butan-2-ol as a colorless oil (75 mg, 94% yield) which was used in the next step without further purification.

¹H NMR (400 MHz, DMSO-d₆) δ ppm: 7.62 (m, 4H), 7.44 (m, 6H), 4.43 (br, 1H), 3.74 (m, 2H), 3.50 (m, 1H), 2.52 (overlapped with DMSO peak, 1H), 2.40 (dd, J=12.4 Hz, 6.8 Hz, 1H), 1.68 (m, 1H), 1.52 (m, 1H), 0.98 (s, 9H).

MS m/z (+ESI): 344.3 [M+H]⁺.

### Step 4: Preparation of (5R)-5-[2-[tert-butyl(diphenyl)silyl]oxyethyl]oxazolidin-2-one

DIPEA (250 μL, 1.41 mmol) was added to a stirred solution of (2R)-1-amino-4-[tert-butyl(diphenyl)silyl]oxy-butan-2-ol (90 mg, 0.23 mmol) in DCM (2 mL). The reaction mixture was then cooled to 0° C. before the addition of triphosgene (35 mg, 0.12 mmol). After 1 h stirring at 0° C., the reaction mixture was extracted with DCM and H₂O. The combined organic layers were dried over Na₂SO₄, filtered, concentrated and purified by column chromatography (silica gel; PE:EA; 2:1; v:v) to afford (5R)-5-[2-[tert-butyl(diphenyl)silyl]oxyethyl]oxazolidin-2-one as a colorless oil (87 mg, 52% yield).

¹H NMR (400 MHz, DMSO-d₆) δ ppm: 7.64 (m, 4H), 7.44 (m, 7H), 4.71 (m, 1H), 3.74 (m, 2H), 3.55 (t, J=8.4 Hz, 1H), 3.15 (t, J=8.0 Hz, 1H), 1.90 (m, 2H), 0.99 (s, 9H).

### Step 5: Preparation of (5R)-5-[2-[tert-butyl(diphenyl)silyl]oxyethyl]-3-[(4-methoxyphenyl)methyl]oxazolidin-2-one

To a solution of (5R)-5-[2-[tert-butyl(diphenyl)silyl]oxyethyl]oxazolidin-2-one (820 mg, 2.00 mmol) in DMF (10 mL) was added cesium carbonate (1.99 g, 5.99 mmol) and 1-(chloromethyl)-4-methoxybenzene (484 mg, 2.99 mmol). The reaction was stirred at 15° C. for 16 h. Then the reaction was diluted with EA and water. After separation, the combined organic layer was dried over Na₂SO₄, filtered, concentrated and purified by column chromatography (silica gel; PE:EA; 2:1; v:v) to afford (R)-5-[2-[tert-butyl(diphenyl)silyl]oxyethyl]-3-[(4-methoxyphenyl)methyl]oxazolidin-2-one as a colorless oil (1.00 g, 92% yield).

¹H NMR (400 MHz, DMSO-d₆) δ ppm: 7.57 (m, 4H), 7.43 (m, 6H), 7.17 (d, J=8.8 Hz, 2H), 6.90 (d, J=8.8 Hz, 2H), 4.64 (m, 1H), 4.24 (s, 2H), 3.73 (s, 3H), 3.71 (m, 2H), 3.48 (t, J=8.8 Hz, 1H), 3.06 (dd, J=8.8 Hz, 6.8 Hz, 1H), 1.86 (m, 2H), 0.95 (s, 9H).

### Step 6: (5R)-5-(2-hydroxyethyl)-3-[(4-methoxyphenyl)methyl]oxazolidin-2-one

To a solution of (5R)-5-[2-[tert-butyl(diphenyl)silyl]oxyethyl]-3-[(4-methoxyphenyl)methyl]oxazolidin-2-one (100 g, 1.84 mmol) in THF (15 mL) was added TBAF (1.70 g, 5.51 mmol). Then the reaction was heated at 70° C. for 1 h. The reaction was diluted with EA and water, the organic phase was separated, dried over Na₂SO₄, filtered and evaporated under vacuum to give the crude material. The residue was purified by column chromatography (silica gel; 100% EA) to afford (5R)-5-(2-hydroxyethyl)-3-[(4-methoxyphenyl)methyl]oxazolidin-2-one as a colorless oil (470 mg, 91% yield).

¹H NMR (400 MHz, DMSO-d₆+D₂O) δ ppm: 7.18 (d, J=8.4 Hz, 2H), 6.91 (d, J=8.4 Hz, 2H), 4.56 (m, 1H), 4.24 (s, 2H), 3.73 (s, 3H), 3.47 (overlapped with H₂O peak, 3H), 3.05 (dd, J=8.8 Hz, 7.2 Hz, 1H), 1.72 (m, 2H).

### Step 7: Preparation of 2-[(5R)-3-[(4-methoxyphenyl)methyl]-2-oxo-oxazolidin-5-yl]ethyl 4-methylbenzenesulfonate

The title compound was prepared as a colorless oil following Scheme 1 and in analogy to Examples 5, 19 and 21 using (5R)-5-(2-hydroxyethyl)-3-[(4-methoxyphenyl)methyl]oxazolidin-2-one as starting material.

¹H NMR (400 MHz, DMSO-d₆) δ ppm: 7.78 (d, J=8.4 Hz, 2H), 7.47 (d, J=8.4 Hz, 2H), 7.16 (d, J=8.4 Hz, 2H), 6.91 (d, J=8.4 Hz, 2H), 4.49 (m, 1H), 4.22 (m, 2H), 4.07 (t, J=6.4 Hz, 2H), 3.74 (s, 3H), 3.41 (t, J=8.8 Hz, 1H), 2.99 (dd, J=8.8 Hz, 6.8 Hz, 1H), 2.41 (s, 3H), 1.93 (m, 2H).

MS m/z (+ESI): 406.1 [M+H]⁺.

### Step 8: Preparation of N-(4-amino-8-methoxy-5,5-dimethyl-6H-benzo[h]quinazolin-7-yl)-2-nitro-benzenesulfonamide

The title compound was prepared as an off-white solid (1.2 g, 71% yield) following Scheme 1 and in analogy to Example 48 (step 1) using 8-methoxy-5,5-dimethyl-6H-benzo[h]quinazoline-4,7-diamine (1.0 g, 3.33 mmol) as starting material.

¹H NMR (400 MHz, DMSO-d₆) δ ppm: 9.47 (s, 1H), 8.26 (s, 1H), 8.03 (d, J=8.8 Hz, 1H), 7.98 (d, J=7.6 Hz, 1H), 7.82 (m, 3H), 6.88 (d, J=8.8 Hz, 1H), 6.47 (br, 2H), 3.21 (s, 3H), 2.82 (s, 2H), 1.25 (s, 6H).

MS m/z (+ESI): 456.2 [M+H]⁺.

### Step 9: Preparation of N-(4-amino-8-methoxy-5,5-dimethyl-6H-benzo[h]quinazolin-7-yl)-N-[2-[(5R)-3-[(4-methoxyphenyl)methyl]-2-oxo-oxazolidin-5-yl]ethyl]-2-nitro-benzenesulfonamide

The title compound was prepared as a yellow solid (800 mg, 76% yield) following Scheme 1 and in analogy to Example 5 (step 3) using N-(4-amino-8-methoxy-5,5-dimethyl-6H-benzo[h]quinazolin-7-yl)-2-nitro-benzenesulfonamide (700 mg, 1.38 mmol) and 2-[(5R)-3-[(4-methoxyphenyl)methyl]-2-oxo-oxazolidin-5-yl]ethyl 4-methylbenzenesulfonate (623 mg, 1.38 mmol) as starting materials.

¹H NMR (400 MHz, DMSO-d₆) δ ppm: 8.27 (d, J=1.6 Hz, 1H), 8.16 (dd, J=8.8 Hz, 3.6 Hz, 1H), 7.90 (m, 2H), 7.76 (m, 2H), 7.17 (d, J=8.4 Hz, 1H), 7.11 (d, J=8.4 Hz, 1H), 6.96 (dd, J=8.8 Hz, 5.6 Hz, 1H), 6.90 (d, J=8.4 Hz, 1H), 6.85 (d, J=8.8 Hz, 1H), 6.47 (br, 2H), 4.55 (m, 1H), 4.24 (m, 2H), 3.98 (m, 1H), 3.71 (d, J=13.6 Hz, 3H), 3.50 (m, 2H), 3.19 (d, J=3.2 Hz, 3H), 2.95 (m, 1H), 2.80 (d, J=8.0 Hz, 2H), 1.90 (m, 1H), 1.62 (m, 1H), 1.38 (d, J=12.4 Hz, 3H), 1.13 (d, J=11.2 Hz, 3H).

MS m/z (+ESI): 689.3 [M+H]⁺.

### Step 10: Preparation of N-(4-amino-8-methoxy-5,5-dimethyl-6H-benzo[h]quinazolin-7-yl)-2-nitro-N-[2-[(5R)-2-oxooxazolidin-5-yl]ethyl]benzenesulfonamide

CAN (1.65 g, 2.98 mmol) was added to a stirred solution of N-(4-amino-8-methoxy-5,5-dimethyl-6H-benzo[h]quinazolin-7-yl)-N-[2-[(5R)-3-[(4-methoxyphenyl)methyl]-2-oxo-oxazolidin-5-yl]ethyl]-2-nitro-benzenesulfonamide (760 mg, 0.99 mmol) in ACN (10 mL) and H₂O (3 mL). After 1 h stirring, the reaction mixture was concentrated and purified by combiflash to afford N-(4-amino-8-methoxy-5,5-dimethyl-6H-benzo[h]quinazolin-7-yl)-2-nitro-N-[2-[(5R)-2-oxooxazolidin-5-yl]ethyl]benzenesulfonamide as a yellow solid (600 mg, 85% yield).

MS m/z (+ESI): 569.2 [M+H]⁺.

### Step 11: Preparation of (5R)-5-[2-[(4-amino-8-methoxy-5,5-dimethyl-6H-benzo[h]quinazolin-7-yl)amino]ethyl]oxazolidin-2-one

The title compound was prepared as a white solid (61 mg, 19% yield) following Scheme 1 and in analogy to Example 48 (step 3) using N-(4-amino-8-methoxy-5,5-dimethyl-6H-benzo[h]quinazolin-7-yl)-2-nitro-N-[2-[(5R)-2-oxooxazolidin-5-yl]ethyl]benzenesulfonamide (580 mg, 0.82 mmol) as starting material.

¹H NMR (400 MHz, DMSO-d₆+D₂O) δ ppm: 8.22 (s, 1H), 7.67 (d, J=8.8 Hz, 1H), 6.90 (d, J=8.8 Hz, 1H), 4.67 (m, 1H), 3.82 (s, 3H), 3.54 (t, J=8.4 Hz, 1H), 3.10 (t, J=8.0 Hz, 1H), 3.00 (m, 2H), 2.72 (s, 2H), 1.78 (m, 2H), 1.26 (s, 3H), 1.25 (s, 3H).

MS m/z (+ESI): 384.1 [M+H]⁺.

### Preparation of Example 90: 4-amino-8-(trans-4-aminocyclohexoxy)-N-(cyanomethyl)-5,5-dimethyl-6H-benzo[h]quinazoline-7-carboxamide

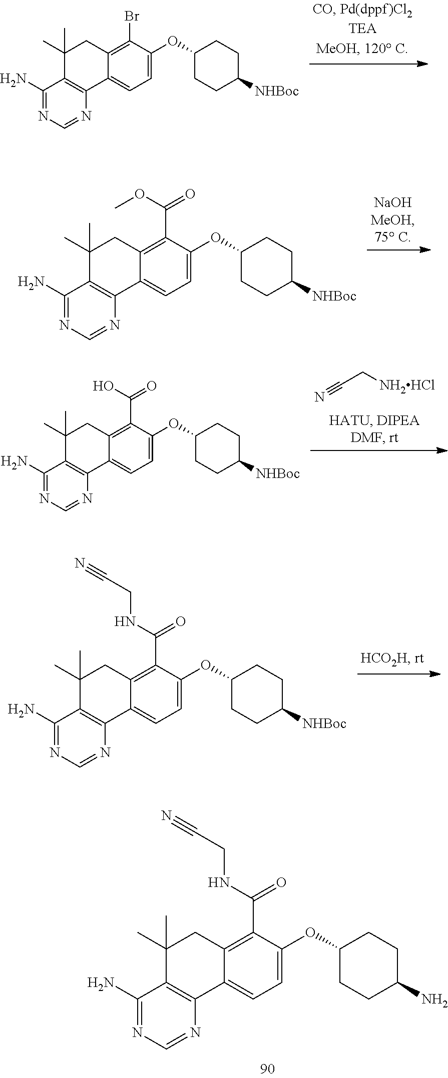

tert-butyl N-[trans-4-[(4-amino-7-bromo-5,5-dimethyl-6H-benzo[h]quinazolin-8-yl)oxy]cyclohexyl]carbamate was prepared as an off-white solid following Scheme 1 and in analogy to Examples 5 and 61 using 4-amino-5,5-dimethyl-6H-benzo[h]quinazolin-8-ol and CAS 167081-25-6 as starting materials.

### Step 1: preparation of methyl 4-amino-8-[trans-4-(tert-butoxycarbonylamino)cyclohexoxy]-5,5-dimethyl-6H-benzo[h]quinazoline-7-carboxylate

Pd(dppf)Cl₂ (110 mg, 0.15 mmol) was added to a stirred solution of tert-butyl N-[trans-4-[(4-amino-7-bromo-5,5-dimethyl-6H-benzo[h]quinazolin-8-yl)oxy]cyclohexyl]carbamate (300 mg, 0.49 mmol) in MeOH (4 mL), followed by TEA (350 μL, 2.46 mmol). After 8 h stirring at 120° C. under CO flow (3.5 MPa), the catalyst was removed by filtration. The solution was concentrated and the residue was purified by combiflash to afford methyl 4-amino-8-[trans-4-(tert-butoxycarbonylamino)cyclohexoxy]-5,5-dimethyl-6H-benzo[h]quinazoline-7-carboxylate as a white solid (190 mg, 70% yield).

MS m/z (+ESI): 497.3 [M+H]⁺.

### Step 2: preparation of 4-amino-8-[trans-4-(tert-butoxycarbonylamino)cyclohexoxy]-5,5-dimethyl-6H-benzo[h]quinazoline-7-carboxylic acid

NaOH (39 mg, 0.98 mmol) was added to a stirred solution of methyl 4-amino-8-[trans-4-(tert-butoxycarbonylamino)cyclohexoxy]-5,5-dimethyl-6H-benzo[h]quinazoline-7-carboxylate (180 mg, 0.33 mmol) in MeOH (4 mL). After 16 h stirring at 75° C., the pH of the reaction mixture was adjusted to 3 with a 1M HCl aqueous solution. The resulting mixture was extracted with EA and H₂O. The combined organic layers were dried over Na₂SO₄, filtered and concentrated. The residue was purified by combiflash to afford 4-amino-8-[trans-4-(tert-butoxycarbonylamino)cyclohexoxy]-5,5-dimethyl-6H-benzo[h]quinazoline-7-carboxylic acid as a white solid (150 mg, 86% yield).

MS m/z (+ESI): 483.3 [M+H]⁺.

### Step 3: Preparation of 4-amino-8-(trans-4-aminocyclohexoxy)-N-(cyanomethyl)-5,5-dimethyl-6H-benzo[h]quinazoline-7-carboxamide

The title compound was prepared as a white solid following Scheme 1 and in analogy to Examples 6, and 9 using 4-amino-8-[trans-4-(tert-butoxycarbonylamino)cyclohexoxy]-5,5-dimethyl-6H-benzo[h]quinazoline-7-carboxylic acid and CAS 6011-14-9 as starting materials.

¹H NMR (400 MHz, DMSO-d₆+D₂O) δ ppm: 9.03 (t, J=5.6 Hz, 1H), 8.55 (s, 1H), 7.98 (d, J=8.4 Hz, 1H), 7.30 (d, J=8.4 Hz, 1H), 4.46 (m, 1H), 4.31 (d, J=5.6 Hz, 2H), 3.08 (m, 1H), 2.66 (s, 2H), 2.10 (m, 2H), 1.98 (m, 2H), 1.49 (m, 4H), 1.26 (s, 6H).

MS m/z (+ESI): 421.1 [M+H]⁺.

### Preparation of Example 92: 3-[4-amino-8-(trans-4-aminocyclohexoxy)-5,5-dimethyl-6H-benzo[h]quinazolin-7-yl]butan-1-ol

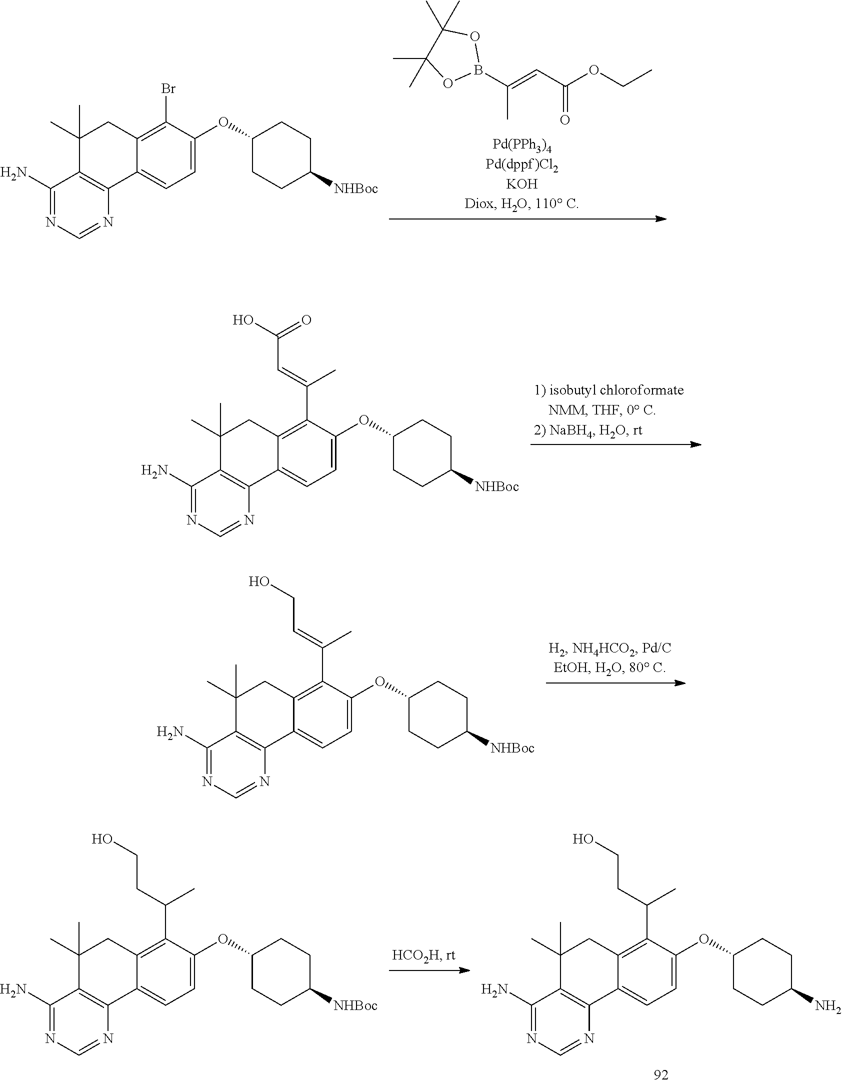 [SMILES: CC(C)(C)OC(=O)N[C@H]1CC[C@H](Oc2ccc3c(c2Br)CC(C)(C)c2c(N)ncnc2-3)CC1 ; CCOC(=O)/C=C(\C)B1OC(C)(C)C(C)(C)O1 ; C/C(=C\C(=O)O)c1c(O[C@H]2CC[C@H](NC(=O)OC(C)(C)C)CC2)ccc2c1CC(C)(C)c1c(N)ncnc1-2 ; C/C(=C\CO)c1c(O[C@H]2CC[C@H](NC(=O)OC(C)(C)C)CC2)ccc2c1CC(C)(C)c1c(N)ncnc1-2 ; CC(CCO)c1c(O[C@H]2CC[C@H](NC(=O)OC(C)(C)C)CC2)ccc2c1CC(C)(C)c1c(N)ncnc1-2 ; CC(CCO)c1c(O[C@H]2CC[C@H](N)CC2)ccc2c1CC(C)(C)c1c(N)ncnc1-2]

### Step 1: preparation of (E)-3-[4-amino-8-[trans-4-(tert-butoxycarbonylamino)cyclohexoxy]-5,5-dimethyl-6H-benzo[h]quinazolin-7-yl]but-2-enoic acid

The title compound was prepared as an off-white solid (360 mg, 71% yield) following Scheme 1 and in analogy to Example 61 (step 3) using tert-butyl N-[trans-4-[(4-amino-7-bromo-5,5-dimethyl-6H-benzo[h]quinazolin-8-yl)oxy]cyclohexyl]carbamate (500 mg, 0.87 mmol) and CAS 448212-00-8 (1.0 g, 3.96 mmol) as starting materials.

MS m/z (+ESI): 523.4 [M+H]⁺.

### Step 2: Preparation of tert-butyl N-[trans-4-[[4-amino-7-[(E)-3-hydroxy-1-methyl-prop-1-enyl]-5,5-dimethyl-6H-benzo[h]quinazolin-8-yl]oxy]cyclohexyl]carbamate

Isobutyl chloroformate (113 μL, 0.83 mmol) was added at 0° C. to a stirred solution of (E)-3-[4-amino-8-[trans-4-(tert-butoxycarbonylamino)cyclohexoxy]-5,5-dimethyl-6H-benzo[h]quinazolin-7-yl]but-2-enoic acid (240 mg, 0.41 mmol) in THF (15 mL), followed by NMM (93 μL, 0.83 mmol). After 30 minutes stirring at 0° C., a solution of NaBH₄ (64 mg, 1.65 mmol) in H₂O (3 mL) was added and the resulting suspension was stirred at 0° C. for 1 h. The reaction mixture was quenched with a saturated NH₄Cl aqueous solution and extracted with EA. The combined organic layers were washed with brine, dried over Na₂SO₄, filtered and concentrated. The residue was purified by combiflash to afford tert-butyl N-[trans-4-[[4-amino-7-[(E)-3-hydroxy-1-methyl-prop-1-enyl]-5,5-dimethyl-6H-benzo[h]quinazolin-8-yl]oxy]cyclohexyl]carbamate as an off-white solid (180 mg, 77% yield).

MS m/z (+ESI): 509.4 [M+H]⁺.

### Step 3: Preparation of 3-[4-amino-8-(trans-4-aminocyclohexoxy)-5,5-dimethyl-6H-benzo[h]quinazolin-7-yl]butan-1-ol

The title compound was prepared as a white solid following Scheme 1 and in analogy to Examples 9 and 61 using as starting material tert-butyl N-[trans-4-[[4-amino-7-[(E)-3-hydroxy-1-methyl-prop-1-enyl]-5,5-dimethyl-6H-benzo[h]quinazolin-8-yl]oxy]cyclohexyl]carbamate.

¹H NMR (400 MHz, DMSO-d₆+D₂O) δ ppm: 8.22 (s, 1H), 7.90 (d, J=8.8 Hz, 1H), 6.95 (d, J=8.8 Hz, 1H), 4.37 (m, 1H), 3.24 (m, 3H), 3.02 (m, 1H), 2.83 (m, 1H), 2.71 (m, 1H), 2.13 (m, 3H), 1.95 (m, 2H), 1.76 (m, 1H), 1.45 (m, 4H), 1.25 (m, 9H).

MS m/z (+ESI): 411.2 [M+H]⁺.

### Preparation of Example 97: 4-[4-amino-8-(trans-4-aminocyclohexoxy)-5,5-dimethyl-6H-benzo[h]quinazolin-7-yl]pentanenitrile

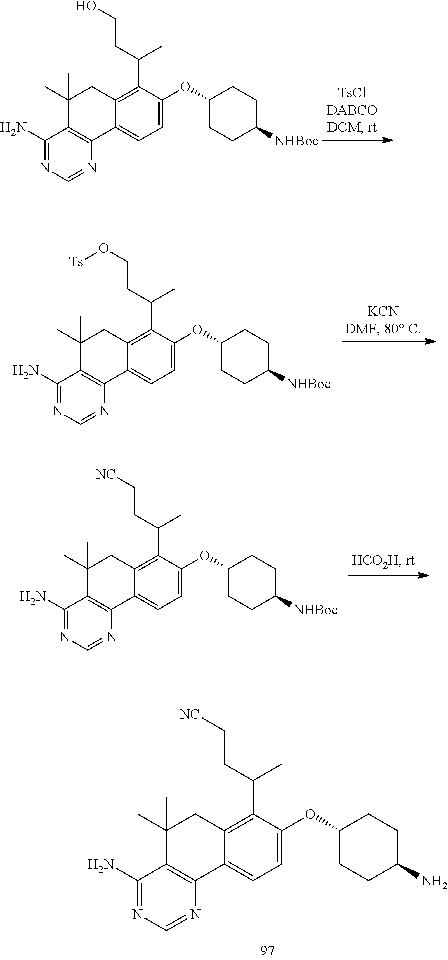 [SMILES: CC(CCO)c1c(O[C@H]2CC[C@H](NC(=O)OC(C)(C)C)CC2)ccc2c1CC(C)(C)c1c(N)ncnc1-2 ; Cc1ccc(S(=O)(=O)OCCC(C)c2c(O[C@H]3CC[C@H](NC(=O)OC(C)(C)C)CC3)ccc3c2CC(C)(C)c2c(N)ncnc2-3)cc1 ; CC(CCC#N)c1c(O[C@H]2CC[C@H](NC(=O)OC(C)(C)C)CC2)ccc2c1CC(C)(C)c1c(N)ncnc1-2 ; CC(CCC#N)c1c(O[C@H]2CC[C@H](N)CC2)ccc2c1CC(C)(C)c1c(N)ncnc1-2]

### Step 1: preparation of 3-[4-amino-8-[trans-4-(tert-butoxycarbonylamino)cyclohexoxy]-5,5-dimethyl-6H-benzo[h]quinazolin-7-yl]butyl 4-methylbenzenesulfonate

TsCl (58 mg, 0.30 mmol) was added to a stirred solution of tert-butyl N-[trans-4-[[4-amino-7-(3-hydroxy-1-methyl-propyl)-5,5-dimethyl-6H-benzo[h]quinazolin-8-yl]oxy]cyclohexyl]carbamate (120 mg, 0.20 mmol) in DCM (10 mL), followed by DABCO (68 mg, 0.60 mmol). After 2 h stirring, the reaction mixture was concentrated and the residue was purified by combiflash to afford 3-[4-amino-8-[trans-4-(tert-butoxycarbonylamino)cyclohexoxy]-5,5-dimethyl-6H-benzo[h]quinazolin-7-yl]butyl 4-methylbenzenesulfonate as a white solid (140 mg, 90% yield).

MS m/z (+ESI): 665.3 [M+H]⁺.

### Step 2: preparation of tert-butyl N-[trans-4-[[4-amino-7-(3-cyano-1-methyl-propyl)-5,5-dimethyl-6H-benzo[h]quinazolin-8-yl]oxy]cyclohexyl]carbamate

KCN (37 mg, 0.54 mmol) was added to a stirred solution of 3-[4-amino-8-[trans-4-(tert-butoxycarbonylamino)cyclohexoxy]-5,5-dimethyl-6H-benzo[h]quinazolin-7-yl]butyl 4-methylbenzenesulfonate (140 mg, 0.18 mmol) in DMF (4 mL). After 1 h stirring at 80° C., the reaction mixture was diluted with H₂O. The resulting suspension was filtered, the cake was washed with H₂O and dried under high vacuum to afford tert-butyl N-[trans-4-[[4-amino-7-(3-cyano-1-methyl-propyl)-5,5-dimethyl-6H-benzo[h]quinazolin-8-yl]oxy]cyclohexyl]carbamate as a white solid (93 mg, 91% yield).

MS m/z (+ESI): 520.3 [M+H]⁺.

### Step 3: preparation of 4-[4-amino-8-(trans-4-aminocyclohexoxy)-5,5-dimethyl-6H-benzo[h]quinazolin-7-yl]pentanenitrile

The title compound was prepared as a white solid (52 mg, 50% yield) following Scheme 1 and in analogy to Example 9 (step 2) using tert-butyl N-[trans-4-[[4-amino-7-(3-cyano-1-methyl-propyl)-5,5-dimethyl-6H-benzo[h]quinazolin-8-yl]oxy]cyclohexyl]carbamate (50 mg, 0.08 mmol) as starting material.

¹H NMR (400 MHz, DMSO-d₆+D₂O) δ ppm: 8.22 (s, 1H), 7.94 (d, J=8.8 Hz, 1H), 6.99 (d, J=8.8 Hz, 1H), 4.39 (m, 1H), 3.30 (m, 1H), 3.04 (m, 1H), 2.79 (m, 2H), 2.28 (m, 3H), 2.15 (m, 2H), 1.96 (m, 3H), 1.48 (m, 4H), 1.26 (m, 9H).

MS m/z (+ESI): 420.2 [M+H]⁺.

### Preparation of Example 108: 3-[[4-amino-8-(trans-4-aminocyclohexoxy)-5,5-dimethyl-6H-benzo[h]quinazolin-7-yl]sulfanyl]propanenitrile

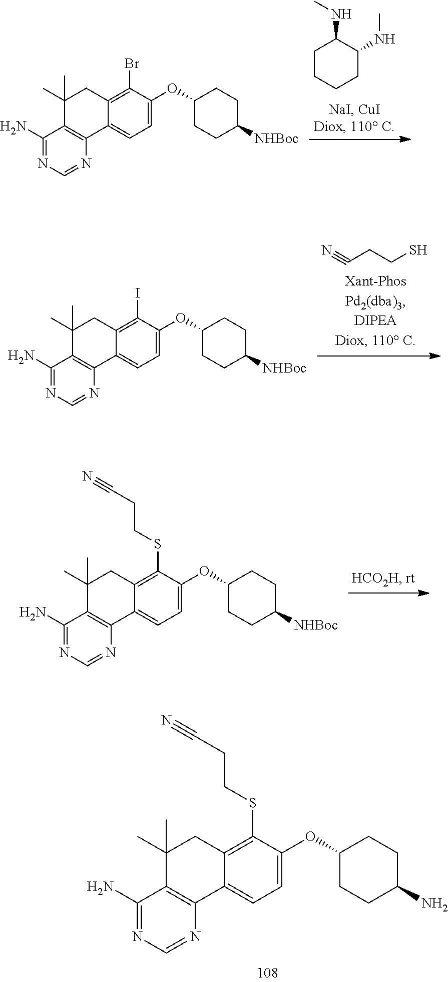

### Step 1: preparation of tert-butyl N-[trans-4-[(4-amino-7-iodo-5,5-dimethyl-6H-benzo[h]quinazolin-8-yl)oxy]cyclohexyl]carbamate

(1R,2R)—N1,N2-dimethylcyclohexane-1,2-diamine (13 mg, 0.09 mmol) was added to a stirred suspension of tert-butyl N-[trans-4-[(4-amino-7-bromo-5,5-dimethyl-6H-benzo[h]quinazolin-8-yl)oxy]cyclohexyl]carbamate (100 mg, 0.147 mmol) in Diox (3 mL), followed by NaI (53 mg, 0.35 mmol) and CuI (17 mg, 0.09 mmol). After 24 h stirring at 110° C., the reaction mixture was filtered and the filtrate was concentrated. The residue was purified by combiflash to afford tert-butyl N-[trans-4-[(4-amino-7-iodo-5,5-dimethyl-6H-benzo[h]quinazolin-8-yl)oxy]cyclohexyl]carbamate as an off-white solid (80 mg, 73% yield).

MS m/z (+ESI): 564.9 [M+H]⁺.

### Step 2: preparation of tert-butyl N-[trans-4-[[4-amino-7-(2-cyanoethylsulfanyl)-5,5-dimethyl-6H-benzo[h]quinazolin-8-yl]oxy]cyclohexyl]carbamate

3-sulfanylpropanenitrile (510 μL, 5.67 mmol) was added to a stirred solution of tert-butyl N-[trans-4-[(4-amino-7-iodo-5,5-dimethyl-6H-benzo[h]quinazolin-8-yl)oxy]cyclohexyl]carbamate (500 mg, 0.71 mmol) in Diox (15 mL), followed by Xant-Phos (167 mg, 0.28 mmol), Pd₂(dba)₃ (132 mg, 0.14 mmol) and DIPEA (504 μL, 0.37 mmol). After 16 h stirring at 110° C., the reaction mixture was filtered and the filtrate was concentrated. The residue was purified by combiflash to afford tert-butyl N-[trans-4-[[4-amino-7-(2-cyanoethylsulfanyl)-5,5-dimethyl-6H-benzo[h]quinazolin-8-yl]oxy]cyclohexyl]carbamate as a yellow solid (300 mg, 0.51 mmol).

MS m/z (+ESI): 524.1 [M+H]⁺.

### Step 3: preparation of 3-[[4-amino-8-(trans-4-aminocyclohexoxy)-5,5-dimethyl-6H-benzo[h]quinazolin-7-yl]sulfanyl]propanenitrile

The title compound was prepared as a white solid (47 mg, 63% yield) following Scheme 1 and in analogy to Example 9 (step 2) using tert-butyl N-[trans-4-[[4-amino-7-(2-cyanoethylsulfanyl)-5,5-dimethyl-6H-benzo[h]quinazolin-8-yl]oxy]cyclohexyl]carbamate (100 mg, 0.17 mmol) as starting material.

¹H NMR (400 MHz, DMSO-d₆+D₂O) δ ppm: 8.24 (s, 1H), 8.08 (d, J=8.8 Hz, 1H), 7.12 (d, J=8.8 Hz, 1H), 4.46 (m, 1H), 3.09 (s, 2H), 3.06 (m, 1H), 3.02 (t, J=6.4 Hz, 2H), 2.55 (t, J=6.4 Hz, 2H), 2.15 (m, 2H), 1.97 (m, 2H), 1.49 (m, 4H), 1.28 (s, 6H).

MS m/z (+ESI): 424.2 [M+H]⁺.

### Preparation of Example 109: 3-[(4-amino-5,5-dimethyl-6H-benzo[h]quinazolin-7-yl)-methyl-amino]propanenitrile

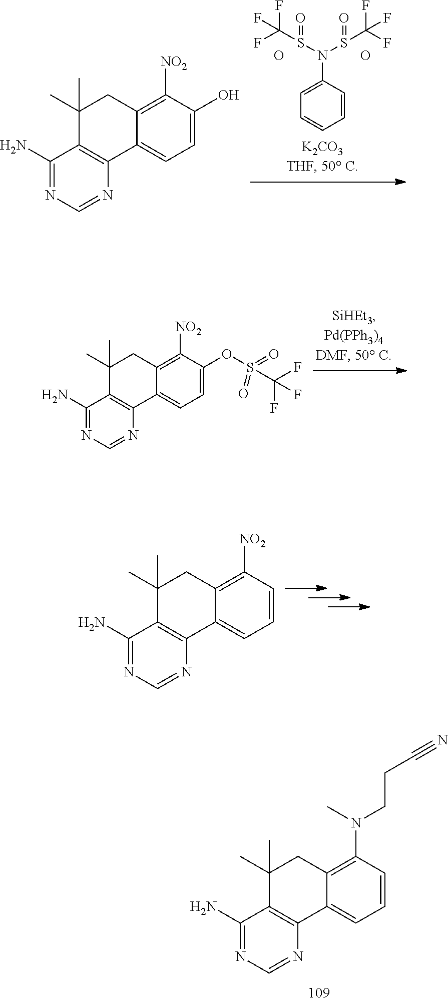

### Step 1: Preparation of(4-amino-5,5-dimethyl-7-nitro-6H-benzo[h]quinazolin-8-yl) trifluoromethanesulfonate

N-phenyl-bis(trifluoromethanesulfonimide) (7.73 g, 21.22 mmol) was added to a stirred solution of 4-amino-5,5-dimethyl-7-nitro-6H-benzo[h]quinazolin-8-ol (4.5 g, 14.15 mmol) in THF (45 mL), followed by K₂CO₃ (5.98 g, 42.44 mmol). After 2 h stirring at 50° C., the reaction mixture was filtered. The filtrate was concentrated and the residue was purified by combiflash to afford (4-amino-5,5-dimethyl-7-nitro-6H-benzo[h]quinazolin-8-yl) trifluoromethanesulfonate as a light yellow solid (3 g, 46% yield).

MS m/z (+ESI): 419.0 [M+H]⁺.

### Step 2: Preparation of 5,5-dimethyl-6H-benzo[h]quinazoline-4,7-diamine

Triethylsilane (1.25 mL, 7.74 mmol) was added to a stirred solution of (4-amino-5,5-dimethyl-7-nitro-6H-benzo[h]quinazolin-8-yl) trifluoromethanesulfonate (1.2 g, 2.58 mmol) in DMF (30 mL), followed by Pd(PPh₃)₄(304 mg, 0.26 mmol). After 2 h stirring at 50° C., the reaction mixture was filtered. The filtrate was concentrated and the residue was purified by combiflash to afford 5,5-dimethyl-6H-benzo[h]quinazoline-4,7-diamine as a light yellow solid (600 mg, 77% yield).

MS m/z (+ESI): 271.1 [M+H]⁺.

### Step 3: Preparation of 3-[(4-amino-5,5-dimethyl-6H-benzo[h]quinazolin-7-yl)-methyl-amino]propanenitrile

The title compound was prepared as a white solid following Scheme 1 and in analogy to Examples 2, 22 and 48 (step 2) using 5,5-dimethyl-6H-benzo[h]quinazoline-4,7-diamine, CAS 98-74-8, CAS 2417-90-5 and CAS 50-00-0 as starting materials.

¹H NMR (400 MHz, DMSO-d₆+D₂O) δ ppm: 8.27 (s, 1H), 7.81 (d, J=6.8 Hz, 1H), 7.26 (m, 2H), 3.12 (t, J=6.4 Hz, 2H), 2.88 (s, 2H), 2.66 (t, J=6.4 Hz, 2H), 2.63 (s, 3H), 1.26 (s, 6H).

MS m/z (+ESI): 308.2 [M+H]⁺.

### Preparation of Example 113: 4-amino-8-(trans-4-aminocyclohexoxy)-N-(cyanomethyl)-N,5,5-trimethyl-6H-benzo[h]quinazoline-7-sulfonamide

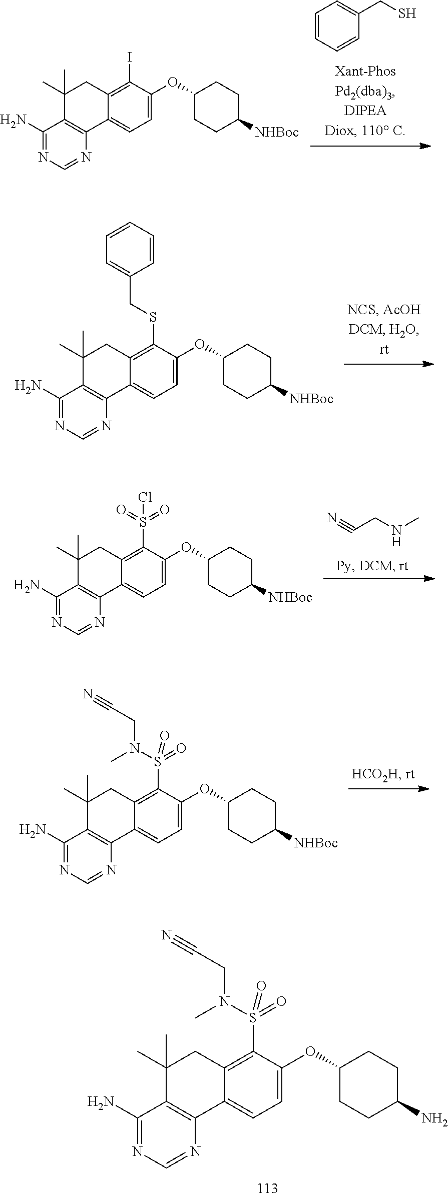

### Step 1: Preparation of tert-butyl N-[trans-4-[(4-amino-7-benzylsulfanyl-5,5-dimethyl-6H-benzo[h]quinazolin-8-yl)oxy]cyclohexyl]carbamate

The title compound was prepared as a yellow solid (200 mg, 71% yield) following Scheme 1 and in analogy to Example 108 (step 2) using tert-butyl N-[trans-4-[(4-amino-7-iodo-5,5-dimethyl-6H-benzo[h]quinazolin-8-yl)oxy]cyclohexyl]carbamate (300 mg, 0.43 mmol) and CAS 100-53-8 (252 μL, 2.12 mmol) as starting materials.

MS m/z (+ESI): 561.1 [M+H]⁺.

### Step 2: Preparation of tert-butyl N-[trans-4-[(4-amino-7-chlorosulfonyl-5,5-dimethyl-6H-benzo[h]quinazolin-8-yl)oxy]cyclohexyl]carbamate

NCS (170 mg, 1.21 mmol) was added to a stirred solution of tert-butyl N-[trans-4-[(4-amino-7-benzylsulfanyl-5,5-dimethyl-6H-benzo[h]quinazolin-8-yl)oxy]cyclohexyl]carbamate (200 mg, 0.30 mmol) in DCM (4 mL) and H₂O (0.1 mL), followed by AcOH (280 μL, 4.80 mmol). After 16 h stirring, the reaction mixture was diluted with DCM (20 mL) and NaHCO₃ (2 g) was added. The resulting suspension was filtered. The filtrate was concentrated and extracted with DCM and H₂O. The combined organic layers were dried over Na₂SO₄, filtered, concentrated to afford tert-butyl N-[trans-4-[(4-amino-7-chlorosulfonyl-5,5-dimethyl-6H-benzo[h]quinazolin-8-yl)oxy]cyclohexyl]carbamate as a yellow liquid (190 mg, 93% yield) which was used in the next step without further purification.

MS m/z (+ESI): 537.0 [M+H]⁺.

### Step 3: Preparation of tert-butyl N-[trans-4-[[4-amino-7-[cyanomethyl(methyl)sulfamoyl]-5,5-dimethyl-6H-benzo[h]quinazolin-8-yl]oxy]cyclohexyl]carbamate

2-(Dimethylamino)acetonitrile (331 μL, 4.24 mmol) was added to a stirred solution of tert-butyl N-[trans-4-[(4-amino-7-chlorosulfonyl-5,5-dimethyl-6H-benzo[h]quinazolin-8-yl)oxy]cyclohexyl]carbamate (190 mg, 0.28 mmol) in DCM (10 mL) and Py (2 mL). After 30 minutes stirring the reaction mixture was concentrated and the residue was purified by preparative HPLC to afford tert-butyl N-[trans-4-[[4-amino-7-[cyanomethyl(methyl)sulfamoyl]-5,5-dimethyl-6H-benzo[h]quinazolin-8-yl]oxy]cyclohexyl]carbamate as an off-white solid (160 mg, 0.25 mmol).

MS m/z (+ESI): 571.0 [M+H]⁺.

### Step 4: Preparation 4-amino-8-(trans-4-aminocyclohexoxy)-N-(cyanomethyl)-N,5,5-trimethyl-6H-benzo[h]quinazoline-7-sulfonamide

The title compound was prepared as a white solid (105 mg, 88% yield) following Scheme 1 and in analogy to Example 9 (step 2) using tert-butyl N-[trans-4-[[4-amino-7-[cyanomethyl(methyl)sulfamoyl]-5,5-dimethyl-6H-benzo[h]quinazolin-8-yl]oxy]cyclohexyl]carbamate (160 mg, 0.25 mmol) as starting material.

¹H NMR (400 MHz, DMSO-d₆+D₂O) δ ppm: 8.31 (d, J=8.8 Hz, 1H), 8.27 (s, 1H), 7.35 (d, J=8.8 Hz, 1H), 4.56 (m, 1H), 4.43 (s, 2H), 3.30 (s, 2H), 3.00 (m, 1H), 2.87 (s, 3H), 2.12 (m, 2H), 1.97 (m, 2H), 1.60 (m, 2H), 1.47 (m, 2H), 1.26 (s, 6H).

MS m/z (+ESI): 471.1 [M+H]⁺.

### Preparation of Example 115: 3-[(4-amino-8-bromo-5,5-dimethyl-6H-benzo[h]quinazolin-7-yl)-methyl-amino]propanenitrile

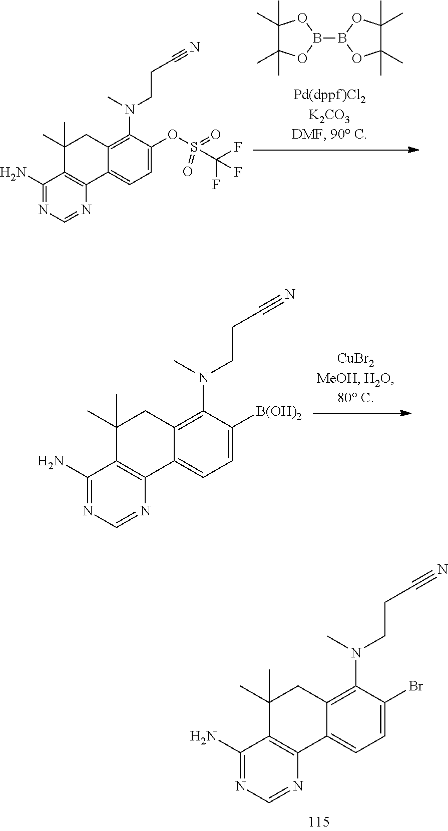

[4-Amino-7-[2-cyanoethyl(methyl)amino]-5,5-dimethyl-6H-benzo[h]quinazolin-8-yl]trifluoromethanesulfonate was prepared as a white solid following Scheme 1 and in analogy to Examples 2, 22 and 48 using 4-amino-5,5-dimethyl-7-nitro-6H-benzo[h]quinazolin-8-ol and CAS 98-74-8, CAS 2417-90-5 and CAS 50-00-0 as starting materials.

MS m/z (+ESI): 456.2 [M+H]⁺.

### Step 1: Preparation of [4-amino-7-[2-cyanoethyl(methyl)amino]-5,5-dimethyl-6H-benzo[h]quinazolin-8-yl]boronic acid

Bis(pinacolato)diboron (2.05 g, 7.90 mmol) was added to a stirred solution of [4-amino-7-[2-cyanoethyl(methyl)amino]-5,5-dimethyl-6H-benzo[h]quinazolin-8-yl]trifluoromethanesulfonate (300 mg, 0.53 mmol) in DMF (15 mL), followed by Pd(dppf)Cl₂ (78 mg, 0.10 mmol) and K₂CO₃ (223 mg, 1.58 mmol). After 8 h stirring at 90° C., the reaction mixture was filtered. The filtrate was concentrated and the residue was purified by combiflash to afford [4-amino-7-[2-cyanoethyl(methyl)amino]-5,5-dimethyl-6H-benzo[h]quinazolin-8-yl]boronic acid as a white solid (80 mg, 39% yield).

¹H NMR (400 MHz, DMSO-d₆+D₂O) δ ppm: 8.27 (s, 1H), 7.84 (d, J=8.0 Hz, 1H), 7.33 (d, J=8.0 Hz, 1H), 3.28 (m, 2H), 2.88 (m, 2H), 2.74 (s, 3H), 2.67 (m, 2H), 1.28 (s, 6H).

MS m/z (+ESI): 352.3 [M+H]⁺.

### Step 2: Preparation of 3-[(4-amino-8-bromo-5,5-dimethyl-6H-benzo[h]quinazolin-7-yl)-methyl-amino]propanenitrile

CuBr₂ (964 mg, 4.30 mmol) was added to a stirred solution of [4-amino-7-[2-cyanoethyl(methyl)amino]-5,5-dimethyl-6H-benzo[h]quinazolin-8-yl]boronic acid (500 mg, 0.43 mmol) in MeOH (10 mL) and H₂O (10 mL). After 10 h stirring at 80° C., the reaction mixture was filtered. The filtrate was concentrated and the residue was extracted with DCM and H₂O. The combined organic layers were dried over Na₂SO₄, filtered, concentrated and the residue was purified by preparative HPLC to afford 3-[(4-amino-8-bromo-5,5-dimethyl-6H-benzo[h]quinazolin-7-yl)-methyl-amino]propanenitrile as a white solid (44 mg, 26% yield).

¹H NMR (400 MHz, DMSO-d₆+D₂O) δ ppm: 8.27 (s, 1H), 7.82 (d, J=8.4 Hz, 1H), 7.55 (d, J=8.4 Hz, 1H), 3.41 (m, 1H), 3.34 (m, 1H), 2.96 (m, 2H), 2.78 (s, 3H), 2.70 (m, 1H), 2.59 (m, 1H), 1.30 (s, 3H), 1.28 (s, 3H).

MS m/z (+ESI): 386.0, 388.1 [M+H]⁺.

### Preparation of Example 118: 3-[[4-amino-5,5-dimethyl-8-(4-methylpiperazin-1-yl)-6H-benzo[h]quinazolin-7-yl]-methyl-amino]propanenitrile

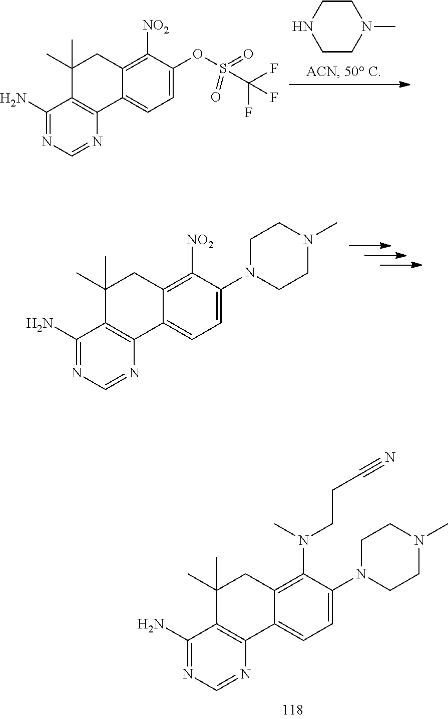

### Step 1: Preparation of 5,5-dimethyl-8-(4-methylpiperazin-1-yl)-7-nitro-6H-benzo[h]quinazolin-4-amine

1-Methylpiperazine (527 mg, 5.16 mmol) was added to a stirred solution of (4-amino-5,5-dimethyl-7-nitro-6H-benzo[h]quinazolin-8-yl) trifluoromethanesulfonate (400 mg, 0.86 mmol) in ACN (40 mL). After 16 h stirring at 50° C., the reaction mixture was concentrated and the residue was extracted with EA and H₂O. The combined organic layers were dried over Na₂SO₄, filtered and concentrated to afford 5,5-dimethyl-8-(4-methylpiperazin-1-yl)-7-nitro-6H-benzo[h]quinazolin-4-amine as a yellow solid (317 mg, 99% yield) which was used in the next step without further purification. 25 ¹H NMR (400 MHz, DMSO-d₆) δ ppm: 8.30 (s, 1H), 8.20 (d, J=8.4 Hz, 1H), 7.35 (d, J=8.4 Hz, 1H), 6.59 (br, 2H), 2.97 (m, 4H), 2.59 (s, 2H), 2.40 (m, 4H), 2.21 (s, 3H), 1.27 (s, 6H).

MS m/z (+ESI): 369.0 [M+H]⁺.

### Step 2: Preparation of 3-[[4-amino-5,5-dimethyl-8-(4-methylpiperazin-1-yl)-6H-benzo[h]quinazolin-7-yl]-methyl-amino]propanenitrile

The title compound was prepared as a white solid following Scheme 1 and in analogy to Examples 2, 22 and 48 using 5,5-dimethyl-8-(4-methylpiperazin-1-yl)-7-nitro-6H-benzo[h]quinazolin-4-amine, CAS 98-74-8, CAS 2417-90-5 and CAS 50-00-0 as starting materials.

¹H NMR (400 MHz, DMSO-d₆+D₂O) δ ppm: 8.24 (s, 1H), 7.83 (d, J=8.4 Hz, 1H), 7.06 (d, J=8.4 Hz, 1H), 3.61 (m, 1H), 3.31 (m, 1H), 2.97 (m, 4H), 2.80 (s, 3H), 2.66 (m, 8H), 2.25 (s, 3H), 1.38 (s, 3H), 1.17 (s, 3H).

MS m/z (+ESI): 406.2 [M+H]⁺.

### Preparation of Example 120: 1-(4-amino-8-bromo-5,5-dimethyl-6H-benzo[h]quinazolin-7-yl)pyrrolidine-3-carbonitrile

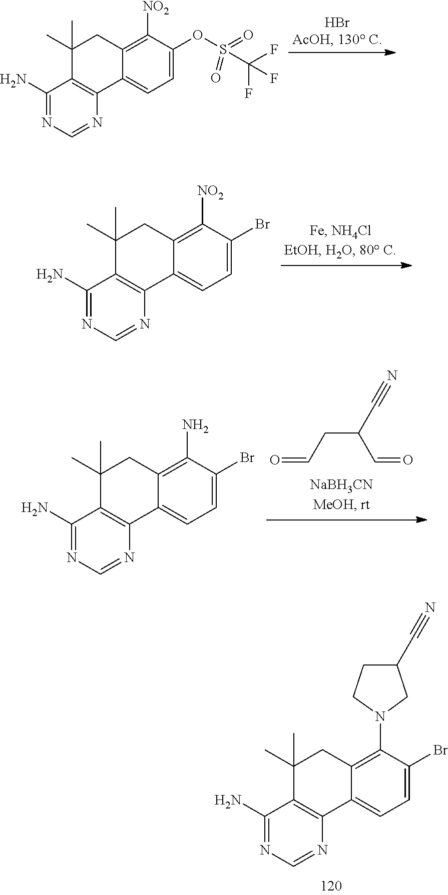

### Step 1: Preparation of 8-bromo-5,5-dimethyl-7-nitro-6H-benzo[h]quinazolin-4-amine

HBr (33% in AcOH, 50 mL) was added to a stirred suspension of (4-amino-5,5-dimethyl-7-nitro-6H-benzo[h]quinazolin-8-yl) trifluoromethanesulfonate (1.4 g, 3.01 mmol) in AcOH (25 mL). After 16 h stirring at 130° C., the reaction mixture was diluted with H₂O (150 mL). The resulting suspension was filtered, the cake was washed with H₂O and dried under high vacuum. The resulting off-white solid was poured in THF (300 mL) and a saturated NaHCO₃ aqueous solution (100 mL). After 10 minutes stirring, the organic layer was separated, washed with H₂O and brine, dried over Na₂SO₄, filtered and concentrated to afford 8-bromo-5,5-dimethyl-7-nitro-6H-benzo[h]quinazolin-4-amine as a yellow solid (950 mg, 72% yield) which was used in the next step without further purification.

¹H NMR (400 MHz, DMSO-d₆) δ ppm: 8.33 (s, 1H), 8.18 (d, J=8.8 Hz, 1H), 7.84 (d, J=8.8 Hz, 1H), 6.73 (br, 2H), 2.71 (s, 2H), 1.28 (s, 6H).

MS m/z (+ESI): 348.9, 350.9 [M+H]⁺.

### Step 2: Preparation of 8-bromo-5,5-dimethyl-6H-benzo[h]quinazoline-4,7-diamine

Iron (1.24 g, 21.77 mmol) was added to a stirred suspension of 8-bromo-5,5-dimethyl-7-nitro-6H-benzo[h]quinazolin-4-amine (950 mg, 2.18 mmol) in EtOH (80 mL) and H₂O (8 mL), followed by NH₄Cl (594 mg, 10.88 mmol). After 1 h stirring at 80° C., the reaction mixture was filtered. The filtrate was concentrated and the residue was poured in H₂O (60 mL). The resulting suspension was sonicated, filtered and the cake was washed with H₂O and dried under high vacuum to afford 8-bromo-5,5-dimethyl-6H-benzo[h]quinazoline-4,7-diamine as an off-white solid (700 mg, 91% yield) which was used in the next step without further purification. 30 ¹H NMR (400 MHz, DMSO-d₆) δ ppm: 8.61 (s, 1H), 7.54 (d, J=8.4 Hz, 1H), 7.04 (d, J=8.4 Hz, 1H), 2.21 (s, 2H), 1.30 (s, 6H).

MS m/z (+ESI): 319.0, 321.0 [M+H]⁺.

### Step 3: Preparation of 1-(4-amino-8-bromo-5,5-dimethyl-6H-benzo[h]quinazolin-7-yl)pyrrolidine-3-carbonitrile

The title compound was prepared as a white solid (50 mg, 15% yield) following Scheme 1 and in analogy to Example 9 (step 1) using 8-bromo-5,5-dimethyl-6H-benzo[h]quinazoline-4,7-diamine (270 mg, 0.76 mmol) and CAS 1823050-70-9 (282 mg, 2.28 mmol) as starting materials.

¹H NMR (400 MHz, DMSO-d₆+D₂O) δ ppm: 8.28 (s, 1H), 7.84 (d, J=8.4 Hz, 1H), 7.57 (d, J=8.4 Hz, 1H), 3.57 (m, 2H), 3.32 (m, 3H), 2.86 (m, 2H), 2.41 (m, 1H), 2.31 (m, 1H), 1.29 (s, 3H), 1.26 (s, 3H).

MS m/z (+ESI): 398.1, 400.1 [M+H]⁺.

### Preparation of Example 121: (5R)-5-[[(4-amino-8-bromo-5,5-dimethyl-6H-benzo[h]quinazolin-7-yl)amino]methyl]oxazolidin-2-one

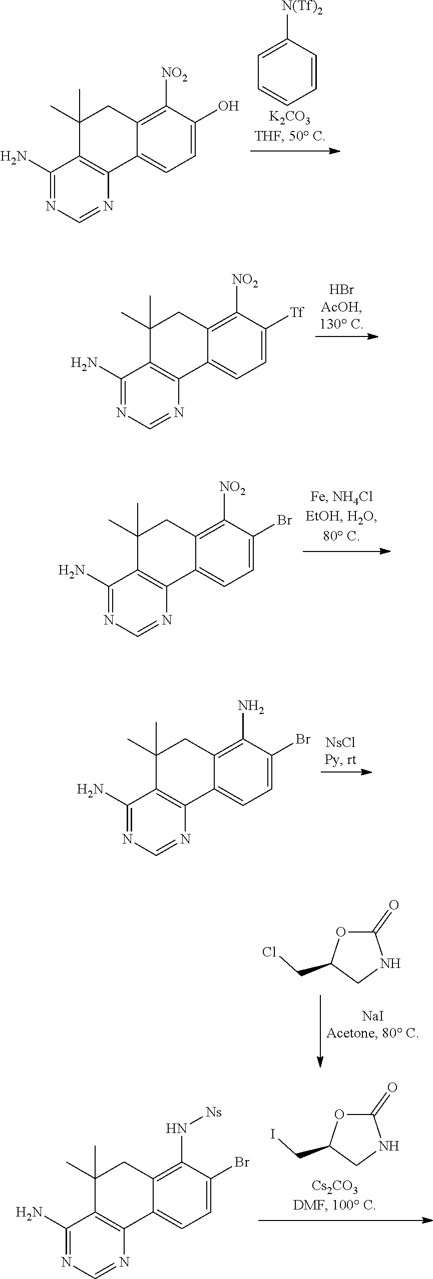

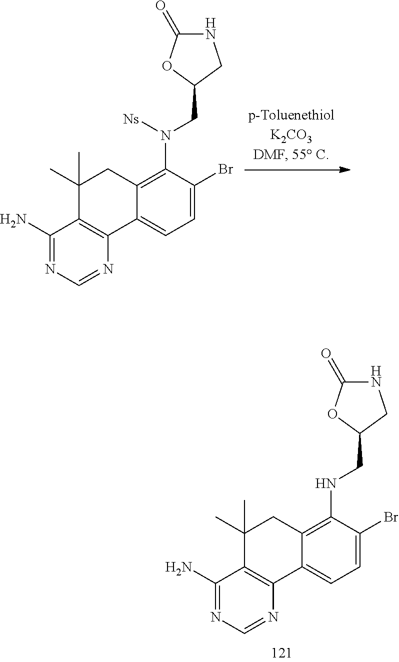

### Step 1: Preparation of (4-amino-5,5-dimethyl-7-nitro-6H-benzo[h]quinazolin-8-yl) trifluoromethanesulfonate

N-phenyl-bis(trifluoromethanesulfonimide) (7.73 g, 21.22 mmol) was added to a stirred solution of 4-amino-5,5-dimethyl-7-nitro-6H-benzo[h]quinazolin-8-ol (4.5 g, 14.15 mmol) in THF (45 mL), followed by K₂CO₃ (5.98 g, 42.44 mmol). After 2 h stirring at 50° C., the reaction mixture was filtered. The filtrate was concentrated and the residue was purified by combiflash to afford (4-amino-5,5-dimethyl-7-nitro-6H-benzo[h]quinazolin-8-yl) trifluoromethanesulfonate as a light yellow solid (3 g, 46% yield).

MS m/z (+ESI): 419.0 [M+H]⁺.

### Step 2: Preparation of 8-bromo-5,5-dimethyl-7-nitro-6H-benzo[h]quinazolin-4-amine

HBr (33% in AcOH, 50 mL) was added to a stirred suspension of (4-amino-5,5-dimethyl-7-nitro-6H-benzo[h]quinazolin-8-yl) trifluoromethanesulfonate (1.4 g, 3.01 mmol) in AcOH (25 mL). After 16 h stirring at 130° C., the reaction mixture was diluted with H₂O (150 mL). The resulting suspension was filtered, the cake was washed with H₂O and dried under high vacuum. The resulting off-white solid was poured in THF (300 mL) and a saturated NaHCO₃ aqueous solution (100 mL). After 10 minutes stirring, the organic layer was separated, washed with H₂O and brine, dried over Na₂SO₄, filtered and concentrated to afford 8-bromo-5,5-dimethyl-7-nitro-6H-benzo[h]quinazolin-4-amine as a yellow solid (950 mg, 72% yield) which was used in the next step without further purification.

¹H NMR (400 MHz, DMSO-d₆) δ ppm: 8.33 (s, 1H), 8.18 (d, J=8.8 Hz, 1H), 7.84 (d, J=8.8 Hz, 1H), 6.73 (br, 2H), 2.71 (s, 2H), 1.28 (s, 6H).

MS m/z (+ESI): 348.9, 350.9 [M+H]⁺.

### Step 3: Preparation of 8-bromo-5,5-dimethyl-6H-benzo[h]quinazoline-4,7-diamine

Iron (1.24 g, 21.77 mmol) was added to a stirred suspension of 8-bromo-5,5-dimethyl-7-nitro-6H-benzo[h]quinazolin-4-amine (950 mg, 2.18 mmol) in EtOH (80 mL) and H₂O (8 mL), followed by NH₄C1 (594 mg, 10.88 mmol). After 1 h stirring at 80° C., the reaction mixture was filtered. The filtrate was concentrated and the residue was poured in H₂O (60 mL). The resulting suspension was sonicated, filtered and the cake was washed with H₂O and dried under high vacuum to afford 8-bromo-5,5-dimethyl-6H-benzo[h]quinazoline-4,7-diamine as an off-white solid (700 mg, 91% yield) which was used in the next step without further purification.

¹H NMR (400 MHz, DMSO-d₆) δ ppm: 8.61 (s, 1H), 7.54 (d, J=8.4 Hz, 1H), 7.04 (d, J=8.4 Hz, 1H), 2.21 (s, 2H), 1.30 (s, 6H).

MS m/z (+ESI): 319.0, 321.0 [M+H]⁺.

### Step 4: Preparation of N-(4-amino-8-bromo-5,5-dimethyl-6H-benzo[h]quinazolin-7-yl)-4-nitro-benzenesulfonamide

NsCl (1.06 g, 4.66 mmol) was added to a stirred solution of 8-bromo-5,5-dimethyl-6H-benzo[h]quinazoline-4,7-diamine (700 mg, 1.86 mmol) in Py (12 mL). After 5 h stirring, the reaction mixture was poured into H₂O. The resulting mixture was concentrated and the residue was purified by combiflash to afford N-(4-amino-8-bromo-5,5-dimethyl-6H-benzo[h]quinazolin-7-yl)-4-nitro-benzenesulfonamide as a yellow solid (320 mg, 22% yield).

¹H NMR (400 MHz, DMSO-d₆) δ ppm: 10.36 (s, 1H), 8.41 (d, J=8.8 Hz, 2H), 8.29 (s, 1H), 8.99 (d, J=8.8 Hz, 2H), 7.94 (d, J=8.8 Hz, 1H), 7.58 (d, J=8.8 Hz, 1H), 6.57 (br, 2H), 2.70 (s, 2H), 1.18 (s, 6H).

MS m/z (+ESI): 504.0, 506.0 [M+H]⁺.

### Step 5: Preparation of (5S)-5-(iodomethyl)oxazolidin-2-one

In a sealed tube, NaI (637 mg, 4.20 mmol) was added to a stirred solution of (5S)-5-(chloromethyl)oxazolidin-2-one (200 mg, 1.40 mmol) in acetone (2 mL). After 16 h stirring at 80° C., the reaction mixture was concentrated and the residue was purified by combiflash to afford (5S)-5-(iodomethyl)oxazolidin-2-one as an off-white solid (120 mg, 34% yield).

¹H NMR (400 MHz, DMSO-d₆) δ ppm: 7.61 (s, 1H), 4.57 (m, 1H), 3.57 (t, J=8.8 Hz, 1H), 3.46 (m, 2H), 3.09 (m, 1H).

MS m/z (+ESI): 228.0 [M+H]⁺.

### Step 6: Preparation of N-(4-amino-8-bromo-5,5-dimethyl-6H-benzo[h]quinazolin-7-yl)-4-nitro-N-[[(5S)-2-oxooxazolidin-5-yl]methyl]benzenesulfonamide

(5S)-5-(iodomethyl)oxazolidin-2-one (4 g, 10.57 mmol) was added to a stirred solution of N-(4-amino-8-bromo-5,5-dimethyl-6H-benzo[h]quinazolin-7-yl)-4-nitro-benzenesulfonamide (300 mg, 0.53 mmol) in DMF (15 mL), followed by Cs₂CO₃ (1.78 g, 5.35 mmol). After 16 h stirring at 100° C., solvent was removed and the residue was extracted with EA and H₂O. The combined organic layers were washed with brine, dried over Na₂SO₄, filtered, concentrated and the residue was purified by combiflash to afford N-(4-amino-8-bromo-5,5-dimethyl-6H-benzo[h]quinazolin-7-yl)-4-nitro-N-[[(5S)-2-oxooxazolidin-5-yl]methyl]benzenesulfonamide as a yellow solid (290 mg, 81% yield).

¹H NMR (400 MHz, DMSO-d₆) δ ppm: 8.40 (d, J=8.8 Hz, 2H), 8.32 (s, 1H), 8.13 (d, J=8.8 Hz, 2H), 8.06 (d, J=8.8 Hz, 1H), 7.61 (m, 2H), 6.62 (s, 2H), 4.89 (m, 1H), 3.82 (m, 1H), 3.60 (m, 1H), 3.47 (m, 1H), 2.98 (m, 1H), 2.90 (m, 1H), 2.67 (m, 1H), 1.36 (s, 3H), 1.29 (s, 3H).

MS m/z (+ESI): 603.1, 605.1 [M+H]⁺.

### Step 7: Preparation of (5R)-5-[[(4-amino-8-bromo-5,5-dimethyl-6H-benzo[h]quinazolin-7-yl)amino]methyl]oxazolidin-2-one

p-Toluenethiol (157 mg, 1.25 mmol) was added to a stirred solution of N-(4-amino-8-bromo-5,5-dimethyl-6H-benzo[h]quinazolin-7-yl)-4-nitro-N-[[(5S)-2-oxooxazolidin-5-yl]methyl]benzenesulfonamide (280 mg, 0.42 mmol) in DMF (6 mL), followed by K₂CO₃ (177 mg, 1.25 mmol). After 2 h stirring at 50° C., solvent was removed and the crude was purified by preparative HPLC to afford (5R)-5-[[(4-amino-8-bromo-5,5-dimethyl-6H-benzo[h]quinazolin-7-yl)amino]methyl]oxazolidin-2-one as a white solid (32 mg, 18% yield).

¹H NMR (400 MHz, DMSO-d₆+D₂O) δ ppm: 8.27 (s, 1H), 7.62 (d, J=8.4 Hz, 1H), 7.50 (d, J=8.4 Hz, 1H), 4.67 (m, 1H), 3.50 (m, 2H), 3.18 (m, 2H), 2.83 (s, 2H), 1.28 (s, 3H), 1.26 (s, 3H).

MS m/z (+ESI): 418.2, 420.2 [M+H]⁺.

### Preparation of Example 122: N7-[[5-(1-fluoroethyl)-1,3,4-oxadiazol-2-yl]methyl]-8-methoxy-5,5-dimethyl-6H-benzo[h]quinazoline-4,7-diamine

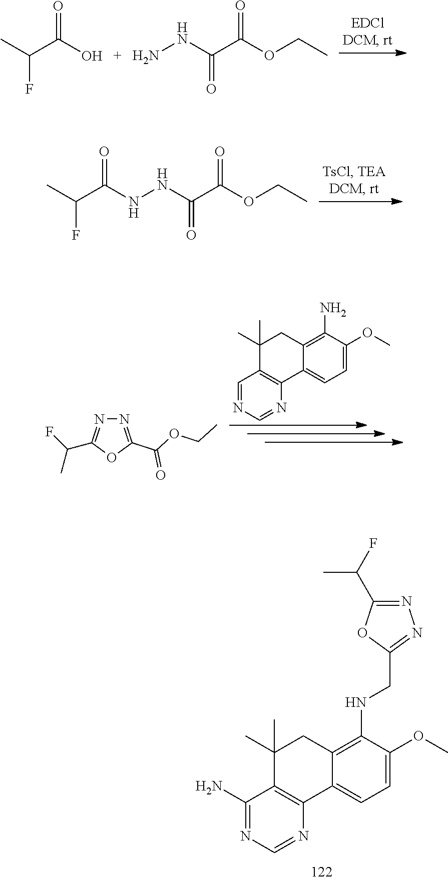

### Step 1: Preparation of ethyl 2-[2-(2-fluoropropanoyl)hydrazino]-2-oxo-acetate

EDCI (10.44 g, 53.93 mmol) was added to a stirred solution of 2-fluoropropanoic acid (3.48 g, 35.95 mmol) and ethyl 2-hydrazino-2-oxo-acetate (5 g, 35.95 mmol) in DCM (50 mL). After 16 h stirring, the reaction mixture was concentrated and the residue was purified by column chromatography (silica gel; DCM:MeOH; 10:0 to 10:1; v:v) to afford ethyl 2-[2-(2-fluoropropanoyl)hydrazino]-2-oxo-acetate as a colorless oil (6.5 g, 79% yield).

¹H NMR (400 MHz, CDCl₃) δ ppm: 9.37 (s, 1H), 8.76 (s, 1H), 5.18 (dq, J=6.8, 48.4 Hz, 1H), 4.41 (q, J=7.2 Hz, 2H), 1.66 (dd, J=6.8, 25.2 Hz, 3H), 1.41 (t, J=7.2 Hz, 3H).

### Step 2: Preparation of ethyl 5-(1-fluoroethyl)-1,3,4-oxadiazole-2-carboxylate

TsCl (6.56 g, 34.05 mmol) was added to a stirred solution of ethyl 2-[2-(2-fluoropropanoyl)hydrazino]-2-oxo-acetate (6.5 g, 28.38 mmol) in DCM (65 mL), followed by TEA (4.78 mL, 34.05 mmol). After 16 h stirring, the reaction mixture was concentrated and the residue was purified by column chromatography (silica gel; DCM:MeOH; 10:0 to 10:1; v:v) to afford ethyl 5-(1-fluoroethyl)-1,3,4-oxadiazole-2-carboxylate as a colorless oil (3.5 g, 59% yield).

¹H NMR (400 MHz, CDCl₃) δ ppm: 5.84 (m, 1H), 4.55 (q, J=7.2 Hz, 2H), 1.88 (m, 3H), 1.48 (t, J=7.2 Hz, 3H).

### Step 3: Preparation of N7-[[5-(1-fluoroethyl)-1,3,4-oxadiazol-2-yl]methyl]-8-methoxy-5,5-dimethyl-6H-benzo[h]quinazoline-4,7-diamine

The title compound was prepared as an off-white solid following Scheme 1 and in analogy to Examples 5, 26 and 48 using 8-methoxy-5,5-dimethyl-6H-benzo[h]quinazoline-4,7-diamine, ethyl 5-(1-fluoroethyl)-1,3,4-oxadiazole-2-carboxylate and CAS 98-74-8 as starting materials.

¹H NMR (400 MHz, DMSO-d₆+D₂O) δ ppm: 8.21 (s, 1H), 7.69 (d, J=8.4 Hz, 1H), 6.87 (d, J=8.4 Hz, 1H), 5.90 (dq, J=6.4, 47.2 Hz, 1H), 4.43 (s, 2H), 3.73 (s, 3H), 2.72 (s, 2H), 1.63 (dd, J=6.4, 24.0 Hz, 3H), 1.23 (s, 6H).

MS m/z (+ESI): 399.3 [M+H]⁺.

### BIOLOGICAL EXAMPLES

### CLK3 Kinase Assay

CLK3 kinase activity was measured using the LANCE® Ultra technology (PerkinElmer) essentially as recommended by the supplier. Briefly, 2.5 μL of test sample at 4× the desired final concentration in 4% DMSO were dispensed into white, low volume, round bottom, 384 well plates (Corning, cat. no. 4512). 5 μL of a mixture containing recombinant human CLK3 (Thermo Fisher Scientific, cat. no. PV3826) and the labeled peptide substrate ULight™-CREBtide (PerkinElmer, cat. no. TRF0107) prepared in 1.33× kinase buffer (Thermo Fisher Scientific, cat. no. PV3189) were then added. Finally, the reactions were started by adding 2.5 μL of a 4× solution of ATP in kinase buffer. The plates were sealed with adhesive foils and incubated for 3 hours at 30° C. in the dark. The final concentrations of CLK3, ULight™-CREBtide, and ATP were 0.5 nM, 50 nM, and 300 μM, respectively. 5 μL of 40 mM EDTA were then added to terminate the reactions. The Eu-anti-phospho-CREB antibody (PerkinElmer, cat. no. TRF0200) diluted in LANCE® detection buffer (PerkinElmer, cat. no. CR97) was then added to a final concentration of 2 nM and the reactions were incubated for 1 hour at room temperature in the dark. The signals were measured using a Synergy 4 reader (BioTek) with a 320/80 nm filter for the excitation and 620/10 nm and 665/8 nm filters for the donor and acceptor emission, respectively. Relative inhibition values were calculated by normalizing the raw data using solvent control wells (0% inhibition) and wells that received no ATP (100% inhibition). IC₅₀ values were calculated by fitting the concentration-response data to a sigmoidal 4-parameter logistic model. Results are shown in Table 2 below. IC50 values for CLK3 are determined from duplicate measurements at ten different concentrations (serial 1:3 dilutions).

### MDA-MB-231 and CAL-51 Proliferation Assay

Cells (MDA-MB-231 (American Type Culture Collection (ATCC), #HTB-26) or CAL-51 (Deutsche Sammlung von Mikroorganismen und Zellkulturen (DSMZ), #ACC302) as applicable) were cultivated in DMEM High Glucose (4.5 g/L) with L-Glutamine (BioConcept, cat. no. 1-26F03-I) supplemented with 1% Na-pyruvate (Sigma, cat. no. S8636), 1% Penicillin-Streptomycin (BioConcept, cat. no. 4-01F00H), and 10% FBS (Sigma, cat. no. F9665) using standard techniques. Cells were seeded in black, clear-bottom 384 well plates for tissue culture (Greiner Bio-One, cat. no. 781091) at a density of 750 cells per well in 20 μL medium and the plates were incubated overnight at 37° C. with 5% CO₂ prior to treatment. Experimental compounds were serially diluted in DMSO to 200× the desired final concentrations. The solutions were then diluted 1:40 with culture medium and 5 μL aliquots were mixed into the wells containing the cells. The final concentration of DMSO was 0.5%. The plates were incubated for 72 hours and cell numbers were then measured using YO-PRO™-1 Iodide (ThermoFisher Scientific, cat. no. Y3603) essentially as recommended by the manufacturer. Briefly, a 12.5 μM solution of YO-PRO™_1 Iodide in 5× YO—PRO buffer (100 mM Na-citrate, 130 mM NaCl, pH 4) was combined with an equal volume of lysis buffer (0.6% NP-40, 30 mM EDTA, 30 mM EGTA, 0.33× YO—PRO buffer) and 12.5 μL of the mixture were dispensed into each well. The plates were incubated at room temperature in the dark for 30 minutes. Fluorescence intensity signals were then measured on a Synergy 4 reader (BioTek) using 485/20 nm and 530/25 nm filters for the excitation and emission, respectively. Relative proliferation values were calculated by normalizing the raw data using DMSO-treated cells (100% proliferation) and the signals obtained from cells that were evaluated at the time of compound addition (0% proliferation). The concentration-response data were fitted to a sigmoidal 4-parameter logistic model with the maximum constrained to 100%. GI₅₀ values were calculated from the curves as the compound concentrations reducing proliferation to 50%. Results are shown in Table 2 below:

TABLE 2
CLK3 biochemical results and growth inhibition
of MDA-MB-231 and CAL-51 cells (nM)

| Example | *CLK3 IC₅₀ | **MDA-MB-231 GI₅₀ | ***CAL-51 GI₅₀ |
|---|---|---|---|
| 1 | +++ | + | ++ |
| 2 | ++ | ++ | ++ |
| 3 | + | + | ++ |
| 4 | ++ | ++ | ++ |
| 5 | +++ | ++ | ++ |
| 6 | + | + | ++ |
| 7 | +++ | ++ | ++ |
| 8 | ++ | ++ | +++ |
| 9 | +++ | ++ | +++ |
| 10 | ++ | ++ | ++ |
| 11 | +++ | + | ++ |
| 12 | +++ | +++ | +++ |
| 13 | ++ | ++ | +++ |
| 14 | +++ | ++ | ++ |
| 15 | +++ | ++ | +++ |
| 16 | +++ | +++ | +++ |
| 17 | +++ | +++ | +++ |
| 18 | + | ++ | ++ |
| 19 | ++ | ++ | ++ |
| 20 | +++ | +++ | +++ |
| 21 | ++ | ++ | ++ |
| 22 | +++ | ++ | +++ |
| 23 | +++ | ++ | ++ |
| 24 | +++ | +++ | +++ |
| 25 | + | ++ | ++ |
| 26 | + | + | + |
| 27 | ++ | ++ | ++ |
| 28 | + | ++ | ++ |
| 29 | + | ++ | ++ |
| 30 | +++ | +++ | +++ |
| 31 | ++ | ++ | +++ |
| 32 | +++ | + | + |
| 33 | +++ | ++ | +++ |
| 34 | + | ++ | ++ |
| 35 | ++ | ++ | ++ |
| 36 | + | ++ | ++ |
| 37 | ++ | ++ | +++ |
| 38 | ++ | + | ++ |
| 39 | + | + | ++ |
| 40 | ++ | ++ | +++ |
| 41 | + | + | + |
| 42 | ++ | + | + |
| 43 | ++ | ++ | ++ |
| 44 | +++ | ++ | ++ |
| 45 | + | + | + |
| 46 | +++ | +++ | +++ |
| 47 | ++ | ++ | +++ |
| 48 | +++ | +++ | +++ |
| 49 | +++ | +++ | +++ |
| 50 | +++ | +++ | +++ |
| 51 | +++ | +++ | +++ |
| 52 | +++ | +++ | +++ |
| 53 | +++ | +++ | +++ |
| 54 | +++ | +++ | +++ |
| 55 | ++ | +++ | +++ |
| 56 | + | + | + |
| 57 | ++ | ++ | +++ |
| 58 | +++ | ++ | +++ |
| 59 | +++ | ++ | +++ |
| 60 | ++ | ++ | ++ |
| 61 | + | ++ | ++ |
| 62 | + | ++ | ++ |
| 63 | ++ | ++ | ++ |
| 64 | +++ | +++ | +++ |
| 65 | ++ | ++ | ++ |
| 66 | +++ | +++ | +++ |
| 67 | +++ | +++ | +++ |
| 68 | ++ | ++ | +++ |
| 69 | +++ | + | + |
| 70 | +++ | ++ | ++ |
| 71 | +++ | ++ | +++ |
| 72 | + | ++ | ++ |
| 73 | ++ | +++ | +++ |
| 74 | +++ | ++ | +++ |
| 75 | +++ | ++ | +++ |
| 76 | + | ++ | ++ |
| 77 | +++ | + | ++ |
| 78 | +++ | +++ | +++ |
| 79 | ++ | + | + |
| 80 | +++ | ++ | ++ |
| 81 | +++ | ++ | +++ |
| 82 | +++ | ++ | +++ |
| 83 | +++ | +++ | +++ |
| 84 | ++ | ++ | +++ |
| 85 | ++ | + | + |
| 86 | +++ | + | ++ |
| 87 | ++ | ++ | ++ |
| 88 | ++ | + | ++ |
| 89 | + | ++ | ++ |
| 90 | +++ | ++ | ++ |
| 91 | +++ | +++ | +++ |
| 92 | +++ | +++ | +++ |
| 93 | +++ | +++ | +++ |
| 94 | + | + | ++ |
| 95 | +++ | +++ | +++ |
| 96 | +++ | +++ | +++ |
| 97 | +++ | +++ | +++ |
| 98 | +++ | ++ | +++ |
| 99 | +++ | +++ | +++ |
| 100 | + | ++ | ++ |
| 101 | + | ++ | ++ |
| 102 | +++ | ++ | +++ |
| 103 | ++ | ++ | ++ |
| 104 | + | + | ++ |
| 105 | + | ++ | ++ |
| 106 | ++ | ++ | ++ |
| 107 | + | ++ | ++ |
| 108 | +++ | ++ | +++ |
| 109 | ++ | ++ | +++ |
| 110 | +++ | ++ | +++ |
| 111 | + | ++ | ++ |
| 112 | ++ | + | ++ |
| 113 | ++ | ++ | ++ |
| 114 | +++ | +++ | +++ |
| 115 | +++ | ++ | +++ |
| 116 | ++ | ++ | ++ |
| 117 | ++ | ++ | +++ |
| 118 | +++ | +++ | +++ |
| 119 | +++ | ++ | ++ |
| 120 | + | ++ | ++ |
| 121 | +++ | +++ | +++ |
| 122 | + | + | ++ |
| 123 | +++ | +++ | +++ |
| 124 | + | ++ | ++ |
| 125 | +++ | +++ | +++ |
| 126 | ++ | ++ | ++ |
| 127 | ++ | ++ | ++ |
| 128 | +++ | + | + |
| 129 | +++ | + | + |
| 130 | +++ | + | ++ |
| 131 | +++ | ++ | ++ |
| 132 | +++ | ++ | ++ |

*For CLK3 IC₅₀: +++ when <30, ++ when 30-100, + when 101-1000 (nM)
**For MDA-MB-231 IC₅₀: +++ when <100, ++ when 100-2000, + when >2000 (nM)
***For CAL-51 IC₅₀: +++ when <100, ++ when 100-2000, + when >2000 (nM)

### Assessment of Inhibition of Phospho-SRSF6 Levels in MDA-MB-468 Cells

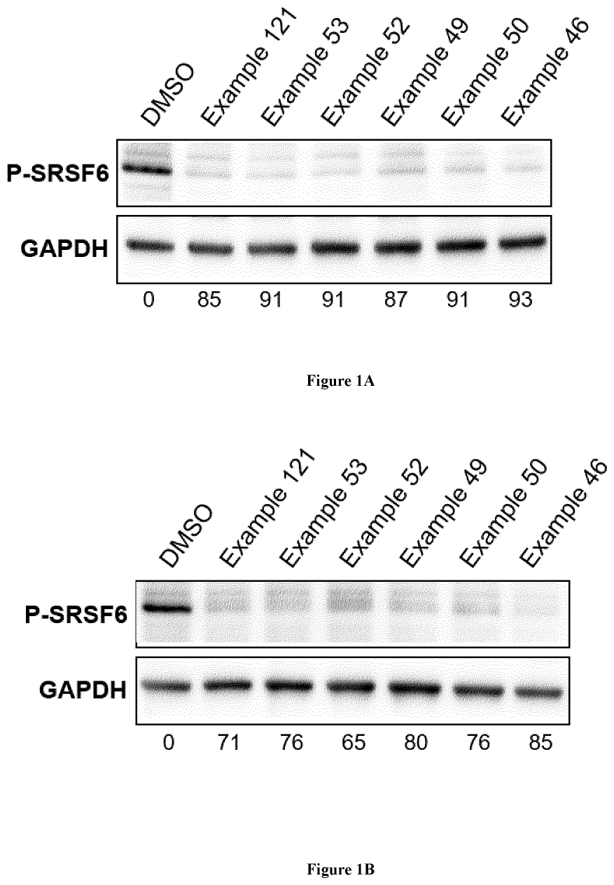

MDA-MB-468 cells (ATCC, HTB-132) were cultured in DMEM (BioConcept cat. no. 1-26F03) supplemented with 10% FBS (Sigma cat. no. F9665), 100 Na-pyruvate (Sigma cat. no. S8636), 1% Penicillin-Streptomycin (BioConcept cat. no. 4-01FOO-H). When cells reached a density of 70% o in 6-well plates (TPP cat. no. 92006), they were treated either with the test compound vehicle DMSO, or with experimental compounds at a final concentration of either 10 or 100 nM for 2 hours. Thereafter, the culture medium was removed and cells were collected by scraping in Lysis Buffer (20 mM Tris HCl pH7.5, 150 mM NaCl, 1 mM EGTA, 1 mM EDTA, 1% o Triton and 1% o NP-40) containing protease and phosphatase inhibitors (Halt™ Protease&Phosphatase inhibitor cocktail 100× ThermioScientific cat. no. 1861281) and 1 mM PMSF (Fluka cat. no. 93482). Cleared lysates were stored in Eppendorfftubes at −80° C. until use, and protein concentration in lysates was determined using Pierce 660 nm Protein Assay Reagent (ThermioScientific cat. no. 22660). 20 μg proteins were diluted in 4× Laemmli buffer (Biorad cat. no. 161-0747) containing 10% beta-mercaptoethanol, separated by SDS-PAGE using 10% gels, and then transferred to PVDF membranes (Trans Blot Turbo Transfer Pack BioRad cat. no. 1704156) by Trans-Blot® Turbo™ System semidry blotting methodology (BioRad). Primary antibodies were incubated in TBS-0.1% Tween-20 buffer containing 3% BSA Bovine Albumin Fr.V (Millipore cat. no. 81-053-3) overnight at 4° C. The following primary antibodies were used: Mouse anti-phosphoepitope SR proteins clone 1H4 (Millipore cat. no. MABE50) diluted 1:5000, and rabbit anti-GAPDH clone 14C10 (Cell Signaling cat. no. 2118S) to control for equal protein loading, diluted 1:2000. HRP-conjugated secondary antibodies were incubated one hour at room temperature diluted 1:5000 in TBS-0.1% Tween-20 buffer containing 500 nonfat-dried bovine milk (Sigma cat. no. M7409). The following secondary antibodies were used: Goat anti-mouse-HRP (Jackson cat. no. 115-035-146) and goat anti-rabbit-HRP (Jackson cat. no. 111-035-144). Membranes were incubated with ECL Prime Western Blot Detection Reagent (Amersham cat. no. RPN2236) to detect specific signals using Fusion SOLO S device (Vilber Lourmat). Pixel intensity of the phopsho-SRSF6 (MW 55 kDa in reference to markers) and GAPDH (MW 37 kDa) signals in the Western blots were determined using Evolution-Capt image analysis software (v17.01, Vilber Lourmat). DMSO control was used to set the phospho-SRSF6 signal at baseline, phospho-SRSF6 signals for each sample were normalized by the respective GAPDH signals and percent intensity of P—SRSF6 signal at 10 and 100 nM compound compared to DMSO control was calculated (see FIG. 1).

Several examples of the invention were tested for inhibition of phospho-SRSF6 levels in MDA-MB-468 cells and showed an inhibition level of more than 50% compared to DMSO control when tested at a concentration of either 100 or 10 nM. Results are shown in Table 3 below.

TABLE 3
Inhibition of phospho-SRSF6 levels in MDA-MB-468 cells.

| | >50% p-SRSF6 | >50% p-SRSF6 |
|---|---|---|
| | reduction | reduction |
| Example | at 100 nM | at 10 nM |
| 7 | + | + |
| 8 | + | − |
| 12 | + | − |
| 13 | + | − |
| 17 | + | − |
| 20 | + | + |
| 16 | + | + |
| 30 | + | + |
| 37 | + | + |
| 40 | + | + |
| 46 | + | + |

+ designates that criteria was met; − designates that criteria was not met.

### Mouse Xenograft Models

The efficacy of Example 13 was assessed in a mouse xenograft model. To this end, the SW480 colorectal cancer xenograft model was used, in which the tumor cells were injected subcutaneously into the flanks of immune-deficient NOD/SCID mice (lack functional/mature T and B cells, functional immature macrophages and residual NK cell function). SW480 cells (ATCC, #CCL-228) were cultured in RPMI high glucose medium supplemented with 10% heat-inactivated fetal bovine serum at 37° C. SW480 cells were harvested when they were growing exponentially.

Female 8-9 weeks of age NOD/SCID mice of about 23 g of weight were used for the study (Jiangsu GemPharmatech Co., Ltd). Mice were inoculated subcutaneously in the flank region with 1×10⁷ SW480 cells in PBS mixed with Matrigel™ (1:1) in a final injection volume of 0.2 mL/mouse. Once tumors reached an average size of 100-150 mm³, a pair match was carried out to distribute mice to the different treatment groups. The mice in the control group were administered the vehicle (20% propylene glycol; 79.2% 10 mM citric acid; 0.8% Tween™ 80) twice a day and twice a week (BID*BIW) for 3 weeks in an application volume of 10 mL/kg. The solution of Example 13 was administered orally in an application volume of 10 mL/kg BID*BIW at 45 mg/kg on days 1-10 and 50 mg/kg on days 11-22 (doses represent free base equivalent).

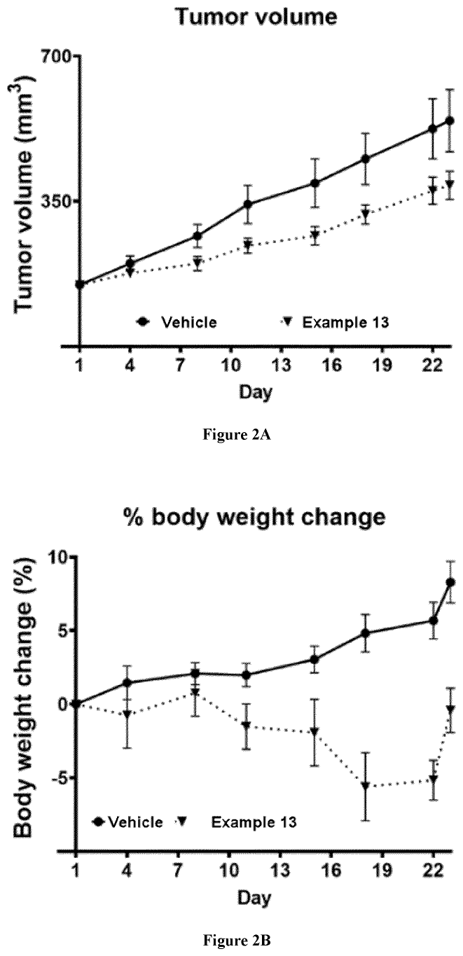

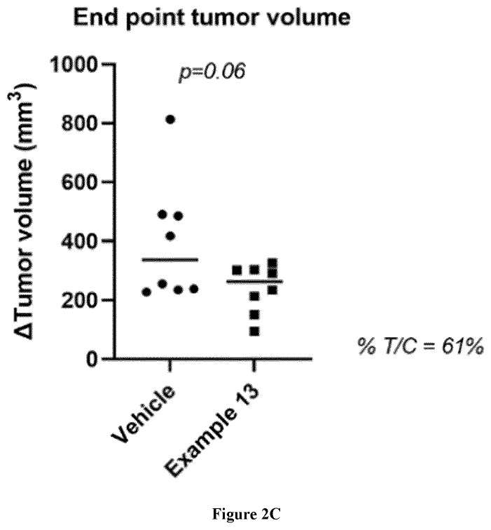

Tumor volumes (expressed in mm³) were measured in two dimensions using a caliper on days 1, 4, 8, 11, 15, 18, 22 and 23 of the treatment period. FIGS. 2A and 2B depict the tumor growth and body weight changes during the treatment period. Results represent the mean±SEM (n=8). FIG. 2C represents the tumor volume changes at the conclusion of the study. Tumor volume changes between vehicle and Example 13 treatment groups were compared by a 2-tailed t-test using GraphPad™ Prism version 9. During the treatment period tumor volumes of mice treated with Example 13 at 45 mg/kg twice a day and twice weekly (BID*BIW) were consistently smaller as compared with tumor volumes of vehicle-treated animals (FIGS. 2A and 2B). At the study endpoint (day 23), tumor volumes treated with Example 13 were 61% of the volume of the vehicle-treated group, (FIG. 2C); p=0.06. The treatment with Example 13 was tolerated with a maximal body weight loss of 5% on day-17 (FIG. 2B).

1. A compound of formula (I) 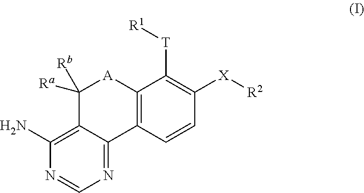
or a pharmaceutically acceptable salt thereof; wherein
Ra and Rb are both —CH₃ or Ra and Rb together form a —CH₂—CH₂—CH₂— or a —CH₂—CH₂—CH₂—CH₂-bridging moiety;
A is —CH₂— or —C(═O)—;
T is —N(R11)-, —N(R11)-C(═O)—, —N(R11)-S(O₂)—, —O—, —C(R12)(R13)-, —C═C(R12)₂, —S—, —S(O)—, —S(O₂)—, —S(O₂)—N(R14)- or —C(═O)—N(R14)-;
R11, when not forming a ring with R1 or R2, is hydrogen, C1-C4alkylene-R11a, C1-C4haloalkylene-R11a or C3-C6cycloalkyl, wherein the cycloalkyl is optionally substituted by one or two R11b;
each R12, when not forming a ring with R2, is independently hydrogen, C1-C4alkylene-R12a, C1-C4haloalkylene-R12a or C3-C6cycloalkyl, wherein the cycloalkyl is optionally substituted by one or two R12b;
R14, when not forming a ring with R1 or R2, is hydrogen, C1-C4alkylene-R14a, C1-C4haloalkylene-R14a or C3-C6cycloalkyl, wherein the cycloalkyl is optionally substituted by one or two R14b;
each R11a, R12a and R14a are independently hydrogen, halogen, —OH, —CN, —NH₂, —O—C1-C2alkyl, —O—C1-C2haloalkyl, —NH(C1-C2alkyl) or —N(C1-C2alkyl)₂;
each R11b, R12b and R14b are independently halogen, —OH, —CN, —NH₂, —O—C1-C2alkyl, —O—C1-C2haloalkyl, —NH(C1-C2alkyl) or —N(C1-C2alkyl)₂;
R13 is hydrogen, C1-C4alkyl or C1-C4haloalkyl;
R2 and R11, R2 and R12 or R2 and R14 may together form a partially unsaturated 6- to 7-membered heterocyclic ring containing no additional heteroatoms, wherein the heterocyclic ring is optionally substituted by one or two R2a;
each R2a is independently halogen, —OH, —CN, —NH₂, —O—C1-C2alkyl, —O—C1-C2haloalkyl, —NH(C1-C2alkyl) or —N(C1-C2alkyl)₂;
R1 and R11 or R1 and R14 may together form a saturated or partially unsaturated 4- to 7-membered heterocyclic ring optionally containing one or two additional heteroatoms as ring members selected from N, O and S, wherein the heterocyclic ring is optionally substituted by one to three R1a, or R1 and R11 or R1 and R14 may together form a 5- to 6-membered heteroaryl ring optionally containing one to three additional heteroatoms as ring members selected from N and O, wherein the heteroaryl ring is optionally substituted by one to three R1b;
each R1a is independently halogen, —OH, —CN, —NH₂, C1-C4alkylene-R1c, C1-C4haloalkylene-R1c, —O—C1-C4alkyl, —O—C1-C4haloalkyl, —NH(C1-C4alkyl), —N(C1-C4alkyl)₂, —C(═O)NH₂, —C(═O)NH(C1-C4alkyl), —C(═O)N(C1-C4alkyl)₂ or oxo;
each R1b is independently halogen, —OH, —CN, —NH₂, C1-C4alkylene-R1c, C1-C4haloalkylene-R1c, —O—C1-C4alkyl, —O—C1-C4haloalkyl, —NH(C1-C4alkyl) or —N(C1-C4alkyl)₂, —C(═O)NH₂, —C(═O)NH(C1-C4alkyl) or —C(═O)N(C1-C4alkyl)₂;
each R1c is independently hydrogen, halogen, —OH, —CN, —NH₂, —O—C1-C2alkyl, —O—C1-C2haloalkyl, —NH(C1-C2alkyl) or —N(C1-C2alkyl)₂;
R1, when not forming a ring with R11 or R14, is hydrogen or —Y—R3;
Y is a bond or C1-C6alkylene wherein one non-terminal —CH₂— moiety may be replaced by —O— and wherein the alkylene moiety may be substituted by one or two moieties independently selected from —OH, —O—C1-C4alkyl and —O—C1-C4haloalkyl and wherein the alkylene moiety may include a 3- to 5-membered saturated or partially unsaturated carbocyclic ring as part of the moiety;
R3 is hydrogen, —CN, —OH, C1-C4haloalkyl, C2-C4alkenylene-R4, C2-C4alkynylene-R4, —O—C1-C4alkylene-R4, —O—C1-C4haloalkyl, —NH₂, —NH(C1-C4alkylene-R4), —N(C1-C4alkylene-R4)₂, —C(═O)—OH, —C(═O)—O—C1-C4alkylene-R4, —C(═O)—NH₂, —C(═O)—NH(C1-C4alkylene-R4), —C(═O)—N(C1-C4alkylene-R4)₂, —NH—C(═O)—C1-C4alkylene-R4, —N(C1-C4alkyl)-C(═O)—C1-C4alkylene-R4, Cycle P, Cycle Q, —O—Cycle P, —O—Cycle Q, —NH—S(O₂)—C1-C4alkylene-R4, —N(C1-C4alkyl)-S(O₂)—C1-C4alkylene-R4, —S—C1-C4alkylene-R4 or —S(O₂)—C1-C4alkyl-R4, providing that the atom in R3 connected to Y is not a heteroatom when (i) Y is a bond and (ii) T is not —N(R11)-, —C(R12)(R13)- or —C═C(R12)₂;
R4 is hydrogen, halogen, —NH₂, —NH(C1-C2alkyl) or —N(C1-C2alkyl)₂;
Cycle P is a 3- to 6-membered saturated or partially unsaturated carbocyclic ring optionally substituted by one to three R5 or is a 3- to 6-membered saturated or partially unsaturated heterocyclic ring containing one or two heteroatoms selected from N and O optionally substituted by one to three R5;
each R5 is independently halogen, —NH₂, —OH, —CN, C1-C4alkylene-R5a, —O—C1-C4alkylene-R5a or oxo;
each R5a is independently hydrogen, halogen, —NH₂, —OH, —CN or —O—C1-C2alkyl;
Cycle Q is phenyl optionally substituted by one to three R6, or is a 5- to 6-membered heteroaryl ring containing one or two heteroatoms selected from N, S and O optionally substituted by one to three R6;
each R6 is independently halogen, —NH₂, —OH, —CN, C1-C4alkylene-R6a or —O—C1-C4alkylene-R6a;
each R6a is independently hydrogen, halogen, —NH₂, —OH, —CN or —O—C1-C2alkyl;
X is —O—, —C(═O)—, —C(═CH₂)—, —N(R10a)- or —CH(R10b)- and wherein X is a bond when R2 is halogen or —CN and wherein when X is —C(═O)— then R2 is not hydrogen, or wherein X—R2 is hydrogen;
R10a, when not forming a ring with R2, is hydrogen or C1-C4alkyl;
R10b is hydrogen or C1-C4alkyl;
R2 and R10a may together form a 4- to 7-membered saturated or partially unsaturated heterocyclic ring optionally containing an —N(R9)- moiety as ring member in addition to the nitrogen atom of X and otherwise containing only carbon atoms as ring members optionally substituted by one or two R8;
R2 and R10b may together form a 4- to 7-membered saturated or partially unsaturated carbocyclic ring or a 4- to 7-membered saturated or partially unsaturated heterocyclic ring containing an —N(R9)- moiety as ring member and otherwise containing only carbon atoms as ring members optionally substituted by one or two R8;
R2, when not forming a ring with R10a, R10b, R11, R12 or R14, is hydrogen, halogen, —CN, C1-C4alkyl optionally substituted by one or two R7, or is a 4- to 7-membered saturated or partially unsaturated carbocyclic ring optionally substituted by one or two R8, or is a 4- to 7-membered saturated or partially unsaturated heterocyclic ring containing one —N(R9)- moiety as a ring member and otherwise containing only carbon atoms as ring members;
each R7 is independently —CN, —OH, halogen, —N(R7a)R7b or —NH(—C(═O)—C1-C4alkyl);
each R7a and R7b, when not together forming a ring, is independently hydrogen, C1-C4alkylene-R7c or C1-C4haloalkylene-R7c;
each R7c is independently hydrogen, halogen, —O—C1-C2alkyl or —O—C1-C2haloalkyl;
R7a and R7b may together form a 4- to 7-membered saturated or partially unsaturated heterocyclic ring optionally containing one or two additional heteroatoms as ring members selected from N, O and S, and wherein the heterocyclic ring is optionally substituted by one or two R7d;
each R7d is independently halogen, —OH, —CN, —NH₂, C1-C2alkyl, C1-C2haloalkyl, —O—C1-C2alkyl, —O—C1-C2haloalkyl, —NH(C1-C2alkyl) or —N(C1-C2alkyl)₂;
each R8 is independently —CN, —OH, halogen, —N(R8a)R8b, —NH(—C(═O)—C1-C4alkyl), C1-C4alkyl or C1-C4haloalkyl;
each R8a and R8b, when not together forming a ring, is independently hydrogen, C1-C4alkylene-R8c or C1-C4haloalkylene-R8c;
R8c is hydrogen, halogen, —O—C1-C2alkyl or —O—C1-C2haloalkyl;
R8a and R8b may together form a 4- to 7-membered saturated or partially unsaturated heterocyclic ring optionally containing one or two additional heteroatoms as ring members selected from N, O and S, and wherein the heterocyclic ring is optionally substituted by one or two R8d;
each R8d is independently halogen, —OH, —CN, —NH₂, C1-C2alkyl, C1-C2haloalkyl, —O—C1-C2alkyl, —O—C1-C2haloalkyl, —NH(C1-C2alkyl) or —N(C1-C2alkyl)₂;
R9 is hydrogen, C1-C4alkylene-R9a, C1-C4haloalkylene-R9a or —C(═O)—C1-C4alkyl; and
R9a is hydrogen, halogen, —O—C1-C2alkyl or —O—C1-C2haloalkyl.

2. The compound of claim 1 or a pharmaceutically acceptable salt thereof, wherein Ra and Rb are both —CH₃ and A is —CH₂—.

3. The compound of claim 1 or claim 2 or a pharmaceutically acceptable salt thereof, wherein T is —N(R11)-, —N(R11)-S(O₂)—, —O—, —C(R12)(R13)-, —C═C(R12)₂, —S— or —C(═O)—N(R14)-.

4. The compound of any one of claim 1 to 3 or a pharmaceutically acceptable salt thereof, wherein the substituent R1 is not hydrogen when T is —N(R11), —O—, —C(R12)(R13)-, —S—, —S(O)— or —S(O₂)—.

5. The compound of any one of claim 1 to 3 or a pharmaceutically acceptable salt thereof, wherein the substituent R1 is not hydrogen.

6. The compound of any one of claim 1 to 5 or a pharmaceutically acceptable salt thereof, wherein the substituent R1 contains at least 1 atom other than hydrogen.

7. The compound of any one of claim 1 to 6 or a pharmaceutically acceptable salt thereof, wherein X is —O—, —C(═O)—, —N(R10a)- or —CH(R10b)- and wherein X is a bond when R2 is halogen or —CN and wherein when X is —C(═O)— then R2 is not hydrogen, or wherein X—R2 is hydrogen.

8. The compound of any one of claim 1 to 7 or a pharmaceutically acceptable salt thereof, wherein R2 and R11, R2 and R12, R2 and R14, R1 and R11, and R1 and R14 do not together form a heterocyclic ring.

9. The compound of any one of claims 1 to 7 or a pharmaceutically acceptable salt thereof, wherein T is —N(R11)-, —N(R11)-C(═O)— or —N(R11)-S(O₂)— and R1 and R11 together form a saturated or partially unsaturated 4- to 7-membered heterocyclic ring optionally containing one or two additional heteroatoms as ring members selected from N, O and S, wherein the heterocyclic ring is optionally substituted by one to three R1a, or R1 and R11 together form a 5- to 6-membered heteroaryl ring optionally containing one to three additional heteroatoms as ring members selected from N and O, wherein the heteroaryl ring is optionally substituted by one to three R1b.

10. The compound of any one of claims 1 to 8 or a pharmaceutically acceptable salt thereof, wherein R1, when not forming a ring with R11 or R14, is hydrogen or C1-C6alkylene-R3, wherein the alkylene moiety may be substituted by one —OH, wherein the alkylene moiety may include a 3-membered saturated carbocyclic ring as part of the moiety, and wherein there are no more than three carbon atom spacers between the connection to T and the connection to R3, or wherein R1 is Cycle P or Cycle Q.

11. The compound of any one of claims 1 to 8 and 10 or a pharmaceutically acceptable salt thereof, wherein R3 is hydrogen, —CN, —OH, C1-C4haloalkyl, —O—C1-C4alkylene-R4, —O—C1-C4haloalkyl, —NH₂, —NH(C1-C4alkylene-R4), —N(C1-C4alkylene-R4)₂, —C(═O)—OH, —C(═O)—NH₂, Cycle P or Cycle Q.

12. The compound of any one of claims 1 to 11 or a pharmaceutically acceptable salt thereof, wherein T is —N(R11)-, —N(R11)-C(═O)— or —N(R11)-S(O₂)—;
R1, when not forming a ring with R11, is —Y—R3;
or wherein R1 and R11 may together form a saturated or partially unsaturated 4- to 7-membered heterocyclic ring optionally containing one or two additional heteroatoms as ring members selected from N, O and S, wherein the heterocyclic ring is optionally substituted by one to three R1a, or R1 and R11 may together form a 5- to 6-membered heteroaryl ring optionally containing one to three additional heteroatoms as ring members selected from N and O, wherein the heteroaryl ring is optionally substituted by one to three R1b; and
R11 is C1-C4alkylene-R11a, C1-C4haloalkylene-R11a or C3-C6cycloalkyl, wherein the cycloalkyl is optionally substituted by one or two R11b.

13. The compound of any one of claims 1 to 11 or a pharmaceutically acceptable salt thereof, wherein T is —N(R11)-, —N(R11)-C(═O)— or —N(R11)-S(O₂)—;
R1, when not forming a ring with R11, is —Y—R3;
Y is C1-C6alkylene wherein one non-terminal —CH₂— moiety may be replaced by —O— and wherein the alkylene moiety may be substituted by one or two moieties independently selected from —OH, —O—C1-C4alkyl and —O—C1-C4haloalkyl and wherein the alkylene moiety may include a 3- to 5-membered saturated or partially unsaturated carbocyclic ring as part of the moiety;
or wherein R1 and R11 may together form a saturated or partially unsaturated 4- to 7-membered heterocyclic ring optionally containing one or two additional heteroatoms as ring members selected from N, O and S, wherein the heterocyclic ring is optionally substituted by one to three R1a, or R1 and R11 or may together form a 5- to 6-membered heteroaryl ring optionally containing one to three additional heteroatoms as ring members selected from N and O, wherein the heteroaryl ring is optionally substituted by one to three R1b; and
R11 is C1-C4alkylene-R11a, C1-C4haloalkylene-R11a or C3-C6cycloalkyl, wherein the cycloalkyl is optionally substituted by one or two R11b.

14. The compound of any one of claims 1 to 11 or a pharmaceutically acceptable salt thereof, wherein
T is —N(R11)- or —N(R11)-S(O₂)—;
R11, when not forming a ring with R1, is C1-C4alkylene-R11a, C1-C4haloalkylene-R11a or C3-C4cycloalkyl;
R11a is hydrogen, halogen, —CN or —OH;
R1 and R11 may together form a 5- to 6-membered saturated heterocyclic ring containing no additional heteroatoms as ring members, wherein the heterocyclic ring is optionally substituted by one R1a, or R1 and R11 may together form a 5-membered heteroaryl ring containing no additional heteroatoms as ring members, and wherein the heteroaryl ring is optionally substituted by one R1b;
R1a is halogen, —OH or —CN;
R1b is halogen, —OH or —CN;
R1, when not forming a ring with R11, is C1-C3alkylene-R3, Cycle P or Cycle Q;
R3 is hydrogen, —CN, —OH, C1-C4haloalkyl, —O—C1-C4alkylene-R4, —O—C1-C4haloalkyl, —NH₂, —NH(C1-C4alkylene-R4) or —N(C1-C4alkylene-R4)₂, —C(═O)—OH, —C(═O)—NH₂, Cycle P or Cycle Q;
Cycle P is oxazolidinyl optionally substituted by one or two R5;
each R5 is independently C1-C2alkyl,-C1-C2alkyl-R5a or oxo, wherein Cycle P is not substituted by two oxo;
Cycle Q is phenyl optionally substituted by one R6, or is a 5- to 6-membered heteroaryl ring containing one to two heteroatoms selected from N and O optionally substituted by one R6;
each R6 is independently —NH₂, —OH, —CN, C1-C2alkylene-R6a or —O—C1-C2alkyl; and
each R6a is independently hydrogen, halogen or —CN.

15. The compound of any one of claims 1 to 14 or a pharmaceutically acceptable salt thereof, wherein R2, when not forming a ring with R10a, R10b, R11, R12 or R14, is C1-C4alkyl or is a 4- or 7-membered saturated carbocyclic ring optionally substituted by one R8, or is a 4- or 7-membered saturated heterocyclic ring containing one —N(R9)- moiety as a ring member and otherwise containing only carbon atoms as ring members, or wherein X—R2 is halogen or hydrogen.

16. The compound of any one of claims 1 to 14 or a pharmaceutically acceptable salt thereof, wherein R2 is C1-C4alkyl or is a 6-membered saturated carbocyclic ring substituted by one R8 at the 3 (“para”) position with respect to X and R8 is —N(R8a)R8b, or wherein X—R2 is bromine.

17. The compound of claim 1 or a pharmaceutically acceptable salt thereof, wherein Ra and Rb are both —CH₃ or together form a —CH₂—CH₂—CH₂—CH₂— bridging moiety;
A is —CH₂— or —C(═O)—;
T is —N(R11)-, —N(R11)-S(O₂)—, —O—, —C(R12)(R13)-, —C═C(R12)₂, —S—, —S(O₂)—, —S(O₂)—N(R14)- or —C(═O)—N(R14)-;
R11, when not forming a ring with R1 or R2, is hydrogen, C1-C4alkylene-R11a, C1-C4haloalkylene-R11a or C3-C6cycloalkyl;
R11a is hydrogen, halogen, —CN or —OH;
each R12 is independently hydrogen or C1-C4alkylene-R12a;
each R12a is independently hydrogen, halogen, —CN or —OH;
R13 is hydrogen;
R14 is hydrogen or C1-C4alkyl;
R2 and R11 may together form a partially unsaturated 6-membered heterocyclic ring containing no additional heteroatoms when T is —N(R11)- and X is —O—;
R1 and R11 may together form a 4- to 6-membered saturated or partially unsaturated heterocyclic ring optionally containing one additional heteroatom as a ring member selected from N and O, wherein the heterocyclic ring is optionally substituted by one or two R1a, or R1 and R11 may together form a 5-membered heteroaryl ring optionally containing one additional heteroatom as a ring member selected from N, and wherein the heteroaryl ring is optionally substituted by one or two R1b;
each R1a is independently halogen, —OH, —CN, C1-C4alkylene-Ric, —O—C1-C4haloalkyl, —C(═O)NH₂ or oxo;
each R1b is independently halogen, —OH, —CN, C1-C4alkylene-R1c or —O—C1-C4haloalkyl;
each R1c is independently hydrogen or —OH;
R1, when not forming a ring with R11, is —Y—R3;
Y is a bond or C1-C6alkylene wherein the alkylene moiety may be substituted by one —OH, wherein the alkylene moiety may include a 3-membered saturated carbocyclic ring as part of the moiety and wherein there are no more than three carbon atom spacers between the connection to T and the connection to R3;
R3 is hydrogen, —CN, —OH, C1-C4haloalkyl, —O—C1-C4alkylene-R4, —O—C1-C4haloalkyl, —NH₂, —NH(C1-C4alkylene-R4), —N(C1-C4alkylene-R4)₂, —C(═O)—NH₂, —C(═O)—NH(C1-C4alkylene-R4), —C(═O)—OH, —C(═O)—O—C1-C4alkylene-R4, —C(═O)—N(C1-C4alkylene-R4)₂, —NH—C(═O)—C1-C4alkylene-R4, —N(C1-C4alkyl)-C(═O)—C1-C4alkylene-R4, Cycle P, Cycle Q, —NH—S(O₂)—C1-C4alkylene-R4, —N(C1-C4alkyl)-S(O₂)—C1-C4alkylene-R4, —S—C1-C4alkylene-R4 or —S(O₂)—C1-C4alkyl-R4;
R4 is hydrogen;
Cycle P is a 3- to 6-membered saturated carbocyclic ring optionally substituted by one to two R5 or is a 5- or 6-membered saturated heterocyclic ring containing one heteroatom selected from N and O optionally substituted by one to two R5;
each R5 is independently —NH₂, C1-C2alkyl,-C1-C2alkyl-R5a or oxo;
each R5a is independently hydrogen, halogen, —NH₂, —OH, —CN or —O—C1-C2alkyl;
Cycle Q is phenyl optionally substituted by one or two R6, or is a 5- to 6-membered heteroaryl ring containing one to two heteroatoms selected from N, S and O optionally substituted by one or two R6;
each R6 is independently —NH₂, —OH, —CN, C1-C2alkylene-R6a or —O—C1-C2alkyl;
each R6a is independently hydrogen, halogen or —CN;
X is —O— or —N(R10a)- or X—R2 is halogen or hydrogen;
R10a is hydrogen or —CH₃ or R2 and R10a may together form a 4- to 7-membered saturated heterocyclic ring optionally containing an —N(R9)- moiety as ring member in addition to the nitrogen atom of X and otherwise containing only carbon atoms as ring members and wherein when not containing an —N(R9)-moiety the heterocyclic ring is optionally substituted by one R8;
R2, when not forming a ring with R10a or R11, is C1-C4alkyl optionally substituted by R7 or is a 4- or 7-membered saturated carbocyclic ring optionally substituted by one R8, or is a 6- or 7-membered saturated heterocyclic ring containing one —N(R9)- moiety as a ring member and otherwise containing only carbon atoms as ring members, or X—R2 is halogen or hydrogen;
R7 is —N(R7a)R7b;
R7a is hydrogen or C1-C4alkyl;
R7b is hydrogen or C1-C4alkyl;
R8 is —N(R8a)R8b;
R8a, when not forming a ring with R8b, is hydrogen or C1-C4alkyl;
R8b when not forming a ring with R8a, is hydrogen or C1-C4alkyl;
R8a and R8b may together form a 6-membered saturated heterocyclic ring optionally containing one or two additional heteroatoms as ring members selected from N and O; and
R9 is hydrogen or C1-C4alkyl.

18. The compound of claim 1 or a pharmaceutically acceptable salt thereof, wherein Ra and Rb are both —CH₃;
A is —CH₂—;
T is —N(R11)-, —N(R11)-S(O₂)—, —C═C(R12)₂, —S— or —C(═O)—N(R14)-;
R11, when not forming a ring with R1, is hydrogen, C1-C4alkylene-R11a, C1-C4haloalkylene-R11a or C3-C4cycloalkyl;
R11a is hydrogen, halogen, —CN or —OH;
each R12 is independently hydrogen or C1-C4alkylene-R12a;
each R12a is independently hydrogen, halogen, —CN or —OH;
R14 is hydrogen or C1-C4alkyl;
R1 and R11 may together form a 5- to 6-membered saturated heterocyclic ring containing no additional heteroatoms as ring members, wherein the heterocyclic ring is optionally substituted by one R1a, or R1 and R11 may together form a 5-membered heteroaryl ring containing no additional heteroatoms as ring members, and wherein the heteroaryl ring is optionally substituted by one R1b;
R1a is halogen, —OH or —CN;
R1b is halogen, —OH or —CN;
R1, when not forming a ring with R11, is hydrogen, C1-C3alkylene-R3, Cycle P or Cycle Q;
R3 is hydrogen, —CN, —OH, C1-C4haloalkyl, —O—C1-C4alkylene-R4, —O—C1-C4haloalkyl, —NH₂, —NH(C1-C4alkylene-R4) or —N(C1-C4alkylene-R4)₂, —C(═O)—OH, —C(═O)—NH₂, Cycle P or Cycle Q;
R4 is hydrogen;
Cycle P is oxazolidinyl optionally substituted by one or two R5;
each R5 is independently C1-C2alkyl,-C1-C2alkyl-R5a or oxo, wherein Cycle P is not substituted by two oxo;
Cycle Q is phenyl optionally substituted by one R6, or is a 5- to 6-membered heteroaryl ring containing one to two heteroatoms selected from N and O optionally substituted by one R6;
R6 is independently —NH₂, —OH, —CN, C1-C2alkylene-R6a or —O—C1-C2alkyl;
R6a is independently hydrogen, halogen or —CN;
X is —O— or —N(R10a)- or X—R2 is halogen;
R10a is hydrogen or —CH₃ or R2 and R10a may together form a 6- to 7-membered saturated heterocyclic ring containing one —N(R9)- moiety as a ring member at the 3 position with respect to the nitrogen atom of X and otherwise containing only carbon atoms as ring members and wherein when not containing an —N(R9)— moiety the heterocyclic ring is substituted by one R8 at the 3 position with respect to the nitrogen atom of X;
R2, when not forming a ring with R10a, is C1-C4alkyl optionally substituted by R7, or is a 6-to 7-membered saturated carbocyclic ring substituted by one R8 at the 3 position with respect to X or R2 is a 6- to 7-membered saturated heterocyclic ring containing one —N(R9)- moiety as a ring member at the 3 position with respect to X and otherwise containing only carbon atoms as ring members, or wherein X—R2 is halogen or hydrogen;
R7 is —N(R7a)R7b;
R7a is hydrogen or C1-C4alkyl-R7c;
R7b is hydrogen or C1-C4alkyl;
R7c is hydrogen, halogen, —O—C1-C2alkyl or —O—C1-C2haloalkyl;
R8 is —N(R8a)R8b;
R8a, when not forming a ring with R8b, is hydrogen or C1-C4alkyl-R8c;
R8b, when not forming a ring with R8a, is hydrogen or C1-C4alkyl;
R8a and R8b may together form a 6-membered saturated heterocyclic ring optionally containing one or two additional heteroatoms as ring members selected from N and O;
R8c is hydrogen, halogen, —O—C1-C2alkyl or —O—C1-C2haloalkyl; and
R9 is hydrogen or C1-C4alkyl.

19. The compound of claim 1 or a pharmaceutically acceptable salt thereof, wherein the compound is selected from one of the following compounds:
8-methoxy-5,5,7-trimethyl-6H-benzo[h]quinazolin-4-amine;
8-methoxy-5,5-dimethyl-6H-benzo[h]quinazoline-4,7-diamine;
6,6-dimethyl-2,3,4,5-tetrahydroquinazolino[7,8-f][1,4]benzoxazin-7-amine;
4,6,6-trimethyl-3,5-dihydro-2H-quinazolino[7,8-f][1,4]benzoxazin-7-amine;
8-(trans-4-aminocyclohexoxy)-5,5-dimethyl-6H-benzo[h]quinazoline-4,7-diamine;
N-[4-amino-8-(trans-4-aminocyclohexoxy)-5,5-dimethyl-6H-benzo[h]quinazolin-7-yl]acetamide;
8-(trans-4-aminocyclohexoxy)-N7,N7-diethyl-5,5-dimethyl-6H-benzo[h]quinazoline-4,7-diamine;
8-(trans-4-aminocyclohexoxy)-N7-ethyl-5,5-dimethyl-6H-benzo[h]quinazoline-4,7-diamine;
8-(trans-4-aminocyclohexoxy)-5,5-dimethyl-7-(1-piperidyl)-6H-benzo[h]quinazolin-4-amine;
8-(trans-4-aminocyclohexoxy)-5,5-dimethyl-7-morpholino-6H-benzo[h]quinazolin-4-amine;
2-[[4-amino-8-(trans-4-aminocyclohexoxy)-5,5-dimethyl-6H-benzo[h]quinazolin-7-yl]-(2-hydroxyethyl)amino]ethanol;
2-[[4-amino-8-(trans-4-aminocyclohexoxy)-5,5-dimethyl-6H-benzo[h]quinazolin-7-yl]-methyl-amino]ethanol;
8-(trans-4-aminocyclohexoxy)-N7-(2-methoxyethyl)-N7,5,5-trimethyl-6H-benzo[h]quinazoline-4,7-diamine;
2-[[4-amino-8-(trans-4-aminocyclohexoxy)-5,5-dimethyl-6H-benzo[h]quinazolin-7-yl]amino]ethanol;
1-[4-amino-8-(trans-4-aminocyclohexoxy)-5,5-dimethyl-6H-benzo[h]quinazolin-7-yl]pyrrolidin-3-ol—isomer 1;
1-[4-amino-8-(trans-4-aminocyclohexoxy)-5,5-dimethyl-6H-benzo[h]quinazolin-7-yl]pyrrolidin-3-ol—isomer 2;
8-(trans-4-aminocyclohexoxy)-5,5-dimethyl-7-pyrrol-1-yl-6H-benzo[h]quinazolin-4-amine;
8-(trans-4-aminocyclohexoxy)-N7-(2-methoxyethyl)-5,5-dimethyl-6H-benzo[h]quinazoline-4,7-diamine;
8-(trans-4-aminocyclohexoxy)-5,5-dimethyl-7-propoxy-6H-benzo[h]quinazolin-4-amine;
8-(trans-4-aminocyclohexoxy)-N7,N7,5,5-tetramethyl-6H-benzo[h]quinazoline-4,7-diamine;
1-(4-amino-8-methoxy-5,5-dimethyl-6H-benzo[h]quinazolin-9-yl)piperidin-4-ol;
2-[[4-amino-8-(cis-4-aminocyclohexoxy)-5,5-dimethyl-6H-benzo[h]quinazolin-7-yl]-ethyl-amino]ethanol;
2-[[4-amino-8-(cis-4-aminocyclohexoxy)-5,5-dimethyl-6H-benzo[h]quinazolin-7-yl]-propyl-amino]ethanol;
2-[[4-amino-8-(cis-4-aminocyclohexoxy)-5,5-dimethyl-6H-benzo[h]quinazolin-7-yl]-methyl-amino]ethanol;
1-(4-amino-8-methoxy-5,5-dimethyl-6H-benzo[h]quinazolin-7-yl)azetidin-3-ol;
2-[[4-amino-8-(cis-4-aminocyclohexoxy)-5,5-dimethyl-6H-benzo[h]quinazolin-7-yl]-methyl-amino]-N-methyl-acetamide;
3-[(4-amino-8-methoxy-5,5-dimethyl-6H-benzo[h]quinazolin-7-yl)amino]propane-1,2-diol;
[1-(4-amino-8-methoxy-5,5-dimethyl-6H-benzo[h]quinazolin-7-yl)azetidin-3-yl]methanol;
8-(cis-4-aminocyclohexoxy)-N7,5,5-trimethyl-N7-(tetrahydropyran-4-ylmethyl)-6H-benzo[h]quinazoline-4,7-diamine;
2-[[4-amino-8-(cis-4-aminocyclohexoxy)-5,5-dimethyl-6H-benzo[h]quinazolin-7-yl]-methyl-amino]acetonitrile;
8-(cis-4-aminocyclohexoxy)-N7,5,5-trimethyl-N7-(2-methylsulfanylethyl)-6H-benzo[h]quinazoline-4,7-diamine;
8-(cis-4-aminocyclohexoxy)-N7,5,5-trimethyl-N7-(2-methylsulfonylethyl)-6H-benzo[h]quinazoline-4,7-diamine;
2-[[4-amino-8-(trans-4-aminocyclohexoxy)-5,5-dimethyl-6H-benzo[h]quinazolin-7-yl]-methyl-amino]acetonitrile;
4,7-diamino-8-(trans-4-aminocyclohexoxy)-5,5-dimethyl-benzo[h]quinazolin-6-one;
2-[[4-amino-8-(trans-4-aminocyclohexoxy)-5,5-dimethyl-6H-benzo[h]quinazolin-7-yl]amino]acetonitrile;
8-(trans-4-aminocyclohexoxy)-N7-(2-ethoxyethyl)-N7,5,5-trimethyl-6H-benzo[h]quinazoline-4,7-diamine;
8-(trans-4-aminocyclohexoxy)spiro[6H-benzo[h]quinazoline-5,1′-cyclopentane]-4,7-diamine;
N-[2-[[4-amino-8-(trans-4-aminocyclohexoxy)-5,5-dimethyl-6H-benzo[h]quinazolin-7-yl]-methyl-amino]ethyl]acetamide;
N-[2-[[4-amino-8-(trans-4-aminocyclohexoxy)-5,5-dimethyl-6H-benzo[h]quinazolin-7-yl]-methyl-amino]ethyl]methanesulfonamide;
1-[[4-amino-8-(trans-4-aminocyclohexoxy)-5,5-dimethyl-6H-benzo[h]quinazolin-7-yl]-methyl-amino]-2-methyl-propan-2-ol;
N-[4-amino-8-(trans-4-aminocyclohexoxy)-5,5-dimethyl-6H-benzo[h]quinazolin-7-yl]-2-hydroxy-N-methyl-acetamide;
N-[4-amino-8-(trans-4-aminocyclohexoxy)-5,5-dimethyl-6H-benzo[h]quinazolin-7-yl]-2-hydroxy-ethanesulfonamide;
8-(trans-4-aminocyclohexoxy)-N7,5,5-trimethyl-N7-(3,3,3-trifluoropropyl)-6H-benzo[h]quinazoline-4,7-diamine;
N-[4-amino-8-(4-trans-aminocyclohexoxy)-5,5-dimethyl-6H-benzo[h]quinazolin-7-yl]-2-hydroxy-N-methyl-ethanesulfonamide;
N-[4-amino-8-(trans-4-aminocyclohexoxy)-5,5-dimethyl-6H-benzo[h]quinazolin-7-yl]-2-hydroxy-acetamide;
3-[[4-amino-8-(trans-4-aminocyclohexoxy)-5,5-dimethyl-6H-benzo[h]quinazolin-7-yl]-methyl-amino]propanenitrile;
8-(trans-4-aminocyclohexoxy)-N7,5,5-trimethyl-N7-[2-(trifluoromethoxy)ethyl]-6H-benzo[h]quinazoline-4,7-diamine;
1-[[[4-amino-8-(trans-4-aminocyclohexoxy)-5,5-dimethyl-6H-benzo[h]quinazolin-7-yl]-methyl-amino]methyl]cyclopropanecarbonitrile;
1-[4-amino-8-(trans-4-aminocyclohexoxy)-5,5-dimethyl-6H-benzo[h]quinazolin-7-yl]pyrrolidine-3-carbonitrile—isomer 1;
1-[4-amino-8-(trans-4-aminocyclohexoxy)-5,5-dimethyl-6H-benzo[h]quinazolin-7-yl]pyrrolidine-3-carbonitrile—isomer 2;
3-[[4-amino-8-(trans-4-aminocyclohexoxy)-5,5-dimethyl-6H-benzo[h]quinazolin-7-yl]-cyclopropyl-amino]propanenitrile;
3-[[4-amino-5,5-dimethyl-8-(trans-4-morpholinocyclohexoxy)-6H-benzo[h]quinazolin-7-yl]-methyl-amino]propanenitrile;
3-[[4-amino-8-[trans-4-(2-methoxyethylamino)cyclohexoxy]-5,5-dimethyl-6H-benzo[h]quinazolin-7-yl]-methyl-amino]propanenitrile;
8-(trans-4-aminocyclohexoxy)-N7-but-3-ynyl-N7,5,5-trimethyl-6H-benzo[h]quinazoline-4,7-diamine;
1-(4-amino-8-methoxy-5,5-dimethyl-6H-benzo[h]quinazolin-7-yl)pyrrolidine-3-carbonitrile;
1-[4-amino-8-(trans-4-aminocyclohexoxy)-5,5-dimethyl-6H-benzo[h]quinazolin-7-yl]-2H-pyrrol-5-one;
8-(trans-4-aminocyclohexoxy)-7-(3-methoxypyrrol-1-yl)-5,5-dimethyl-6H-benzo[h]quinazolin-4-amine;
4-[[4-amino-8-(4-trans-aminocyclohexoxy)-5,5-dimethyl-6H-benzo[h]quinazolin-7-yl]-methyl-amino]butanenitrile;
2-[[4-amino-8-(4-trans-aminocyclohexoxy)spiro[6H-benzo[h]quinazoline-5,1′-cyclopentane]-7-yl]-methyl-amino]ethanol;
2-[[4-amino-8-(4-trans-aminocyclohexoxy)spiro[6H-benzo[h]quinazoline-5,1′-cyclopentane]-7-yl]amino]ethanol;
8-methoxy-7-(3-methoxypropyl)-5,5-dimethyl-6H-benzo[h]quinazolin-4-amine;
8-(trans-4-aminocyclohexoxy)-N7-(2-methoxyethyl)spiro[6H-benzo[h]quinazoline-5,1′-cyclopentane]-4,7-diamine;
8-(trans-4-aminocyclohexoxy)-7-(3-methoxypyrrolidin-1-yl)-5,5-dimethyl-6H-benzo[h]quinazolin-4-amine—isomer 1;
N-[4-amino-8-(trans-4-aminocyclohexoxy)-5,5-dimethyl-6H-benzo[h]quinazolin-7-yl]-2-methoxy-N-methyl-ethanesulfonamide;
ethyl 2-[[4-amino-8-(cis-4-aminocyclohexoxy)spiro[6H-benzo[h]quinazoline-5,1′-cyclopentane]-7-yl]amino]acetate;
1-[4-amino-8-(trans-4-aminocyclohexoxy)-5,5-dimethyl-6H-benzo[h]quinazolin-7-yl]pyrrolidine-3-carbonitrile;
8-(trans-4-aminocyclohexoxy)-N7-ethyl-N7,5,5-trimethyl-6H-benzo[h]quinazoline-4,7-diamine;
8-(trans-4-aminocyclohexoxy)-N7,5,5-trimethyl-6H-benzo[h]quinazoline-4,7-diamine;
3-[[4-amino-8-(trans-4-aminocyclohexoxy)-5,5-dimethyl-6H-benzo[h]quinazolin-7-yl]-methyl-sulfamoyl]propanoic acid;
2-[[4-amino-8-(cis-4-aminocyclohexoxy)spiro[6H-benzo[h]quinazoline-5,1′-cyclopentane]-7-yl]amino]ethanol;
3-[[4-amino-8-(trans-4-aminocyclohexoxy)-5,5-dimethyl-6H-benzo[h]quinazolin-7-yl]oxy]propanenitrile;
8-(trans-4-aminocyclohexoxy)-N7-(2-methoxyethyl)-N7-methyl-spiro[6H-benzo[h]quinazoline-5,1′-cyclopentane]-4,7-diamine;
8-(trans-4-aminocyclohexoxy)-7-(3-methoxypyrrolidin-1-yl)-5,5-dimethyl-6H-benzo[h]quinazolin-4-amine—isomer 2;
N-[4-amino-8-(trans-4-aminocyclohexoxy)-5,5-dimethyl-6H-benzo[h]quinazolin-7-yl]-2-cyano-N-methyl-ethanesulfonamide;
8-(cis-4-aminocyclohexoxy)-N7-(2-methoxyethyl)-N7-methyl-spiro[6H-benzo[h]quinazoline-5,1′-cyclopentane]-4,7-diamine;
1-[[[4-amino-8-(trans-4-aminocyclohexoxy)-5,5-dimethyl-6H-benzo[h]quinazolin-7-yl]amino]methyl]cyclopropanecarbonitrile;
1-[4-amino-8-(trans-4-aminocyclohexoxy)-5,5-dimethyl-6H-benzo[h]quinazolin-7-yl]pyrrolidine-3-carboxamide;
3-[[4-amino-8-(azepan-4-yloxy)-5,5-dimethyl-6H-benzo[h]quinazolin-7-yl]-methyl-amino]propanenitrile;
2-[[4-amino-8-(cis-4-aminocyclohexoxy)spiro[6H-benzo[h]quinazoline-5,1′-cyclopentane]-7-yl]amino]acetic acid;
8-(trans-4-aminocyclohexoxy)-N7-(2-aminoethyl)-N7,5,5-trimethyl-6H-benzo[h]quinazoline-4,7-diamine;
4-[4-amino-8-(trans-4-aminocyclohexoxy)-5,5-dimethyl-6H-benzo[h]quinazolin-7-yl]butanenitrile;
(5R)-5-[[(4-amino-8-methoxy-5,5-dimethyl-6H-benzo[h]quinazolin-7-yl)amino]methyl]oxazolidin-2-one;
2-[[4-amino-8-(cis-4-aminocyclohexoxy)spiro[6H-benzo[h]quinazoline-5,1′-cyclopentane]-7-yl]-methyl-amino]ethanol;
8-(trans-4-aminocyclohexoxy)-N7-cyclopropyl-5,5-dimethyl-6H-benzo[h]quinazoline-4,7-diamine;
3-[[4-amino-8-(trans-4-aminocyclohexoxy)-5,5-dimethyl-6H-benzo[h]quinazolin-7-yl]-methyl-amino]propanoic acid;
3-[[4-amino-8-(trans-4-aminocyclohexoxy)-5,5-dimethyl-6H-benzo[h]quinazolin-7-yl]-methyl-sulfamoyl]propanamide;
8-(cis-4-aminocyclohexoxy)-N7-(2-methoxyethyl)spiro[6H-benzo[h]quinazoline-5,1′-cyclopentane]-4,7-diamine;
3-[[4-amino-8-(trans-4-aminocyclohexoxy)-5,5-dimethyl-6H-benzo[h]quinazolin-7-yl]-methyl-amino]-2,2-dimethyl-propanamide;
(5R)-5-[2-[(4-amino-8-methoxy-5,5-dimethyl-6H-benzo[h]quinazolin-7-yl)amino]ethyl]oxazolidin-2-one;
4-amino-8-(trans-4-aminocyclohexoxy)-N-(cyanomethyl)-5,5-dimethyl-6H-benzo[h]quinazoline-7-carboxamide;
3-[[4-amino-8-(trans-4-aminocyclohexoxy)-5,5-dimethyl-6H-benzo[h]quinazolin-7-yl]-methyl-amino]-2,2-dimethyl-propanenitrile;
3-[4-amino-8-(trans-4-aminocyclohexoxy)-5,5-dimethyl-6H-benzo[h]quinazolin-7-yl]butan-1-ol;
3-[[4-amino-5,5-dimethyl-8-(4-piperidyloxy)-6H-benzo[h]quinazolin-7-yl]-methyl-amino]propanenitrile;
4-amino-8-(trans-4-aminocyclohexoxy)-N-(cyanomethyl)-N,5,5-trimethyl-6H-benzo[h]quinazoline-7-carboxamide;
3-[(4-amino-8-methoxy-5,5-dimethyl-6H-benzo[h]quinazolin-7-yl)-methyl-amino]propanenitrile;
3-[[4-amino-8-trans-4-(dimethylamino)cyclohexoxy]-5,5-dimethyl-6H-benzo[h]quinazolin-7-yl]-methyl-amino]propanenitrile;
4-[4-amino-8-(trans-4-aminocyclohexoxy)-5,5-dimethyl-6H-benzo[h]quinazolin-7-yl]pentanenitrile;
(E)-3-[4-amino-8-(trans-4-aminocyclohexoxy)-5,5-dimethyl-6H-benzo[h]quinazolin-7-yl]but-2-en-1-ol;
3-[[4-amino-5,5-dimethyl-8-[trans-4-(methylamino)cyclohexoxy]-6H-benzo[h]quinazolin-7-yl]-methyl-amino]propanenitrile;
8-(trans-4-aminocyclohexoxy)-N7-butyl-N7,5,5-trimethyl-6H-benzo[h]quinazoline-4,7-diamine;
8-(trans-4-aminocyclohexoxy)-N7,5,5-trimethyl-N7-(4-pyridylmethyl)-6H-benzo[h]quinazoline-4,7-diamine;
8-(trans-4-aminocyclohexoxy)-N7-isopropyl-N7,5,5-trimethyl-6H-benzo[h]quinazoline-4,7-diamine;
8-(trans-4-aminocyclohexoxy)-N7-(1H-imidazol-4-ylmethyl)-N7,5,5-trimethyl-6H-benzo[h]quinazoline-4,7-diamine;
8-(trans-4-aminocyclohexoxy)-N7-isopentyl-N7,5,5-trimethyl-6H-benzo[h]quinazoline-4,7-diamine;
8-(trans-4-aminocyclohexoxy)-N7,5,5-trimethyl-N7-(oxazol-4-ylmethyl)-6H-benzo[h]quinazoline-4,7-diamine;
8-(trans-4-aminocyclohexoxy)-N7-isobutyl-N7,5,5-trimethyl-6H-benzo[h]quinazoline-4,7-diamine;
8-(trans-4-aminocyclohexoxy)-N7,5,5-trimethyl-N7-(3-pyridylmethyl)-6H-benzo[h]quinazoline-4,7-diamine;
3-[[4-amino-8-(trans-4-aminocyclohexoxy)-5,5-dimethyl-6H-benzo[h]quinazolin-7-yl]sulfanyl]propanenitrile;
3-[(4-amino-5,5-dimethyl-6H-benzo[h]quinazolin-7-yl)-methyl-amino]propanenitrile;
3-[[4-amino-8-(trans-4-aminocyclohexoxy)-5,5-dimethyl-6H-benzo[h]quinazolin-7-yl]-methyl-amino]-2,2-dimethyl-propan-1-ol;
4-[[4-amino-8-(trans-4-aminocyclohexoxy)-5,5-dimethyl-6H-benzo[h]quinazolin-7-yl]-methyl-amino]-2-methyl-butan-2-ol;
3-[[4-amino-8-(trans-4-aminocyclohexoxy)-5,5-dimethyl-6H-benzo[h]quinazolin-7-yl]sulfonyl]propanenitrile;
4-amino-8-(trans-4-aminocyclohexoxy)-N-(cyanomethyl)-N,5,5-trimethyl-6H-benzo[h]quinazoline-7-sulfonamide;
N-[4-amino-8-(trans-4-aminocyclohexoxy)-5,5-dimethyl-6H-benzo[h]quinazolin-7-yl]-4-fluoro-N-methyl-benzenesulfonamide;
3-[(4-amino-8-bromo-5,5-dimethyl-6H-benzo[h]quinazolin-7-yl)-methyl-amino]propanenitrile;
4-amino-8-(trans-4-aminocyclohexoxy)-N-(cyanomethyl)-5,5-dimethyl-6H-benzo[h]quinazoline-7-sulfonamide;
3-(7-amino-6,6-dimethyl-3,5-dihydro-2H-quinazolino[7,8-f][1,4]benzoxazin-4-yl)propanenitrile;
3-[[4-amino-5,5-dimethyl-8-(4-methylpiperazin-1-yl)-6H-benzo[h]quinazolin-7-yl]-methyl-amino]propanenitrile;
8-(trans-4-aminocyclohexoxy)-7-(1,1-dioxo-1,2-thiazolidin-2-yl)-5,5-dimethyl-6H-benzo[h]quinazolin-4-amine;
1-(4-amino-8-bromo-5,5-dimethyl-6H-benzo[h]quinazolin-7-yl)pyrrolidine-3-carbonitrile;
(5R)-5-[[(4-amino-8-bromo-5,5-dimethyl-6H-benzo[h]quinazolin-7-yl)amino]methyl]oxazolidin-2-one;
N7-[[5-(1-fluoroethyl)-1,3,4-oxadiazol-2-yl]methyl]-8-methoxy-5,5-dimethyl-6H-benzo[h]quinazoline-4,7-diamine;
3-[[4-amino-8-[2-(dimethylamino)ethoxy]-5,5-dimethyl-6H-benzo[h]quinazolin-7-yl]-methyl-amino]propanenitrile;
3-[(4-amino-8-methoxy-5,5-dimethyl-6H-benzo[h]quinazolin-7-yl)-[[5-(1-fluoroethyl)-1,3,4-oxadiazol-2-yl]methyl]amino]propanenitrile;
3-[(4-amino-5,5-dimethyl-8-piperazin-1-yl-6H-benzo[h]quinazolin-7-yl)-methyl-amino]propanenitrile;
(5S)-5-[[(4-amino-8-bromo-5,5-dimethyl-6H-benzo[h]quinazolin-7-yl)-methyl-amino]methyl]oxazolidin-2-one;
(5R)-5-[[(4-amino-8-bromo-5,5-dimethyl-6H-benzo[h]quinazolin-7-yl)amino]methyl]-3-(2-hydroxyethyl)oxazolidin-2-one;
(5R)-5-[[[4-amino-8-(cis-4-aminocyclohexoxy)-5,5-dimethyl-6H-benzo[h]quinazolin-7-yl]amino]methyl]oxazolidin-2-one;
(5R)-5-[[[4-amino-8-(trans-4-aminocyclohexoxy)-5,5-dimethyl-6H-benzo[h]quinazolin-7-yl]amino]methyl]oxazolidin-2-one;
(5R)-5-[[[4-amino-5,5-dimethyl-8-(cis-4-morpholinocyclohexoxy)-6H-benzo[h]quinazolin-7-yl]amino]methyl]oxazolidin-2-one;
(5R)-5-[[[4-amino-5,5-dimethyl-8-(trans-4-morpholinocyclohexoxy)-6H-benzo[h]quinazolin-7-yl]amino]methyl]oxazolidin-2-one; and
3-[(4-amino-8-bromo-5,5-dimethyl-6H-benzo[h]quinazolin-7-yl)-[[(5S)-2-oxooxazolidin-5-yl]methyl]amino]propanenitrile.

20. The compound of any one of claims 1 to 19 or a pharmaceutically acceptable salt thereof, wherein the compound inhibits CLK3 in an in vitro assay with an IC50 of at most 100 nM.

21. A compound of formula (I) as defined in any one of claims 1 to 20 or a pharmaceutically acceptable salt thereof, for use in the treatment of a neoplastic disease, e.g. cancer, in a subject selected from a mammal, in particular a human.

22. Use of a compound of formula (I) as defined in any one of claims 1 to 20 or a pharmaceutically acceptable salt thereof in the manufacture of a medicament for the treatment of a neoplastic disease, e.g. cancer, in a subject selected from a mammal, in particular a human.

23. A method of treating a neoplastic disease, e.g. cancer, in a subject selected from a mammal, in particular a human, comprising administering a compound of formula (I) as defined in any one of claims 1 to 20 or a pharmaceutically acceptable salt thereof, in a therapeutically effective amount, to said subject.

24. A pharmaceutical composition comprising a compound of formula (I) as defined in any one of claims 1 to 20 or a pharmaceutically acceptable salt thereof and optionally a pharmaceutically acceptable excipient.

25. A compound of formula (B) 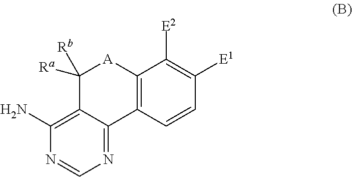
or a salt thereof, wherein
A, Ra and Rb are as defined for the compound of formula (I) in any one of claims 1 to 20;
E1 is —OH, -halogen, —O—C1-C4alkyl, boronic acid, a boronic ester or O-L1;
L1 is methanesulfonyl, p-toluenesulfonyl or trifluoromethanesulfonyl; and
E2 is —OH, halogen, —NO₂ or —SO₂Cl; or
a compound of formula (C) 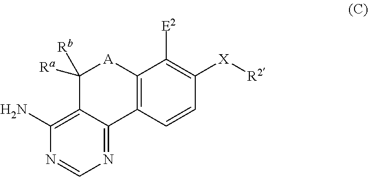
or a salt thereof, wherein
A, X, Ra and Rb are as defined for the compound of formula (I) in any one of claims 1 to 20;
E2 is —OH, halogen, —NO₂ or —SO₂C1; and
R2′ is as defined for R2 for the compound of formula (I) in any one of claims 1 to 20, wherein any functional group within R2 is optionally protected with a suitable protecting group; or
a compound of formula (D) 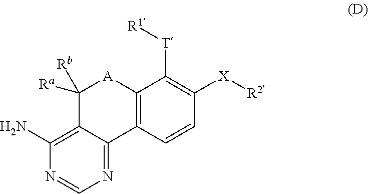
or a salt thereof, wherein
A, Ra and Rb are as defined for the compound of formula (I) in any one of claims 1 to 20;
T′ is —N(R11′)—, —N(R11′)—C(═O)—, —N(R11′)—S(O₂)—, —O—, —C(R12′)(R13)-, —C═C(R12′)₂, —S—, —S(O)—, —S(O₂)—, —S(O₂)—N(R14′)— or —C(═O)—N(R14′)—;
R11′ is as defined for R11 in the compound of formula (I) in any one of claims 1 to 20, wherein any functional group within R11 is optionally protected with a suitable protecting group;
R12′ is as defined for R12 in the compound of formula (I) in any one of claims 1 to 20, wherein any functional group within R12 is optionally protected with a suitable protecting group;
R13 is as defined for the compound of formula (I) in any one of claims 1 to 20;
R14′ is as defined for R14 in the compound of formula (I) in any one of claims 1 to 20, wherein any functional group within R14 is optionally protected with a suitable protecting group;
R1′ is as defined for R1 in the compound of formula (I) in any one of claims 1 to 20, wherein any functional group within R1 is optionally protected with a suitable protecting group; and
R2′ is as defined for R2 in the compound of formula (I) in any one of claims 1 to 20, wherein any functional group within R2 is optionally protected with a suitable protecting group; or
a compound of formula (E) 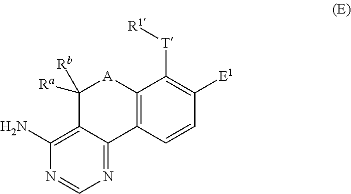
or a salt thereof, wherein
A, Ra and Rb are as defined for the compound of formula (I) in any one of claims 1 to 20;
T′ is —N(R11′)—, —N(R11′)—C(═O)—, —N(R11′)—S(O₂)—, —O—, —C(R12′)(R13)-, —C═C(R12′)₂, —S—, —S(O)—, —S(O₂)—, —S(O₂)—N(R14′)— or —C(═O)—N(R14′)—;
R11′ is as defined for R11 in the compound of formula (I) in any one of claims 1 to 20, wherein any functional group within R11 is optionally protected with a suitable protecting group;
R12′ is as defined for R12 in the compound of formula (I) in any one of claims 1 to 20, wherein any functional group within R12 is optionally protected with a suitable protecting group;
R13 is as defined for the compound of formula (I) in any one of claims 1 to 20;
R14′ is as defined for R14 in the compound of formula (I) in any one of claims 1 to 20, wherein any functional group within R14 is optionally protected with a suitable protecting group;
R1′ is as defined for R1 in the compound of formula (I) in any one of claims 1 to 20, wherein any functional group within R1 is optionally protected with a suitable protecting group; and
E1 is —OH, -halogen, —O—C1-C4alkyl, boronic acid, a boronic ester or O-L1; and
L1 is methanesulfonyl, p-toluenesulfonyl or trifluoromethanesulfonyl.
